(19) United States
(12) Patent Application Publication
(10) Pub. No.: US 2024/0417454 A1
(43) Pub. Date: Dec. 19, 2024

(54) METHODS OF ISOLATING ALLERGEN-SPECIFIC ANTIBODIES FROM HUMANS AND USES THEREOF

(71) Applicants: CZ Biohub SF, LLC, San Francisco, CA (US); The Board of Trustees of the Leland Stanford Junior University, Stanford, CA (US)

(72) Inventors: Derek Croote, San Francisco, CA (US); Stephen R. Quake, Stanford, CA (US); Kari Nadeau, Stanford, CA (US); Spyros Darmanis, San Francisco, CA (US); David N. Cornfield, Stanford, CA (US)

(73) Assignees: CZ Biohub SF, LLC, San Francisco, CA (US); The Board of Trustees of the Leland Stanford Junior University, Stanford, CA (US)

(21) Appl. No.: 18/812,424

(22) Filed: Aug. 22, 2024

Related U.S. Application Data
(63) Continuation of application No. 17/056,384, filed on Nov. 17, 2020, now Pat. No. 12,103,964.
(60) Provisional application No. 62/673,713, filed on May 18, 2018.

(51) Int. Cl. C07K 16/18; A61K 39/395; A61K 49/00; C07K 16/14; C07K 16/16; C12N 5/0781; G01N 33/50; G01N 33/569

(52) U.S. Cl. CPC C07K 16/18; A61K 39/39575; A61K 49/0004; C07K 16/14; C07K 16/16; C12N 5/0635; G01N 33/5052; G01N 33/56961; C07K 2317/21; C07K 2317/33; C07K 2317/34; C07K 2317/52; C07K 2317/565; C07K 2317/92

(57) ABSTRACT

In one aspect, methods of generating human monoclonal antibodies that specifically binds to an allergen are provided. In some embodiments, the monoclonal antibodies are generated from sequences identified from isolated single B cells from a human subject who is allergic to the allergen.

### REFERENCE TO RELATED APPLICATIONS

This application is a continuation of U.S. application Ser. No. 17/056,384, filed Nov. 17, 2020, which is a national stage application of PCT Application No. PCT/US2019/032951, filed May 17, 2019, which claims benefit of U.S. Provisional Patent Application No. 62/673,713, filed May 18, 2018, which applications are incorporated herein by reference in their entirety.

### REFERENCE TO AN ELECTRONIC SEQUENCE LISTING

The contents of the electronic sequence listing (110221-1458081-000710US1.xml; 1,277,006 bytes; and Date of Creation Aug. 21, 2024) is herein incorporated by reference in its entirety.

### BACKGROUND OF THE INVENTION

Allergies are a growing health concern worldwide and are characterized by a misdirected adaptive immune response towards otherwise harmless proteins. For food allergies in particular, individuals must be diligent in avoiding allergen exposure or otherwise risk potentially fatal allergic reactions. No cure for allergies exist, and although desensitization regimens such as immunotherapy have shown some clinical benefit, there is a need for a fast, effective intervention that can improve the quality of life for allergic individuals.

### BRIEF SUMMARY OF THE INVENTION

In one aspect, the present disclosure provides methods of generating a human monoclonal antibody that specifically binds to an allergen. In some embodiments, the method comprises:
(a) isolating single B cells from a sample from a human subject, wherein the subject is allergic to the allergen;
(b) generating cDNAs from the single B cells of step (a), wherein the cDNA sequences comprise a first sequence that encodes all or part of an immunoglobulin heavy chain and a second sequence that encodes all or part of an immunoglobulin light chain;
(c) determining the sequences of the cDNAs from step (b);
(d) analyzing the sequences determined in step (c) to identify single B cells comprising a first sequence that comprises an IgE constant region or an IgG4 constant region;
(e) identifying, from the single B cells of step (d), (i) a heavy chain variable region sequence in the immunoglobulin heavy chain that comprises an IgE constant region or an IgG4 constant region, and (ii) a light chain variable region sequence in an immunoglobulin light chain that is co-expressed with the immunoglobulin heavy chain in the same single B cell;
(f) expressing antibodies comprising the heavy chain variable region sequence and the light chain variable region sequence from step (e); and
(g) identifying one or more antibodies from step (f) that specifically bind to the allergen.

In some embodiments, step (a) comprises sorting cells in the sample by fluorescent activated cell sorting (FACS). In some embodiments, step (a) comprises selecting single B cells for expression of cell surface IgE and/or cell surface IgG4. In some embodiments, step (a) comprises isolating antibody-secreting B cells and/or memory B cells.

In some embodiments, the method comprises isolating single B cells that are selected for expression of cell surface IgE and identifying a sequence encoding an immunoglobulin heavy chain that comprises an IgE constant region. In some embodiments, step (a) comprises contacting cells from the sample with an anti-human CD19 antibody and an anti-human IgE antibody and selecting for CD19⁺ IgE-expressing B cells. In some embodiments, the method comprises isolating single B cells that express a B cell marker and that are negative for non-IgE isotypes. In some embodiments, step (a) comprises contacting cells from the sample with an anti-human CD19 antibody, an anti-human IgM antibody, an anti-human IgG antibody, an anti-human IgA antibody, and an anti-human IgD antibody and selecting for CD19⁺IgM⁻IgG⁻IgA⁻IgD⁻ B cells.

In some embodiments, the method comprises isolating single B cells that are selected for expression of cell surface IgG4 and identifying a sequence encoding an immunoglobulin heavy chain that comprises an IgG4 constant region. In some embodiments, step (a) comprises contacting cells from the sample with an anti-human CD19 antibody and an anti-human IgG4 antibody and selecting for CD19⁺ IgG4-expressing B cells. In some embodiments, the method comprises isolating single B cells that express a B cell marker and that are negative for non-IgG4 isotypes. In some embodiments, step (a) comprises contacting cells from the sample with an anti-human CD19 antibody, an anti-human IgM antibody, an anti-human IgE antibody, an anti-human IgA antibody, an anti-human IgD antibody, an anti-human IgG1 antibody, an anti-human IgG2 antibody, and an anti-human IgG3 antibody and selecting for CD19⁺IgM⁻IgE⁻IgA⁻IgD⁻IgG1⁻IgG2⁻IgG3⁻ B cells.

In some embodiments, step (b) comprises reverse transcribing cDNAs from RNA from the single B cells and amplifying the cDNAs. In some embodiments, the RNA is mRNA. In some embodiments, the method comprises amplifying immunoglobulin heavy chain and light chain sequences from the single B cells. In some embodiments, the method comprises amplifying the transcriptomes of the single B cells.

In some embodiments, step (f) comprises expressing the heavy chain variable region sequence and the light chain variable region sequence from step (e) in a host cell and purifying the antibodies. In some embodiments, step (f) comprises expressing antibodies comprising the heavy chain variable region sequence and the light chain variable region sequence from step (e) and an IgG4 constant region or an IgG1 constant region.

In some embodiments, the method further comprises substituting the constant region of an antibody identified in step (g) with a wild-type IgG4 constant region or a modified IgG4 constant region.

In some embodiments, the sample comprises peripheral blood. In some embodiments, the sample comprises tissue (e.g., tonsil tissue).

In some embodiments, the allergen is a food allergen, a plant allergen, a fungal allergen, an animal allergen, a drug allergen, a cosmetic allergen, or a latex allergen. In some embodiments, the allergen is a food allergen selected from the group consisting of a milk allergen, an egg allergen, a nut allergen, a fish allergen, a shellfish allergen, a soy allergen, a legume allergen, a seed allergen, and a wheat allergen. In some embodiments, the food allergen is a peanut allergen. In some embodiments, the food allergen is a tree nut allergen. In some embodiments, the food allergen is a milk allergen. In some embodiments, the allergen is a fungal allergen. In some embodiments, the fungal allergen is an Aspergillus allergen.

In another aspect, monoclonal antibodies produced according to a method disclosed herein are provided.

In yet another aspect, pharmaceutical compositions comprising a monoclonal antibody produced according to a method disclosed herein are provided. In some embodiments, the pharmaceutical composition comprises a plurality of monoclonal antibodies, wherein each monoclonal antibody is produced according to a method disclosed herein and wherein the monoclonal antibodies recognize different epitopes or specifically bind to different antigens (e.g., different allergens within the same type or class of allergen or in different types or classes of allergens).

In still another aspect, monoclonal antibodies, or antigen-binding portions thereof, that specifically bind to an allergen are provided. In some embodiments, the antibody comprises:
(a) a heavy chain variable region sequence that is derived from an immunoglobulin heavy chain from an IgE- or IgG4-producing single B cell from a human subject who is allergic to the allergen;
(b) a heavy chain IgG constant region sequence;
(c) a light chain variable region sequence that is derived from an immunoglobulin light chain from the IgE-producing or IgG4-producing single B cell from a human subject; and
(d) a light chain constant region sequence that is of the same class as the immunoglobulin light chain of (c).

In some embodiments, the antibody or the antigen-binding portion thereof specifically binds to an allergen with a binding affinity (K_{D}) of less than 1 nM. In some embodiments the antibody or the antigen-binding portion thereof specifically binds to an allergen with a binding affinity (KD) of less than 250 nM, less than 100 nM, less than 50 nM, less than 10 nM, or less than 5 nM. In some embodiments, the antibody binds to the allergen with a binding affinity (KD) from 1 nM to 250 nM. In some embodiments, the allergen is a food allergen, a plant allergen, a fungal allergen, an animal allergen, a dust mite allergen, a drug allergen, a cosmetic allergen, or a latex allergen. In some embodiments, the allergen is a food allergen selected from the group consisting of a milk allergen, an egg allergen, a nut allergen, a fish allergen, a shellfish allergen, a soy allergen, a legume allergen, a seed allergen, and a wheat allergen. In some embodiments, the food allergen is a peanut allergen or a tree nut allergen. In some embodiments, the food allergen is a milk allergen. In some embodiments, the allergen is a fungal allergen. In some embodiments, the fungal allergen is an Aspergillus antigen. In some embodiments, the antibody is cross-reactive with two different antigens. In some embodiments, the antibody is cross-reactive with a first antigen of a first allergen type and a second antigen of a second allergen type that is different from the first allergen type. In some embodiments, the antibody is cross-reactive with a peanut allergen and a tree nut allergen. In some embodiments, the antibody is cross-reactive with two or more tree nut antigens.

In some embodiments, the antibody comprises a heavy chain variable region sequence and a light chain variable region sequence that are derived from an IgE-producing human B cell or from an IgG4-producing human B cell.

In yet another aspect, monoclonal antibodies, or antigen-binding portions thereof, that specifically binds to a peanut allergen are provided. In some embodiments, the antibody binds to the peanut allergen with a binding affinity (K_{D}) of less than 1 nM. In some embodiments the antibody or the antigen-binding portion thereof specifically binds to an peanut allergen with a binding affinity (KD) of less than 250 nM, less than 100 nM, less than 50 nM, less than 10 nM, or less than 5 nM. In some embodiments, the antibody binds to the peanut allergen with a binding affinity (KD) from 1 nM to 250 nM.

In some embodiments, the antibody or the antigen-binding portion thereof specifically binds to the peanut allergen Ara h 2, Ara h 3, or Ara h 1. In some embodiments, the antibody or the antigen-binding portion thereof specifically binds to Ara h 2 with a K_{D} of less than 100 pM. In some embodiments, the antibody or the antigen-binding portion thereof is cross-reactive with at least two peanut allergens. In some embodiments, the antibody or the antigen-binding portion thereof is cross-reactive with Ara h 2 and Ara h 3. In some embodiments, the antibody or the antigen-binding portion thereof specifically binds to Ara h 2 with a K_{D} of less than 1 nM and specifically binds to Ara h 3 with a K_{D} of less than 1 IpM. In some embodiments, the antibody or the antigen-binding portion thereof binds to an epitope that comprises the amino acid motif DPYSPS (SEQ ID NO:704). Furthermore, additional peanut-specific antibodies were discovered during these experiments. Antibodies PA12P3E09 and PA12P3E11 bound peanut extract with little to no binding to natural peanut allergen Ara h 2, while antibodies PA12P1D02, PA12P1G11, PA13P1H03, PA12P3C01, and PA12P3E04 bound strongly to both peanut extract and natural peanut allergen Ara h 2. In some embodiments the peanut specific antibody binds to peanut extract, but does not bind natural peanut allergen Ara h 2.

In some embodiments, the antibody comprises:
a heavy chain CDR1 comprising the amino acid sequence of any one of SEQ ID NOs:2, 10, 17, 25, 33, 41, 47, 58, 113, 129, 199, 341, 348, 409, 459, or 593;
a heavy chain CDR2 comprising the amino acid sequence of any one of SEQ ID NOs:3, 11, 18, 26, 34, 48, 59, 130, 200, 342, 349, 410, 460, 539, or 594;
a heavy chain CDR3 comprising the amino acid sequence of any one of SEQ ID NOs:4, 12, 19, 27, 35, 42, 49, 55, 60, 131, 201, 350, 411, 461, 540, or 595;
a light chain CDR1 comprising the amino acid sequence of any one of SEQ ID NOs:6, 14, 21, 29, 37, 44, 51, 62, 133, 203, 343, 352, 413, 463, 542, or 597;
a light chain CDR2 comprising the amino acid sequence of any one of SEQ ID NOs:7, 15, 22, 30, 38, 52, 78, 86, 126, 149, 196, 345, 353, or 598; and
a light chain CDR3 comprising the amino acid sequence of any one of SEQ ID NOs:8, 23, 31, 39, 45, 53, 63, 134, 204, 346, 354, 414, 464, 543, or 599.

In some embodiments, the antibody comprises heavy chain and light chain CDR sequences contained within the heavy chain variable region and light chain variable region sequence pairs selected from the group consisting of SEQ ID NOs: 1 and 5; 9 and 13; 16 and 20; 24 and 28; 32 and 36; 40 and 43; 46 and 50; 54 and 56; 57 and 61; 57 and 5; 1 and 61; 64 and 5; 65 and 5; 66 and 5; 67 and 5; 128 and 132; 340 and 344; 347 and 351; 406 and 407; 408 and 412; 458 and 462; 538 and 541; and 592 and 596. In some embodiments, the antibody comprises a heavy chain CDR1-3 and a light chain CDR1-3 comprising the amino acid sequences of:
(a) SEQ ID NOs: 2, 3, 4, 6, 7, and 8, respectively; or
(b) SEQ ID NOs: 10, 11, 12, 14, 15, and 8, respectively; or
(c) SEQ ID NOs: 17, 18, 19, 21, 22, and 23, respectively; or
(d) SEQ ID NOs: 25, 26, 27, 29, 30, and 31, respectively; or
(e) SEQ ID NOs: 33, 34, 35, 37, 38, and 39, respectively; or
(f) SEQ ID NOs: 41, 34, 35, 37, 38, and 39, respectively; or
(g) SEQ ID NOs: 47, 48, 49, 51, 52, and 53, respectively; or
(h) SEQ ID NOs: 47, 48, 55, 51, 52, and 53, respectively; or
(i) SEQ ID NOs: 58, 59, 60, 62, 30, and 63, respectively; or
(j) SEQ ID NOs: 58, 59, 60, 6, 7, and 8, respectively; or
(k) SEQ ID NOs: 2, 3, 4, 62, 30, and 63, respectively; or
(l) SEQ ID NOs: 58, 3, 4, 6, 7, and 8, respectively; or
(m) SEQ ID NOs: 2, 59, 4, 6, 7, and 8, respectively; or
(n) SEQ ID NOs: 2, 3, 60, 6, 7, and 8, respectively; or
(o) SEQ ID NOs:129, 130, 131, 133, 126, and 134, respectively; or
(p) SEQ ID NOs:341, 342, 343, 345, 78, and 346, respectively; or
(q) SEQ ID NOs:348, 349, 350, 352, 353, and 354, respectively; or
(r) SEQ ID NOs:199, 200, 201, 203, 149, and 204, respectively; or
(s) SEQ ID NOs:409, 410, 411, 413, 86, and 414, respectively; or
(t) SEQ ID NOs:459, 460, 461, 463, 196, and 464, respectively; or
(u) SEQ ID NOs:113, 539, 540, 542, 196, and 543, respectively; or
(v) SEQ ID NOs:593, 594, 595, 597, 598, and 599, respectively.

In some embodiments, the antibody comprises a heavy chain variable region comprising an amino acid sequence that has at least 90% sequence identity to any one of SEQ ID NOs:1, 9, 16, 24, 32, 40, 46, 54, 57, 64, 65, 66, 67, 128, 340, 347, 406, 408, 458, 538, or 592. In some embodiments, the antibody comprises a light chain variable region comprising an amino acid sequence that has at least 90% sequence identity to any one of SEQ ID NOs:5, 13, 20, 28, 36, 43, 50, 56, 61, 132, 344, 351, 407, 412, 462, 541, or 596.

In some embodiments, the antibody comprises:
(a) a heavy chain variable region comprising an amino acid sequence that has at least 90% sequence identity to SEQ ID NO:1 and a light chain variable region comprising an amino acid sequence that has at least 90% sequence identity to SEQ ID NO:5; or
(b) a heavy chain variable region comprising an amino acid sequence that has at least 90% sequence identity to SEQ ID NO:9 and a light chain variable region comprising an amino acid sequence that has at least 90% sequence identity to SEQ ID NO:13; or
(c) a heavy chain variable region comprising an amino acid sequence that has at least 90% sequence identity to SEQ ID NO:16 and a light chain variable region comprising an amino acid sequence that has at least 90% sequence identity to SEQ ID NO:20; or
(d) a heavy chain variable region comprising an amino acid sequence that has at least 90% sequence identity to SEQ ID NO:24 and a light chain variable region comprising an amino acid sequence that has at least 90% sequence identity to SEQ ID NO:28; or
(e) a heavy chain variable region comprising an amino acid sequence that has at least 90% sequence identity to SEQ ID NO:32 and a light chain variable region comprising an amino acid sequence that has at least 90% sequence identity to SEQ ID NO:36; or
(f) a heavy chain variable region comprising an amino acid sequence that has at least 90% sequence identity to SEQ ID NO:40 and a light chain variable region comprising an amino acid sequence that has at least 90% sequence identity to SEQ ID NO:43; or
(g) a heavy chain variable region comprising an amino acid sequence that has at least 90% sequence identity to SEQ ID NO:46 and a light chain variable region comprising an amino acid sequence that has at least 90% sequence identity to SEQ ID NO:50; or
(h) a heavy chain variable region comprising an amino acid sequence that has at least 90% sequence identity to SEQ ID NO:54 and a light chain variable region comprising an amino acid sequence that has at least 90% sequence identity to SEQ ID NO:56; or
(i) a heavy chain variable region comprising an amino acid sequence that has at least 90% sequence identity to SEQ ID NO:57 and a light chain variable region comprising an amino acid sequence that has at least 90% sequence identity to SEQ ID NO:61; or
(j) a heavy chain variable region comprising an amino acid sequence that has at least 90% sequence identity to SEQ ID NO:57 and a light chain variable region comprising an amino acid sequence that has at least 90% sequence identity to SEQ ID NO:5; or
(k) a heavy chain variable region comprising an amino acid sequence that has at least 90% sequence identity to SEQ ID NO:1 and a light chain variable region comprising an amino acid sequence that has at least 90% sequence identity to SEQ ID NO:61; or
(l) a heavy chain variable region comprising an amino acid sequence that has at least 90% sequence identity to SEQ ID NO:64 and a light chain variable region comprising an amino acid sequence that has at least 90% sequence identity to SEQ ID NO:5; or
(m) a heavy chain variable region comprising an amino acid sequence that has at least 90% sequence identity to SEQ ID NO:65 and a light chain variable region comprising an amino acid sequence that has at least 90% sequence identity to SEQ ID NO:5; or
(n) a heavy chain variable region comprising an amino acid sequence that has at least 90% sequence identity to SEQ ID NO:66 and a light chain variable region comprising an amino acid sequence that has at least 90% sequence identity to SEQ ID NO:5; or
(o) a heavy chain variable region comprising an amino acid sequence that has at least 90% sequence identity to SEQ ID NO:67 and a light chain variable region comprising an amino acid sequence that has at least 90% sequence identity to SEQ ID NO:5; or
(p) a heavy chain variable region comprising an amino acid sequence that has at least 90% sequence identity to SEQ ID NO:128 and a light chain variable region comprising an amino acid sequence that has at least 90% sequence identity to SEQ ID NO:132; or
(q) a heavy chain variable region comprising an amino acid sequence that has at least 90% sequence identity to SEQ ID NO:340 and a light chain variable region comprising an amino acid sequence that has at least 90% sequence identity to SEQ ID NO:344; or
(r) a heavy chain variable region comprising an amino acid sequence that has at least 90% sequence identity to SEQ ID NO:347 and a light chain variable region comprising an amino acid sequence that has at least 90% sequence identity to SEQ ID NO:351; or
(s) a heavy chain variable region comprising an amino acid sequence that has at least 90% sequence identity to SEQ ID NO:406 and a light chain variable region comprising an amino acid sequence that has at least 90% sequence identity to SEQ ID NO:407; or
(t) a heavy chain variable region comprising an amino acid sequence that has at least 90% sequence identity to SEQ ID NO:408 and a light chain variable region comprising an amino acid sequence that has at least 90% sequence identity to SEQ ID NO:412; or
(u) a heavy chain variable region comprising an amino acid sequence that has at least 90% sequence identity to SEQ ID NO:458 and a light chain variable region comprising an amino acid sequence that has at least 90% sequence identity to SEQ ID NO:462; or
(v) a heavy chain variable region comprising an amino acid sequence that has at least 90% sequence identity to SEQ ID NO:538 and a light chain variable region comprising an amino acid sequence that has at least 90% sequence identity to SEQ ID NO:541; or
(w) a heavy chain variable region comprising an amino acid sequence that has at least 90% sequence identity to SEQ ID NO:592 and a light chain variable region comprising an amino acid sequence that has at least 90% sequence identity to SEQ ID NO:596.

In still another aspect, monoclonal antibodies, or antigen-binding portions thereof, that specifically binds to a tree nut allergen are provided. In some embodiments, the antibody binds to the tree nut allergen with a binding affinity (KD) of less than 250 nM, less than 100 nM, less than 50 nM, less than 10 nM, or less than 5 nM. In some embodiments, the antibody binds to the tree nut allergen with a binding affinity (KD) of less than 1 nM. In some embodiments, the antibody binds to the tree nut allergen with a binding affinity (KD) from 1 nM to 250 nM. In some embodiments, the tree nut allergen is cashew, pistachio, pecan, walnut, hazelnut, and/or macadamia nut.

In some embodiments, the antibody comprises: a heavy chain CDR1 comprising the amino acid sequence of any one of SEQ ID NOs:113, 167, 175, 227, 311, 318, 438, 466, 621, 665, or 692;
a heavy chain CDR2 comprising the amino acid sequence of any one of SEQ ID NOs: 168, 176, 200, 312, 319, 439, 539, 666, or 693;
a heavy chain CDR3 comprising the amino acid sequence of any one of SEQ ID NOs: 169, 177, 228, 313, 320, 440, 467, 540, 667, or 694;
a light chain CDR1 comprising the amino acid sequence of any one of SEQ ID NOs: 171, 179, 230, 315, 322, 442, 469, 542, 623, 669, or 696;
a light chain CDR2 comprising the amino acid sequence of any one of SEQ ID NOs: 30, 94, 149, 172, 180, 196, 323, or 670; and
a light chain CDR3 comprising the amino acid sequence of any one of SEQ ID NOs: 173, 181, 231, 316, 324, 443, 470, 543, 624, 671, or 697.

In some embodiments, the antibody comprises a heavy chain CDR1-3 and a light chain CDR1-3 comprising the amino acid sequences of:
(a) SEQ ID NOs: 692, 693, 694, 696, 94, and 697, respectively; or
(b) SEQ ID NOs:318, 319, 320, 322, 323, and 324, respectively; or
(c) SEQ ID NOs:227, 200, 228, 230, 149, and 231, respectively; or
(d) SEQ ID NOs:113, 539, 540, 542, 196, and 543, respectively; or
(e) SEQ ID NOs:311, 312, 313, 315, 94, and 316, respectively; or
(f) SEQ ID NOs:665, 666, 667, 669, 670, and 671, respectively; or
(g) SEQ ID NOs:466, 200, 467, 469, 149, and 470, respectively; or
(h) SEQ ID NOs:167, 168, 169, 171, 172, and 173, respectively; or
(i) SEQ ID NOs:621, 176, 177, 623, 180, and 624, respectively; or
(j) SEQ ID NOs:175, 176, 177, 179, 180, and 181, respectively; or
(k) SEQ ID NOs:438, 439, 440, 442, 30, and 443, respectively.

In some embodiments, the antibody comprises a heavy chain variable region comprising an amino acid sequence that has at least 90% sequence identity to any one of SEQ ID NOs:166, 174, 226, 310, 317, 437, 465, 538, 620, 664, or 691. In some embodiments, the antibody comprises a light chain variable region comprising an amino acid sequence that has at least 90% sequence identity to any one of SEQ ID NOs:170, 178, 229, 314, 321, 441, 468, 541, 622, 668, or 695.

In some embodiments, the antibody comprises:
(a) a heavy chain variable region comprising an amino acid sequence that has at least 90% sequence identity to SEQ ID NO:691 and a light chain variable region comprising an amino acid sequence that has at least 90% sequence identity to SEQ ID NO:695; or
(b) a heavy chain variable region comprising an amino acid sequence that has at least 90% sequence identity to SEQ ID NO:317 and a light chain variable region comprising an amino acid sequence that has at least 90% sequence identity to SEQ ID NO:321; or
(c) a heavy chain variable region comprising an amino acid sequence that has at least 90% sequence identity to SEQ ID NO:226 and a light chain variable region comprising an amino acid sequence that has at least 90% sequence identity to SEQ ID NO:229; or
(d) a heavy chain variable region comprising an amino acid sequence that has at least 90% sequence identity to SEQ ID NO:538 and a light chain variable region comprising an amino acid sequence that has at least 90% sequence identity to SEQ ID NO:541; or
(e) a heavy chain variable region comprising an amino acid sequence that has at least 90% sequence identity to SEQ ID NO:310 and a light chain variable region comprising an amino acid sequence that has at least 90% sequence identity to SEQ ID NO:314; or
(f) a heavy chain variable region comprising an amino acid sequence that has at least 90% sequence identity to SEQ ID NO:664 and a light chain variable region comprising an amino acid sequence that has at least 90% sequence identity to SEQ ID NO:668; or
(g) a heavy chain variable region comprising an amino acid sequence that has at least 90% sequence identity to SEQ ID NO:465 and a light chain variable region comprising an amino acid sequence that has at least 90% sequence identity to SEQ ID NO:468; or
(h) a heavy chain variable region comprising an amino acid sequence that has at least 90% sequence identity to SEQ ID NO:166 and a light chain variable region comprising an amino acid sequence that has at least 90% sequence identity to SEQ ID NO:170; or
(i) a heavy chain variable region comprising an amino acid sequence that has at least 90% sequence identity to SEQ ID NO:620 and a light chain variable region comprising an amino acid sequence that has at least 90% sequence identity to SEQ ID NO:622; or
(j) a heavy chain variable region comprising an amino acid sequence that has at least 90% sequence identity to SEQ ID NO:174 and a light chain variable region comprising an amino acid sequence that has at least 90% sequence identity to SEQ ID NO:178; or
(k) a heavy chain variable region comprising an amino acid sequence that has at least 90% sequence identity to SEQ ID NO:437 and a light chain variable region comprising an amino acid sequence that has at least 90% sequence identity to SEQ ID NO:441.

In yet another aspect, monoclonal antibodies, or antigen-binding portions thereof, that specifically binds to a milk allergen are provided. In some embodiments, the antibody binds to the milk allergen with a binding affinity (KD) of less than 250 nM, less than 100 nM, less than 50 nM, less than 10 nM, or less than 5 nM. In some embodiments, the antibody binds to the milk allergen with a binding affinity (K_{D}) of less than 1 nM. In some embodiments, the antibody binds to the milk allergen with a binding affinity (K_{D}) from 1 nM to 250 nM.

In some embodiments, the antibody comprises:
a heavy chain CDR1 comprising the amino acid sequence of any one of SEQ ID NOs: 121, 750, 757, 765, 772, 779, 785, 793, 800, 807, 814, 821, 833, 838, 846, 853, 860, 868, 874, 881, 889, 895, 903, 911, 918, or 926;
a heavy chain CDR2 comprising the amino acid sequence of any one of SEQ ID NOs: 378, 532, 751, 758, 766, 773, 786, 794, 801, 808, 815, 822, 826, 839, 847, 854, 861, 875, 882, 890, 896, 904, 912, 919, or 927;
a heavy chain CDR3 comprising the amino acid sequence of any one of SEQ ID NOs: 752, 759, 767, 774, 780, 787, 795, 802, 809, 816, 827, 840, 848, 855, 862, 869, 876, 883, 891, 897, 905, 913, 920, or 928;
a light chain CDR1 comprising the amino acid sequence of any one of SEQ ID NOs: 523, 754, 761, 769, 776, 782, 789, 797, 804, 811, 818, 829, 835, 842, 850, 857, 864, 871, 878, 885, 899, 907, 915, 922, or 930;
a light chain CDR2 comprising the amino acid sequence of any one of SEQ ID NOs: 22, 30, 94, 110, 149, 186, 196, 389, 404, 509, 662, 682, 762, 790, 830, 843, 865, 886, 900, 908, 923, or 931; and
a light chain CDR3 comprising the amino acid sequence of any one of SEQ ID NOs: 755, 763, 770, 777, 783, 791, 798, 805, 812, 819, 824, 831, 836, 844, 851, 858, 866, 872, 879, 887, 893, 901, 909, 916, 924, or 932.

In some embodiments, the antibody comprises a heavy chain CDR1-3 and a light chain CDR1-3 comprising the amino acid sequences of:
(a) SEQ ID NOs: 800, 801, 802, 804, 110, and 805, respectively; or
(b) SEQ ID NOs:121, 826, 827, 829, 830, and 831, respectively; or
(c) SEQ ID NOs:833, 826, 827, 835, 149, and 836, respectively; or
(d) SEQ ID NOs:853, 854, 855, 857, 662, and 858, respectively; or
(e) SEQ ID NOs:860, 861, 862, 864, 865, and 866, respectively; or
(f) SEQ ID NOs:868, 378, 869, 871, 682, and 782, respectively.

In some embodiments, the antibody comprises a heavy chain variable region comprising an amino acid sequence that has at least 90% sequence identity to any one of SEQ ID NOs:749, 756, 764, 771, 778, 784, 792, 799, 806, 813, 820, 825, 832, 837, 845, 852, 859, 867, 873, 880, 888, 894, 902, 910, 917, or 925. In some embodiments, the antibody comprises a light chain variable region comprising an amino acid sequence that has at least 90% sequence identity to any one of SEQ ID NOs:753, 760, 768, 775, 781, 788, 796, 803, 810, 817, 823, 828, 834, 841, 849, 856, 863, 870, 877, 884, 892, 898, 906, 914, 921, or 929.

In some embodiments, the antibody comprises:
(a) a heavy chain variable region comprising an amino acid sequence that has at least 90% sequence identity to SEQ ID NO:799 and a light chain variable region comprising an amino acid sequence that has at least 90% sequence identity to SEQ ID NO:803; or
(b) a heavy chain variable region comprising an amino acid sequence that has at least 90% sequence identity to SEQ ID NO:825 and a light chain variable region comprising an amino acid sequence that has at least 90% sequence identity to SEQ ID NO:828; or
(c) a heavy chain variable region comprising an amino acid sequence that has at least 90% sequence identity to SEQ ID NO:832 and a light chain variable region comprising an amino acid sequence that has at least 90% sequence identity to SEQ ID NO:834; or
(d) a heavy chain variable region comprising an amino acid sequence that has at least 90% sequence identity to SEQ ID NO:852 and a light chain variable region comprising an amino acid sequence that has at least 90% sequence identity to SEQ ID NO:856; or
(e) a heavy chain variable region comprising an amino acid sequence that has at least 90% sequence identity to SEQ ID NO:859 and a light chain variable region comprising an amino acid sequence that has at least 90% sequence identity to SEQ ID NO:863; or
(f) a heavy chain variable region comprising an amino acid sequence that has at least 90% sequence identity to SEQ ID NO:867 and a light chain variable region comprising an amino acid sequence that has at least 90% sequence identity to SEQ ID NO:870.

In some embodiments, the antibody is an antibody that competes with a monoclonal antibody as disclosed herein for binding to an allergen (e.g., for binding to a food allergen such as a peanut allergen, a tree nut allergen, or a milk allergen).

In yet another aspect, monoclonal antibodies, or antigen-binding portions thereof, that specifically binds to a fungal allergen are provided. In some embodiments, the fungal allergen is an Aspergillus allergen. In some embodiments, the antibody binds to the fungal allergen (e.g., Aspergillus allergen) with a binding affinity (K_{D}) of less than 250 nM, less than 100 nM, less than 50 nM, less than 10 nM, or less than 5 nM. In some embodiments, the antibody binds to the fungal allergen (e.g., Aspergillus allergen) with a binding affinity (K_{D}) of less than 1 nM. In some embodiments, the antibody binds to the fungal allergen with a binding affinity (K_{D}) from 1 nM to 250 nM.

In some embodiments, the antibody or the antigen-binding portion thereof specifically binds to the allergen Aspergillus fumigatus, Aspergillus niger, and/or Aspergillus nidulans. In some embodiments, the antibody specifically binds to the allergen Aspergillus fumigatus 1 (Asp f1).

In some embodiments, the antibody comprises: a heavy chain CDR1 comprising the amino acid sequence of any one of SEQ ID NOs:710, 718, 726, 734, or 742;
a heavy chain CDR2 comprising the amino acid sequence of any one of SEQ ID NOs:711, 719, 727, 735, or 743;
a heavy chain CDR3 comprising the amino acid sequence of any one of SEQ ID NOs:712, 720, 728, 736, or 744;
a light chain CDR1 comprising the amino acid sequence of any one of SEQ ID NOs:714, 722, 730, 738, or 746;
a light chain CDR2 comprising the amino acid sequence of any one of SEQ ID NOs:715, 723, 731, 739, or 747; and
a light chain CDR3 comprising the amino acid sequence of any one of SEQ ID NOs:716, 724, 732, 740, or 748.

In some embodiments, the antibody comprises heavy chain and light chain CDR sequences contained within the heavy chain variable region and light chain variable region sequence pairs selected from the group consisting of SEQ ID NOs:709 and 713; 717 and 721; 725 and 729; 733 and 737; and 741 and 745. In some embodiments, the antibody comprises a heavy chain CDR1-3 and a light chain CDR1-3 comprising the amino acid sequences of:
(a) SEQ ID NOs: 710, 711, 712, 714, 715, and 716, respectively; or
(b) SEQ ID NOs:718, 719, 720, 722, 723, and 724, respectively; or
(c) SEQ ID NOs:726, 7272, 728, 730, 731, and 732, respectively; or
(d) SEQ ID NOs:734, 735, 736, 738, 739, and 740, respectively; or
(e) SEQ ID NOs:742, 743, 744, 746, 747, and 748, respectively.

In some embodiments, the antibody comprises a heavy chain variable region comprising an amino acid sequence that has at least 90% sequence identity to any one of SEQ ID NOs:709, 717, 725, 733, or 741. In some embodiments, the antibody comprises a light chain variable region comprising an amino acid sequence that has at least 90% sequence identity to any one of SEQ ID NOs:713, 721, 729, 737, or 745.

In some embodiments, the antibody comprises: (a) a heavy chain variable region comprising an amino acid sequence that has at least 90% sequence identity to SEQ ID NO:709 and a light chain variable region comprising an amino acid sequence that has at least 90% sequence identity to SEQ ID NO:713; or
(b) a heavy chain variable region comprising an amino acid sequence that has at least 90% sequence identity to SEQ ID NO:717 and a light chain variable region comprising an amino acid sequence that has at least 90% sequence identity to SEQ ID NO:721; or
(c) a heavy chain variable region comprising an amino acid sequence that has at least 90% sequence identity to SEQ ID NO:725 and a light chain variable region comprising an amino acid sequence that has at least 90% sequence identity to SEQ ID NO:729; or
(d) a heavy chain variable region comprising an amino acid sequence that has at least 90% sequence identity to SEQ ID NO:733 and a light chain variable region comprising an amino acid sequence that has at least 90% sequence identity to SEQ ID NO:737; or
(e) a heavy chain variable region comprising an amino acid sequence that has at least 90% sequence identity to SEQ ID NO:741 and a light chain variable region comprising an amino acid sequence that has at least 90% sequence identity to SEQ ID NO:745.

In some embodiments, the antibody is an antibody that competes with a monoclonal antibody as disclosed herein for binding to a fungal allergen (e.g., for binding to an Aspergillus allergen).

In another aspect, pharmaceutical compositions comprising a monoclonal antibody or antigen-binding portion as disclosed herein are provided. In some embodiments, the pharmaceutical composition comprises a plurality of monoclonal antibodies as disclosed herein, wherein the monoclonal antibodies recognize different epitopes or specifically bind to different antigens (e.g., different allergens within the same type or class of allergen or in different types or classes of allergens).

In another aspect, antibody-drug conjugates comprising a monoclonal antibody or antigen-binding portion thereof as disclosed herein are provided. In some embodiments, the antibody-drug conjugate comprises a monoclonal antibody or antigen-binding portion thereof that specifically binds to a fungal allergen as disclosed herein and further comprises an anti-fungal agent. In some embodiments, the anti-fungal agent is Amphotericin B.

In still another aspect, isolated polynucleotides comprising a nucleotide sequence encoding a monoclonal antibody as disclosed herein. Also provided herein are vectors and host cells comprising a polynucleotide as disclosed herein.

In another aspect, therapeutic methods are provided. In some embodiments, the therapeutic method is a method of reducing one or more allergy symptoms in a subject. In some embodiments, the therapeutic method is a method of reducing one or more allergy symptoms in a subject having a peanut allergy. In some embodiments, the therapeutic method is a method of reducing one or more allergy symptoms in a subject having a tree nut allergy. In some embodiments, the therapeutic method is a method of reducing one or more allergy symptoms in a subject having a fungal allergy. In some embodiments, the therapeutic method is a method of reducing one or more allergy symptoms in a subject having a milk allergy. In some embodiments, the method comprises administering to the subject a therapeutically effective amount of a monoclonal antibody or pharmaceutical composition as disclosed herein.

In another aspect, kits are provided. In some embodiments, the kit comprises a monoclonal antibody or pharmaceutical composition as disclosed herein. In some embodiments, the kit is for use in practicing a therapeutic method as disclosed herein.

### BRIEF DESCRIPTION OF THE DRAWINGS

FIG. 1. Flowchart of exemplary workflow for method of generating allergen-specific monoclonal antibodies.

FIGS. 2A-2G. Characterization of single B cells isolated from fresh human peripheral blood. (A) Principal component analysis (left) separates naive/memory (black dots) and plasmablast (PB, gray dots) B cell subsets identified by expression of established transcription factors and marker genes (right; non-striped for naive/memory B cells and striped for plasmablast (PB) B cells). (B) Isotype of B cells, black dots for IGHE and gray dots for other isotypes. (C) Number of cells belonging to each subtype in (A) by isotype (non-striped for naive/memory B cells and striped for plasmablast (PB) B cells). (D-G) Analysis of clonal families (CFs). (D) Distribution of the number of cells per CF. (E) Fraction of cells of each isotype that belong to a multi-member CF. (F) Isotype, B cell subtype, mutational frequency, and patient of origin of each cell within multi-member CFs. CFs referred to in the text are labeled. (G)(SEQ ID NOs: 4, 8, 12, 19, 23, 27, 31, 35, 39, 42, 45, 49, 53, 55, 138, 142, 177, 181, 193, 197, 199, 201, 204, 249, 252, 292, 295, 298, 302, 418, 422, 480, 528, 604, 612, 624, 683, 975-1016 and 1019) Heavy (right) and light (left) chain CDR3 sequences and similarity heatmap for CFs in (F).

FIGS. 3A-3G. Characterization of 89 IgE antibodies from single B cells. (A) Phylogenetic depiction of antibody heavy chains arranged by IGHV gene (background pattern), patient of origin (node pattern), and mutation frequency (node size). (B) Heatmap indicating number of cells with a given heavy and light chain CDR3 length. (C) Heavy and light chain mutation frequency of each cell. (D) Silent (S) and replacement (R) mutations by region within heavy and light chains. (E) Differential gene expression between IgE PBs and PBs of other isotypes. Positive log fold change indicates genes enriched in IgE PBs. (F) Heavy chain constant region coverage histograms for naïve/memory B cells (top) and PBs (bottom) by isotype, with loci oriented in the 5′ to 3′ direction. Mean normalized read depth and 95% confidence interval are indicated by solid lines and shaded area, respectively, for the number of cells (n) inscribed. Membrane exons are the two most 3′ exons of each isotype. (G) Summary of (F), but depicting the fraction of cells of each isotype with any membrane exon coverage.

FIGS. 4A-4C. High affinity, cross-reactive IgE antibody convergence in two unrelated individuals (PA12 & PA13). Antibody patterns are conserved among panels. (A) Frequency of silent (S) and replacement (R) mutations by region. (B) Mutation frequency percentiles compared to all class-switched antibodies. (C) Dissociation constants (KDs) for major allergenic peanut proteins Ara h 2 and Ara h 3 for six convergent antibodies as well as eight variants of PA13P1H08.

Shortened antibody variant names are designated as “heavy-light,” using the following abbreviations: N=native, R=reverted, FWRs=framework regions. An “r” prefix indicates only that region has been reverted.

FIGS. 5A-5E. Germline transcription reveals class switch priming in single B cells. (A) Identity of Cε germline transcript splice donors along with the number of cells, by isotype, expressing each. (B) Fraction of cells expressing EGLTs by isotype. (C) Example from individual PA11 where identification of phased variants within IgE constant region exons enables subsequent verification of biallelic GLT expression in other B cells from the same patient. (D) Global germline transcription state heatmap indicating the fraction of cells of a given isotype expressing a given GLT. Above: GLT isotype expression frequency relative to all GLT isotypes; excludes self-isotype GLT expression. (E) Histogram of the number of non-self GLT isotypes expressed in each cell.

FIG. 6. Study overview. Plasma was extracted from fresh blood to measure circulating IgE levels, while the cellular fraction was enriched for B cells prior to FACS. Single cells were sorted into individual wells of a 96 well plate and processed with scRNA-seq, generating sequencing reads that were aligned to the genome to calculate gene expression and assess splicing as well as assembled in order to reconstruct heavy and light chain sequences. Specificity and affinity data were generated for recombinantly expressed antibodies.

FIGS. 7A-7C. Plasma IgE levels. (A) Allergen-specific and allergen component (hazelnut, peanut) concentrations. (B) Total IgE concentration. (C) Positive correlation between total plasma IgE level and the number confirmed IgE+ B cells. Each point is an individual.

FIGS. 8A-8C. FACS gating and analysis. (A) Gating strategy for sorting single B cells. IgE+ B cells have been overlaid as dark gray dots. (B) Isotype identity within the final IgE gate as determined by heavy chain transcript assembly. ND=not determined. (C) For reference, putative basophils (CD19−IgE+) display higher IgE surface expression than IgE+ B cells.

FIGS. 9A-9G. Single cell RNA-seq data overview and quality control. (A) Cells were sequenced in 5 libraries to a depth of ^{˜}1-2 million reads/cell. (B) Genes per cell histogram. Cells expressing fewer than 950 genes were discarded. (C) Rarefaction curve depicting the number of genes detected as a function of sequencing depth for eight randomly selected cells in each B cell subtype. Solid lines and shaded area represent mean and 95% confidence interval for the gene count, respectively. (D) Read mapping distribution for retained cells. Most reads mapped uniquely (Ensembl reference annotation) and multimapped reads largely belonged to RNA18S5 repeats on chr21 and unplaced scaffolds. (E) Read mapping across gene bodies showed minimal 3′ or 5′ bias. (F) V gene assembly length histogram by chain. (G) PCA on the top 500 most variable genes before (top) and after (bottom) batch correction.

FIGS. 10A-10D. Auxiliary data supporting B cell subtype classification. PBs (striped) have greater FACS forward and side scatter (A), more cDNA after SmartSeq2 preamplification (B), and have greater gene expression of antibody light and heavy chains (C) as compared to the naive/memory B cell subset (non-striped). (D) Top differentially expressed genes for each subset.

FIGS. 11A-11K. B cell comparisons across isotypes. (A-I) Number of cells with a given V and J gene by isotype and chain. (A) IGHM. (B) IGHD. (C) IGHG3. (D) IGHG1. (E) IGHA1. (F) IGHG2. (G) IGHG4. (H) IGHE. (I) IGHA2. (J) Heavy chain mutation frequency by isotype. (K) Relative utilization of the lambda and kappa light chain by isotype.

FIGS. 12A-12H. Antibody specificity and affinity characterizations. (A) Semi-quantitative indirect ELISAs of convergent antibodies, controls, PA13P1H08 variants, and antibodies from other clonal families. Human IgG isotype control (abcam #ab206195) served as a negative control, while positive controls included anti-Ara h mouse monoclonal antibodies purchased from Indoor Biotechnologies. (B-G) Kinetic characterization of antibody binding to Ara h 2 (B-D) and Ara h 3 (E-G) using biolayer interferometry. Antibodies are named and described herein. (H) Indirect ELISA showing binding of recombinant monoclonal antibodies from subjects PA11, PA12, PA13, PA14, PA15, and PA16 (rows) to allergen extracts, natural peanut allergen Ara h 2, and bovine serum albumin (BSA) (columns). The isotype of each antibody is shown to the left. Higher values indicate stronger binding. OD=optical density. Depicted values represent those after subtraction of human IgG isotype control. Only the tested antibodies with any OD value above 0.25 are shown.

FIGS. 13A-13E. Sequences of heavy and light chains used in constructing PA13P1H08 antibody variants. (A) (SEQ ID Nos: 933-952) Derivation of the inferred naïve heavy chain CDR3 and surrounding amino acids. (B) (SEQ ID NOs: 1, 57 and 64-67) Native and reverted heavy chain sequences, in addition to sequences where region(s) of the heavy chain have been reverted to the inferred naïve rearrangement. Labels with an “r” prefix indicate only that region has been reverted. FWRs=frameworks. (C) (SEQ ID NOs: 953-969) Derivation of the inferred naïve light chain CDR3 and surrounding amino acids. (D) (SEQ ID NOs: 5 and 61) Native and reverted light chain sequences. (E) (SEQ ID NO: 970) Sequence of a light chain taken from another antibody, PA12P4H03, which we confirmed did not to bind any peanut allergens by ELISA.

FIG. 14. GLT splicing for all isotypes. Note that only the first three constant region exons are shown for each isotype for clarity.

FIG. 15. IGV coverage histograms and splice junctions for the ighe constant region locus showing εGLTs in single murine B cells stimulated with IL-4, LPS, and BAFF (Wu et al. 2017). Arrows indicate ighe GLT splice donors.

FIG. 16. (SEQ ID Nos:971-973) Pairwise CDR3 sequence identity of the heavy chain CDR3 sequences from clones PA12P3F10, PA12P3D08, PA12P1C07, PA13P1E10, PA13P3G09, and PA13P1H08 (SEQ ID NOs: 19, 35, 42, 12, 27, 4) and three heavy chain CDR3 sequences derived from multiple patients in a separate peanut allergy immune repertoire sequencing study (62). Each sequence from this separate study has an identity of at least 70% with one or more sequences from the present study. All sequences share the IGHV3-30 and IGHJ6 gene segments and have CDR3s 17 amino acids in length.

FIG. 17A. Microarray scan confirming the absence of background interactions of the secondary goat anti-human IgG and the control mouse monoclonal anti-HA antibody with antigen-derived peptides. The control antibody gave rise to the expected HA control spot pattern framing the peptide microarray (white dots).

FIGS. 17B and 17C. Microarray epitope mapping of PA13P1H08 to Ara h 2 (“Ah2”) and Ara h 3 (“Ah3”) peptides. (B) Microarray scan illustrating antibody binding to antigen peptides (light gray) as well as the expected HA control spot pattern (white dots). (C) (SEQ ID Nos: 705-707) Microarray fluorescence intensity by antigen peptide. Ara h 2 motifs with high intensity are annotated.

FIG. 18. Plasma IgE levels for milk allergic subject PA01 and Aspergillus study subject 10033201 against common food allergens as well as Aspergillus fumigatus and Aspergillus niger. NP=not performed. Total IgE was 353 kU/L and 3528 kU/L for PA01 and 10033201, respectively. The assay was performed by CLIA-licensed Johns Hopkins University Dermatology, Allergy, and Clinical Immunology Reference Laboratory using the ImmunoCAP system.

FIG. 19. Indirect ELISA showing binding of recombinant monoclonal antibodies (columns) to antigens (rows). Antigens include extracts of Aspergillus fumigatus, Aspergillus niger, and Aspergillus nidulans, as well as a purified recombinant allergen Aspergillus fumigatus 1 (rAsp f 1). Bovine serum albumin (BSA) serves as a negative antigen control. A monoclonal antibody against Asp f 1 (“anti-Asp f 1”) serves as a positive control against this allergen. Higher values indicate stronger binding. OD=optical density. hlgG=human IgG isotype control.

FIG. 20. Indirect ELISA showing binding of recombinant monoclonal antibodies from subject PA01 (rows) to allergen extracts, natural peanut allergen Ara h 2, and BSA (columns). The isotype of each antibody is shown to the left. Higher values indicate stronger binding. OD=optical density. Depicted values represent those after subtraction of human IgG isotype control.

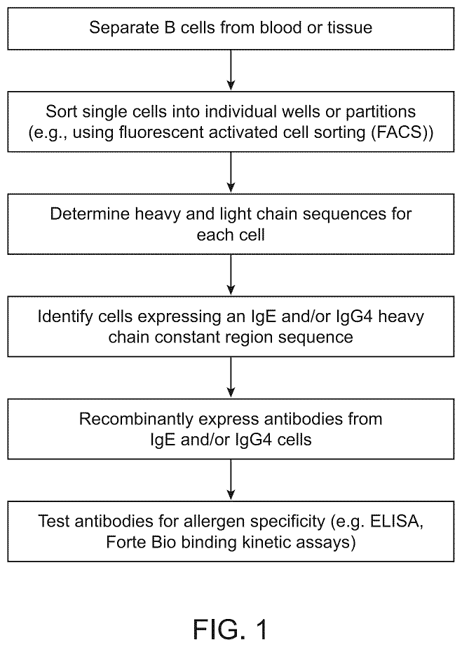

### Table 1

Table 1 includes protein and nucleic acid sequences discussed herein. Polypeptide sequences are provided using the standard one-letter code. One of ordinary skill in the art provided with an amino acid sequence will understand that the amino acid sequence may be encoded by a defined set of nucleotide sequences such that the reader and inventors have possession of the nucleotide sequences encoding each amino acid sequence. A nucleic acid sequence encoding a polynucleotide may be a naturally occurring human sequence. In some embodiments a nucleic acid sequence encoding a polynucleotide is not a naturally occurring human sequence. A nucleic acid sequence encoding a polynucleotide may be a sequence that is codon optimized for expression in human cells or specific cell types, eukaryotic cells, bacteria cells, or otherwise. Codon optimization, which replaces one codon by another codon encoding the same amino acid and having a higher frequency of occurrence in the particular host cell, can be performed to improve the ability of the host to produce the polypeptide encoded by the nucleic acid (see, e.g., Mauro, BioDrugs 32(1):69-81, 2018 and Kato, Inti Mol Sci. 20(4), 2019).

In certain embodiments it is contemplated that variant sequences may be used in methods and compositions disclosed herein. For example, in one aspect, an antibody with a heavy chain variable region comprising an amino acid sequence that has at least 90% sequence identity to SEQ ID NO:1 and a light chain variable region comprising an amino acid sequence that has at least 90% sequence identity to SEQ ID NO:5 is described. A degree of sequence identity or similarity can be determined using art-known methods. In one approach, the identity of two nucleotide or polypeptide sequences or subsequences is calculated as the percentage of positions that are identical or equivalent after the sequences have been aligned, introducing gaps, if necessary, to achieve maximum percent sequence identity. Methods of sequence alignment are art-known methods, and include, but are not limited to the Needleman-Wunsch global alignment algorithm (Needleman and Wunsch (1970) J. Mol. Biol. 48(3):443-453 (30)), the Smith and Waterman local homology search algorithm (Smith, Temple F. & Waterman, Michael S. (1981) J. Mol. Biol. 147 (1): 195-197.), manual alignment and inspection, or computerized implementations of these algorithms, such as the “needle” program, distributed as part of the EMBOSS software package (Rice, P. et al., Trends in Genetics 16(6): 276-277 (31), versions 6.3.1 available from EMBnet at various sources).

It is contemplated that, in certain embodiments, a method or composition described herein will differ from a polypeptide sequence provided herein (e.g., in Table 1) by one or more amino acid substitutions. In some embodiments a sequence will have at least 90% sequence identity (or other degree of sequence identity disclosed hereinbelow) to a sequence or combination of sequences described herein. In one embodiment the polypeptide sequence differs from a reference sequence (e.g., in Table 1) by one amino acid substitution. In one embodiment the polypeptide sequence differs from a reference sequence (e.g., in Table 1) by two amino acid substitutions. In one embodiment the polypeptide sequence differs from a reference sequence by two amino acid substitutions. In one embodiment the polypeptide sequence differs from a reference sequence by three amino acid substitutions. In one embodiment the polypeptide sequence differs from a reference sequence by four amino acid substitutions. In one embodiment the polypeptide sequence differs from a reference sequence by five amino acid substitutions. In one embodiment the polypeptide sequence differs from a reference sequence by six amino acid substitutions. In one embodiment the polypeptide sequence differs from a reference sequence by seven amino acid substitutions. In one embodiment the polypeptide sequence differs from a reference sequence by eight amino acid substitutions. In one embodiment the polypeptide sequence differs from a reference sequence by nine amino acid substitutions. In one embodiment the polypeptide sequence differs from a reference sequence by ten amino acid substitutions. In certain embodiments the polypeptide sequence differs from a reference sequence by 1-10 amino acid substitutions, sometimes 1-5 amino acid substitutions. In some cases, amino acid substitutions are selected that do not change a basic property (e.g., binding specificity) relative to the reference sequence. In some cases, amino acid substitutions are selected that change binding affinity but not binding specificity. In some cases substitutions are selected to change a property (e.g., substitutions that affect effector function or half-life) as known in the art or described herein below. In some embodiments the amino acid substitutions are conservative substitutions. A conservative amino acid substitution is recognized in the art as a substitution of one amino acid for another amino acid that has similar properties, such as polarity, charge, hydrophobicity, and aromaticity. A conservative amino acid substitution can also be made based on the side chain characteristics of the amino acid, such as containing sulfur, hydroxyl, or amide. Non-limiting examples of conservative amino acid substitutions are set out below.

| Amino acid property | Amino acid |
|---|---|
| Polar-uncharged | Cys, Ser, Thr, Met, Asn, Gln |
| Polar-charged | Asp, Glu, Lys, Arg |
| Non-polar | Gly, Ala, Pro, Ile, Leu, Val |
| Aromatic | His, Phe, Trp, Tyr |
| Aliphatic | Ala, Leu, Ile, Val, Pro |
| Positively charged | Lys, Arg, His |
| Negatively charged | Asp, Glu |
| Sulfur-containing | Met |
| Hydroxyl-containing | Ser, Thr, Tyr |
| Amide-containing | Asn, Gln |
| Sulfhydryl containing | Cys |

### DETAILED DESCRIPTION OF THE INVENTION

### 1. Introduction

In one aspect, the present disclosure provides human allergen-specific monoclonal antibodies and methods for generating human allergen-specific monoclonal antibodies from single IgE- or IgG4-expressing human B cells. IgE antibodies, the least abundant class of antibodies in humans, are known to cause the symptoms of allergic reactions. For example, food allergy symptoms ranging from urticaria to potentially fatal anaphylaxis result from the degranulation of mast cells and basophils induced by the recognition of allergic food proteins by surface-bound IgE antibodies. Despite this central role in immunity and allergic disease, human IgE antibodies remain poorly characterized due to their scarcity. Fitzsimmons et al., Front Immunol., 2014, 5:61. Similarly, there is a lack of knowledge, but growing interest, surrounding the IgG4 isotype due to its potential role in mediating the reduced clinical allergen reactivity that accompanies immunotherapy and early allergen exposure through antigen blocking. Tordesillas et al., Immunity, 2017, 47:32-50.

The present disclosure provides therapeutic methods for treating a human subject having an allergy or reducing one or more allergy symptoms in a human subject with one or more of the allergen-specific monoclonal antibodies or antigen-binding portions thereof as disclosed herein. In some embodiments and without intending to be bound by a particular mechanism, the allergen-specific monoclonal antibodies disclosed herein are used therapeutically as blocking antibodies, which is often referred to as passive immunotherapy.

As described herein, the methods of the disclosure can be used to generate, from a sample from a human subject having an allergy to an antigen of interest, a pool of genotype-confirmed IgE or IgG4 single B cells that are candidates for producing antibodies having high affinity for an allergen of interest. As described in the Examples section below, it has been found that analyzing the cDNA sequences of immunoglobulin heavy chain constant regions to identify the isotype of single B cells avoids the problem of isotype mischaracterization that is known to occur when B cell isotype is determined based on sorting cells by cell surface markers (e.g., as is typically done in FACS cell surface staining). This problem of isotype mischaracterization is known to be especially pervasive for IgE B cells because the marker CD23 is a “low-affinity” IgE receptor that captures IgE on the surface of non-IgE B cells. See, Berkowska et al., J Allergy Clin Immunol, 2014, 134:688-697. Thus, the methods of the present disclosure generate a pool of single B cells that are much more likely to produce antibodies having high affinity for the allergen. Furthermore, it has been found that antibodies generated according to the methods disclosed herein are among the highest affinity native human antibodies discovered to date and exhibit cross-reactivity to different antigens.

### II. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

The term “allergen” refers to a substance that induces an immune response in a subject that results in an allergic reaction by the subject.

As used herein, the term “antibody” refers to a polypeptide encoded by an immunoglobulin gene or functional fragments thereof that specifically binds and recognizes an antigen. The term “antibody,” as used herein, also includes antibody fragments that retain binding specificity, including but not limited to Fab, F(ab′)₂, Fv, and scFv. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain” or “VH” refer to the variable region of an immunoglobulin heavy chain, including an Fv, scFv, dsFv or Fab; while the terms “variable light chain” or “VL” refer to the variable region of an immunoglobulin light chain, including an Fv, scFv, dsFv or Fab.

The term “variable region” refers to a domain in an antibody heavy chain or light chain that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

As noted, the part of a variable region not contained in the CDRs is called the framework. The “framework regions” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well known definitions in the art. The position and length of the CDRs have been precisely defined by Kabat et al., Sequences of Proteins of Immunological Interest, U.S. Department of Health and Human Services, 1983, 1987, and others. See, e.g., Johnson and Wu, Nucleic Acids Res. 2000 Jan. 1; 28(1): 214-218; Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; AI-Lazikani et al., J.Mol.Biol 1997, 273(4)); and MacCallum et al., J. Mol. Biol., 262:732-745 (1996). Also see international ImMunoGeneTics database (IMGT), AbM, and observed antigen contacts.

The terms “antigen-binding portion” and “antigen-binding fragment” are used interchangeably herein and refer to one or more fragments of an antibody that retains the ability to specifically bind to an antigen (e.g., an allergen, e.g., Ara h 2 or Ara h 3). Examples of antibody-binding fragments include, but are not limited to, a Fab fragment (a monovalent fragment consisting of the VL, VH, CL, and CH1 domains), F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen. Antibodies and antigen-binding portions thereof include domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains. Exemplary configurations of variable and constant domains that may be found within an antigen-binding fragment of an antibody of the present invention include: (a) VH-CHI; (b) VH-CH2; (c) VH-CH3; (d) VH-CH1-CH2; (e) VH-Ch1-Ch2-Ch3; (f) VH-Ch2-Ch3; (g) VH-CL; (h) VL-CH1; (i) VL-Ch2; (X) VL-Ch3; (j) VL-CH1-CH2; (k) VL-CH1-CH2-CH3; (I) VL-CH2-CH3; and (m) VL-CL (see, e.g., FUNDAMENTAL IMMUNOLOGY (Paul ed., 4th ed. 2001), Gruber et al. (1994) J Immunol. 152:5368-5374; McCartney, et al., 1995 Protein Eng. 8:301-314; Shukra et al., 2014, “Production of recombinant antibodies using bacteriophages” Eur J Microbiol Immunol (Bp). 4(2): 91-98; Todorovska, 2001, “Design and application of diabodies, triabodies and tetrabodies for cancer targeting” J Immunol Methods; 248(1-2):47-66; Salvador et al., 2019, “Nanobody: outstanding features for diagnostic and therapeutic applications” Anal Bioanal Chem. 411(9):1703-1713; Gill et al., 2006, “Biopharmaceutical drug discovery using novel protein scaffolds.” Curr Opin Biotechnol., (6):653-8; and Ubah et al., 2016, “Phage Display Derived IgNAR V Region Binding Domains for Therapeutic Development” Curr Pharm Des. 22(43):6519-6526, each of which is incorporated by reference herein.

The term “epitope” refers to the area or region of an antigen to which an antibody specifically binds, i.e., an area or region in physical contact with the antibody, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

A “monoclonal antibody” refers to antibodies produced by a single clone of cells or a single cell line and consisting of or consisting essentially of antibody molecules that are identical in their primary amino acid sequence. In some embodiments, a monoclonal antibody preparation comprises a population of antibodies that are identical and bind to the same epitope of an antigen, except for mutations that arise during monoclonal antibody production. Unless otherwise specified or clear from context, the term ‘monoclonal antibody’ includes synthetic antibodies and antigen binding fragments thereof.

A “human antibody” refers to an antibody having variable and constant regions derived from human germline immunoglobulin sequences. A human antibody of the present disclosure may include amino acid residues not encoded by human germline immunoglobulin sequences (e.g., mutations introduced by random or site-directed mutagenesis in vitro or by somatic mutations in vivo). The term “human antibody” is not intended to include chimeric or humanized antibodies in which CDR sequences derived from the germline or immune cells of a non-human species (e.g., mouse) have been grafted onto human framework sequences.

The term “specifically binds” refers to a molecule (e.g., an antibody or antibody fragment) that binds to a target with greater affinity, avidity, more readily, and/or with greater duration to that target in a sample than it binds to a non-target compound. In some embodiments, an antibody or antigen-binding portion thereof that specifically binds a target (e.g., an allergen, e.g., Ara h 2 or Ara h 3) is an antibody or antigen-binding portion that binds to the target with at least 2-fold greater affinity than non-target compounds, e.g., at least 3-fold, 4-fold, 5-fold, 6-fold, 7-fold, 8-fold, 9-fold, 10-fold, 20-fold, 25-fold, 50-fold or greater affinity. For example, in some embodiments, an antibody that specifically binds to an allergen target, such as Ara h 2 or Ara h 3, will typically bind to the allergen target with at least a 2-fold greater affinity than to a non-allergen target. It will be understood by a person of ordinary skill in the art that an antibody that specifically or preferentially binds to a first target may or may not specifically or preferentially bind to a second target.

The term “binding affinity,” as used herein, refers to the strength of a non-covalent interaction between two molecules, e.g., an antibody (or an antigen-binding fragment thereof) and an antigen. Thus, for example, the term may refer to 1:1 interactions between an antibody (or an antigen-binding fragment thereof) and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding fragment thereof) and an antigen, but also apparent affinities for which K_{D}s are calculated that may reflect avid binding.

The term “cross-reacts,” as used herein, refers to the ability of an antibody to bind to two or more antigens. As a non-limiting example, in some embodiments, an antibody that specifically binds to a first allergen target (e.g., a first peanut allergen, such as Ara h 2) can exhibit cross-reactivity with a second allergen target (e.g., a second peanut allergen, such as Ara h 3).

The term “isolated,” as used with reference to a nucleic acid or protein (e.g., antibody), denotes that the nucleic acid or protein is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins, wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “nucleic acid” and “polynucleotide” are used interchangeably. Use of the term “polynucleotide” includes oligonucleotides (i.e., short polynucleotides). This term also refers to deoxyribonucleotides, ribonucleotides, and naturally occurring variants, and can also refer to synthetic and/or non-naturally occurring nucleic acids (i.e., comprising nucleic acid analogues or modified backbone residues or linkages), such as, for example and without limitation, phosphorothioates, phosphoramidates, methyl phosphonates, chiral-methyl phosphonates, 2-O-methyl ribonucleotides, peptide-nucleic acids (PNAs), and the like. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (see, e.g., Batzer et al., Nucleic Acid Res. 19:5081 (1991); Ohtsuka et al, J. Biol. Chem. 260:2605-2608 (1985); Rossolini et al, Mol. Cell. Probes 8:91-98 (1994)).

The term “sample,” as used herein, refers to a biological sample obtained from a human or non-human mammalian subject. In some embodiments, a sample comprises blood, blood fractions or blood products (e.g., serum, plasma, platelets, red blood cells, peripheral blood mononuclear cells and the like); sputum or saliva; stool, urine, other biological fluids (e.g., lymph, saliva, prostatic fluid, gastric fluid, intestinal fluid, renal fluid, lung fluid, cerebrospinal fluid, and the like), tissue (e.g., kidney, lung, liver, heart, brain, nervous tissue, thyroid, eye, skeletal muscle, cartilage, or bone tissue), cultured cells (e.g., primary cultures, explants, transformed cells, or stem cells), or a biopsy sample.

The terms “subject” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject or patient is a human.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; making the final point of degeneration less debilitating; and/or improving a subject's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutical composition” refers to a composition suitable for administration to a subject. In general, a pharmaceutical composition is sterile, and preferably free of contaminants that are capable of eliciting an undesirable response with the subject. Pharmaceutical compositions can be designed for administration to subjects in need thereof via a number of different routes of administration, including oral, intravenous, buccal, rectal, parenteral, intraperitoneal, intradermal, intratracheal, intramuscular, subcutaneous, inhalational, and the like.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent (e.g., a monoclonal antibody as disclosed herein) is an amount of the agent that treats, ameliorates, abates, remits, improves patient survival, increases survival time or rate, diminishes symptoms, makes an injury, disease, or condition (e.g., an allergy) more tolerable, slows the rate of degeneration or decline, or improves a patient's physical or mental well-being. For example, for the given parameter, a therapeutically effective amount will show an increase or decrease of therapeutic effect at least 5%, 10%, 15%, 20%, 25%, 40%, 50%, 60%, 75%, 80%, 90%, or 100%. Therapeutic efficacy can also be expressed as “-fold” increase or decrease. For example, a therapeutically effective amount can have at least a 1.2-fold, 1.5-fold, 2-fold, 5-fold, or more effect over a control.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, rectal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, PA.

### III. Methods of Generating Allergen-Specific Antibodies

In one aspect, methods of generating allergen-specific monoclonal antibodies from a human sample are provided. In some embodiments, the method comprises:
(a) isolating single B cells from a sample from a human subject, wherein the subject is allergic to the allergen;
(b) generating cDNAs from the single B cells of step (a), wherein the cDNA sequences comprise a first sequence that encodes all or part of an immunoglobulin heavy chain and a second sequence that encodes all or part of an immunoglobulin light chain;
(c) determining the sequences of the cDNAs from step (b);
(d) analyzing the sequences determined in step (c) to identify single B cells comprising a first sequence that comprises an IgE constant region or an IgG4 constant region;
(e) identifying, from the single B cells of step (d), (i) a heavy chain variable region sequence in the immunoglobulin heavy chain that comprises an IgE constant region or an IgG4 constant region, and (ii) a light chain variable region sequence in an immunoglobulin light chain that is co-expressed with the immunoglobulin heavy chain in the same single B cell;
(f) expressing antibodies comprising the heavy chain variable region sequence and the light chain variable region sequence from step (e); and
(g) identifying one or more antibodies from step (f) that specifically bind to the allergen.

### Subject Populations and Samples

In some embodiments, the method of generating allergen-specific monoclonal antibodies comprises isolating B cells from a biological sample from a human subject. In some embodiments, the sample comprises whole blood, peripheral blood, or a leukapheresis product.

In some embodiments, the sample comprises peripheral blood mononuclear cells (PBMCs). In some embodiments, the sample comprises a tissue from the human subject, e.g., tonsil tissue, spleen, or bone marrow. Methods of isolating B cells from blood and tissue samples are described in the art. See, e.g., Heine et al., Curr Protoc. Immunol., 2011, 94:7.5.1-7.5.14; and Zuccolo et al., BMC Immunol, 2009, 10:30, doi:10.1186/1471-2172-10-30.

In some embodiments, the allergen-specific antibodies are generated from a human subject having an allergy to the allergen. In some embodiments, the human subject having an allergy is an adult. In some embodiments, the human subject having an allergy is a juvenile. In some embodiments, the human subject has an allergy to a food allergen, a plant allergen, a fungal allergen, an animal allergen, a dust mite allergen, a drug allergen, a cosmetic allergen, or a latex allergen. In some embodiments, the human subject has allergies to two or more allergens, e.g., to two or more of a food allergen, a plant allergen, a fungal allergen, an animal allergen, a dust mite allergen, a drug allergen, a cosmetic allergen, or a latex allergen. In some embodiments, the human subject has allergies to 2, 3, 4, 5, 6, 7, 8, 9, 10 or more allergens. In some embodiments, the human subject has allergies to two or more different types of antigens (allergens) in a class of allergen, e.g., allergies to two or more different food allergens (e.g., allergies to two or more different peanut antigens, or allergies to a peanut allergen and a non-peanut allergen such as a tree nut, egg, or milk allergen), or allergies to two more different fungal allergens (e.g., allergies to two or more different species of Aspergillus). In some embodiments, the human subject has allergies to two more different classes of allergens (e.g., allergies to one or more food allergens and to one or more plant allergens). In some embodiments, the human subject has allergies to only one class of allergens (e.g., the subject has allergies to one or more food allergens but not to non-food allergens, or the subject has allergies to one or more fungal allergens but not to non-fungal allergens).

In some embodiments, the human subject has an allergy to a food allergen. In some embodiments, the food allergen is a milk allergen, an egg allergen, a nut allergen, a tree nut allergen, a fish allergen, a shellfish allergen, a soy allergen, a legume allergen, a seed allergen, or a wheat allergen. In some embodiments, the food allergen is a peanut allergen.

In some embodiments, the human subject has an allergy to a plant allergen or a fungal allergen. In some embodiments, the allergen is a fungal allergen (e.g., Aspergillus, e.g., Asp. fumigatus, Asp. niger, or Asp. nidulans). In some embodiments, the allergen is a pollen allergen (e.g., tree pollen, grass pollen, or weed pollen) or a mold allergen. In some embodiments, the human subject has an allergy to an animal allergen. In some embodiments, the allergen is a dander allergen or an insect sting.

In some embodiments, the method of generating allergen-specific monoclonal antibodies does not comprise immunizing the human subject with the allergen or exposing the human subject to the allergen prior to obtaining the sample from the subject.

### B Cell Isolation and Screening

In some embodiments, single B cells are isolated from the sample from the subject having an allergy to the allergen. In some embodiments, the single B cells are separated into separate partitions, e.g., separate wells of a multi-well plate, encapsulated into droplets, or dispersed into microwells. In some embodiments, at least 10, 50, 100, 500, 1,000, 5,000, or 10,000 B cells or more are isolated from a sample and are separated into separate partitions.

In some embodiments, the isolating step comprises sorting cells in the sample by fluorescent activated cell sorting (FACS). FACS sorting can be used to sort cells based on cell surface marker expression, cell size, and/or granularity and deliver cells individually to a well, e.g., a 96-well or 384-well tissue culture or PCR plate. Methods of isolating and purifying cell populations by FACS are described in the art. See, e.g., Basu et al., J Vis Exp, 2010, 41:1546, doi:10.3791/1546.

In some embodiments, a droplet microfluidic platform can be used to dispense single B cells into separate droplets. In some embodiments, the nucleic acids (e.g., mRNA) of a single cell in a droplet is labeled with a nucleotide sequence that is unique to the droplet, e.g., a Unique Molecular Identifier barcode nucleotide sequence, thereby enabling downstream processing steps for the sequences from multiple B cells to be performed in a single reaction container. Methods of encapsulating single cells in droplets are described in the art. See, e.g., Macosko et al., Cell, 2015, 161: 1202-1214; Zhang et al., Scientific Reports, 2017, 7:41192, doi:10.1038/srep41192.

In some embodiments, cells are dispersed into microwells designed to trap a single cell. Methods of single cell microwell trapping are described in the art. See, e.g. Han et al., Cell, 2018, 172:5, doi:10.1016/j.cell.2018.02.001.

In some embodiments, cells from the sample are screened for the presence, absence, or level of expression of one or more markers and single B cells are isolated based on the presence or level of expression of the one or more B cell markers (e.g., one, two, three, four, five, six, seven, eight, or more markers). In some embodiments, cells are screened for the presence, absence, or level of expression of one or more cell surface B cell markers, such as but not limited to CD19, CD20, CD21, CD22, CD23, CD24, CD40, CD72, or CD79. In some embodiments, a cell is determined to be a B cell if the cell is positive for one or more of the B cell markers, e.g., is positive for one or more of CD19, CD20, CD21, CD22, CD23, CD24, CD40, CD72, or CD79. In some embodiments, single CD19⁺ B cells are isolated.

In some embodiments, cells from the sample are screened for the presence, absence, or level of expression of one or more immunoglobulin isotypes, such as but not limited to IgE, IgG, IgM, IgA, or IgD or a subclass thereof. In some embodiments, the single B cells that are isolated are selected for expression of cell surface IgE and/or for expression of cell surface IgG4. In some embodiments, single B cells are isolated without selecting for expression of one or more immunoglobulin isotypes (e.g., without selecting for expression of cell surface IgE and/or for expression of cell surface IgG4).

### IgE Selection

In some embodiments, the method comprises isolating single B cells that are selected for expression of cell surface IgE. In some embodiments, the isolating step comprises contacting cells of the sample with an anti-human IgE antibody and selecting for cells that express IgE on the cell surface. In some embodiments, the isolating step comprises contacting cells of the sample with antibody against a B cell marker (e.g., an antibody against CD19, CD20, CD21, CD22, CD23, CD24, CD40, CD72, or CD79) and an anti-human IgE antibody and selecting for cells that express the B cell marker and that express IgE on the cell surface. In some embodiments, the isolating step comprises contacting cells of the sample with an anti-human CD19 antibody and an anti-human IgE antibody and selecting for CD19⁺ IgE-expressing B cells.

In some embodiments, the isolating step comprises contacting cells of the sample with antibody against a B cell marker (e.g., an antibody against CD19, CD20, CD21, CD22, CD23, CD24, CD40, CD72, or CD79), an anti-human IgE antibody, and an antibody against one or more immunoglobulin isotypes (e.g., an anti-human IgG antibody, an anti-human IgM antibody, an anti-human IgA antibody, and/or an anti-human IgD antibody) or subclass thereof and selecting for cells that express the B cell marker, that express IgE on the cell surface, and that do not express detectable levels of the one or more other immunoglobulin isotypes being screened for. In some embodiments, the isolating step comprises contacting cells of the sample with an anti-human CD19 antibody, an anti-human IgE antibody, and one or more of an anti-human IgG antibody, an anti-human IgM antibody, an anti-human IgA antibody, or an anti-human IgD antibody, and selecting for CD19⁺ IgE-expressing B cells that are negative for IgG, IgM, IgA, or IgD cell surface expression.

In some embodiments, the isolating step comprises contacting cells from the sample with an anti-human CD19 antibody, an anti-human IgE antibody, an anti-human IgM antibody, and an anti-human IgG antibody and selecting for CD19⁺ IgM⁻IgG⁻ IgE-expressing B cells. In some embodiments, the isolating step comprises contacting cells from the sample with an anti-human CD19 antibody, an anti-human IgE antibody, an anti-human IgM antibody, an anti-human IgG antibody, an anti-human IgA antibody, and an anti-human IgD antibody and selecting for CD19⁺IgM⁻IgG⁻IgA⁻IgD⁻ IgE-expressing B cells.

In some embodiments, the isolating step comprises contacting cells of the sample with antibody against a B cell marker (e.g., an antibody against CD19, CD20, CD21, CD22, CD23, CD24, CD40, CD72, or CD79) and antibodies against non-IgE isotypes (e.g., an anti-human IgG antibody, an anti-human IgM antibody, an anti-human IgA antibody, and an anti-human IgD antibody) or subclass thereof, and selecting for cells that express the B cell marker and that do not express detectable levels of the non-IgE isotypes. In some embodiments, the isolating step comprises contacting cells from the sample with an anti-human CD19 antibody, an anti-human IgM antibody, an anti-human IgG antibody, an anti-human IgA antibody, and an anti-human IgD antibody and selecting for CD19⁺IgM⁻IgG⁻IgA⁻IgD⁻ B cells.

In some embodiments, the method comprises isolating IgE-expressing B cells that are antibody-secreting B cells (e.g., plasmablasts or plasma cells). In some embodiments, the method comprises isolating IgE-expressing B cells that are memory B cells. In some embodiments, the method comprises isolating IgE-expressing antibody-secreting B cells and IgE-expressing memory B cells.

### IgG4 Selection

In some embodiments, the method comprises isolating single B cells that are selected for expression of cell surface IgG4. In some embodiments, the isolating step comprises contacting cells of the sample with an anti-human IgG4 antibody and selecting for cells that express IgG4 on the cell surface. In some embodiments, the isolating step comprises contacting cells of the sample with an antibody against a B cell marker (e.g., an antibody against CD19, CD20, CD21, CD22, CD23, CD24, CD40, CD72, or CD79) and an anti-human IgG4 antibody and selecting for cells that express the B cell marker and that express IgG4 on the cell surface. In some embodiments, the isolating step comprises contacting cells of the sample with an anti-human CD19 antibody and an anti-human IgG4 antibody and selecting for CD19⁺ IgG4-expressing B cells.

In some embodiments, the isolating step comprises contacting cells of the sample with an antibody against a B cell marker (e.g., an antibody against CD19, CD20, CD21, CD22, CD23, CD24, CD40, CD72, or CD79), an anti-human IgG4 antibody, and an antibody against one or more IgG subclasses (e.g., an anti-human IgG1 antibody, an anti-human IgG2 antibody, an anti-human IgG3 antibody) and selecting for cells that express the B cell marker, that express IgG4 on the cell surface, and that do not express detectable levels of the one or more other IgG subclasses being screened for. In some embodiments, the isolating step comprises contacting cells from the sample with an anti-human CD19 antibody, an anti-human IgG1 antibody, an anti-human IgG2 antibody, an anti-human IgG3 antibody, and an anti-human IgG4 antibody and selecting for CD19⁺IgG1⁻IgG2⁻IgG3⁻ IgG4-expressing B cells.

In some embodiments, the isolating step comprises contacting cells of the sample with an antibody against a B cell marker (e.g., an antibody against CD19, CD20, CD21, CD22, CD23, CD24, CD40, CD72, or CD79), an anti-human IgG4 antibody, and an antibody against one or more immunoglobulin isotypes (e.g., an anti-human IgE antibody, an anti-human IgM antibody, an anti-human IgA antibody, and/or an anti-human IgD antibody) or subclass thereof and selecting for cells that express the B cell marker, that express IgG4 on the cell surface, and that do not express detectable levels of the one or more other immunoglobulin isotypes being screened for. In some embodiments, the isolating step comprises contacting cells of the sample with an anti-human CD19 antibody, an anti-human IgG4 antibody, and one or more of an anti-human IgE antibody, an anti-human IgM antibody, an anti-human IgA antibody, or an anti-human IgD antibody, and selecting for CD19⁺ IgG4-expressing B cells that are negative for IgE, IgM, IgA, or IgD cell surface expression.

In some embodiments, the isolating step comprises contacting cells from the sample with an anti-human CD19 antibody, an anti-human IgG4 antibody, an anti-human IgM antibody, and an anti-human IgG antibody and selecting for CD19⁺IgM⁻IgE⁻IgG4-expressing B cells. In some embodiments, the isolating step comprises contacting cells from the sample with an anti-human CD19 antibody, an anti-human IgG4 antibody, an anti-human IgM antibody, an anti-human IgE antibody, an anti-human IgA antibody, and an anti-human IgD antibody and selecting for CD19⁺IgM⁻IgE⁻IgA⁻IgD⁻ IgG4-expressing B cells.

In some embodiments, the isolating step comprises contacting cells of the sample with an antibody against a B cell marker (e.g., an antibody against CD19, CD20, CD21, CD22, CD23, CD24, CD40, CD72, or CD79) and antibodies against non-IgG isotypes (e.g., an anti-human IgE antibody, an anti-human IgM antibody, an anti-human IgA antibody, and an anti-human IgD antibody) or non-IgG4 isotypes thereof, and selecting for cells that express the B cell marker and that do not express detectable levels of the non-IgG or non-IgG4 isotypes. In some embodiments, the isolating step comprises contacting cells from the sample with an anti-human CD19 antibody, an anti-human IgM antibody, an anti-human IgE antibody, an anti-human IgA antibody, and an anti-human IgD antibody and selecting for CD19⁺IgM⁻IgE⁻IgA⁻IgD⁻ B cells. In some embodiments, the isolating step comprises contacting cells from the sample with an anti-human CD19 antibody, an anti-human IgM antibody, an anti-human IgE antibody, an anti-human IgA antibody, an anti-human IgD antibody, an anti-human IgG1 antibody, an anti-human IgG2 antibody, and an anti-human IgG3 antibody and selecting for CD19⁺IgM⁻IgE⁻IgA⁻IgD⁻IgG1⁻IgG2⁻IgG3⁻ B cells.

In some embodiments, the method comprises isolating IgG4-expressing B cells that are antibody-secreting B cells. In some embodiments, the method comprises isolating IgG4-expressing B cells that are memory B cells. In some embodiments, the method comprises isolating IgG4-expressing antibody-secreting B cells and IgG4-expressing memory B cells.

Generating and Sequencing cDNAs

In some embodiments, cDNAs are generated from the isolated single B cells from the sample (e.g., from single B cells that have been screened for expression of an immunoglobulin isotype such as IgE or IgG4, or from single B cells that have not been screened for expression of an immunoglobulin isotype). In some embodiments, cDNA libraries are prepared from the single B cells. In some embodiments, for the cDNAs that are generated for each single B cell, the cDNA sequences comprise a sequence that encodes an immunoglobulin heavy chain and a sequence that encodes an immunoglobulin light chain.

In some embodiments, cDNAs are generated by reverse transcribing cDNA sequences from RNA (e.g., total RNA or mRNA) from the single B cell and amplifying the cDNA sequences. For generating cDNAs, in some embodiments, the single B cells are lysed and cDNA sequences are reverse transcribed from mRNA present in the cell lysate. In some embodiments, RNA is isolated from the single B cell and cDNAs are reverse transcribed from the isolated RNA.

In some embodiments, the method comprises amplifying the transcriptome of the single B cell. For example, in some embodiments, the method comprises reverse transcribing RNA (e.g., polyadenylated mRNA) to synthesize cDNAs, then amplifying the cDNA, e.g., by PCR. Exemplary methods for reverse transcribing polyadenylated mRNA and amplifying the transcriptome of a single cell are described in Darmanis et al., Cell Reports, 2017, 21:1399-1410, and in Picelli et al., Nature Protocols, 9, 2014, 171-181.

In some embodiments, the method comprises amplifying immunoglobulin heavy chain and light chain sequences from the single B cells. For example, in some embodiments, the method comprises reverse transcribing RNA (e.g., total RNA) to synthesize cDNAs, then amplifying the cDNAs, e.g., by PCR, using primers for immunoglobulin heavy chain variable regions and constant regions. In some embodiments, the method comprises reverse transcribing RNA using immunoglobulin-specific primers (e.g., constant region-specific primers) to synthesize cDNAs comprising immunoglobulin sequences, then amplifying the cDNAs using primers for immunoglobulin heavy chain variable regions and constant regions. An exemplary method for amplifying immunoglobulin heavy chain and light chain sequences from a single cell is described in Tiller et al., J. Immunol. Methods, 2008, 329:112-124.

After the cDNAs are generated, in some embodiments, the method comprises determining the sequences of the cDNAs. In some embodiments, the cDNAs are subjected to sequencing. In some embodiments, the method comprises sequencing the transcriptomes of the single B cells. In some embodiments, the method comprises sequencing target genes (e.g., immunoglobulin genes, e.g., immunoglobulin heavy chain variable regions and constant regions and immunoglobulin light chain variable regions and constant regions).

Sequencing methods, including methods for high-throughput sequencing, are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

In some embodiments, sequencing comprises high-throughput sequencing. In high-throughput sequencing, parallel sequencing reactions using multiple templates and multiple primers allows rapid sequencing of genomes or large portions of genomes. High throughput sequencing methods include methods that typically use template amplification and those that do not. Sequencing methods that utilize amplification include pyrosequencing commercialized by Roche as the 454 technology platforms (e.g., GS 20 and GS FLX), clonal array formation and sequencing by synthesis (SBS) chemistry commercialized by Illumina with systems such as the NextSeq, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

In some embodiments, an Illumina sequencing platform, such as NextSeq, is used. This sequencing technology utilizes clonal array formation and sequencing by synthesis to produce sequences on a large scale. In this method, sequencing templates are immobilized on a flow cell surface, then solid-phase amplification creates copies of each template molecule (up to 1,000 identical copies) in close proximity, forming dense “clusters” of polynucleotide sequences. For sequencing the clusters, fluorescently-labeled nucleotides are used to sequences the clusters on the flow cell surface in parallel. For each sequencing cycle, a single labeled reversible terminator-bound dNTP is added to the nucleic acid chain. The sequence of incorporated nucleotides is determined by detection of post-incorporation fluorescence, then the fluorescent dye is removed prior to the next cycle of dNTP addition, resulting in base-by-base sequencing. Typically sequence read length ranges from about 30 nucleotides to over 150 nucleotides. For a target cDNA of interest having a longer length, the sequence can be bioinformatically reassembled based on overlaps between the short sequencing reads to determine the sequence of the full-length target cDNA.

### Identifying B Cells Having an IgE or IgG4 Isotype

In some embodiments, after the sequences of cDNAs have been determined for the single B cells, the method comprises analyzing the sequences of the cDNAs to identify single B cells that express an immunoglobulin heavy chain having a constant region that is of the IgE isotype and/or of the IgG4 isotype. As described herein, it has been found that determining the isotype of the B cell based on the sequence of the heavy chain transcript, rather than FACS immunoglobulin surface staining, substantially reduces the number of false positive IgE cells in the B cell population, and thus results in a population of B cells that is much more likely to yield antibodies that specifically bind to the allergen to which the human subject who is the source of the B cells is allergic.

In some embodiments, the method comprises identifying a sequence encoding an immunoglobulin heavy chain that comprises an IgE constant region. In some embodiments, the method comprises identifying a sequence encoding an immunoglobulin heavy chain that comprises an IgG4 constant region. In some embodiments, the cDNA sequence is analyzed by comparing the sequence to a known IgE constant region sequence or to a known IgG4 constant region sequence. For example, a comparison of a cDNA sequence of interest (e.g., a “test” sequence from a B cell) can be compared to a known IgE or IgG4 constant region sequence (e.g., a “reference” sequence) by aligning the sequences. Methods of alignment of sequences for comparison are known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site.

For comparing a test sequence to an IgE or IgG4 constant region reference sequence, in some embodiments, the reference sequence is published sequence such as an IgE or IgG4 constant region sequence that is publicly available in the ImMunoGeneTics (IMGT) database. See, e.g., Camacho et al., BMC Bioinformatics, 2009, 10:421; Lefranc et al., Nucleic Acids Res, 2009, 37:D1006-1012. Methods of analyzing test sequences to identify sequences comprising an immunoglobulin heavy chain constant region that is of the IgE isotype and/or of the IgG4 isotype are also described in Table 1 below.

In some embodiments, in addition to analyzing sequences to identify and select single B cells comprising an immunoglobulin heavy chain sequence that comprises an IgE constant region or an IgG4 constant region, the method further comprises determining the sequences and/or levels of expression of one or more other genes in the single B cell. For example, in some embodiments, the method comprises determining the sequences and/or levels of expression of a set of genes that are a “signature” for a particular type of B cell.

In some embodiments, for a B cell that is identified as having an immunoglobulin heavy chain that comprises an IgE constant region or an IgG4 constant region, the method further comprises identifying, from the same B cell, the heavy chain variable region sequence that is expressed by the cell and the light chain variable region sequence that is expressed by the cell.

### Antibody Expression

Typically, for a single B cell that is identified as having a cDNA that comprises an IgE or IgG4 constant region sequence, the heavy chain variable region and light chain variable region sequences from the single B cell are candidate antibody sequences for having specificity to the allergen of interest. Thus, in some embodiments, the method comprises expressing antibodies comprising the heavy chain variable region and light chain variable region sequences from the single B cell and identifying whether the expressed antibody specifically binds to the allergen of interest. Methods for the expression and purification of recombinant antibodies are described in the art. See, e.g., Frenzel et al., Front Immunol., 2013, 4:217, doi:10.3389/fimmu.2013.00217; Siegemund et al., Methods Mol Biol., 2014, 1131:273-295.

In some embodiments, the heavy chain variable region and light chain variable region sequences from the single B cell are amplified from the single B cell and cloned into an expression vector. In some embodiments, the heavy chain variable region and light chain variable region sequences from the single B cell are synthesized. In some embodiments, the heavy chain variable region sequence and/or light chain variable region sequence is codon-optimized, e.g., to increase antibody expression by the expression system. See, e.g., Ayyar et al., Methods, 2017, 116:51-62.

The heavy chain variable region and light chain variable region sequences from the single B cell can be expressed using any number of expression systems, including prokaryotic and eukaryotic expression systems. In some embodiments, the expression system is a mammalian cell expression, such as a hybridoma, or a CHO or HEK293 cell expression system. Many such systems are widely available from commercial suppliers. Cell expression systems are also described in the art. See, e.g., Kunert and Reinhart, 2016, “Advances in recombinant antibody manufacturing” App/Microbiol Biotechnol. 100:3451-61; Jager et al., BMC Proc., 2015, 9:P40, doi:10.1186/1753-6561-9-S9-P40; and references cited therein. In some embodiments, the heavy chain and light chain are expressed using a single vector, e.g., in a di-cistronic expression unit, or under the control of different promoters. In other embodiments, the heavy chain and light chain are be expressed using separate vectors. In some embodiments, an expression vector for expressing heavy chain variable region sequence and/or light chain variable region sequence as disclosed herein is a vector that comprises a constant region of a desired heavy chain isotype or light chain subclass. For example, a heavy chain variable region sequence as disclosed herein can be cloned into a vector that comprises a human IgG (e.g., IgG1, IgG2, IgG3, or IgG4) heavy chain constant region, and a light chain variable region sequence as disclosed herein can be cloned into a vector that comprises a human lambda or kappa light chain constant region.

After an antibody comprising a heavy chain variable region sequence and a light chain variable region sequence from the single B cell as disclosed herein is expressed and purified, in some embodiments, the method comprises determining whether the antibody specifically binds to the allergen. Methods for analyzing binding affinity and binding kinetics are known in the art. See, e.g., Ernst et al., Determination of Equilibrium Dissociation Constants, Therapeutic Monoclonal Antibodies (Wiley & Sons ed. 2009). These methods include, but are not limited to, solid-phase binding assays (e.g., ELISA assay), immunoprecipitation, surface plasmon resonance (SPR, e.g., Biacore™ (GE Healthcare, Piscataway, NJ)), kinetic exclusion assays (e.g. KinExA®), flow cytometry, fluorescence-activated cell sorting (FACS), BioLayer interferometry (e.g., Octet™ (FortéBio, Inc., Menlo Park, CA)), and Western blot analysis. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given wavelength is shined on the surface, the changes to the optical reflectivity of the gold indicate binding, and the kinetics of binding. In some embodiments, kinetic exclusion assays are used to determine affinity. This technique is described, e.g., in Darling et al., Assay and Drug Development Technologies Vol. 2, number 6 647-657 (2004). In some embodiments, BioLayer interferometry assays are used to determine affinity. This technique is described, e.g., in Wilson et al., Biochemistry and Molecular Biology Education, 38:400-407 (2010); Dysinger et al., J. Immunol. Methods, 379:30-41 (2012).

In some embodiments, the expressed antibody specifically binds to the allergen with high affinity. In some embodiments, the antibody has a binding affinity (K_{D}) for the allergen that is less than 250 nM, less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM. In some embodiments, the antibody binds to the allergen with a binding affinity (K_{D}) from 1 nM to 250 nM.

### Nucleic Acids, Vectors, and Host Cells

In some embodiments, the allergen-specific monoclonal antibodies as described herein are prepared using recombinant methods. Accordingly, in some aspects, the invention provides isolated nucleic acids comprising a nucleic acid sequence encoding any of the allergen-specific monoclonal antibodies as described herein (e.g., any one or more of the CDRs described herein); vectors comprising such nucleic acids; and host cells into which the nucleic acids are introduced that are used to replicate the antibody-encoding nucleic acids and/or to express the antibodies. In some embodiments, the host cell is eukaryotic, e.g., a human cell such as HEK-293.

In some embodiments, a polynucleotide (e.g., an isolated polynucleotide) comprises a nucleotide sequence encoding an antibody or antigen-binding portion thereof as described herein (e.g., as described in Section IV below). In some embodiments, the polynucleotide comprises a nucleotide sequence encoding one or more amino acid sequences (e.g., CDR, heavy chain variable region, or light chain variable region) disclosed in Table 1 below.

In a further aspect, methods of making an allergen-specific monoclonal antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. Cloning vectors are available from commercial vendors such as BioRad, Stratagene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

### IV. Monoclonal Antibodies that Specifically Bind to Allergens

In another aspect, allergen-specific monoclonal antibodies, and antigen-binding portions thereof, that are generated from a human sample according to a method disclosed herein are provided. In some embodiments, the monoclonal antibody is an antibody that is generated according to the methods disclosed in Section III above. In some embodiments, the monoclonal antibody is an antibody that is generated from a sample from a human subject having an allergy to a food allergen, a plant allergen, a fungal allergen, an animal allergen, a dust mite allergen, a drug allergen, a cosmetic allergen, or a latex allergen, and the monoclonal antibody specifically binds to the food allergen, plant allergen, fungal allergen, animal allergen, dust mite allergen, drug allergen, cosmetic allergen, or latex allergen.

In some embodiments, an antibody described herein is a full-length antibody, a Fab, a Fab′, a F(ab′)2, a Fab′-SH, an Fv, a single-chain antibody, or a single chain Fv (scFv) antibody. In some embodiments, an antibody described herein comprises an IgG4 constant region. In some embodiments, an antibody described herein is a monospecific antibody. In some embodiments, an antibody described herein is a multispecific antibody. In particular, an antibody described herein can be a bispecific antibody that binds to two different allergens. For example, in some embodiments, an antibody described herein can bind to a peanut allergen and a tree nut allergen. In some embodiments, an antibody described herein can bind to a peanut allergen and a milk allergen. In some embodiments, an antibody described herein can bind to a peanut allergen and a fungal allergen. In some embodiments, an antibody described herein can bind to a tree nut allergen and a milk allergen. In some embodiments, an antibody described herein can bind to a tree nut allergen and a fungal allergen. In some embodiments, an antibody described herein can bind to a milk allergen and a fungal allergen.

In some embodiments, the monoclonal antibody or antigen-binding portion thereof is an allergen-specific antibody that comprises a heavy chain variable region sequence and a light chain variable region sequence that are identified according to a process comprising:
(a) isolating single B cells from a sample from a human subject, wherein the subject is allergic to the allergen;
(b) generating cDNAs from the single B cells of step (a), wherein the cDNA sequences comprise a first sequence that encodes all or part of an immunoglobulin heavy chain and a second sequence that encodes all or part of an immunoglobulin light chain;
(c) determining the sequences of the cDNAs from step (b);
(d) analyzing the sequences determined in step (c) to identify single B cells comprising a first sequence that comprises an IgE constant region or an IgG4 constant region;
(e) identifying, from the single B cells of step (d), (i) a heavy chain variable region sequence in the immunoglobulin heavy chain that comprises an IgE constant region or an IgG4 constant region, and (ii) a light chain variable region sequence in an immunoglobulin light chain that is co-expressed with the immunoglobulin heavy chain in the same single B cell.

In some embodiments, the heavy chain variable region and the light chain variable region are from a B cell comprising an immunoglobulin that comprises an IgE constant region. In some embodiments, the heavy chain variable region and the light chain variable region are from a B cell comprising an immunoglobulin that comprises an IgG4 constant region.

In some embodiments, the monoclonal antibody or antigen-binding portion thereof is an allergen-specific antibody that comprises:
(a) a heavy chain variable region sequence that is derived from an immunoglobulin heavy chain from an IgE- or IgG4-producing single B cell from a human subject who is allergic to the allergen;
(b) a heavy chain IgG constant region sequence;
(c) a light chain variable region sequence that is derived from an immunoglobulin light chain from the IgE- or IgG4-producing single B cell from a human subject;
(d) a light chain constant region sequence that is of the same class as the immunoglobulin light chain of (c).

In some embodiments, the monoclonal antibody comprises a heavy chain variable region sequence and a light chain variable region sequence that are derived from an IgE-producing human B cell. In some embodiments, the monoclonal antibody comprises a heavy chain variable region sequence and a light chain variable region sequence that are derived from an IgG4-producing human B cell.

### Characteristics of Allergen-Specific Monoclonal Antibodies

In some embodiments, the monoclonal antibody is an antibody that specifically binds to a food allergen, a plant allergen, a fungal allergen, an animal allergen, a dust mite allergen, a drug allergen, a cosmetic allergen, or a latex allergen. In some embodiments, the monoclonal antibody is an antibody that specifically binds to a food allergen, such as a milk allergen, an egg allergen, a nut allergen, a fish allergen, a shellfish allergen, a soy allergen, a legume allergen, a seed allergen, or a wheat allergen. In some embodiments, the monoclonal antibody specifically binds to a peanut allergen. In some embodiments, the monoclonal antibody specifically binds to a milk allergen. In some embodiments, the monoclonal antibody specifically binds to an egg allergen.

In some embodiments, the monoclonal antibody specifically binds to the allergen (e.g., a food allergen, a plant allergen, a fungal allergen, an animal allergen, a dust mite allergen, a drug allergen, a cosmetic allergen, or a latex allergen) with a binding affinity (K_{D}) of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM. In some embodiments, the antibody binds to the allergen with a binding affinity (K_{D}) from 1 nM to 250 nM.

In some embodiments, the monoclonal antibody exhibits cross-reactivity with at least two different antigens (e.g., allergens), e.g., at least two food allergens, at least two plant allergens, at least two fungal allergens, at least two animal allergens, at least two dust mite allergens, at least two drug allergens, at least two cosmetic allergens, or at least two latex allergens. In some embodiments, the monoclonal antibody exhibits cross-reactivity with at least two milk allergens, at least two egg allergens, at least two nut allergens, at least two fish allergens, at least two shellfish allergens, at least two soy allergens, at least two legume allergens, at least two seed allergens, or at least two wheat allergens. It will be appreciated by a person of ordinary skill in the art that many different allergens, such as many plant food allergens, can be grouped within a small number of protein families. For example, more than half of all plant food allergens can be categorized into one of the following four structural protein families: the prolamin superfamily, the cupin superfamily, profilins, and Bet v-1-related proteins. It will also be appreciated by a person of ordinary skill in the art that for a particular type of allergen (e.g., a “peanut” allergen), there can be more than one peptide or protein that is an allergen. As a non-limiting example, there are 12 known peanut allergens. See, Mueller et al., Curr Allergy Asthma Rep, 2014, 14:429. In some embodiments, the monoclonal antibody exhibits cross-reactivity with two or more different antigens that are different types or classes of antigens. As a non-limiting example, in some embodiments, a monoclonal antibody exhibits cross-reactivity with an antigen that is a peanut allergen and an antigen that is a nut (e.g., tree nut) allergen.

In embodiments in which the monoclonal antibody exhibits cross-reactivity with at least two different antigens (e.g., allergens), in some embodiments the monoclonal antibody specifically binds to at least one of the allergens with a K_{D} of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM. In some embodiments, the monoclonal antibody specifically binds to the first antigen (e.g., first allergen) with a K_{D} of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM. In some embodiments, the monoclonal antibody specifically binds to the second antigen (e.g., second allergen) with a K_{D} of less than 1 pM, less than 500 nM, less than 100 nM, less than 10 nM, or less than 1 nM.

### Engineered Variations in Variable Regions

In some embodiments, the heavy chain variable region and/or the light chain variable region of the monoclonal antibody has an identical sequence to the heavy chain variable region and/or the light chain variable region encoded by the IgE-producing or IgG4-producing single B cell from the human subject having an allergy to the allergen. In some embodiments, the heavy chain variable region and/or the light chain variable region of the monoclonal antibody comprises one or more modifications, e.g., amino acid substitutions, deletions, or insertions.

As described in the Examples section below, the heavy chain variable region sequence and/or light chain variable region sequence of an antibody described herein (e.g., a peanut allergen-specific monoclonal antibody such as Clone PA13P1H08, Clone PA13P1E10, Clone PA12P3F10, Clone PA13P3G09, Clone PA12P3D08, Clone PA12P1C07, Clone PA15P1D12, Clone PA15P1D05, or a Clone PA13P1H08 variant) can be engineered to comprise one or more variations in the heavy chain variable region sequence and/or light chain variable region sequence. In some embodiments, the engineered variation(s) improves the binding affinity of the antibody for the allergen. In some embodiments, the engineered variation(s) improves the cross-reactivity of the antibody for a second allergen.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655.

In some embodiments, affinity maturation is used to engineer further mutations that enhance the binding affinity of the antibody for the allergen or enhance the cross-reactivity of the antibody for a second allergen. Methods for performing affinity maturation are known in the art. See, e.g., Renaut et al., Methods Mol Biol, 2012, 907:451-461.

### Constant Regions and Isotype Switching

In some embodiments, the monoclonal antibody comprises a heavy chain variable region sequence and a light chain variable region sequence that are derived from an IgE-producing human B cell or from an IgG4-producing human B cell, and further comprises a kappa or lambda light chain constant region. In some embodiments, the light chain constant region (kappa or lambda) is from the same type of light chain (i.e., kappa or lambda) as the light chain variable region that was derived from the IgE-producing human B cell or from an IgG4-producing human B cell; as a non-limiting example, if an IgE-producing human B cell comprises a kappa light chain, then the monoclonal antibody that is produced comprises the light chain variable region from the IgE-producing B cell and further comprises a kappa light chain constant region.

In some embodiments, the monoclonal antibody comprises a heavy chain variable region sequence and a light chain variable region sequence that are derived from an IgE-producing human B cell or from an IgG4-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the monoclonal antibody comprises a native (i.e., wild-type) human IgG, IgA, IgM, or IgD constant region. In some embodiments, the monoclonal antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the monoclonal antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469.

In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the monoclonal antibody comprises a human IgM constant region, human IgD constant region, human IgG constant region that is derived from IgG1, IgG2, IgG3, or IgG4, or human IgA constant region that is derived from IgA1 or IgA2. In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions includes variations (e.g., one, two, three, four, five, six, seven, eight, nine, ten or more amino acid substitutions) that reduce effector function.

In some embodiments, a monoclonal antibody comprises CDR sequences, a heavy chain variable region, and/or a light chain variable region from an antibody from an IgE or IgG4 B cell as described herein (e.g., as disclosed in Table 1 below) and further comprises a heavy chain constant region and/or a light chain constant region that is heterologous to the antibody from the IgE or IgG4 B cell from which the CDR sequences and/or variable region sequences are derived. For example, in some embodiments, the monoclonal antibody comprises the CDR sequences and/or variable region sequences of an antibody from an IgE B cell, and further comprises a heavy chain constant region and a light chain constant region that is heterologous to the antibody from the IgE B cell (e.g., the heavy chain constant region and/or light chain constant region is a wild-type or modified IgG1, IgG2, IgG3, or IgG4 constant region, or the heavy chain constant region and/or light chain constant region comprises one or more modifications (e.g., amino acid substitutions) relative to the native constant region of the antibody from the IgE B cell).

Antibodies that Specifically Bind to Peanut and/or Tree Nut Allergens

In some embodiments, a monoclonal antibody or antigen-binding portion thereof as disclosed herein specifically binds to a peanut allergen and/or a tree nut allergen. In some embodiments, the monoclonal antibody specifically binds to a peanut allergen. In some embodiments, the monoclonal antibody specifically binds to a peanut allergen that is Ara h 1, Ara h 2, Ara h 3, or Ara h 6.

In some embodiments, the monoclonal antibody exhibits cross-reactivity with at least two peanut allergens. In some embodiments, the monoclonal antibody exhibits cross-reactivity with two or more of the peanut allergens Ara h 1, Ara h 2, Ara h 3, and Ara h 6. In some embodiments, the monoclonal antibody specifically binds to at least one of the peanut allergens with a K_{D} of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM. In some embodiments, the monoclonal antibody specifically binds to a first peanut allergen with a K_{D} of less than 100 nM (e.g., less than 50 nM, less than 10 nM, less than 5 nM, or less than 1 nM) and specifically binds to a second peanut allergen with a K_{D} of less than 1 pM (e.g., less than 500 nM, less than 100 nM, less than 10 nM, or less than 1 nM). In some embodiments, the monoclonal antibody specifically binds to Ara h 2 with a K_{D} of less than 100 nM (e.g., less than 50 nM, less than 10 nM, less than 5 nM, or less than 1 nM) and specifically binds to Ara h 1, Ara h 3, or Ara h 6 with a K_{D} of less than 1 IpM (e.g., less than 500 nM, less than 100 nM, less than 10 nM, or less than 1 nM).

In some embodiments, the monoclonal antibody recognizes an epitope that comprises or consists of the amino acid motif DPYSPS (SEQ ID NO:704). In some embodiments, the monoclonal antibody recognizes an epitope that comprises or consists of the amino acid sequence DSYGRDPYSPS (SEQ ID NO:705), YSPSQDPYSPS (SEQ ID NO:706), or PDRRDPYSPS (SEQ ID NO:707).

In some embodiments, the monoclonal antibody or antigen-binding portion thereof specifically binds to a tree nut allergen. In some embodiments, the tree nut allergen is a cashew, pistachio, almond, pine nut, pecan, walnut, hazelnut, or macadamia nut allergen. In some embodiments, the monoclonal antibody exhibits cross-reactivity with at least two tree nut allergens. In some embodiments, the monoclonal antibody exhibits cross-reactivity with both cashew and pistachio allergens. In some embodiments, the monoclonal antibody exhibits cross-reactivity with both pecan and walnut allergens. In some embodiments, the monoclonal antibody exhibits cross-reactivity with two or more of pecan, walnut, hazelnut, and macadamia nut allergens. In some embodiments, the monoclonal antibody specifically binds to at least one of the tree nut allergens with a K_{D} of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM.

In some embodiments, the monoclonal antibody or antigen-binding portion thereof specifically binds to a peanut allergen and to a tree nut allergen. In some embodiments, the monoclonal antibody specifically binds to a peanut allergen and to one or more (e.g., 1, 2, 3, 4, or more) of a cashew, pistachio, almond, pine nut, pecan, walnut, hazelnut, or macadamia nut allergen.

### Peanut-Specific Antibody Sequences

In some embodiments, a monoclonal antibody that specifically binds to a peanut allergen (e.g., that specifically binds to Ara h 1, Ara h 2, Ara h 3, or Ara h 6) comprises heavy chain CDRs and/or light chain CDRs that are disclosed in Table 1 below. In some embodiments, a monoclonal antibody that specifically binds to a peanut allergen comprises a light chain variable region sequence and/or a heavy chain variable region sequence sequence that is disclosed in Table 1 below. In some embodiments, a monoclonal antibody that specifically binds to a peanut allergen comprises: a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to a heavy chain variable region sequence disclosed in Table 1 and comprises a CDR1, a CDR2, and a CDR3 that is identical to the CDRs of that heavy chain variable region sequence, and a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity to a light chain variable region sequence disclosed in Table 1 and comprises a CDR1, a CDR2, and a CDR3 that is identical to the CDRs of that light chain variable region sequence.

In some embodiments, a monoclonal antibody that specifically binds to a peanut allergen (e.g., that specifically binds to Ara h 1, Ara h 2, Ara h 3, or Ara h 6) comprises a light chain sequence, or a portion thereof, and/or a heavy chain sequence, or a portion thereof, derived from any of the following antibodies described herein: Clone PA13P1H08, Clone PA13P1E10, Clone PA12P3F10, Clone PA13P3G09, Clone PA12P3D08, Clone PA12P1C07, Clone PA15P1D12, Clone PA15P1D05, a variant of Clone PA13P1H08 (e.g., an R-R variant, an R-N variant, an N-R variant, an rCDR1-N variant, an rCDR2-N variant, an rCDR3-N variant, or an rFWRs-N variant of Clone PA13P1H08), Clone PA12P4D02, Clone PA12P3E09, Clone PA12P3E11, Clone PA12P1D02, Clone PA12P1G11, Clone PA13P1H03, Clone PA12P3C01, or Clone PA12P3E04. The amino acid sequences of the CDR, light chain variable domain (VL), and heavy chain variable domain (VH) of Clone PA13P1H08, Clone PA13P1E10, Clone PA12P3F10, Clone PA13P3G09, Clone PA12P3D08, Clone PA12P1C07, Clone PA15P1D12, Clone PA15P1D05, Clone PA13P1H08 variants, Clone PA12P4D02, Clone PA12P3E09, Clone PA12P3E11, Clone PA12P1D02, Clone PA12P1G11, Clone PA13P1H03, Clone PA12P3C01, and Clone PA12P3E04 are set forth in Table 1 below.

In some embodiments, a monoclonal antibody that specifically binds to a peanut allergen comprises one or more (e.g., one, two, three, four, five, or all six) of:
(a) a heavy chain CDR1 comprising the amino acid sequence of any one of SEQ ID NOs:2, 10, 17, 25, 33, 41, 47, 58, 113, 129, 199, 341, 348, 409, 459, or 593;
(b) a heavy chain CDR2 comprising the amino acid sequence of any one of SEQ ID NOs:3, 11, 18, 26, 34, 48, 59, 130, 200, 342, 349, 410, 460, 539, or 594;
(c) a heavy chain CDR3 comprising the amino acid sequence of any one of SEQ ID NOs:4, 12, 19, 27, 35, 42, 49, 55, 60, 131, 201, 350, 411, 461, 540, or 595;
(d) a light chain CDR1 comprising the amino acid sequence of any one of SEQ ID NOs:6, 14, 21, 29, 37, 44, 51, 62, 133, 203, 343, 352, 413, 463, 542, or 597;
(e) a light chain CDR2 comprising the amino acid sequence of any one of SEQ ID NOs:7, 15, 22, 30, 38, 52, 78, 86, 126, 149, 196, 345, 353, or 598; and
(f) a light chain CDR3 comprising the amino acid sequence of any one of SEQ ID NOs:8, 23, 31, 39, 45, 53, 63, 134, 204, 346, 354, 414, 464, 543, or 599.

In some embodiments, a monoclonal antibody that specifically binds to a peanut allergen comprises a heavy chain CDR1-3 and a light chain CDR1-3 comprising the amino acid sequences of:
(a) SEQ ID NOs: 2, 3, 4, 6, 7, and 8, respectively; or
(b) SEQ ID NOs: 10, 11, 12, 14, 15, and 8, respectively; or
(c) SEQ ID NOs: 17, 18, 19, 21, 22, and 23, respectively; or
(d) SEQ ID NOs: 25, 26, 27, 29, 30, and 31, respectively; or
(e) SEQ ID NOs: 33, 34, 35, 37, 38, and 39, respectively; or
(f) SEQ ID NOs: 41, 34, 35, 37, 38, and 39, respectively; or
(g) SEQ ID NOs: 47, 48, 49, 51, 52, and 53, respectively; or
(h) SEQ ID NOs: 47, 48, 55, 51, 52, and 53, respectively; or
(i) SEQ ID NOs: 58, 59, 60, 62, 30, and 63, respectively; or
(j) SEQ ID NOs: 58, 59, 60, 6, 7, and 8, respectively; or
(k) SEQ ID NOs: 2, 3, 4, 62, 30, and 63, respectively; or
(l) SEQ ID NOs: 58, 3, 4, 6, 7, and 8, respectively; or
(m) SEQ ID NOs: 2, 59, 4, 6, 7, and 8, respectively; or
(n) SEQ ID NOs: 2, 3, 60, 6, 7, and 8, respectively; or
(o) SEQ ID NOs:129, 130, 131, 133, 126, and 134, respectively; or
(p) SEQ ID NOs:341, 342, 343, 345, 78, and 346, respectively; or
(q) SEQ ID NOs:348, 349, 350, 352, 353, and 354, respectively; or
(r) SEQ ID NOs:199, 200, 201, 203, 149, and 204, respectively; or
(s) SEQ ID NOs:409, 410, 411, 413, 86, and 414, respectively; or
(t) SEQ ID NOs:459, 460, 461, 463, 196, and 464, respectively; or
(u) SEQ ID NOs:113, 539, 540, 542, 196, and 543, respectively; or
(v) SEQ ID NOs:593, 594, 595, 597, 598, and 599, respectively.

In some embodiments, a monoclonal antibody that specifically binds to a peanut allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:1, 9, 16, 24, 32, 40, 46, 54, 57, 64, 65, 66, 67, 128, 340, 347, 406, 408, 458, 538, or 592. In some embodiments, a monoclonal antibody that specifically binds to a peanut allergen comprises a light chain variable region comprising an amino acid sequence that has at least 75% sequence identity (e.g., at least 75%, 80%, 85%, 90%, 91%, 92%, 93%, 94%, 95%, 96%, 97%, 98%, or 99% sequence identity) to any one of SEQ ID NOs:5, 13, 20, 28, 36, 43, 50, 56, 61, 132, 344, 351, 407, 412, 462, 541, or 596. In some embodiments, a monoclonal antibody that specifically binds to a peanut allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:1, 9, 16, 24, 32, 40, 46, 54, 57, 64, 65, 66, 67, 128, 340, 347, 406, 408, 458, 538, or 592, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs: 5, 13, 20, 28, 36, 43, 50, 56, 61, 132, 344, 351, 407, 412, 462, 541, or 596.

In some embodiments, a monoclonal antibody that specifically binds to a peanut allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs: 1, 9, 16, 24, 32, 40, 46, 54, 57, 64, 65, 66, 67, 128, 340, 347, 406, 408, 458, 538, or 592 and comprises a CDR1, a CDR2, and a CDR3 that is identical to the CDRs of that SEQ ID NO. In some embodiments, a monoclonal antibody that specifically binds to a peanut allergen comprises a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity) to any one of SEQ ID NOs: 5, 13, 20, 28, 36, 43, 50, 56, 61, 132, 344, 351, 407, 412, 462, 541, or 596 and comprises a CDR1, a CDR2, and a CDR3 that is identical to the CDRs of that SEQ ID NO. In some embodiments, a monoclonal antibody that specifically binds to a peanut allergen comprises:
(a) a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs: 1, 9, 16, 24, 32, 40, 46, 54, 57, 64, 65, 66, 67, 128, 340, 347, 406, 408, 458, 538, or 592 and that comprises a CDR1, a CDR2, and a CDR3 that is identical to the CDRs of that SEQ ID NO; and
(b) a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs: 5, 13, 20, 28, 36, 43, 50, 56, 61, 132, 344, 351, 407, 412, 462, 541, or 596 and that comprises a CDR1, a CDR2, and a CDR3 that is identical to the CDRs of that SEQ ID NO.

### Clone PA13P1H08

In some embodiments, a monoclonal antibody that specifically binds to a peanut allergen comprises a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, and that comprises a heavy chain CDR1, CDR2, and CDR3 comprising the amino acid sequences of SEQ ID NOs:2, 3, and 4, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:5, and that comprises the light chain CDR1, CDR2, and CDR3 comprising the amino acid sequences of SEQ ID NOs:6, 7, and 8, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:1, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:5. In some embodiments, the antibody comprises a heavy chain variable region comprising the amino acid sequence of SEQ ID NO:1 and comprises a light chain variable region comprising the amino acid sequence of SEQ ID NO:5.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:2, 3, 4, 6, 7, and 8, respectively, or an antibody comprising a heavy chain variable region comprising the amino acid sequence of SEQ ID NO:1 and further comprising a light chain variable region comprising the amino acid sequence of SEQ ID NO:5).

### Clone PA13P1E10

In some embodiments, a monoclonal antibody that specifically binds to a peanut allergen comprises a heavy chain CDR1 comprising the amino acid sequence of SEQ ID NO:10, a heavy chain CDR2 comprising the amino acid sequence of SEQ ID NO:11, a heavy chain CDR3 comprising the amino acid sequence of SEQ ID NO:12, a light chain CDR1 comprising the amino acid sequence of SEQ ID NO:14, a light chain CDR2 comprising the amino acid sequence of SEQ ID NO:15, and a light chain CDR3 comprising the amino acid sequence of SEQ ID NO:8.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:9, and that comprises a heavy chain CDR1, CDR2, and CDR3 comprising the amino acid sequences of SEQ ID NOs:10, 11, and 12, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:13, and that comprises the light chain CDR1, CDR2, and CDR3 comprising the amino acid sequences of SEQ ID NOs:14, 15, and 8, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:9, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:13. In some embodiments, the antibody comprises a heavy chain variable region comprising the amino acid sequence of SEQ ID NO:9 and comprises a light chain variable region comprising the amino acid sequence of SEQ ID NO:13.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:10, 11, 12, 14, 15, and 8 respectively, or an antibody comprising a heavy chain variable region comprising the amino acid sequence of SEQ ID NO:9 and further comprising a light chain variable region comprising the amino acid sequence of SEQ ID NO:13).

### Clone PA12P3F10

In some embodiments, a monoclonal antibody that specifically binds to a peanut allergen comprises a heavy chain CDR1 comprising the amino acid sequence of SEQ ID NO:17, a heavy chain CDR2 comprising the amino acid sequence of SEQ ID NO:18, a heavy chain CDR3 comprising the amino acid sequence of SEQ ID NO:19, a light chain CDR1 comprising the amino acid sequence of SEQ ID NO:21, a light chain CDR2 comprising the amino acid sequence of SEQ ID NO:22, and a light chain CDR3 comprising the amino acid sequence of SEQ ID NO:23.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:16, and that comprises a heavy chain CDR1, CDR2, and CDR3 comprising the amino acid sequences of SEQ ID NOs:17, 18, and 19, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:20, and that comprises the light chain CDR1, CDR2, and CDR3 comprising the amino acid sequences of SEQ ID NOs:21, 22, and 23, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:16, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:20. In some embodiments, the antibody comprises a heavy chain variable region comprising the amino acid sequence of SEQ ID NO:16 and comprises a light chain variable region comprising the amino acid sequence of SEQ ID NO:20.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:17, 18, 19, 21, 22, and 23, respectively, or an antibody comprising a heavy chain variable region comprising the amino acid sequence of SEQ ID NO:16 and further comprising a light chain variable region comprising the amino acid sequence of SEQ ID NO:20).

### Clone PA13P3G09

In some embodiments, a monoclonal antibody that specifically binds to a peanut allergen comprises a heavy chain CDR1 comprising the amino acid sequence of SEQ ID NO:25, a heavy chain CDR2 comprising the amino acid sequence of SEQ ID NO:26, a heavy chain CDR3 comprising the amino acid sequence of SEQ ID NO:27, a light chain CDR1 comprising the amino acid sequence of SEQ ID NO:29, a light chain CDR2 comprising the amino acid sequence of SEQ ID NO:30, and a light chain CDR3 comprising the amino acid sequence of SEQ ID NO:31.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:24, and that comprises a heavy chain CDR1, CDR2, and CDR3 comprising the amino acid sequences of SEQ ID NOs:25, 26, and 27, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:28, and that comprises the light chain CDR1, CDR2, and CDR3 comprising the amino acid sequences of SEQ ID NOs:29, 30, and 31, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:24, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:28. In some embodiments, the antibody comprises a heavy chain variable region comprising the amino acid sequence of SEQ ID NO:24 and comprises a light chain variable region comprising the amino acid sequence of SEQ ID NO:28.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:25, 26, 27, 29, 30, and 31, respectively, or an antibody comprising a heavy chain variable region comprising the amino acid sequence of SEQ ID NO:24 and further comprising a light chain variable region comprising the amino acid sequence of SEQ ID NO:28).

### Clone PA12P3D08

In some embodiments, a monoclonal antibody that specifically binds to a peanut allergen comprises a heavy chain CDR1 comprising the amino acid sequence of SEQ ID NO:33, a heavy chain CDR2 comprising the amino acid sequence of SEQ ID NO:34, a heavy chain CDR3 comprising the amino acid sequence of SEQ ID NO:35, a light chain CDR1 comprising the amino acid sequence of SEQ ID NO:37, a light chain CDR2 comprising the amino acid sequence of SEQ ID NO:38, and a light chain CDR3 comprising the amino acid sequence of SEQ ID NO:39.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:32, and that comprises a heavy chain CDR1, CDR2, and CDR3 comprising the amino acid sequences of SEQ ID NOs:33, 34, and 35, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:36, and that comprises the light chain CDR1, CDR2, and CDR3 comprising the amino acid sequences of SEQ ID NOs:37, 38, and 39, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:32, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:36. In some embodiments, the antibody comprises a heavy chain variable region comprising the amino acid sequence of SEQ ID NO:32 and comprises a light chain variable region comprising the amino acid sequence of SEQ ID NO:36.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:33, 34, 35, 37, 38, and 39, respectively, or an antibody comprising a heavy chain variable region comprising the amino acid sequence of SEQ ID NO:32 and further comprising a light chain variable region comprising the amino acid sequence of SEQ ID NO:36).

### Clone PA12P1C07

In some embodiments, a monoclonal antibody that specifically binds to a peanut allergen comprises a heavy chain CDR1 comprising the amino acid sequence of SEQ ID NO:41, a heavy chain CDR2 comprising the amino acid sequence of SEQ ID NO:34, a heavy chain CDR3 comprising the amino acid sequence of SEQ ID NO:42, a light chain CDR1 comprising the amino acid sequence of SEQ ID NO:44, a light chain CDR2 comprising the amino acid sequence of SEQ ID NO:30, and a light chain CDR3 comprising the amino acid sequence of SEQ ID NO:45.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:40, and that comprises a heavy chain CDR1, CDR2, and CDR3 comprising the amino acid sequences of SEQ ID NOs:41, 34, and 42, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:43, and that comprises the light chain CDR1, CDR2, and CDR3 comprising the amino acid sequences of SEQ ID NOs:44, 30, and 45, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:40, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:43. In some embodiments, the antibody comprises a heavy chain variable region comprising the amino acid sequence of SEQ ID NO:40 and comprises a light chain variable region comprising the amino acid sequence of SEQ ID NO:43.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:41, 34, 42, 44, 30, and 45, respectively, or an antibody comprising a heavy chain variable region comprising the amino acid sequence of SEQ ID NO:40 and further comprising a light chain variable region comprising the amino acid sequence of SEQ ID NO:43).

### Clone PA15P1D12

In some embodiments, a monoclonal antibody that specifically binds to a peanut allergen comprises a heavy chain CDR1 comprising the amino acid sequence of SEQ ID NO:47, a heavy chain CDR2 comprising the amino acid sequence of SEQ ID NO:48, a heavy chain CDR3 comprising the amino acid sequence of SEQ ID NO:49, a light chain CDR1 comprising the amino acid sequence of SEQ ID NO:51, a light chain CDR2 comprising the amino acid sequence of SEQ ID NO:52, and a light chain CDR3 comprising the amino acid sequence of SEQ ID NO:53.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:46, and that comprises a heavy chain CDR1, CDR2, and CDR3 comprising the amino acid sequences of SEQ ID NOs:47, 48, and 49, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:50, and that comprises the light chain CDR1, CDR2, and CDR3 comprising the amino acid sequences of SEQ ID NOs:51, 52, and 53, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:46, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:50. In some embodiments, the antibody comprises a heavy chain variable region comprising the amino acid sequence of SEQ ID NO:46 and comprises a light chain variable region comprising the amino acid sequence of SEQ ID NO:50.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:41, 34, 42, 44, 30, and 45, respectively, or an antibody comprising a heavy chain variable region comprising the amino acid sequence of SEQ ID NO:46 and further comprising a light chain variable region comprising the amino acid sequence of SEQ ID NO:50).

### Clone PA15P1D05

In some embodiments, a monoclonal antibody that specifically binds to a peanut allergen comprises a heavy chain CDR1 comprising the amino acid sequence of SEQ ID NO:47, a heavy chain CDR2 comprising the amino acid sequence of SEQ ID NO:48, a heavy chain CDR3 comprising the amino acid sequence of SEQ ID NO:55, a light chain CDR1 comprising the amino acid sequence of SEQ ID NO:51, a light chain CDR2 comprising the amino acid sequence of SEQ ID NO:52, and a light chain CDR3 comprising the amino acid sequence of SEQ ID NO:53.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:54, and that comprises a heavy chain CDR1, CDR2, and CDR3 comprising the amino acid sequences of SEQ ID NOs:47, 48, and 55, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:56, and that comprises the light chain CDR1, CDR2, and CDR3 comprising the amino acid sequences of SEQ ID NOs:51, 52, and 53, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:54, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:56. In some embodiments, the antibody comprises a heavy chain variable region comprising the amino acid sequence of SEQ ID NO:54 and comprises a light chain variable region comprising the amino acid sequence of SEQ ID NO:56.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:47, 48, 55, 51, 52, and 53, respectively, or an antibody comprising a heavy chain variable region comprising the amino acid sequence of SEQ ID NO:54 and further comprising a light chain variable region comprising the amino acid sequence of SEQ ID NO:56).

### Variant Sequences

In some embodiments, the allergen-specific monoclonal antibody comprises one or more variations (e.g., amino acid substitutions) in one or more CDR, heavy chain, and/or light chain sequences as disclosed herein (e.g., one or more mutations in one or more CDR, heavy chain, and/or light chain sequences of Clone PA13P1H08, Clone PA13P1E10, Clone PA12P3F10, Clone PA13P3G09, Clone PA12P3D08, Clone PA12P1C07, Clone PA15P1D12, Clone PA15P1D05).

In some embodiments, one or more substitutions are made in a CDR, heavy chain, or light chain sequence of Clone PA13P1H08. As described in the Examples section below, variants were made of Clone PA13P1H08, in which one or more regions in the heavy chain and/or light chain were reverted to the inferred naïve rearrangement. It was found that antibody sequences comprising a naïve light chain or heavy chain sequence were capable of binding to the peanut allergen Ara h 2. It was also surprisingly found that a variant of Clone PA13P1H08 comprising a reverted CDR-H2 sequence exhibited significantly improved cross-reactivity to a second peanut allergen (Ara h 3) in addition to having sub-nanomolar affinity for the Ara h 2 peanut allergen. Thus, in some embodiments, the mutation is an amino acid substitution that reverts at least a portion of the sequence of the clone from its “native” form (i.e., the CDR, heavy chain variable region, or light chain variable region sequence of the clone as disclosed in Table 1) to the inferred naïve immunoglobulin sequence.

In some embodiments, an allergen-specific monoclonal antibody comprises one or more variant sequences of a Clone PA13P1H08 variant as disclosed herein. In some embodiments, the antibody comprises one of the sequences of Clone PA13P1H08 variant “R-R,” in which both the heavy chain variable region and the light chain variable region of Clone PA13P1H08 are reverted back to the inferred naïve rearrangement. In some embodiments, the antibody comprises a reverted heavy chain variable region sequence comprising SEQ ID NO:57. In some embodiments, the antibody comprises a reverted light chain variable region sequence comprising SEQ ID NO:61.

In some embodiments, the antibody comprises one of the sequences of Clone PA13P1H08 variant “R-N,” in which the heavy chain variable region of Clone PA13P1H08 is reverted back to the inferred naïve rearrangement and the light chain variable region retains the native sequence of Clone PA13P1H08 (i.e., SEQ ID NO:5). In some embodiments, the antibody comprises one of the sequences of Clone PA13P1H08 variant “N-R,” in which the heavy chain variable region retains the native sequence of Clone PA13P1H08 (i.e., SEQ ID NO:1), and the light chain variable region is reverted back to the inferred naïve rearrangement of Clone PA13P1H08.

In some embodiments, the antibody comprises one or more reverted CDR sequences, e.g., one or more reverted heavy chain CDR sequences, and/or one or more reverted light chain CDR sequences. In some embodiments, the antibody comprises one or more of a reverted CDR-H1 comprising SEQ ID NO:58, a reverted CDR-H2 comprising SEQ ID NO:59, or a reverted CDR-H3 comprising SEQ ID NO:60. In some embodiments, the antibody comprises one or more of a reverted CDR-L1 comprising SEQ ID NO:62, a reverted CDR-L2 comprising SEQ ID NO:30, or a reverted CDR-L3 comprising SEQ ID NO:63. In some embodiments, the antibody comprises one or more reverted framework regions, e.g., the heavy chain variable region comprising reverted framework regions of SEQ ID NO:67.

In some embodiments, a monoclonal antibody that specifically binds to a peanut allergen comprises a heavy chain CDR1 comprising the amino acid sequence of SEQ ID NO:2 or SEQ ID NO:58, a heavy chain CDR2 comprising the amino acid sequence of SEQ ID NO:3 or SEQ ID NO:59, a heavy chain CDR3 comprising the amino acid sequence of SEQ ID NO:4 or SEQ ID NO:60, a light chain CDR1 comprising the amino acid sequence of SEQ ID NO:6 or SEQ ID NO:62, a light chain CDR2 comprising the amino acid sequence of SEQ ID NO:7 or SEQ ID NO:30, and a light chain CDR3 comprising the amino acid sequence of SEQ ID NO:8 or SEQ ID NO:63.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:1, SEQ ID NO:57, SEQ ID NO:64, SEQ ID NO:65, SEQ ID NO:66, or SEQ ID NO:67, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:5 or SEQ ID NO:61. In some embodiments, the antibody comprises a heavy chain variable region comprising the amino acid sequence of SEQ ID NO:57 and comprises a light chain variable region comprising the amino acid sequence of SEQ ID NO:61. In some embodiments, the antibody comprises a heavy chain variable region comprising the amino acid sequence of SEQ ID NO:57 and comprises a light chain variable region comprising the amino acid sequence of SEQ ID NO:5. In some embodiments, the antibody comprises a heavy chain variable region comprising the amino acid sequence of SEQ ID NO:1 and comprises a light chain variable region comprising the amino acid sequence of SEQ ID NO:61. In some embodiments, the antibody comprises a heavy chain variable region comprising the amino acid sequence of SEQ ID NO:64 and comprises a light chain variable region comprising the amino acid sequence of SEQ ID NO:5. In some embodiments, the antibody comprises a heavy chain variable region comprising the amino acid sequence of SEQ ID NO:65 and comprises a light chain variable region comprising the amino acid sequence of SEQ ID NO:5. In some embodiments, the antibody comprises a heavy chain variable region comprising the amino acid sequence of SEQ ID NO:66 and comprises a light chain variable region comprising the amino acid sequence of SEQ ID NO:5. In some embodiments, the antibody comprises a heavy chain variable region comprising the amino acid sequence of SEQ ID NO:67 and comprises a light chain variable region comprising the amino acid sequence of SEQ ID NO:5.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:65, and that comprises a heavy chain CDR1, CDR2, and CDR3 comprising the amino acid sequences of SEQ ID NOs:2, 59, and 4, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:5, and that comprises the light chain CDR1, CDR2, and CDR3 comprising the amino acid sequences of SEQ ID NOs:6, 7, and 8, respectively.

### Tree Nut-Specific Antibody Sequences

In some embodiments, a monoclonal antibody that specifically binds to a tree nut allergen (e.g., that specifically binds to a cashew, pistachio, almond, pine nut, pecan, walnut, hazelnut, or macadamia nut allergen) comprises heavy chain CDRs and/or light chain CDRs that are disclosed in Table 1 below. In some embodiments, a monoclonal antibody that specifically binds to a tree nut allergen comprises a light chain variable region sequence and/or a heavy chain variable region sequence sequence that is disclosed in Table 1 below. In some embodiments, a monoclonal antibody that specifically binds to a tree nut allergen comprises: a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to a heavy chain variable region sequence disclosed in Table 1 and comprises a CDR1, a CDR2, and a CDR3 that is identical to the CDRs of that heavy chain variable region sequence, and a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity to a light chain variable region sequence disclosed in Table 1 and comprises a CDR1, a CDR2, and a CDR3 that is identical to the CDRs of that light chain variable region sequence.

In some embodiments, a monoclonal antibody that specifically binds to a tree nut allergen comprises a light chain sequence, or a portion thereof, and/or a heavy chain sequence, or a portion thereof, derived from any of the following antibodies described herein: Clone PA14P3H08, Clone PA11P1D11, Clone PA11P1G10, Clone PA12P4D02, Clone PA11P1D12, Clone PA11P1F03, Clone PA11P1C04, Clone PA11P1G04, Clone PA11P1E01, Clone PA11P1C11, or Clone PA11P1C03. The amino acid sequences of the CDR, light chain variable domain (VL), and heavy chain variable domain (VH) of Clone PA14P3H08, Clone PA11P1D11, Clone PA11P1G10, Clone PA12P4D02, Clone PA11P1D12, Clone PA11P1F03, Clone PA11P1C04, Clone PA11P1G04, Clone PA11P1E01, Clone PA11P1C11, and Clone PA11P1C03 are set forth in Table 1 below.

In some embodiments, a monoclonal antibody that specifically binds to a tree nut allergen comprises one or more (e.g., one, two, three, four, five, or all six) of:
(a) a heavy chain CDR1 comprising the amino acid sequence of any one of SEQ ID NOs: 113, 167, 175, 227, 311, 318, 438, 466, 621, 665, or 692;
(b) a heavy chain CDR2 comprising the amino acid sequence of any one of SEQ ID NOs: 168, 176, 200, 312, 319, 439, 539, 666, or 693;
(c) a heavy chain CDR3 comprising the amino acid sequence of any one of SEQ ID NOs: 169, 177, 228, 313, 320, 440, 467, 540, 667, or 694;
(d) a light chain CDR1 comprising the amino acid sequence of any one of SEQ ID NOs: 171, 179, 230, 315, 322, 442, 469, 542, 623, 669, or 696;
(e) a light chain CDR2 comprising the amino acid sequence of any one of SEQ ID NOs: 30, 94, 149, 172, 180, 196, 323, or 670; and
(f) a light chain CDR3 comprising the amino acid sequence of any one of SEQ ID NOs: 173, 181, 231, 316, 324, 443, 470, 543, 624, 671, or 697.

In some embodiments, a monoclonal antibody that specifically binds to a tree nut allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs:166, 174, 226, 310, 317, 437, 465, 538, 620, 664, or 691. In some embodiments, a monoclonal antibody that specifically binds to a peanut allergen comprises a light chain variable region comprising an amino acid sequence that comprises the sequence of, or has at least 75% sequence identity (e.g., at least 75%, 80%, 85%, 90%, 91%, 92%, 93%, 94%, 95%, 96%, 97%, 98%, or 99% sequence identity) to, any one of SEQ ID NOs:170, 178, 229, 314, 321, 441, 468, 541, 622, 668, or 695. In some embodiments, a monoclonal antibody that specifically binds to a peanut allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity to, any one of SEQ ID NOs:166, 174, 226, 310, 317, 437, 465, 538, 620, 664, or 691, and comprises a light chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs:170, 178, 229, 314, 321, 441, 468, 541, 622, 668, or 695.

In some embodiments, a monoclonal antibody that specifically binds to a tree nut allergen comprises:
(a) a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:166, 174, 226, 310, 317, 437, 465, 538, 620, 664, or 691 and that comprises a CDR1, a CDR2, and a CDR3 that is identical to the CDRs of that SEQ ID NO; and
(b) a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:170, 178, 229, 314, 321, 441, 468, 541, 622, 668, or 695 and that comprises a CDR1, a CDR2, and a CDR3 that is identical to the CDRs of that SEQ ID NO.

In some embodiments, the monoclonal antibody specifically binds to a pecan, walnut, hazelnut, and/or macadamia nut allergen and comprises one or more (e.g., one, two, three, four, five, or all six) of:
(a) a heavy chain CDR1 comprising the amino acid sequence of any one of SEQ ID NOs: 113, 227, 311, 318, 665, or 692;
(b) a heavy chain CDR2 comprising the amino acid sequence of any one of SEQ ID NOs: 200, 312, 319, 539, 666, or 693;
(c) a heavy chain CDR3 comprising the amino acid sequence of any one of SEQ ID NOs: 228, 313, 320, 540, 667, or 694;
(d) a light chain CDR1 comprising the amino acid sequence of any one of SEQ ID NOs: 230, 315, 322, 542, 669, or 696;
(e) a light chain CDR2 comprising the amino acid sequence of any one of SEQ ID NOs: 94, 149, 196, 323, or 670; and
(f) a light chain CDR3 comprising the amino acid sequence of any one of SEQ ID NOs: 231, 316, 324, 543, 671, or 697.

In some embodiments, a monoclonal antibody that specifically binds to a pecan, walnut, hazelnut, and/or macadamia nut allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs: 226, 310, 317, 538, 664, or 691. In some embodiments, a monoclonal antibody that specifically binds to a pecan, walnut, hazelnut, and/or macadamia nut allergen comprises a light chain variable region comprising an amino acid sequence that comprises the sequence of, or has at least 75% sequence identity (e.g., at least 75%, 80%, 85%, 90%, 91%, 92%, 93%, 94%, 95%, 96%, 97%, 98%, or 99% sequence identity) to, any one of SEQ ID NOs: 229, 314, 321, 541, 668, or 695. In some embodiments, a monoclonal antibody that specifically binds to a pecan, walnut, hazelnut, and/or macadamia nut allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity to, any one of SEQ ID NOs: 226, 310, 317, 437, 538, 664, or 691, and comprises a light chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs: 229, 314, 321, 541, 668, or 695.

In some embodiments, the monoclonal antibody specifically binds to a cashew and/or pistachio allergen and comprises one or more (e.g., one, two, three, four, five, or all six) of:
(a) a heavy chain CDR1 comprising the amino acid sequence of any one of SEQ ID NOs: 167, 175, 227, 438, 466, or 621;
(b) a heavy chain CDR2 comprising the amino acid sequence of any one of SEQ ID NOs: 168, 176, 200, or 439;
(c) a heavy chain CDR3 comprising the amino acid sequence of any one of SEQ ID NOs: 169, 177, 440, or 467;
(d) a light chain CDR1 comprising the amino acid sequence of any one of SEQ ID NOs: 171, 179, 442, 469, or 623;
(e) a light chain CDR2 comprising the amino acid sequence of any one of SEQ ID NOs: 30, 149, 172, or 180; and
(f) a light chain CDR3 comprising the amino acid sequence of any one of SEQ ID NOs: 173, 181, 443, 470, or 624.

In some embodiments, a monoclonal antibody that specifically binds to a cashew and/or pistachio allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs:166, 174, 437, 465, or 620. In some embodiments, a monoclonal antibody that specifically binds to a cashew and/or pistachio allergen comprises a light chain variable region comprising an amino acid sequence that comprises the sequence of, or has at least 75% sequence identity (e.g., at least 75%, 80%, 85%, 90%, 91%, 92%, 93%, 94%, 95%, 96%, 97%, 98%, or 99% sequence identity) to, any one of SEQ ID NOs:170, 178, 441, 468, or 622. In some embodiments, a monoclonal antibody that specifically binds to a cashew and/or pistachio allergen comprises a heavy chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity to, any one of SEQ ID NOs:166, 174, 437, 465, or 620, and comprises a light chain variable region comprising an amino acid sequence that comprises the sequence of, or has at least 75% sequence identity (e.g., at least 80%, 85%, 90%, 91%, 92%, 93%, 94%, 95%, 96%, 97%, 98%, or 99% sequence identity) to, any one of SEQ ID NOs:170, 178, 441, 468, or 622.

In some embodiments, a monoclonal antibody that specifically binds to a tree nut allergen comprises a heavy chain CDR1-3 and a light chain CDR1-3 comprising the amino acid sequences of:
(a) SEQ ID NOs:692, 693, 694, 696, 94, and 697, respectively; or
(b) SEQ ID NOs:318, 319, 320, 322, 323, and 324, respectively; or
(c) SEQ ID NOs:227, 200, 228, 230, 149, and 231, respectively; or
(d) SEQ ID NOs:113, 539, 540, 542, 196, and 543, respectively; or
(e) SEQ ID NOs:311, 312, 313, 315, 94, and 316, respectively; or
(f) SEQ ID NOs:665, 666, 667, 669, 670, and 671, respectively; or
(g) SEQ ID NOs:466, 200, 467, 469, 149, and 470, respectively; or
(h) SEQ ID NOs:167, 168, 169, 171, 172, and 173, respectively; or
(i) SEQ ID NOs:621, 176, 177, 623, 180, and 624, respectively; or
(j) SEQ ID NOs:175, 176, 177, 179, 180, and 181, respectively; or
(k) SEQ ID NOs:438, 439, 440, 442, 30, and 443, respectively.

### Clone PA14P3H08

In some embodiments, a monoclonal antibody that specifically binds to a tree nut allergen comprises a heavy chain CDR1 comprising the amino acid sequence of SEQ ID NO:692, a heavy chain CDR2 comprising the amino acid sequence of SEQ ID NO:693, a heavy chain CDR3 comprising the amino acid sequence of SEQ ID NO:694, a light chain CDR1 comprising the amino acid sequence of SEQ ID NO:696, a light chain CDR2 comprising the amino acid sequence of SEQ ID NO:94, and a light chain CDR3 comprising the amino acid sequence of SEQ ID NO:697. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:691, and that comprises a heavy chain CDR1, CDR2, and CDR3 comprising the amino acid sequences of SEQ ID NOs:692, 693, and 694, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:695, and that comprises the light chain CDR1, CDR2, and CDR3 comprising the amino acid sequences of SEQ ID NOs:696, 94, and 697, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:691, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:695. In some embodiments, the antibody comprises a heavy chain variable region comprising the amino acid sequence of SEQ ID NO:691 and comprises a light chain variable region comprising the amino acid sequence of SEQ ID NO:695.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:692, 693, 694, 696, 95, and 697, respectively, or an antibody comprising a heavy chain variable region comprising the amino acid sequence of SEQ ID NO:691 and further comprising a light chain variable region comprising the amino acid sequence of SEQ ID NO:695).

### Clone PA11P1D11

In some embodiments, a monoclonal antibody that specifically binds to a tree nut allergen comprises a heavy chain CDR1 comprising the amino acid sequence of SEQ ID NO:318, a heavy chain CDR2 comprising the amino acid sequence of SEQ ID NO:319, a heavy chain CDR3 comprising the amino acid sequence of SEQ ID NO:320, a light chain CDR1 comprising the amino acid sequence of SEQ ID NO:322, a light chain CDR2 comprising the amino acid sequence of SEQ ID NO:323, and a light chain CDR3 comprising the amino acid sequence of SEQ ID NO:324. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:317, and that comprises a heavy chain CDR1, CDR2, and CDR3 comprising the amino acid sequences of SEQ ID NOs:318, 319, and 320, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:321, and that comprises the light chain CDR1, CDR2, and CDR3 comprising the amino acid sequences of SEQ ID NOs:322, 323, and 324, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:317, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:321. In some embodiments, the antibody comprises a heavy chain variable region comprising the amino acid sequence of SEQ ID NO:317 and comprises a light chain variable region comprising the amino acid sequence of SEQ ID NO:321.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:318, 319, 320, 322, 323, and 324, respectively, or an antibody comprising a heavy chain variable region comprising the amino acid sequence of SEQ ID NO:317 and further comprising a light chain variable region comprising the amino acid sequence of SEQ ID NO:321).

### Clone PA11P1G10

In some embodiments, a monoclonal antibody that specifically binds to a tree nut allergen comprises a heavy chain CDR1 comprising the amino acid sequence of SEQ ID NO:227, a heavy chain CDR2 comprising the amino acid sequence of SEQ ID NO:200, a heavy chain CDR3 comprising the amino acid sequence of SEQ ID NO:228, a light chain CDR1 comprising the amino acid sequence of SEQ ID NO:230, a light chain CDR2 comprising the amino acid sequence of SEQ ID NO:149, and a light chain CDR3 comprising the amino acid sequence of SEQ ID NO:231. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:226, and that comprises a heavy chain CDR1, CDR2, and CDR3 comprising the amino acid sequences of SEQ ID NOs:227, 200, and 228, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:229, and that comprises the light chain CDR1, CDR2, and CDR3 comprising the amino acid sequences of SEQ ID NOs:230, 149, and 231, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:226, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:229. In some embodiments, the antibody comprises a heavy chain variable region comprising the amino acid sequence of SEQ ID NO:226 and comprises a light chain variable region comprising the amino acid sequence of SEQ ID NO:229.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:227, 200, 228, 230, 149, and 231, respectively, or an antibody comprising a heavy chain variable region comprising the amino acid sequence of SEQ ID NO:226 and further comprising a light chain variable region comprising the amino acid sequence of SEQ ID NO:229).

### Clone PA12P4D02

In some embodiments, a monoclonal antibody that specifically binds to a tree nut allergen comprises a heavy chain CDR1 comprising the amino acid sequence of SEQ ID NO:113, a heavy chain CDR2 comprising the amino acid sequence of SEQ ID NO:539, a heavy chain CDR3 comprising the amino acid sequence of SEQ ID NO:540, a light chain CDR1 comprising the amino acid sequence of SEQ ID NO:542, a light chain CDR2 comprising the amino acid sequence of SEQ ID NO:196, and a light chain CDR3 comprising the amino acid sequence of SEQ ID NO:543. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:538, and that comprises a heavy chain CDR1, CDR2, and CDR3 comprising the amino acid sequences of SEQ ID NOs:113, 539, and 540, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:541, and that comprises the light chain CDR1, CDR2, and CDR3 comprising the amino acid sequences of SEQ ID NOs:542, 196, and 543, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:538, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:541. In some embodiments, the antibody comprises a heavy chain variable region comprising the amino acid sequence of SEQ ID NO:538 and comprises a light chain variable region comprising the amino acid sequence of SEQ ID NO:541.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:113, 539, 540, 542, 196, and 543, respectively, or an antibody comprising a heavy chain variable region comprising the amino acid sequence of SEQ ID NO:538 and further comprising a light chain variable region comprising the amino acid sequence of SEQ ID NO:541).

### Clone PA11P1D12

In some embodiments, a monoclonal antibody that specifically binds to a tree nut allergen comprises a heavy chain CDR1 comprising the amino acid sequence of SEQ ID NO:692, a heavy chain CDR2 comprising the amino acid sequence of SEQ ID NO:693, a heavy chain CDR3 comprising the amino acid sequence of SEQ ID NO:694, a light chain CDR1 comprising the amino acid sequence of SEQ ID NO:696, a light chain CDR2 comprising the amino acid sequence of SEQ ID NO:94, and a light chain CDR3 comprising the amino acid sequence of SEQ ID NO:697. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:310, and that comprises a heavy chain CDR1, CDR2, and CDR3 comprising the amino acid sequences of SEQ ID NOs:311, 312, and 313, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:314, and that comprises the light chain CDR1, CDR2, and CDR3 comprising the amino acid sequences of SEQ ID NOs:315, 94, and 316, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:310, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:314. In some embodiments, the antibody comprises a heavy chain variable region comprising the amino acid sequence of SEQ ID NO:310 and comprises a light chain variable region comprising the amino acid sequence of SEQ ID NO:314.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:311, 312, 313, 315, 94, and 316, respectively, or an antibody comprising a heavy chain variable region comprising the amino acid sequence of SEQ ID NO:310 and further comprising a light chain variable region comprising the amino acid sequence of SEQ ID NO:314).

### Clone PA11P1F03

In some embodiments, a monoclonal antibody that specifically binds to a tree nut allergen comprises a heavy chain CDR1 comprising the amino acid sequence of SEQ ID NO:665, a heavy chain CDR2 comprising the amino acid sequence of SEQ ID NO:666, a heavy chain CDR3 comprising the amino acid sequence of SEQ ID NO:667, a light chain CDR1 comprising the amino acid sequence of SEQ ID NO:669, a light chain CDR2 comprising the amino acid sequence of SEQ ID NO:670, and a light chain CDR3 comprising the amino acid sequence of SEQ ID NO:671. In some embodiments, the monoclonal antibody binds to two or more tree nut allergens. In some embodiments, the monoclonal antibody binds to a pecan, walnut, hazelnut, and/or macadamia nut allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:664, and that comprises a heavy chain CDR1, CDR2, and CDR3 comprising the amino acid sequences of SEQ ID NOs:665, 666, and 667, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:668, and that comprises the light chain CDR1, CDR2, and CDR3 comprising the amino acid sequences of SEQ ID NOs:669, 670, and 671, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:664, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:668. In some embodiments, the antibody comprises a heavy chain variable region comprising the amino acid sequence of SEQ ID NO:664 and comprises a light chain variable region comprising the amino acid sequence of SEQ ID NO:668.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:665, 666, 667, 669, 670, and 671, respectively, or an antibody comprising a heavy chain variable region comprising the amino acid sequence of SEQ ID NO:664 and further comprising a light chain variable region comprising the amino acid sequence of SEQ ID NO:668).

### Clone PA11P1C04

In some embodiments, a monoclonal antibody that specifically binds to a tree nut allergen comprises a heavy chain CDR1 comprising the amino acid sequence of SEQ ID NO:466, a heavy chain CDR2 comprising the amino acid sequence of SEQ ID NO:200, a heavy chain CDR3 comprising the amino acid sequence of SEQ ID NO:467, a light chain CDR1 comprising the amino acid sequence of SEQ ID NO:469, a light chain CDR2 comprising the amino acid sequence of SEQ ID NO:149, and a light chain CDR3 comprising the amino acid sequence of SEQ ID NO:470. In some embodiments, the monoclonal antibody binds to two or more tree nut allergens. In some embodiments, the monoclonal antibody binds to a cashew and/or a pistachio allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:465, and that comprises a heavy chain CDR1, CDR2, and CDR3 comprising the amino acid sequences of SEQ ID NOs:466, 200, and 467, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:468, and that comprises the light chain CDR1, CDR2, and CDR3 comprising the amino acid sequences of SEQ ID NOs:469, 149, and 470, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:465, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:468. In some embodiments, the antibody comprises a heavy chain variable region comprising the amino acid sequence of SEQ ID NO:465 and comprises a light chain variable region comprising the amino acid sequence of SEQ ID NO:468.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:466, 200, 467, 469, 149, and 470, respectively, or an antibody comprising a heavy chain variable region comprising the amino acid sequence of SEQ ID NO:465 and further comprising a light chain variable region comprising the amino acid sequence of SEQ ID NO:468).

### Clone PA11P1G04

In some embodiments, a monoclonal antibody that specifically binds to a tree nut allergen comprises a heavy chain CDR1 comprising the amino acid sequence of SEQ ID NO:167, a heavy chain CDR2 comprising the amino acid sequence of SEQ ID NO:168, a heavy chain CDR3 comprising the amino acid sequence of SEQ ID NO:169, a light chain CDR1 comprising the amino acid sequence of SEQ ID NO:171, a light chain CDR2 comprising the amino acid sequence of SEQ ID NO:172, and a light chain CDR3 comprising the amino acid sequence of SEQ ID NO:173. In some embodiments, the monoclonal antibody binds to two or more tree nut allergens. In some embodiments, the monoclonal antibody binds to a cashew and/or a pistachio allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:166, and that comprises a heavy chain CDR1, CDR2, and CDR3 comprising the amino acid sequences of SEQ ID NOs:167, 168, and 169, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:170, and that comprises the light chain CDR1, CDR2, and CDR3 comprising the amino acid sequences of SEQ ID NOs:171, 172, and 173, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:166, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:170. In some embodiments, the antibody comprises a heavy chain variable region comprising the amino acid sequence of SEQ ID NO:166 and comprises a light chain variable region comprising the amino acid sequence of SEQ ID NO:170.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:167, 168, 169, 171, 172, and 173, respectively, or an antibody comprising a heavy chain variable region comprising the amino acid sequence of SEQ ID NO:166 and further comprising a light chain variable region comprising the amino acid sequence of SEQ ID NO:170).

### Clone PA11P1E01

In some embodiments, a monoclonal antibody that specifically binds to a tree nut allergen comprises a heavy chain CDR1 comprising the amino acid sequence of SEQ ID NO:621, a heavy chain CDR2 comprising the amino acid sequence of SEQ ID NO:176, a heavy chain CDR3 comprising the amino acid sequence of SEQ ID NO:177, a light chain CDR1 comprising the amino acid sequence of SEQ ID NO:623, a light chain CDR2 comprising the amino acid sequence of SEQ ID NO:180, and a light chain CDR3 comprising the amino acid sequence of SEQ ID NO:624. In some embodiments, the monoclonal antibody binds to two or more tree nut allergens. In some embodiments, the monoclonal antibody binds to a cashew and/or a pistachio allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:620, and that comprises a heavy chain CDR1, CDR2, and CDR3 comprising the amino acid sequences of SEQ ID NOs:621, 176, and 177, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:622, and that comprises the light chain CDR1, CDR2, and CDR3 comprising the amino acid sequences of SEQ ID NOs:623, 180, and 624, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:620, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:622. In some embodiments, the antibody comprises a heavy chain variable region comprising the amino acid sequence of SEQ ID NO:620 and comprises a light chain variable region comprising the amino acid sequence of SEQ ID NO:622.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:621, 176, 177, 623, 180, and 624, respectively, or an antibody comprising a heavy chain variable region comprising the amino acid sequence of SEQ ID NO:620 and further comprising a light chain variable region comprising the amino acid sequence of SEQ ID NO:622).

### Clone PA11P1C11

In some embodiments, a monoclonal antibody that specifically binds to a tree nut allergen comprises a heavy chain CDR1 comprising the amino acid sequence of SEQ ID NO:175, a heavy chain CDR2 comprising the amino acid sequence of SEQ ID NO:176, a heavy chain CDR3 comprising the amino acid sequence of SEQ ID NO:177, a light chain CDR1 comprising the amino acid sequence of SEQ ID NO:179, a light chain CDR2 comprising the amino acid sequence of SEQ ID NO:180, and a light chain CDR3 comprising the amino acid sequence of SEQ ID NO:181. In some embodiments, the monoclonal antibody binds to two or more tree nut allergens. In some embodiments, the monoclonal antibody binds to a cashew and/or a pistachio allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:174, and that comprises a heavy chain CDR1, CDR2, and CDR3 comprising the amino acid sequences of SEQ ID NOs:175, 176, and 177, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:178, and that comprises the light chain CDR1, CDR2, and CDR3 comprising the amino acid sequences of SEQ ID NOs:179, 180, and 181, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:174, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:178. In some embodiments, the antibody comprises a heavy chain variable region comprising the amino acid sequence of SEQ ID NO:174 and comprises a light chain variable region comprising the amino acid sequence of SEQ ID NO:178.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:175, 176, 177, 179, 180, and 181, respectively, or an antibody comprising a heavy chain variable region comprising the amino acid sequence of SEQ ID NO:174 and further comprising a light chain variable region comprising the amino acid sequence of SEQ ID NO:178).

### Clone PA11P1C03

In some embodiments, a monoclonal antibody that specifically binds to a tree nut allergen comprises a heavy chain CDR1 comprising the amino acid sequence of SEQ ID NO:438, a heavy chain CDR2 comprising the amino acid sequence of SEQ ID NO:439, a heavy chain CDR3 comprising the amino acid sequence of SEQ ID NO:440, a light chain CDR1 comprising the amino acid sequence of SEQ ID NO:442, a light chain CDR2 comprising the amino acid sequence of SEQ ID NO:30, and a light chain CDR3 comprising the amino acid sequence of SEQ ID NO:443. In some embodiments, the monoclonal antibody binds to two or more tree nut allergens. In some embodiments, the monoclonal antibody binds to a cashew and/or a pistachio allergen.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:437, and that comprises a heavy chain CDR1, CDR2, and CDR3 comprising the amino acid sequences of SEQ ID NOs:438, 439, and 440, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:441, and that comprises the light chain CDR1, CDR2, and CDR3 comprising the amino acid sequences of SEQ ID NOs:442, 30, and 443, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:437, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:441. In some embodiments, the antibody comprises a heavy chain variable region comprising the amino acid sequence of SEQ ID NO:437 and comprises a light chain variable region comprising the amino acid sequence of SEQ ID NO:441.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:438, 439, 440, 442, 30, and 443, respectively, or an antibody comprising a heavy chain variable region comprising the amino acid sequence of SEQ ID NO:437 and further comprising a light chain variable region comprising the amino acid sequence of SEQ ID NO:441).

Antibodies that Specifically Bind to Milk Allergens

In some embodiments, the monoclonal antibody or antigen-binding portion thereof specifically binds to a milk allergen (e.g., cow's milk allergen). In some embodiments, a monoclonal antibody that specifically binds to a milk allergen comprises heavy chain CDRs and/or light chain CDRs that are disclosed in Table 1 below. In some embodiments, a monoclonal antibody that specifically binds to a milk allergen comprises a light chain variable region sequence and/or a heavy chain variable region sequence sequence that is disclosed in Table 1 below. In some embodiments, a monoclonal antibody that specifically binds to a milk allergen comprises: a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to a heavy chain variable region sequence disclosed in Table 1 and comprises a CDR1, a CDR2, and a CDR3 that is identical to the CDRs of that heavy chain variable region sequence, and a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity to a light chain variable region sequence disclosed in Table 1 and comprises a CDR1, a CDR2, and a CDR3 that is identical to the CDRs of that light chain variable region sequence.

In some embodiments, a monoclonal antibody that specifically binds to a milk allergen comprises a light chain sequence, or a portion thereof, and/or a heavy chain sequence, or a portion thereof, derived from any of the following antibodies described herein: Clone PA01P2C05, Clone PA01P2B03, Clone PA01P2A12, Clone PA01P2C12, Clone PA01P2E10, Clone PA01P2C09, Clone PA01P2D06, Clone PA01P2E08, Clone PA01P2A05, Clone PA01P2B04, Clone PA01P2E05, Clone PA01P2D04, Clone PA01P2B12, Clone PA01P2D11, Clone PA01P2B10, Clone PA01P2D10, Clone PA01P2D09, Clone PA01P2B05, Clone PA01P4C11, Clone PA01P3E08, Clone PA01P2E06, Clone PA01P2E07, Clone PA01P2G07, Clone PA01P2B09, Clone PA01P2C04, or Clone PA01P2H08. The amino acid sequences of the CDR, light chain variable domain (VL), and heavy chain variable domain (VH) of Clone PA01P2C05, Clone PA01P2B03, Clone PA01P2A12, Clone PA01P2C12, Clone PA01P2E10, Clone PA01P2C09, Clone PA01P2D06, Clone PA01P2E08, Clone PA01P2A05, Clone PA01P2B04, Clone PA01P2E05, Clone PA01P2D04, Clone PA01P2B12, Clone PA01P2D11, Clone PA01P2B10, Clone PA01P2D10, Clone PA01P2D09, Clone PA01P2B05, Clone PA01P4C11, Clone PA01P3E08, Clone PA01P2E06, Clone PA01P2E07, Clone PA01P2G07, Clone PA01P2B09, Clone PA01P2C04, and Clone PA01P2H08 are set forth in Table 1 below.

In some embodiments, a monoclonal antibody that specifically binds to a peanut allergen comprises one or more (e.g., one, two, three, four, five, or all six) of:
(a) a heavy chain CDR1 comprising the amino acid sequence of any one of SEQ ID NOs: 121, 750, 757, 765, 772, 779, 785, 793, 800, 807, 814, 821, 833, 838, 846, 853, 860, 868, 874, 881, 889, 895, 903, 911, 918, or 926;
(b) a heavy chain CDR2 comprising the amino acid sequence of any one of SEQ ID NOs: 378, 532, 751, 758, 766, 773, 786, 794, 801, 808, 815, 822, 826, 839, 847, 854, 861, 875, 882, 890, 896, 904, 912, 919, or 927;
(c) a heavy chain CDR3 comprising the amino acid sequence of any one of SEQ ID NOs: 752, 759, 767, 774, 780, 787, 795, 802, 809, 816, 827, 840, 848, 855, 862, 869, 876, 883, 891, 897, 905, 913, 920, or 928;
(d) a light chain CDR1 comprising the amino acid sequence of any one of SEQ ID NOs: 523, 754, 761, 769, 776, 782, 789, 797, 804, 811, 818, 829, 835, 842, 850, 857, 864, 871, 878, 885, 899, 907, 915, 922, or 930;
(e) a light chain CDR2 comprising the amino acid sequence of any one of SEQ ID NOs: 22, 30, 94, 110, 149, 186, 196, 389, 404, 509, 662, 682, 762, 790, 830, 843, 865, 886, 900, 908, 923, or 931; and
(f) a light chain CDR3 comprising the amino acid sequence of any one of SEQ ID NOs: 755, 763, 770, 777, 783, 791, 798, 805, 812, 819, 824, 831, 836, 844, 851, 858, 866, 872, 879, 887, 893, 901, 909, 916, 924, or 932.

In some embodiments, a monoclonal antibody that specifically binds to a milk allergen comprises a heavy chain CDR1-3 and a light chain CDR1-3 comprising the amino acid sequences of:
(a) SEQ ID NOs:750, 751, 752, 754, 196, and 755, respectively; or
(b) SEQ ID NOs:757, 758, 759, 761, 762, and 763, respectively; or
(c) SEQ ID NOs:765, 766, 767, 769, 30, and 770, respectively; or
(d) SEQ ID NOs:772, 773, 774, 776, 389, and 777, respectively; or
(e) SEQ ID NOs:779, 532, 780, 782, 404, and 783, respectively; or
(f) SEQ ID NOs:785, 786, 787, 789, 790, and 791, respectively; or
(g) SEQ ID NOs:793, 794, 795, 797, 30, and 798, respectively; or
(h) SEQ ID NOs:800, 801, 802, 804, 110, and 805, respectively; or
(i) SEQ ID NOs:807, 808, 809, 811, 509, and 812, respectively; or
(j) SEQ ID NOs:814, 815, 816, 818, 94, and 819, respectively; or
(k) SEQ ID NOs:821, 822, 787, 776, 790, and 824, respectively; or
(l) SEQ ID NOs:121, 826, 827, 829, 830, and 831, respectively; or
(m) SEQ ID NOs:833, 826, 827, 835, 149, and 836, respectively; or
(n) SEQ ID NOs:838, 839, 840, 842, 843, and 844, respectively; or
(o) SEQ ID NOs:846, 847, 848, 850, 196, and 851, respectively; or
(p) SEQ ID NOs:853, 854, 855, 857, 662, and 858, respectively; or
(q) SEQ ID NOs:860, 861, 862, 864, 865, and 866, respectively; or
(r) SEQ ID NOs:868, 378, 869, 871, 682, and 872, respectively; or
(s) SEQ ID NOs:874, 875, 876, 878, 22, and 879, respectively; or
(t) SEQ ID NOs:881, 882, 883, 885, 886, and 887, respectively; or
(u) SEQ ID NOs:889, 890, 891, 523, 762, and 893, respectively; or
(v) SEQ ID NOs:895, 896, 897, 899, 900, and 901, respectively; or
(w) SEQ ID NOs:903, 904, 905, 907, 908, and 909, respectively; or
(x) SEQ ID NOs:911, 912, 913, 915, 149, and 916, respectively; or
(y) SEQ ID NOs:918, 919, 920, 922, 923, and 924, respectively; or
(z) SEQ ID NOs:926, 927, 928, 930, 931, and 932, respectively.

In some embodiments, a monoclonal antibody that specifically binds to a milk allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:749, 756, 764, 771, 778, 784, 792, 799, 806, 813, 820, 825, 832, 837, 845, 852, 859, 867, 873, 880, 888, 894, 902, 910, 917, or 925. In some embodiments, a monoclonal antibody that specifically binds to a milk allergen comprises a light chain variable region comprising an amino acid sequence that has at least 75% sequence identity (e.g., at least 75%, 80%, 85%, 90%, 91%, 92%, 93%, 94%, 95%, 96%, 97%, 98%, or 99% sequence identity) to any one of SEQ ID NOs:753, 760, 768, 775, 781, 788, 796, 803, 810, 817, 823, 828, 834, 841, 849, 856, 863, 870, 877, 884, 892, 898, 906, 914, 921, or 929. In some embodiments, a monoclonal antibody that specifically binds to a milk allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:749, 756, 764, 771, 778, 784, 792, 799, 806, 813, 820, 825, 832, 837, 845, 852, 859, 867, 873, 880, 888, 894, 902, 910, 917, or 925, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:753, 760, 768, 775, 781, 788, 796, 803, 810, 817, 823, 828, 834, 841, 849, 856, 863, 870, 877, 884, 892, 898, 906, 914, 921, or 929.

In some embodiments, a monoclonal antibody that specifically binds to a milk allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity) to any one of SEQ ID NOs:749, 756, 764, 771, 778, 784, 792, 799, 806, 813, 820, 825, 832, 837, 845, 852, 859, 867, 873, 880, 888, 894, 902, 910, 917, or 925 and comprises a CDR1, a CDR2, and a CDR3 that is identical to the CDRs of that SEQ ID NO. In some embodiments, a monoclonal antibody that specifically binds to a milk allergen comprises a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity) to any one of SEQ ID NOs:753, 760, 768, 775, 781, 788, 796, 803, 810, 817, 823, 828, 834, 841, 849, 856, 863, 870, 877, 884, 892, 898, 906, 914, 921, or 929 and comprises a CDR1, a CDR2, and a CDR3 that is identical to the CDRs of that SEQ ID NO.

In some embodiments, a monoclonal antibody that specifically binds to a milk allergen comprises:
(a) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:749 or that has the sequence of SEQ ID NO:749, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:753 or that has the sequence of SEQ ID NO:753; or
(b) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:756 or that has the sequence of SEQ ID NO:756, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:760 or that has the sequence of SEQ ID NO:760; or
(c) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:764 or that has the sequence of SEQ ID NO:764, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:768 or that has the sequence of SEQ ID NO:768; or
(d) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:771 or that has the sequence of SEQ ID NO:771, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:775 or that has the sequence of SEQ ID NO:775; or
(e) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:778 or that has the sequence of SEQ ID NO:778, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:781 or that has the sequence of SEQ ID NO:781; or
(f) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:784 or that has the sequence of SEQ ID NO:784, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:788 or that has the sequence of SEQ ID NO:788; or
(g) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:792 or that has the sequence of SEQ ID NO:792, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:796 or that has the sequence of SEQ ID NO:796; or
(h) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:799 or that has the sequence of SEQ ID NO:799, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:803 or that has the sequence of SEQ ID NO:803; or
(i) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:806 or that has the sequence of SEQ ID NO:806, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:810 or that has the sequence of SEQ ID NO:810; or
(j) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:813 or that has the sequence of SEQ ID NO:813, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:817 or that has the sequence of SEQ ID NO:817; or
(k) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:820 or that has the sequence of SEQ ID NO:820, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:823 or that has the sequence of SEQ ID NO:823; or
(l) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:825 or that has the sequence of SEQ ID NO:825, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:828 or that has the sequence of SEQ ID NO:828; or
(m) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:832 or that has the sequence of SEQ ID NO:832, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:834 or that has the sequence of SEQ ID NO:834; or
(n) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:837 or that has the sequence of SEQ ID NO:837, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:841 or that has the sequence of SEQ ID NO:841; or
(o) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:845 or that has the sequence of SEQ ID NO:845, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:849 or that has the sequence of SEQ ID NO:849; or
(p) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:852 or that has the sequence of SEQ ID NO:852, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:856 or that has the sequence of SEQ ID NO:856; or
(q) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:859 or that has the sequence of SEQ ID NO:859, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:863 or that has the sequence of SEQ ID NO:863; or
(r) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:867 or that has the sequence of SEQ ID NO:867, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:870 or that has the sequence of SEQ ID NO:870; or
(s) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:873 or that has the sequence of SEQ ID NO:873, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:877 or that has the sequence of SEQ ID NO:877; or
(t) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:880 or that has the sequence of SEQ ID NO:880, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:884 or that has the sequence of SEQ ID NO:884; or
(u) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:888 or that has the sequence of SEQ ID NO:888, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:892 or that has the sequence of SEQ ID NO:892; or
(v) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:894 or that has the sequence of SEQ ID NO:894, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:898 or that has the sequence of SEQ ID NO:898; or
(w) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:902 or that has the sequence of SEQ ID NO:902, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:906 or that has the sequence of SEQ ID NO:906; or
(x) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:910 or that has the sequence of SEQ ID NO:910, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:914 or that has the sequence of SEQ ID NO:914; or
(y) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:917 or that has the sequence of SEQ ID NO:917, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:921 or that has the sequence of SEQ ID NO:921; or
(z) a heavy chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:925 or that has the sequence of SEQ ID NO:925, and a light chain variable region comprising an amino acid sequence that has at least 90% (e.g., at least 75%, 80%, 85%, 90%, 91%, 92%, 93%, 94%, 95%, 96%, 97%, 98%, or 99%) sequence identity to SEQ ID NO:929 or that has the sequence of SEQ ID NO:929.

### Clone PA01P2D09

In some embodiments, a monoclonal antibody that specifically binds to a milk allergen comprises a heavy chain CDR1 comprising the amino acid sequence of SEQ ID NO:860, a heavy chain CDR2 comprising the amino acid sequence of SEQ ID NO:861, a heavy chain CDR3 comprising the amino acid sequence of SEQ ID NO:862, a light chain CDR1 comprising the amino acid sequence of SEQ ID NO:864, a light chain CDR2 comprising the amino acid sequence of SEQ ID NO:865, and a light chain CDR3 comprising the amino acid sequence of SEQ ID NO:866.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:859, and that comprises a heavy chain CDR1, CDR2, and CDR3 comprising the amino acid sequences of SEQ ID NOs:860, 861, and 862, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:863, and that comprises the light chain CDR1, CDR2, and CDR3 comprising the amino acid sequences of SEQ ID NOs:864, 865, and 866, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:859, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:863. In some embodiments, the antibody comprises a heavy chain variable region comprising the amino acid sequence of SEQ ID NO:859 and comprises a light chain variable region comprising the amino acid sequence of SEQ ID NO:863.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:860, 861, 862, 864, 865, and 866, respectively, or an antibody comprising a heavy chain variable region comprising the amino acid sequence of SEQ ID NO:859 and further comprising a light chain variable region comprising the amino acid sequence of SEQ ID NO:863).

### Clone PA01P2D04

In some embodiments, a monoclonal antibody that specifically binds to a milk allergen comprises a heavy chain CDR1 comprising the amino acid sequence of SEQ ID NO:121, a heavy chain CDR2 comprising the amino acid sequence of SEQ ID NO:826, a heavy chain CDR3 comprising the amino acid sequence of SEQ ID NO:827, a light chain CDR1 comprising the amino acid sequence of SEQ ID NO:829, a light chain CDR2 comprising the amino acid sequence of SEQ ID NO:830, and a light chain CDR3 comprising the amino acid sequence of SEQ ID NO:831.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:825, and that comprises a heavy chain CDR1, CDR2, and CDR3 comprising the amino acid sequences of SEQ ID NOs:121, 826, and 827, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:828, and that comprises the light chain CDR1, CDR2, and CDR3 comprising the amino acid sequences of SEQ ID NOs:829, 830, and 831, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:825, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:828. In some embodiments, the antibody comprises a heavy chain variable region comprising the amino acid sequence of SEQ ID NO:825 and comprises a light chain variable region comprising the amino acid sequence of SEQ ID NO:828.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:121, 826, 827, 829, 830, and 831, respectively, or an antibody comprising a heavy chain variable region comprising the amino acid sequence of SEQ ID NO:825 and further comprising a light chain variable region comprising the amino acid sequence of SEQ ID NO:828).

### Clone PA01P2B12

In some embodiments, a monoclonal antibody that specifically binds to a milk allergen comprises a heavy chain CDR1 comprising the amino acid sequence of SEQ ID NO:833, a heavy chain CDR2 comprising the amino acid sequence of SEQ ID NO:826, a heavy chain CDR3 comprising the amino acid sequence of SEQ ID NO:827, a light chain CDR1 comprising the amino acid sequence of SEQ ID NO:835, a light chain CDR2 comprising the amino acid sequence of SEQ ID NO:149, and a light chain CDR3 comprising the amino acid sequence of SEQ ID NO:836.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:832, and that comprises a heavy chain CDR1, CDR2, and CDR3 comprising the amino acid sequences of SEQ ID NOs:833, 826, and 827, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:834, and that comprises the light chain CDR1, CDR2, and CDR3 comprising the amino acid sequences of SEQ ID NOs:835, 149, and 836, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:832, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:834. In some embodiments, the antibody comprises a heavy chain variable region comprising the amino acid sequence of SEQ ID NO:832 and comprises a light chain variable region comprising the amino acid sequence of SEQ ID NO:834.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:833, 826, 827, 835, 149, and 836, respectively, or an antibody comprising a heavy chain variable region comprising the amino acid sequence of SEQ ID NO:832 and further comprising a light chain variable region comprising the amino acid sequence of SEQ ID NO:834).

### Clone PA01P2B05

In some embodiments, a monoclonal antibody that specifically binds to a milk allergen comprises a heavy chain CDR1 comprising the amino acid sequence of SEQ ID NO:868, a heavy chain CDR2 comprising the amino acid sequence of SEQ ID NO:378, a heavy chain CDR3 comprising the amino acid sequence of SEQ ID NO:869, a light chain CDR1 comprising the amino acid sequence of SEQ ID NO:871, a light chain CDR2 comprising the amino acid sequence of SEQ ID NO:682, and a light chain CDR3 comprising the amino acid sequence of SEQ ID NO:872.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:867, and that comprises a heavy chain CDR1, CDR2, and CDR3 comprising the amino acid sequences of SEQ ID NOs:868, 378, and 869, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:870, and that comprises the light chain CDR1, CDR2, and CDR3 comprising the amino acid sequences of SEQ ID NOs:871, 682, and 872, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:867, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:870. In some embodiments, the antibody comprises a heavy chain variable region comprising the amino acid sequence of SEQ ID NO:867 and comprises a light chain variable region comprising the amino acid sequence of SEQ ID NO:870.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:868, 378, 869, 871, 682, and 872, respectively, or an antibody comprising a heavy chain variable region comprising the amino acid sequence of SEQ ID NO:867 and further comprising a light chain variable region comprising the amino acid sequence of SEQ ID NO:870).

### Clone PA01P2D10

In some embodiments, a monoclonal antibody that specifically binds to a milk allergen comprises a heavy chain CDR1 comprising the amino acid sequence of SEQ ID NO:853, a heavy chain CDR2 comprising the amino acid sequence of SEQ ID NO:854, a heavy chain CDR3 comprising the amino acid sequence of SEQ ID NO:855, a light chain CDR1 comprising the amino acid sequence of SEQ ID NO:857, a light chain CDR2 comprising the amino acid sequence of SEQ ID NO:662, and a light chain CDR3 comprising the amino acid sequence of SEQ ID NO:858.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:852, and that comprises a heavy chain CDR1, CDR2, and CDR3 comprising the amino acid sequences of SEQ ID NOs:853, 854, and 855, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:856, and that comprises the light chain CDR1, CDR2, and CDR3 comprising the amino acid sequences of SEQ ID NOs:857, 662, and 858, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:852, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:856. In some embodiments, the antibody comprises a heavy chain variable region comprising the amino acid sequence of SEQ ID NO:852 and comprises a light chain variable region comprising the amino acid sequence of SEQ ID NO:856.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:853, 854, 855, 857, 662, and 858, respectively, or an antibody comprising a heavy chain variable region comprising the amino acid sequence of SEQ ID NO:852 and further comprising a light chain variable region comprising the amino acid sequence of SEQ ID NO:856).

### Clone PA01P2E08

In some embodiments, a monoclonal antibody that specifically binds to a milk allergen comprises a heavy chain CDR1 comprising the amino acid sequence of SEQ ID NO:800, a heavy chain CDR2 comprising the amino acid sequence of SEQ ID NO:801, a heavy chain CDR3 comprising the amino acid sequence of SEQ ID NO:802, a light chain CDR1 comprising the amino acid sequence of SEQ ID NO:804, a light chain CDR2 comprising the amino acid sequence of SEQ ID NO:110, and a light chain CDR3 comprising the amino acid sequence of SEQ ID NO:805.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:799, and that comprises a heavy chain CDR1, CDR2, and CDR3 comprising the amino acid sequences of SEQ ID NOs:800, 801, and 802, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:803, and that comprises the light chain CDR1, CDR2, and CDR3 comprising the amino acid sequences of SEQ ID NOs:804, 110, and 805, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:799, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:803. In some embodiments, the antibody comprises a heavy chain variable region comprising the amino acid sequence of SEQ ID NO:799 and comprises a light chain variable region comprising the amino acid sequence of SEQ ID NO:803

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:800, 801, 802, 804, 110, and 805, respectively, or an antibody comprising a heavy chain variable region comprising the amino acid sequence of SEQ ID NO:799 and further comprising a light chain variable region comprising the amino acid sequence of SEQ ID NO:803).

Antibodies that Specifically Bind to Fungal Allergens

In some embodiments, the monoclonal antibody or antigen-binding portion thereof specifically binds to a fungal allergen. In some embodiments, the monoclonal antibody specifically binds to a fungal allergen that is an Aspergillus fumigatus, Aspergillus niger, or Aspergillus nidulans allergen (e.g., an extract of Aspergillus fumigatus, Aspergillus niger, or Aspergillus nidulans). In some embodiments, the fungal allergen is Aspergillus fumigatus 1 (Asp f 1), e.g., a purified recombinant allergen Aspergillus fumigatus 1 (rAspf1).

In some embodiments, the monoclonal antibody exhibits cross-reactivity with at least two fungal allergens. In some embodiments, the monoclonal antibody exhibits cross-reactivity with two or more Aspergillus allergens (e.g., two or more species of Aspergillus). In some embodiments, the monoclonal antibody exhibits cross-reactivity with two or more of the fungal allergens Aspergillus fumigatus, Aspergillus niger, and Aspergillus nidulans. In some embodiments, the monoclonal antibody specifically binds to at least one of the fungal allergens with a K_{D} of less than 100 nM, less than 50 nM, less than 10 nM, less than 5 nM, less than 1 nM, less than 500 pM, less than 250 pM, less than 150 pM, less than 100 pM, less than 50 pM, less than 40 pM, less than 30 pM, less than 20 pM, less than about 10 pM, or less than 1 pM.

### Antibody Sequences

In some embodiments, a monoclonal antibody that specifically binds to a fungal allergen (e.g., that specifically binds to an Aspergillus allergen) comprises heavy chain CDRs and/or light chain CDRs that are disclosed in Table 1 below. In some embodiments, a monoclonal antibody that specifically binds to a fungal allergen (e.g., an Aspergillus allergen) comprises a light chain variable region sequence and/or a heavy chain variable region sequence sequence that is disclosed in Table 1 below. In some embodiments, a monoclonal antibody that specifically binds to a fungal allergen (e.g., an Aspergillus allergen) comprises: a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to a heavy chain variable region sequence disclosed in Table 1 and comprises a CDR1, a CDR2, and a CDR3 that is identical to the CDRs of that heavy chain variable region sequence, and a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity to a light chain variable region sequence disclosed in Table 1 and comprises a CDR1, a CDR2, and a CDR3 that is identical to the CDRs of that light chain variable region sequence.

In some embodiments, a monoclonal antibody that specifically binds to a fungal allergen (e.g., that specifically binds to an Aspergillus allergen) comprises a light chain sequence, or a portion thereof, and/or a heavy chain sequence, or a portion thereof, derived from any of the following antibodies described herein: Clone 1003320101_D6, Clone 1003320105_D6, Clone 1003320107_C5, Clone 1003320107_F3, or Clone 1003320107_F8. The amino acid sequences of the CDR, light chain variable domain (VL), and heavy chain variable domain (VH) of Clone 1003320101_D6, Clone 1003320105_D6, Clone 1003320107_C5, Clone 1003320107_F3, and Clone 1003320107_F8 are set forth in Table 1 below.

In some embodiments, a monoclonal antibody that specifically binds to a fungal allergen comprises one or more (e.g., one, two, three, four, five, or all six) of:
(a) a heavy chain CDR1 comprising the amino acid sequence of any one of SEQ ID NOs:710, 718, 726, 734, or 742;
a heavy chain CDR2 comprising the amino acid sequence of any one of SEQ ID NOs:711, 719, 727, 735, or 743;
a heavy chain CDR3 comprising the amino acid sequence of any one of SEQ ID NOs:712, 720, 728, 736, or 744;
a light chain CDR1 comprising the amino acid sequence of any one of SEQ ID NOs:714, 722, 730, 738, or 746;
a light chain CDR2 comprising the amino acid sequence of any one of SEQ ID NOs:715, 723, 731, 739, or 747; and a light chain CDR3 comprising the amino acid sequence of any one of SEQ ID NOs:716, 724, 732, 740, or 748.

In some embodiments, a monoclonal antibody that specifically binds to a fungal allergen comprises a heavy chain CDR1-3 and a light chain CDR1-3 comprising the amino acid sequences of:
(a) SEQ ID NOs: 710, 711, 712, 714, 715, and 716, respectively; or
(b) SEQ ID NOs:718, 719, 720, 722, 723, and 724, respectively; or
(c) SEQ ID NOs:726, 7272, 728, 730, 731, and 732, respectively; or
(d) SEQ ID NOs:734, 735, 736, 738, 739, and 740, respectively; or
(e) SEQ ID NOs:742, 743, 744, 746, 747, and 748, respectively.

In some embodiments, a monoclonal antibody that specifically binds to a fungal allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:709, 717, 725, 733, or 741. In some embodiments, a monoclonal antibody that specifically binds to a fungal allergen comprises a light chain variable region comprising an amino acid sequence that has at least 75% sequence identity (e.g., at least 75%, 80%, 85%, 90%, 91%, 92%, 93%, 94%, 95%, 96%, 97%, 98%, or 99% sequence identity to any one of SEQ ID NOs:713, 721, 729, 737, or 745. In some embodiments, a monoclonal antibody that specifically binds to a fungal allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:709, 717, 725, 733, or 741, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:713, 721, 729, 737, or 745.

In some embodiments, a monoclonal antibody that specifically binds to a fungal allergen comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:709, 717, 725, 733, or 741 and comprises a CDR1, a CDR2, and a CDR3 that is identical to the CDRs of that SEQ ID NO. In some embodiments, a monoclonal antibody that specifically binds to a fungal allergen comprises a light chain variable region comprising an amino acid sequence that has at least 70% sequence identity (e.g., at least 75%, 80%, 85%, 90%, 91%, 92%, 93%, 94%, 95%, 96%, 97%, 98%, or 99% sequence identity to any one of SEQ ID NOs:713, 721, 729, 737, or 745 and comprises a CDR1, a CDR2, and a CDR3 that is identical to the CDRs of that SEQ ID NO. In some embodiments, a monoclonal antibody that specifically binds to a fungal allergen comprises:
(a) a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:709, 717, 725, 733, or 741 and that comprises a CDR1, a CDR2, and a CDR3 that is identical to the CDRs of that SEQ ID NO; and
(b) a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to any one of SEQ ID NOs:713, 721, 729, 737, or 745 and that comprises a CDR1, a CDR2, and a CDR3 that is identical to the CDRs of that SEQ ID NO.

Clone 1003320101_D6

In some embodiments, a monoclonal antibody that specifically binds to a fungal allergen (e.g., Aspergillus allergen) comprises a heavy chain CDR1 comprising the amino acid sequence of SEQ ID NO:710, a heavy chain CDR2 comprising the amino acid sequence of SEQ ID NO:711, a heavy chain CDR3 comprising the amino acid sequence of SEQ ID NO:712, a light chain CDR1 comprising the amino acid sequence of SEQ ID NO:714, a light chain CDR2 comprising the amino acid sequence of SEQ ID NO:715, and a light chain CDR3 comprising the amino acid sequence of SEQ ID NO:716. In some embodiments, the antibody specifically binds to the fungal allergen Aspergillus fumigatus.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:709, and that comprises a heavy chain CDR1, CDR2, and CDR3 comprising the amino acid sequences of SEQ ID NOs:710, 711, and 712, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:713, and that comprises the light chain CDR1, CDR2, and CDR3 comprising the amino acid sequences of SEQ ID NOs:714, 715, and 716, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:709, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:713. In some embodiments, the antibody comprises a heavy chain variable region comprising the amino acid sequence of SEQ ID NO:709 and comprises a light chain variable region comprising the amino acid sequence of SEQ ID NO:713.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:710, 711, 712, 714, 715, and 716, respectively, or an antibody comprising a heavy chain variable region comprising the amino acid sequence of SEQ ID NO:709 and further comprising a light chain variable region comprising the amino acid sequence of SEQ ID NO:713).

### Clone 1003320105_D6

In some embodiments, a monoclonal antibody that specifically binds to a fungal allergen (e.g., Aspergillus allergen) comprises a heavy chain CDR1 comprising the amino acid sequence of SEQ ID NO:718, a heavy chain CDR2 comprising the amino acid sequence of SEQ ID NO:719, a heavy chain CDR3 comprising the amino acid sequence of SEQ ID NO:720, a light chain CDR1 comprising the amino acid sequence of SEQ ID NO:722, a light chain CDR2 comprising the amino acid sequence of SEQ ID NO:723, and a light chain CDR3 comprising the amino acid sequence of SEQ ID NO:724. In some embodiments, the antibody specifically binds to the fungal allergen Aspergillus fumigatus.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:717, and that comprises a heavy chain CDR1, CDR2, and CDR3 comprising the amino acid sequences of SEQ ID NOs:718, 719, and 720, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:721, and that comprises the light chain CDR1, CDR2, and CDR3 comprising the amino acid sequences of SEQ ID NOs:722, 723, and 724, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:717, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:721. In some embodiments, the antibody comprises a heavy chain variable region comprising the amino acid sequence of SEQ ID NO:717 and comprises a light chain variable region comprising the amino acid sequence of SEQ ID NO:721.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:718, 719, 720, 722, 723, and 724, respectively, or an antibody comprising a heavy chain variable region comprising the amino acid sequence of SEQ ID NO:717 and further comprising a light chain variable region comprising the amino acid sequence of SEQ ID NO:721).

### Clone 1003320107_C5

In some embodiments, a monoclonal antibody that specifically binds to a fungal allergen (e.g., Aspergillus allergen) comprises a heavy chain CDR1 comprising the amino acid sequence of SEQ ID NO:726, a heavy chain CDR2 comprising the amino acid sequence of SEQ ID NO:727, a heavy chain CDR3 comprising the amino acid sequence of SEQ ID NO:728, a light chain CDR1 comprising the amino acid sequence of SEQ ID NO:730, a light chain CDR2 comprising the amino acid sequence of SEQ ID NO:731, and a light chain CDR3 comprising the amino acid sequence of SEQ ID NO:732. In some embodiments, the antibody specifically binds to the fungal allergen Aspergillus fumigatus. In some embodiments, the antibody specifically binds to the fungal allergen Aspergillus niger. In some embodiments, the antibody specifically binds to the fungal allergen Aspergillus nidulans. In some embodiments, the antibody specifically binds to a recombinant Aspergillus antigen (e.g., rAsp f 1). In some embodiments, the antibody specifically binds cross-reactively to more than one of Aspergillus fumigatus, Aspergillus niger, Aspergillus nidulans, or a recombinant Aspergillus antigen (e.g., rAspf1).

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:725, and that comprises a heavy chain CDR1, CDR2, and CDR3 comprising the amino acid sequences of SEQ ID NOs:726, 727, and 728, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:729, and that comprises the light chain CDR1, CDR2, and CDR3 comprising the amino acid sequences of SEQ ID NOs:730, 731, and 732, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:725, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:729. In some embodiments, the antibody comprises a heavy chain variable region comprising the amino acid sequence of SEQ ID NO:725 and comprises a light chain variable region comprising the amino acid sequence of SEQ ID NO:729.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:726, 727, 728, 730, 731, and 732, respectively, or an antibody comprising a heavy chain variable region comprising the amino acid sequence of SEQ ID NO:725 and further comprising a light chain variable region comprising the amino acid sequence of SEQ ID NO:729).

### Clone 1003320107_F3

In some embodiments, a monoclonal antibody that specifically binds to a fungal allergen (e.g., Aspergillus allergen) comprises a heavy chain CDR1 comprising the amino acid sequence of SEQ ID NO:734, a heavy chain CDR2 comprising the amino acid sequence of SEQ ID NO:735, a heavy chain CDR3 comprising the amino acid sequence of SEQ ID NO:736, a light chain CDR1 comprising the amino acid sequence of SEQ ID NO:738, a light chain CDR2 comprising the amino acid sequence of SEQ ID NO:739, and a light chain CDR3 comprising the amino acid sequence of SEQ ID NO:740. In some embodiments, the antibody specifically binds to a recombinant Aspergillus antigen (e.g., rAspf1).

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:733, and that comprises a heavy chain CDR1, CDR2, and CDR3 comprising the amino acid sequences of SEQ ID NOs:734, 735, and 736, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:737, and that comprises the light chain CDR1, CDR2, and CDR3 comprising the amino acid sequences of SEQ ID NOs:738, 739, and 740, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:733, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:737. In some embodiments, the antibody comprises a heavy chain variable region comprising the amino acid sequence of SEQ ID NO:733 and comprises a light chain variable region comprising the amino acid sequence of SEQ ID NO:737.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:734, 735, 736, 738, 739, and 740, respectively, or an antibody comprising a heavy chain variable region comprising the amino acid sequence of SEQ ID NO:733 and further comprising a light chain variable region comprising the amino acid sequence of SEQ ID NO:737).

### Clone 1003320107_F8

In some embodiments, a monoclonal antibody that specifically binds to a fungal allergen (e.g., Aspergillus allergen) comprises a heavy chain CDR1 comprising the amino acid sequence of SEQ ID NO:742, a heavy chain CDR2 comprising the amino acid sequence of SEQ ID NO:743, a heavy chain CDR3 comprising the amino acid sequence of SEQ ID NO:744, a light chain CDR1 comprising the amino acid sequence of SEQ ID NO:746, a light chain CDR2 comprising the amino acid sequence of SEQ ID NO:747, and a light chain CDR3 comprising the amino acid sequence of SEQ ID NO:748. In some embodiments, the antibody specifically binds to the fungal allergen Aspergillus fumigatus.

In some embodiments, the antibody comprises:
(a) a heavy chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:741, and that comprises a heavy chain CDR1, CDR2, and CDR3 comprising the amino acid sequences of SEQ ID NOs:742, 743, and 744, respectively; and
(b) a light chain variable region that comprises an amino acid sequence that has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:745, and that comprises the light chain CDR1, CDR2, and CDR3 comprising the amino acid sequences of SEQ ID NOs:746, 747, and 748, respectively.

In some embodiments, the antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:741, and comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:745. In some embodiments, the antibody comprises a heavy chain variable region comprising the amino acid sequence of SEQ ID NO:741 and comprises a light chain variable region comprising the amino acid sequence of SEQ ID NO:745.

In some embodiments, the antibody is an antibody that competes for binding with an antibody as described herein (e.g., an antibody comprising a heavy chain CDR1-3 and a light chain CDR1-3 comprising the amino acid sequences of SEQ ID NOs:742, 743, 744, 746, 747, and 748, respectively, or an antibody comprising a heavy chain variable region comprising the amino acid sequence of SEQ ID NO:741 and further comprising a light chain variable region comprising the amino acid sequence of SEQ ID NO:745).

### Antigen-Binding Fragments

In some embodiments, an antibody as disclosed herein (e.g., an antibody as disclosed in Section IV that binds to a food allergen, plant allergen, fungal allergen, animal allergen, dust mite allergen, drug allergen, cosmetic allergen, or latex allergen) is an antigen-binding portion (also referred to herein as an antigen-binding fragment). Examples of antigen-binding fragments include, but are not limited to, a Fab, a F(ab′)₂, a Fv, a scFv, a bivalent scFv, a single domain antibody, or a diabody. Various techniques have been developed for the production of antigen-binding fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, antigen-binding fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)₂ fragments can be isolated directly from recombinant host cell culture. Other techniques for the production of antigen-binding fragments are known in the art.

### Antibody Conjugates

In some embodiments, the antibody or antigen-binding fragment can be conjugated to another molecule, e.g., polyethylene glycol (PEGylation) or serum albumin, to provide an extended half-life in vivo. Examples of PEGylation of antigen-binding fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

In some embodiments, antibody-drug conjugates comprising a monoclonal antibody or antigen-binding fragment as described herein are provided. In some embodiments, a monoclonal antibody or antigen-binding fragment (e.g., an antibody or antigen-binding fragment that specifically binds to a food allergen or a fungal allergen) is covalently linked to a cytotoxic drug. In some embodiments, the antibody or antigen-binding fragment is an antibody that specifically binds to a fungal allergen and the drug is an anti-fungal drug. Suitable anti-fungal drugs include, but are not limited to, Amphotericin B, azole anti-fungals (e.g., ketoconazole, fluconazole, isavuconazole, itraconazole, posaconazole, or voriconazole), echinocandins (e.g., anidulafungin, caspofungin, or micafungin), and flucytosine. Methods for making antibody-drug conjugates are described, e.g., in Chudasama et al., Nature Chemistry, 2016, 8:114-119; WO 2013/068874; and U.S. Pat. No. 8,535,678.

### Synthetic Antibodies, Antibody Compositions, and Antibody-Producing Cells

Certain antibodies described herein are derived from B cells isolated from human subjects who have been exposed to allergen(s). In certain embodiments, antibodies, antibody compositions, and cells of the invention are distinguishable from naturally occurring antibodies, compositions and cells in one or more respects. The distinguishable antibodies, compositions, and cells may be referred to as “synthetic,” or may be identified by the proviso that the antibody or composition “is not naturally occurring” or affirmatively as “non-naturally occurring.” As used herein the terms “corresponding antibody,” and “corresponding to” describes the relationship between (1) an antibody characterized by six specific CDR sequences and produced by immune cells of a study subject described in the Examples below and (2) a synthetic antibody comprising the same six CDR sequences.

### Synthetic Antibodies

Synthetic antibodies of the invention may differ in structure from naturally occurring antibodies with the same CDRs. That is, synthetic antibodies identified by specified CDRs may be structurally different from antibodies comprising the specified CDRs that are produced by cells of the study subject described in the Examples below. Possible differences include:

Variable Region Sequences that Differ Corresponding Naturally Occurring Antibodies

In one approach, an antibody heavy chain comprises the CDRs of a clone described herein (e.g., PA13P1E10) with the proviso that the antibody heavy chain does not comprise the heavy chain variable region sequence associated with the clone described herein. For illustration, in one embodiment an antibody that comprises the CDRs of Clone PA13P1E10 does not have a heavy chain variable region that comprises SEQ ID NO:9. In another approach, an antibody light chain comprises the CDRs of a clone described herein (e.g., PA13P1E10) with the proviso that the antibody light chain does not comprise the light chain variable region sequence associated with the clone described herein. For illustration, in one embodiment an antibody that comprises the CDRs of Clone PA13P1E10 does not have a light chain variable region that comprises SEQ ID NO:13). In one approach both the heavy chain and the light chain variable region of an antibody of the invention have an amino acid sequence other than the sequence disclosed herein.

### Lambda and Kappa Light Chains

In some embodiments the synthetic antibody comprises lambda type light chains. In some embodiments the synthetic antibody comprises kappa type light chains.

### Isotypes

In some embodiments the synthetic antibody with specified CDRs is an isotype other the isotype(s) found associated with the study subject from which B cells with the specified CDRs was derived. In some embodiments the antibody disclosed herein is an isotype other than IgG1. In some embodiments the antibody disclosed herein is an isotype other than IgG2. In some embodiments the antibody disclosed herein is an isotype other than IgG3. In some embodiments the antibody disclosed herein is an isotype other than IgG4. In some embodiments the antibody disclosed herein is an isotype other than IgM. In some embodiments the antibody disclosed herein is an isotype other than IgA.

### Allotypes

In some embodiments the synthetic antibody with specified CDRs is an allotype other the allotype(s) found associated with the study subject from which B cells with the specified CDRs was derived. In some embodiments, the synthetic antibody of the invention comprises an allotype selected from those listed in Table 2, below, which is different from an allotype of antibodies from the corresponding study subject. In some embodiments the synthetic antibody of the invention comprises any individual allotype selected from those listed in Table 2, with the proviso that the allotype differs from the corresponding allotype of antibodies from a study subject.

TABLE 2
Human immunoglobulin allotypes

| | Heavy chains | | | | Light |
|---|---|---|---|---|---|
| Isotype/type | IgG1 | IgG2 | IgG3 | IgA | chains |
| Allotypes | G1m | G2m | G3m | A2m | Km |
| | 1 (a) | 23 (n) | 21 (g1) | 1 | 1 |
| | 2 (x) | | 28 (g5) | 2 | 2 |
| | 3 (f) | | 11 (b0) | | 3 |
| | 17 (z) | | 5 (b1) | | |
| | | | 13 (b3) | | |
| | | | 14 (b4) | | |
| | | | 10 (b5) | | |
| | | | 15 (s) | | |
| | | | 16 (t) | | |
| | | | 6 (c3) | | |
| | | | 24 (c5) | | |
| | | | 26 (u) | | |
| | | | 27 (v) | | |

NB: Alphabetical notation given within brackets.
From: Jefferis and Marie-Paule Lefranc, 2009, “Human immunoglobulin allotypes: Possible implications for immunogenicity” mAbs 1(4): 332-338, incorporated herein by reference.

### Constant Domain Variants

Synthetic antibodies of the invention may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-like, modulate coengagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

### Synthetic Antibody Compositions

Synthetic antibody compositions of the invention may differ from naturally occurring compositions in at least one or more of the following respects: (i) composition comprises antibodies that are purified, i.e., separated from tissue or cellular material with which they are associated in the human body, and optionally in an manufactured excipient or medium; and/or (ii) antibody compositions of the invention contain a single species of antibody (are monoclonal) such that all antibodies in the composition have the same structure and specificity;

### Synthetic Antibody-Producing Cells

Antibodies described herein may be produced by recombinant expression in a human or non-human cell. Synthetic antibody-producing cells include non-human cells expressing heavy chains, light chains, or both heavy and light chains; human cells that are not immune cells heavy chains, light chains, or both heavy and light chains; and human B cells that produce heavy chains or light chains, but not both heavy and light chains. Synthetic antibodies of the invention may be heterologously expressed, in vitro or in vivo, in cells other than human B cells, such as non-human cells and human cells other than B cells, optionally other than immune cells, and optionally in cells other than cells in a B cell lineage.

### V. Methods of Use

In another aspect, the present disclosure provides therapeutic methods for treating a human subject with one or more of the allergen-specific monoclonal antibodies or antigen-binding portions thereof as disclosed herein. In some embodiments, methods of treating an allergy are provided. In some embodiments, methods of reducing one or more allergy symptoms in a subject are provided. In some embodiments, the allergen-specific monoclonal antibodies disclosed herein are used therapeutically as blocking antibodies, which is often referred to as passive immunotherapy. Without being bound to a particular theory, it is hypothesized that the allergen-specific monoclonal antibodies disclosed herein block allergen binding to IgE or outcompete endogenous IgE for allergen binding, which in turns prevents or reduces initiation of the allergic cascade. Without intending to be bound by a particular mechanism in some embodiments antibodies of the invention provide therapeutic benefit by binding inhibitory receptors on mast cells and/or basophils.

In some embodiments, the method comprises administering to the subject a therapeutically effective amount of one or more allergen-specific monoclonal antibodies as disclosed herein (e.g., one or more allergen-specific monoclonal antibodies as disclosed in Section IV above). In some embodiments, the method comprises administering to the subject a therapeutically effective amount of a pharmaceutical composition comprising one or more allergen-specific monoclonal antibodies as disclosed herein (e.g., a pharmaceutical composition as disclosed in Section VI below).

In some embodiments, the method comprises administering to the subject a therapeutically effective amount of an allergen-specific monoclonal antibody that is a human IgG isotype, such as a human IgG4 isotype, or antigen-binding portion thereof comprising at least a portion of a human IgG or IgG4 isotype constant region sequence.

In some embodiments, the method comprises administering to the subject a therapeutically effective amount of an allergen-specific monoclonal antibody or antigen-binding portion thereof. In some embodiments, the method comprises administering to the subject two or more allergen-specific monoclonal antibodies (e.g., in a pharmaceutical composition comprising the two or more allergen-specific monoclonal antibodies). In some embodiments, the method comprises administering two or more antibodies that specifically bind to the same allergen. In some embodiments, the method comprises administering two or more antibodies that specifically bind to different epitopes of the same allergen. In some embodiments, the method comprises administering two or more antibodies that specifically bind to two or more different allergens.

In some embodiments, the therapeutic antibody is an antibody that comprises CDR sequences, a heavy chain variable region, and/or a light chain variable region as described herein (e.g., as disclosed in Table 1 below) and further comprises a native or modified IgM, IgD, IgG3, IgG1, IgA1, IgG2, IgG4, or IgA2 heavy chain constant region.

In some embodiments, the therapeutic antibody is conjugated to a drug, e.g., as described in Section IV above.

In some embodiments, the human subject to be treated is an adult. In some embodiments, the human subject is a juvenile.

In some embodiments, a human subject to be treated has an allergy to a food allergen, a plant allergen, a fungal allergen, an animal allergen, a dust mite allergen, a drug allergen, a cosmetic allergen, or a latex allergen. In some embodiments, the human subject has an allergy to a food allergen. In some embodiments, the food allergen is a milk allergen, an egg allergen, a nut allergen, a fish allergen, a shellfish allergen, a soy allergen, a legume allergen, a seed allergen, or a wheat allergen. In some embodiments, the food allergen is a peanut allergen. In some embodiments, the food allergen is a milk allergen. In some embodiments, the food allergen is an egg allergen. In some embodiments, the human subject has an allergy to a plant allergen or a fungal allergen (e.g., an Aspergillus allergen). In some embodiments, the allergen is a pollen allergen (e.g., tree pollen, grass pollen, or weed pollen) or a mold allergen. In some embodiments, the human subject has an allergy to an animal allergen. In some embodiments, the allergen is a dander allergen or an insect sting.

In some embodiments, the human subject to be treated has allergies to two or more allergens, e.g., to two or more of a food allergen, a plant allergen, a fungal allergen, an animal allergen, a dust mite allergen, a drug allergen, a cosmetic allergen, or a latex allergen. In some embodiments, the human subject has allergies to 2, 3, 4, 5, 6, 7, 8, 9, 10 or more allergens. In some embodiments, the human subject has allergies to two or more different types of antigens (allergens) in a class of allergen, e.g., allergies to two or more different food allergens (e.g., allergies to two or more different peanut antigens, or allergies to a peanut allergen and a non-peanut allergen such as an egg or milk allergen). In some embodiments, the human subject has allergies to two more different classes of allergens (e.g., allergies to one or more food allergens and to one or more plant allergens). In some embodiments, a human subject has an allergy to 1, 2, 3, 4, 5, 6, 7, 8, 9, 10 or more allergens in the same class of allergen but does not have any known allergies to allergens in other classes of allergens. For example, in some embodiments, a human subject has an allergy to 1, 2, 3, 4, 5, 6, 7, 8, 9, 10 or more food allergens but does not have any known allergies to non-food allergens. In some embodiments, a human subject has an allergy to 1, 2, 3, 4, 5, 6, 7, 8, 9, 10 or more fungal allergens but does not have any known allergies to non-fungal allergens.

In some embodiments, the therapeutic methods disclosed herein reduce one or more symptoms of the allergy in the subject. It will be appreciated by a person of ordinary skill in the art that the symptom(s) associated with an allergic reaction can vary depending upon the type of allergen that induces the allergic reaction. Examples of allergic reaction symptoms include, but are not limited to, hives, rashes, eczema flare, redness of skin, itchy mouth, itchy eyes, nausea, vomiting, diarrhea, stomach pain, nasal congestion, runny nose, stuffy nose, sneezing, cough, fatigue, sore throat, swelling of the lips, tongue, or throat, headaches, trouble swallowing, shortness of breath, wheezing, drop in blood pressure, or weak pulse. In some embodiments, the therapeutic methods disclosed herein reduce the severity of one or more symptoms of the allergy. In some embodiments of the therapeutic methods described herein, the allergy symptoms in the subject comprise one or more of runny nose, skin hives, skin redness, skin swelling, itching or tingling in or around the mouth and/or throat, difficulty swallowing, watery eyes, diarrhea, stomach cramps, nausea, vomiting, tightening of the throat, shortness of breath or wheezing, shortness of breath, and anaphylaxis. In some embodiments, the therapeutic methods disclosed herein reduce the length of duration of one or more symptoms of the allergy.

In some embodiments, the therapeutic methods disclosed herein reduce one or more symptoms of allergic reaction to an allergen such as a food allergen (e.g., a peanut allergen), such as but not limited to hives, rashes, eczema flare, redness of skin, itchy mouth, nausea, vomiting, diarrhea, stomach pain, nasal congestion, runny nose, sneezing, dry cough, swelling of the lips, tongue, or throat, trouble swallowing, shortness of breath, wheezing, drop in blood pressure, or weak pulse. In some embodiments, administration of one or more allergen-specific monoclonal antibodies as disclosed herein reduces the severity of one or more of the symptoms and/or reduces the length of duration of one or more of the symptoms.

In some embodiments, an allergen-specific monoclonal antibody as disclosed herein is administered to a human subject at a therapeutically effective amount or dose. In some embodiments, a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an antibody or composition comprising an antibody as described herein can be dermal or transdermal, inhalational, intestinal, intravenous, intramuscular, intraperitoneal, intrathecal, intralesional, intrabronchial, nasal, ocular or otic delivery, oral, rectal, subcutaneous, topical, transmucosal, or any other methods known in the art. In some embodiments, the antibody or composition is administered by infusion (e.g., intravenously) or by injection (e.g., subcutaneously). In some embodiments, the route of administration of an antibody or composition comprising an antibody in any of the methods described herein is subcutaneous, intravenous, or intranasal.

In some embodiments, administration of a single dose of an antibody or composition comprising an antibody as described herein is effective to treat the allergy or reduce one or more symptoms of the allergy. In some embodiments, multiple doses of the antibody or composition are administered. In some embodiments, a second dose is administered at least 1 day, 2 days, 3 days, 4 days, 5 days, 6 days, 7 days or longer, e.g., at least 1 week, 2 weeks, 3 weeks, 4 weeks, 5 weeks, 6 weeks, 7 weeks, 8 weeks, or longer, after administration of the first dose. In some embodiments, an antibody or composition comprising an antibody as described herein is administered to a subject about every 1, 2, 3, 4, 5, 6, 7, 8, 9, or 10 week(s). In some embodiments, an antibody or composition comprising an antibody as described herein is administered to a subject over an extended period of time, e.g., for at least 30, 40, 50, 60, 70, 80, 90, 100, 150, 200, 250, 300, 350 days or longer.

In some embodiments, in any of the methods described herein, the subject is further administered an additional agent, e.g., an antihistamine, an epinephrine, a decongestant, a bronchial dilator, or a corticosteroid. In some embodiments, the monoclonal antibody and the additional agent are administered substantially simultaneously, i.e., in the same pharmaceutical composition or in separation pharmaceutical compositions that are administered at substantially the same time (e.g., administered within seconds of each other). In some embodiments, the monoclonal antibody and the additional agent are administered separately. In some embodiments, the monoclonal antibody is administered first, followed by administering of the additional agent. In some embodiments, the additional agent is administered first, followed by administering of the monoclonal antibody.

In some embodiments, in any of the methods described herein, the methods can further comprise a step of assessing the reduction of the allergy symptoms (e.g., allergy symptoms related to a peanut allergy, a tree nut allergy, a milk allergy, or a fungal allergy) in the subject. In some embodiments, the reduction of the allergy symptoms can be measured by a Total Nasal Symptom Score (TNSS), which is made from patient assessment of four symptoms graded on a 0 (none) to 3 (severe) scale for congestion, itching, and rhinorrhea, and 0 (none) to 3 (5 or more sneezes) for sneezing. Each of the four symptoms is evaluated using the following scale of 0=None, 1=Mild, 2=Moderate, or 3=Severe. The TNSS has a possible score of 0-12. In other embodiments, the reduction of the allergy symptoms can be measured by a Visual Analog Scale (VAS) nasal symptoms score, which is often used to classify allergy burden into mild, moderate, and severe. A VAS nasal symptoms score ranging from 0 (no nasal symptoms) to 100 (maximal nasal symptoms) can be used to assess the severity of combined nasal symptoms. In other embodiments, the reduction of the allergy symptoms can be measured by peak nasal inspiratory flow (PNIF), which uses a nasal spirometer to measure the nasal airflow (measured as 1/min) in a patient. In yet other embodiments, the reduction of the allergy symptoms can be measured by an allergen skin test, such as a skin prick test (SPT), which uses the presence and degree of cutaneous reactivity as a marker for sensitization within target organs, such as eyes, nose, lung, gut and skin. When relevant allergens (e.g., a peanut allergen, a tree nut allergen, a milk allergen, or a fungal allergen) are introduced into the skin, allergic reactions on the skin produce a wheal and flare response that can be quantitated, for example, using the diameter of the wheal. In yet other embodiments, the reduction of the allergy symptoms can be measured by basophil activation test, which utilizes flow cytometry to quantify the expression of markers of activation on the surface of basophils following allergen stimulation. In yet other embodiments, the reduction of the allergy symptoms can be measured by oral food challenge, which involves administering escalating doses of an allergen to an allergic individual under the supervision of a trained allergist or immunologist. An oral food challenge may be conducted according to an open, single-blind, or double-blind format, with the gold-standard being both double-blind and placebo-controlled.

In yet another aspect, the present disclosure provides diagnostic and detection methods using one or more of the allergen-specific monoclonal antibodies or antigen-binding portions thereof as disclosed herein. In some embodiments, an allergen-specific monoclonal antibody or antigen-binding portion thereof is used to detect whether a sample from a subject has allergic reactivity to an allergen (e.g., a food allergen such as a peanut allergen, tree nut allergen, or milk allergen), a plant allergen, a fungal allergen, an animal allergen, a dust mite allergen, a drug allergen, a cosmetic allergen, or a latex allergen. In some embodiments, the allergen-specific monoclonal antibody or antigen-binding portion thereof is used to detect whether a sample from a subject has allergic reactivity to a specific epitope of the allergen (e.g., using an antibody that is known to bind to a specific epitope of the allergen). In some embodiments, the method comprises contacting a sample from the subject (e.g., a blood or plasma sample) with an allergen-specific monoclonal antibody or antigen-binding portion as disclosed herein.

### VI. Compositions and Kits

In another aspect, compositions and kits comprising one or more allergen-specific monoclonal antibodies or antigen-binding portions thereof that are generated from human B cells are provided.

### Pharmaceutical Compositions

In some embodiments, pharmaceutical compositions comprising one or more allergen-specific monoclonal antibodies or antigen-binding portions thereof are provided. In some embodiments, the pharmaceutical composition comprises a monoclonal antibody as described herein, e.g., as disclosed in Section IV above. In some embodiments, the pharmaceutical composition is for use in a method of reducing one or more allergy symptoms in a subject (e.g., allergy symptoms due to an allergy to a food allergen, a plant allergen, a fungal allergen, an animal allergen, a dust mite allergen, a drug allergen, a cosmetic allergen, or a latex allergen). In some embodiments, the pharmaceutical composition is for use in a method of reducing one or more allergy symptoms in a subject having a food allergy, e.g., a peanut allergy. In some embodiments, the pharmaceutical composition is for use in a method of reducing one or more allergy symptoms in a subject having an allergy to two more allergens (e.g., two or more food allergens, e.g., peanut allergy and tree nut allergy). In some embodiments, the pharmaceutical composition is for use in a method of reducing one or more allergy symptoms in a subject having a fungal allergy.

In some embodiments, the pharmaceutical composition comprises two or more monoclonal antibodies or antigen-binding portions thereof as described herein (e.g., 2, 3, 4, 5, 6, 7, 8, 9, 10 or more antibodies or antigen-binding portions thereof). In some embodiments, the composition comprises two or more monoclonal antibodies that specifically bind to the same allergen. In some embodiments, the composition comprises two or more monoclonal antibodies that specifically bind to different epitopes of the same allergen. In some embodiments, the composition comprises two or more monoclonal antibodies that specifically bind to two or more different allergens. It will also be appreciated by a person of ordinary skill in the art that for a particular type or class of allergen, e.g., a type of food allergen such as a peanut allergen, there can be more than one substance (e.g., peptide or protein) within that type or class of allergen that induces an allergic response. In some embodiments, a composition comprises two or more monoclonal antibodies that specifically bind to different allergens within a particular type or class of allergen, e.g., two or more different peptides or proteins that are allergens of the same type or class (e.g., two or more different proteins that are peanut allergens). In some embodiments, the composition comprises two or more monoclonal antibodies that specifically bind to the same first allergen and further comprises one or more monoclonal antibodies that specifically bind to a second allergen.

Guidance for preparing formulations can be found in any number of handbooks for pharmaceutical preparation and formulation that are known to those of skill in the art. See, e.g., Remington: The Science and Practice of Pharmacy, 21st Edition, Philadelphia, PA. Lippincott Williams & Wilkins, 2005.

In some embodiments, the pharmaceutical composition further comprises one or more pharmaceutically acceptable carriers, adjuvants, and/or vehicles appropriate for the particular route of administration for which the composition is to be employed. In some embodiments, the carrier, adjuvant, and/or vehicle is suitable for intravenous, intramuscular, oral, intraperitoneal, transdermal, topical, or subcutaneous administration. In some embodiments, the pharmaceutical composition is formulated for intravenous or subcutaneous administration. Methods of formulating antibodies for injection or infusion (e.g., subcutaneous or intramuscular injection or by intravenous infusion) are also described in the art. See, e.g., US 2013/0209465, Pharmaceutically acceptable carriers are well-known in the art. See, e.g., Handbook of Pharmaceutical Excipients (5^{th} ed., Ed. Rowe et al., Pharmaceutical Press, Washington, D.C.). Examples of pharmaceutically acceptable carriers include, but are not limited to, aqueous solutions, e.g., water or physiologically compatible buffers such as Hanks's solution, Ringer's solution, or physiological saline buffer.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described in Section V above.

In some embodiments, an antibody formulation comprising one or more allergen-specific monoclonal antibodies or antigen-binding portions thereof as disclosed herein are provided. In some embodiments, the antibody formulation comprises an antibody or antigen-binding portion thereof; and a buffer.

In some embodiments, the buffer is an acetate, citrate, histidine, succinate, phosphate, or hydroxymethylaminomethane buffer. In some embodiments, the antibody formulation further comprises one or more additional excipients such as a salt, a surfactant, polyol/disaccharide/polysaccharides, amino acids, and/or an antioxidant. In some embodiments, the antibody formulation comprises a surfactant such as polysorbate 80 (Tween 80), polysorbate 20 (Tween 20), or poloxamer 188. In some embodiments, the antibody formulation comprises a polyol/disaccharide/polysaccharide such as mannitol, sorbitol, sucrose, trehalose, or dextran 40. In some embodiments, the antibody formulation comprises a salt such as sodium chloride. In some embodiments, the antibody formulation comprises an amino acid such as glycine or arginine. In some embodiments, the antibody formulation comprises an antioxidant such as ascorbic acid, methionine, or ethylenediaminetetraacetic acid (EDTA). In some embodiments, the antibody formulation is a lyophilized formulation. In some embodiments, the antibody formulation is a liquid formulation.

### Kits

In some embodiments, kits comprising one or more allergen-specific monoclonal antibodies or antigen-binding portions thereof as disclosed herein, or a pharmaceutical composition comprising one or more allergen-specific monoclonal antibodies or antigen-binding portions thereof as disclosed herein, are provided. In some embodiments, the kit comprises a monoclonal antibody as described herein, e.g., as disclosed in Section IV above. In some embodiments, the kit comprises two or more monoclonal antibodies or antigen-binding portions thereof (e.g., 2, 3, 4, 5, 6, 7, 8, 9, 10 or more antibodies or antigen-binding portions thereof) as described herein. In some embodiments, the kit is for use in a method of reducing one or more allergy symptoms in a subject (e.g., allergy symptoms due to an allergy to a food allergen, a plant allergen, a fungal allergen, an animal allergen, a dust mite allergen, a drug allergen, a cosmetic allergen, or a latex allergen). In some embodiments, the kit is for use in a method of reducing one or more allergy symptoms in a subject having a food allergy, e.g., a peanut allergy. In some embodiments, the kit is for use in a method of reducing one or more allergy symptoms in a subject having a fungal allergy. In some embodiments, the kit is for use in a method of reducing one or more allergy symptoms in a subject having an allergy to two or more allergens (e.g., two or more food allergens, e.g., a peanut allergen and a tree nut allergen).

In some embodiments, the kits can further comprise instructional materials containing directions (i.e., protocols) for the practice of the methods of this invention (e.g., instructions for using the kit for treating an allergy). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### VII. Examples

The following examples are offered to illustrate, but not to limit, the claimed invention.

### Example 1—Single Cell Transcriptomic Analysis of Human IgE Producing Cells and their Antibodies from Allergic Individuals Abstract

IgE antibodies provide immunity from helminth infections, but also can cause life-threatening allergic reactions. Despite their importance to human health, these antibodies and the cells that produce them remain enigmatic due to their scarcity in humans; much of our knowledge of their properties is derived from model organisms. Herein the isolation of IgE producing B cells from the blood of individuals with food allergies is described, followed by a detailed study of their properties by single cell RNA sequencing (scRNAseq). It has been discovered that IgE B cells are deficient in membrane immunoglobulin expression and that the IgE plasmablast state is more immature than that of other antibody producing cells. Through recombinant expression of monoclonal antibodies derived from single cells, cross-reactive IgE antibodies specific for both major peanut allergens Ara h 2 and Ara h 3 were discovered and characterized; these are among the highest affinity native human antibodies discovered to date. Surprisingly, an example of convergent evolution in unrelated individuals who independently evolved nearly identical antibodies to peanut allergens was found. Finally, it was discovered that splicing within B cells of all isotypes reveals polarized germline transcription of the IgE, but not IgG4, isotype as well as several examples of biallelic expression of germline transcripts. These results offer insights into IgE B cell transcriptomics, clonality and regulation, provide a striking example of adaptive immune convergence, and offer an approach for accelerating mechanistic disease understanding by characterizing a rare B cell population underlying IgE-mediated disease at single cell resolution.

### Introduction

The IgE antibody class is the least abundant of all isotypes in humans and plays an important role in host defense against parasitic worm infections (1), but it can also become misdirected towards otherwise harmless antigens. Food allergies are one example of this misdirection, where symptoms ranging from urticaria to potentially fatal anaphylaxis result from the degranulation of mast cells and basophils induced by the recognition of allergic food proteins by surface-bound IgE antibodies. Despite this central role in immunity and allergic disease, human IgE antibodies remain poorly characterized due to their scarcity (2). Bulk epitope mapping experiments have revealed that IgE antibodies are polyclonal and epitopes are heterogeneous (3); however, individuals with the same allergy tend to recognize a core set of one or a few allergenic proteins (4). Recent studies applying bulk fluorescence activated cell sorting (FACS) immunophenotyping (5,6) and immune repertoire deep sequencing (7) have inferred IgE B cell origins, while studies performing peanut allergen specific single cell sorting (8,9) have described clonal families to which IgE antibodies belong. However, none have successfully isolated single IgE producing cells or the paired heavy and light chain sequences that comprise individual IgE antibodies, leaving unanswered questions as to the functional properties of such antibodies, transcriptional programs of these cells, and the degree to which any of these features are shared across individuals. Similarly, there is a lack of knowledge, but growing interest, surrounding the IgG4 isotype due to its potential role in mediating the reduced clinical allergen reactivity that accompanies immunotherapy and early allergen exposure through antigen blocking (10). Here we report the first successful isolation and transcriptomic characterization of single IgE and IgG4 producing B cells from humans. We combined single cell RNA sequencing (scRNA-seq) with functional antibody assays to elucidate mechanisms underlying the regulation of IgE and to discover high affinity, cross-reactive peanut specific antibodies in unrelated individuals.

Characterization of Single B Cells from Peripheral Blood

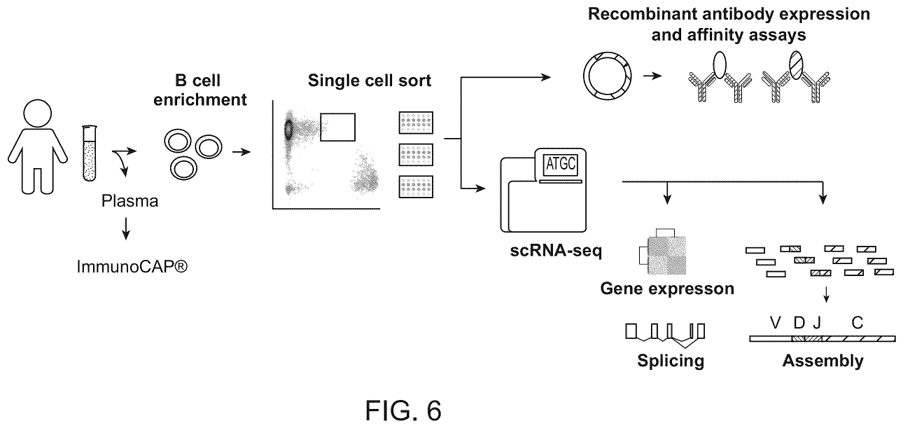

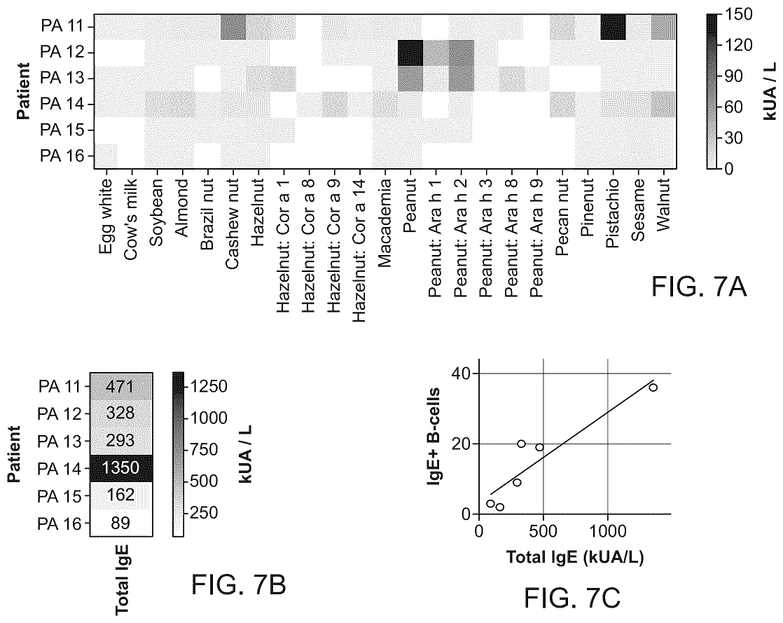

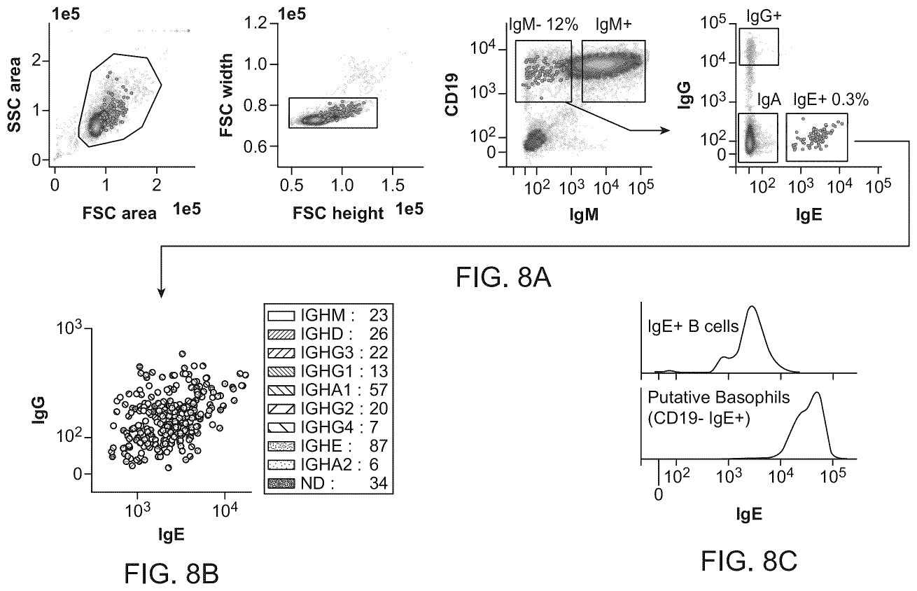

We performed scRNA-seq on B cells isolated from the peripheral blood of food allergic individuals, which enabled us to characterize each cell's gene expression, splice variants, and heavy and light chain antibody sequences (FIG. 6). Fresh peripheral blood from six peanut allergic individuals was first separated into plasma and cellular fractions; plasma was stored and later used for allergen-specific IgE concentration measurements (FIGS. 7A-7C), while the cellular fraction was enriched for B cells prior to FACS (see Materials and Methods). CD19+ B cells of all isotypes were sorted exclusively based on immunoglobulin surface expression, but with an emphasis on maximizing IgE B cell capture (FIGS. 8A-8C). Because cellular identity was determined from scRNA-seq rather than a complex, many-color gating scheme, we were able to sort and capture cells with high sensitivity. This approach makes the prospect of IgE B cell capture accessible for many laboratories without stringent requirements on FACS gate purity.

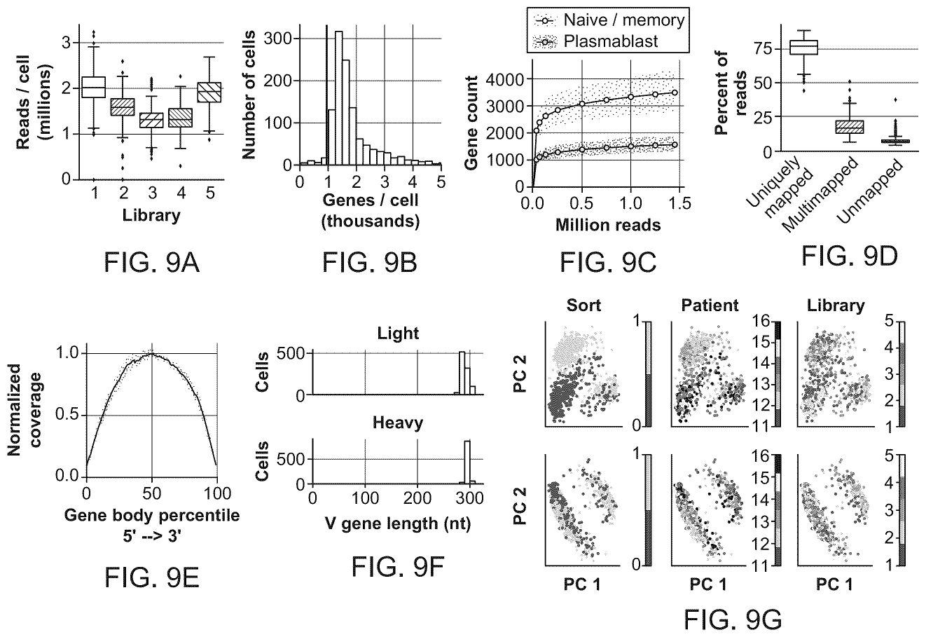

Single cells were sorted into 96 well plates, processed using a modified version of the SmartSeq2 protocol (11) and sequenced on an Illumina NextSeq with 2×150 bp reads to an average depth of 1-2 million reads per cell (FIGS. 9A-9G). Sequencing reads were independently aligned and assembled to produce a gene expression count table and reconstruct antibody heavy and light chains, respectively (FIG. 6, Materials and Methods). Using STAR (12) for alignment also facilitated the assessment of splicing within single cells. Cells were stringently filtered to remove those of low quality, putative basophils, and those lacking a single productive heavy and light chain, yielding a total of 973 cells for further analysis (Materials and Methods). The isotype identity of each cell was determined by its productive heavy chain assembly, which avoids misclassification of isotype based on FACS immunoglobulin surface staining (FIG. 8B), a problem which is especially pervasive for IgE B cells due to CD23, the “low-affinity” IgE receptor that captures IgE on the surface of non-IgE B cells (6).

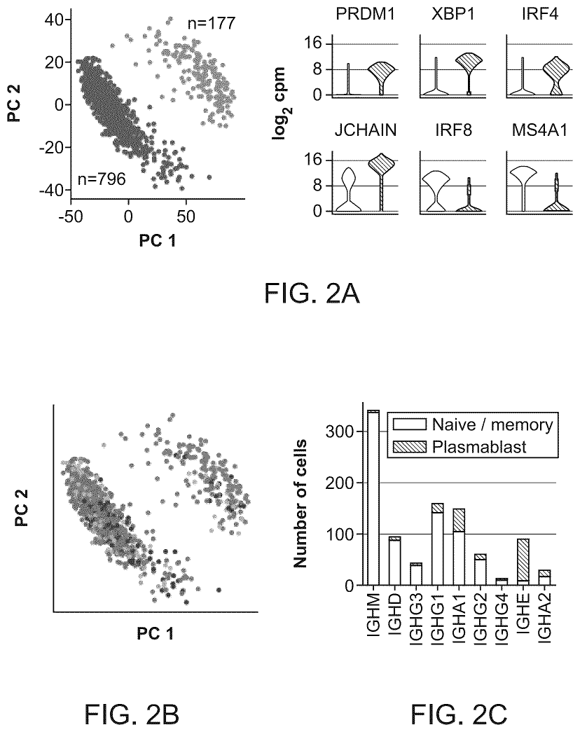

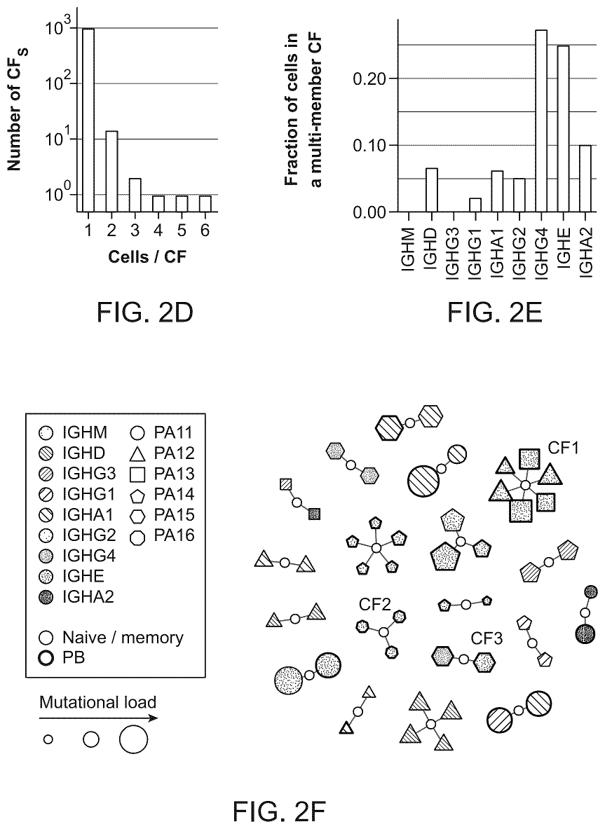

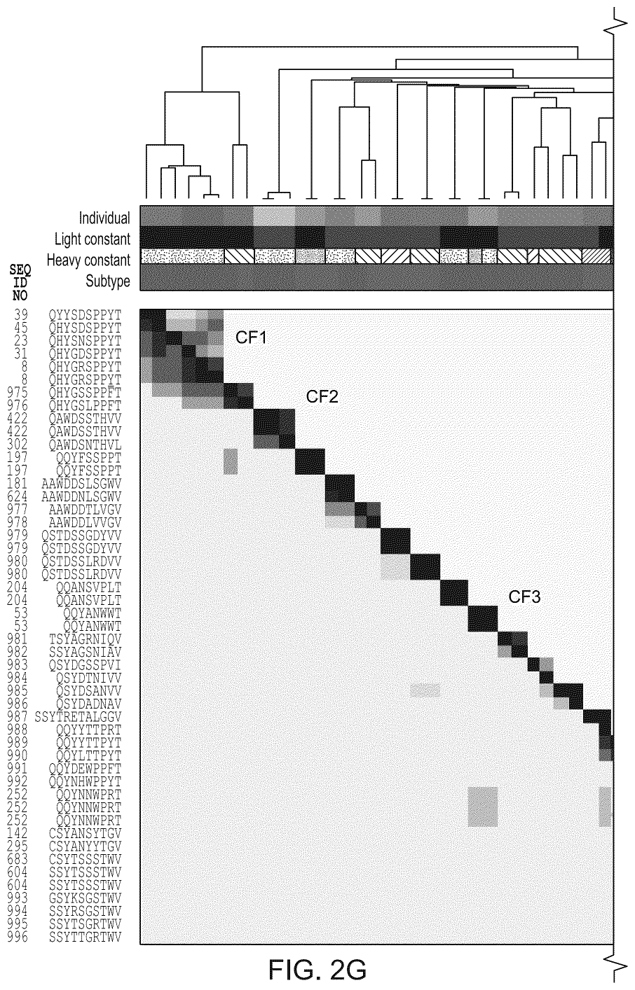

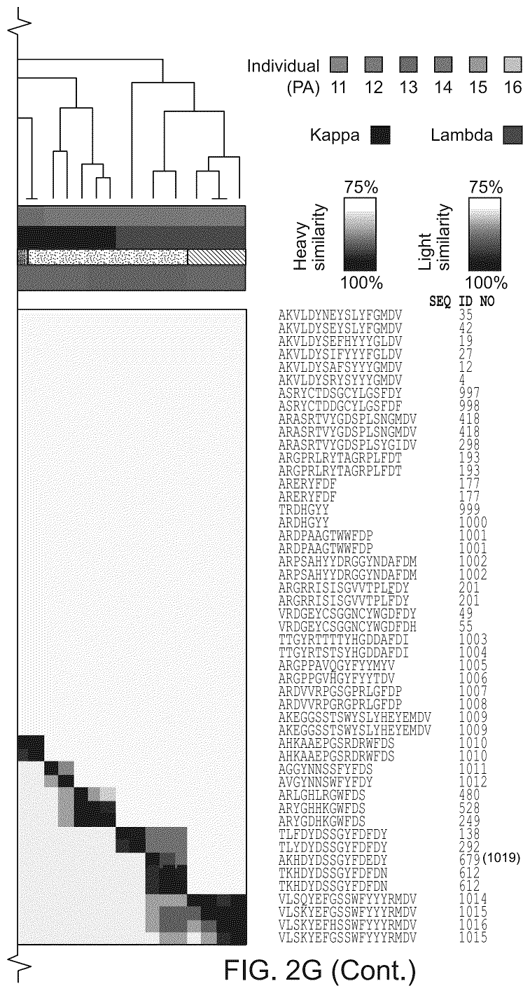

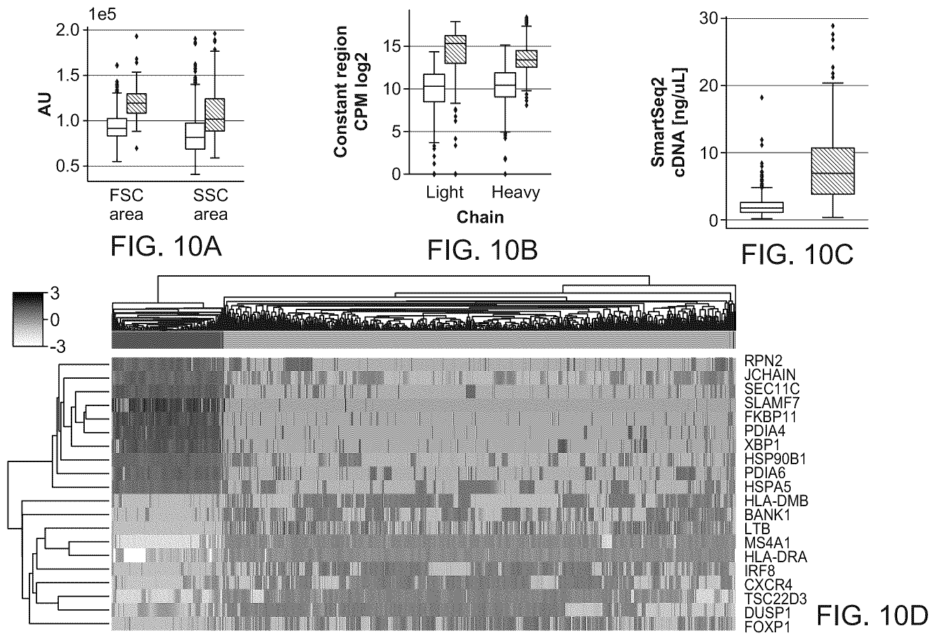

Principal component analysis of normalized gene expression following batch effect correction (FIGS. 9A-9G and Materials and Methods) separated cells into two distinct clusters (FIG. 2A) identifiable as plasmablasts (PBs) and naive/memory B cells. PBs expressed the triad of transcription factors BLIMP1 (PRDM1), XBP1, and IRF4 that drive plasma cell differentiation (13), as well as genes associated with antibody secretion, such as the J chain, while naive and memory cells expressed the canonical mature B cell surface marker CD20 (MS4A1), as well as transcription factor IRF8, which antagonizes the PB fate and instead promotes a germinal center response (14). Additional data corroborated this cell subtype assignment; PBs had greater FACS forward and side scatter in agreement with their larger size and increased granularity, PB cDNA concentrations were higher following preamplification, and PBs expressed more antibody heavy and light chain transcripts (FIGS. 10A-10D).

We assessed isotype distribution within each B cell subtype and found that, in stark contrast to other isotypes, IgE B cells overwhelmingly belonged to the PB subtype (FIG. 2B-2C). This discovery is consistent with observations of preferential differentiation of IgE B cells into PBs in mice (15). Subtype proportions for other isotypes followed expectations: IgM B cells, which are primarily naive, had the lowest PB percentages, while IgA B cells had the highest in accordance with their secretory role in maintaining mucosal homeostasis. Interestingly, we found that the number of circulating IgE B cells for each individual correlated with total plasma IgE levels (FIG. 7C); a similar phenomenon has been noted in cases of hyper-IgE syndrome (16).

By clustering antibodies into clonal families (CFs) we were able to observe elements of classical germinal center phenomenon such as somatic hypermutation, class switching, and fate determination in our data. Antibody heavy chain sequences were first divided by V and J genes and were clustered if their amino acid CDR3 sequences shared at least 75% similarity. Only 49 heavy chains formed CFs with multiple members, although this was not surprising given the vast diversity of potential immunoglobulin gene rearrangements (FIG. 2D). We found that in contrast to other isotypes, IgE and IgG4 were surprisingly clonal as over 20% of IgE and IgG4 antibodies belonged to such multi-member CFs (FIG. 2E). Multi-member CFs were diverse; they contained between two and six sequences, had variable isotype membership (node pattern), and had a comprehensive distribution of mutational frequency (node size). CFs were specific to an individual (node shape), with the exception of one CF (CF1) that contained six heavily mutated IgE PB sequences: three each from individuals PA12 and PA13, as discussed in depth later. Four CFs illustrated the two possible terminal differentiation pathways of germinal centers in that they contained both PBs and memory B cells. Other CFs contained cells belonging to multiple isotypes, with one of particular interest (CF3), discussed later, that contained an IgE PB and an IgG4 PB. Validating the premise of CFs as a collection of cells with similar origin, we found light chain CDR3 sequences were often comparable within families (FIG. 2G).

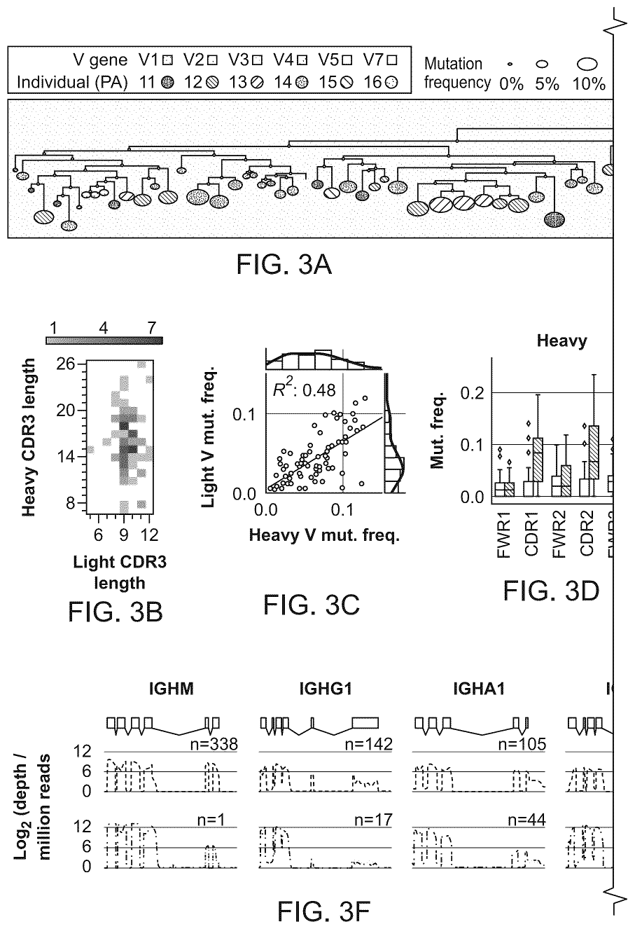

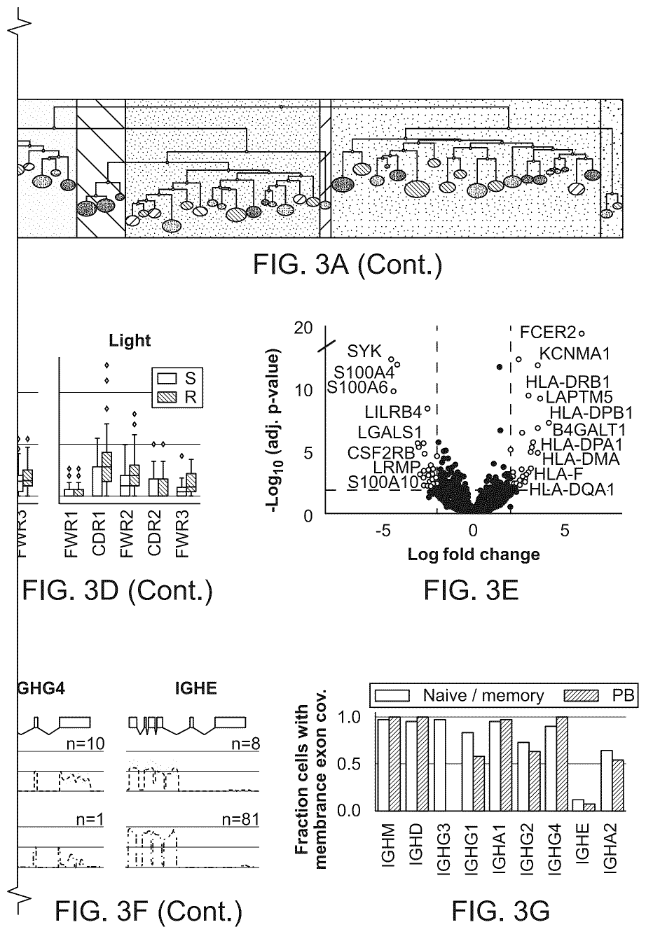

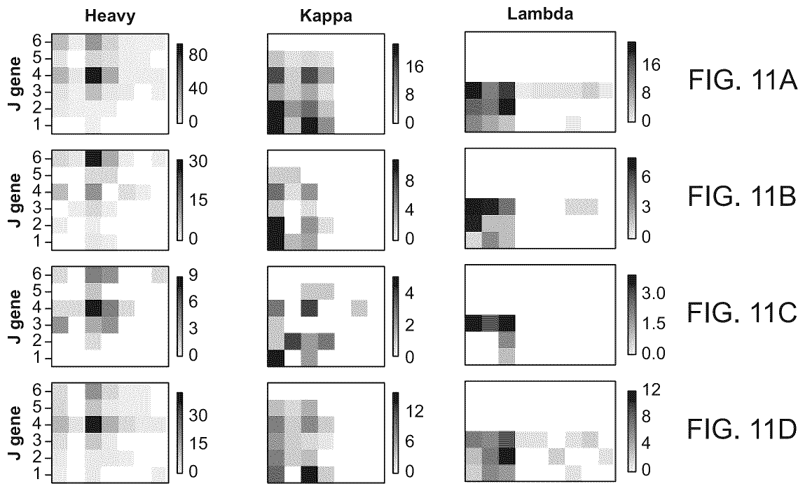

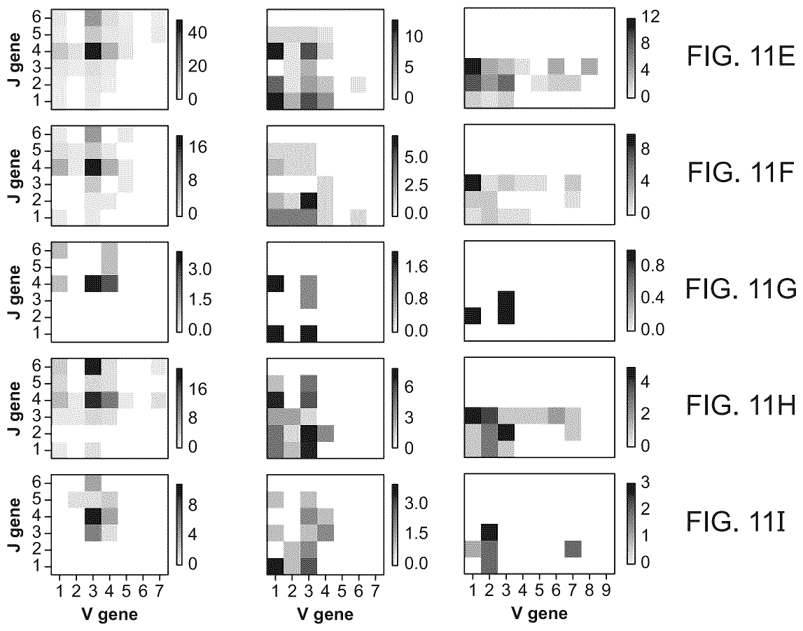

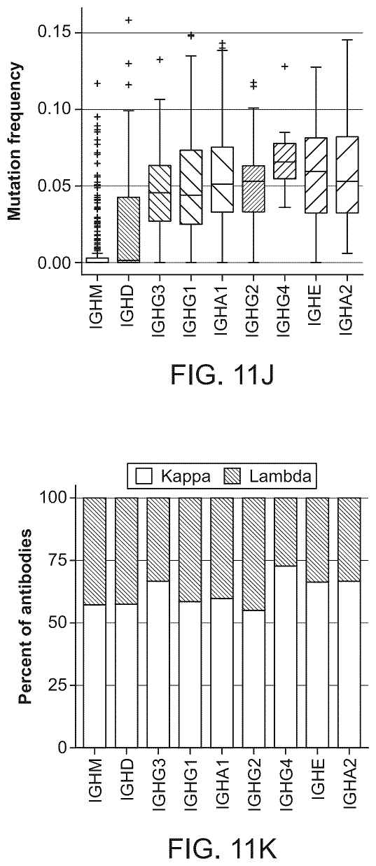

IgE antibodies varied widely in gene usage, CDR3 lengths, and mutation frequency (FIGS. 3A-3C). There was moderate correlation between the mutation frequency of heavy and light chains within single cells (FIG. 3C), with evidence of selection via an enrichment of replacement mutations relative to silent mutations in the heavy chain CDR1 and CDR2 that was absent in framework (FWR) regions. Light chains were similarly enriched for replacement mutations in the CDR1 and, to a lesser degree, FWR1 (FIG. 3D). Compared to other isotypes, IgE B cells had a similar distribution of heavy chain mutation frequency, relative utilization of the lambda versus kappa light chains, and heavy chain V and J gene usage (FIGS. 11A-11K).

### B Cell Intrinsic Factors Define IgE Cell State

To elucidate B cell intrinsic factors affecting PB activation, survival, and differentiation, we assessed genes differentially expressed between IgE PBs and PBs of other isotypes (FIG. 3E). A host of MHC genes were robustly upregulated in IgE PBs, suggesting a more immature transcriptional program given the established loss of MHC-II during the maturation of PBs to plasma cells (17) (18) (19). FCER2 (CD23), the “low-affinity” IgE receptor was also highly upregulated, although its precise role within IgE PBs is unclear; autoinhibition of IgE production could result from membrane CD23-mediated co-ligation of membrane IgE (mIgE) and CD21 (20). IgE production could also be upregulated through antagonistic effects of soluble CD23 (21), which is produced following cleavage by ADAM10 (22), a disintegrin and metalloproteinase domain-containing protein that we find is co-expressed in a subset of IgE PBs. LAPTM5, a negative regulator of B cell activation, BCR expression, and antibody production (23), was upregulated, which suggests compromised activation and proliferation capacity of IgE PBs. Downregulated genes included LILRB4 (24), galectin 1 (LGALS1), which supports plasma cell survival (25), and the S100 proteins S100A4, S100A6, and S100A10, which may indicate reduced proliferative and survival signaling (26,27). One of the most significantly downregulated genes in IgE PBs was spleen associated tyrosine kinase (SYK), which plays an essential role in BCR signal transduction (28) and is necessary for naïve differentiation into plasma cells and for memory B cell survival (29). Taken together, this gene expression program shows that the IgE PB cell state is immature relative to other PBs with weakened activation, proliferation, and survival capacity. It also provides a potential transcriptomic mechanism for the hypothesized short-lived IgE PB phenotype described in mouse models of allergy (15).

B cell intrinsic factors also regulate IgE production in murine models via impaired memory formation (30,31). Indeed, we found human IgE B cells belonging to the naïve/memory subset were deficient in heavy chain membrane immunoglobulin exon splicing compared to other common isotypes. Furthermore, membrane exon splicing was detected at low levels in non-IgE PBs, but not in IgE PBs (FIGS. 3F and 3G). In fact, the absence of mIgE splicing rendered us unable to assess the relative utilization of the two splice variants of mIgE known to have distinct signaling characteristics (32,33). The lack of mature mIgE transcripts could be explained by poor processing of pre-mRNA (34) and is consistent with low IgE surface protein we measured by FACS; indeed, mIgE surface protein levels on true IgE B cells did not exceed those of some non-IgE B cells presumably displaying surface IgE as a result of CD23-mediated capture (FIG. 8B). Together, these results suggest that the scarcity of circulating memory IgE B cells in vivo could result from impaired membrane IgE expression that compromises IgE B cell entry into the memory compartment and/or memory B cell survival. Murine studies support such a hypothesis, having shown IgE responses are reduced by removal or modification of mIgE domains, but augmented by the exchange of these domains for those of IgG1 (35).

### Characterization of Peanut-Specific IgE and IgG4 Antibodies

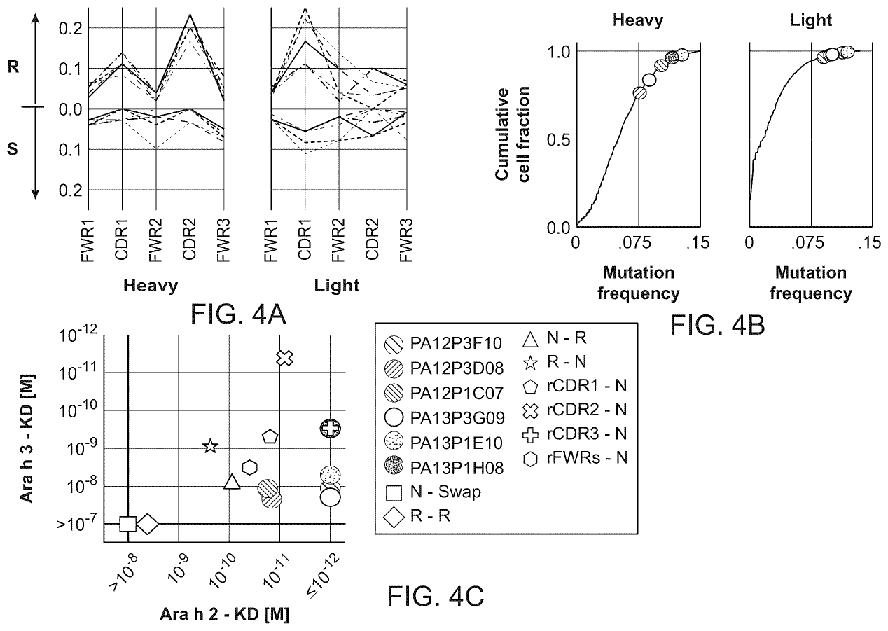

Surprisingly, our clonal analysis produced one CF of cells belonging to multiple individuals (CF1, FIGS. 2F and 2G), which contained three IgE PBs from individual PA12 and three IgE PBs from individual PA13. The antibodies produced by these six cells were highly similar as all utilized the IGHV3-30*18 and IGHJ6*02 heavy chain genes as well as the IGKV3-20*01 and IGKJ2*01 light chain genes, with pairwise CDR3 amino acid sequence identity ranging from 65% to 94% for the heavy chain and 70% to 100% for the light chain. These antibodies were also highly mutated and enriched in replacement mutations within the complementarity determining regions of both chains (FIG. 4A). In fact, compared to all other class switched antibodies, these were amongst the most mutated: the heavy chains were in the 76th percentile or above for mutation frequency, while all of the light chains were in the 96th percentile or above (FIG. 4B).

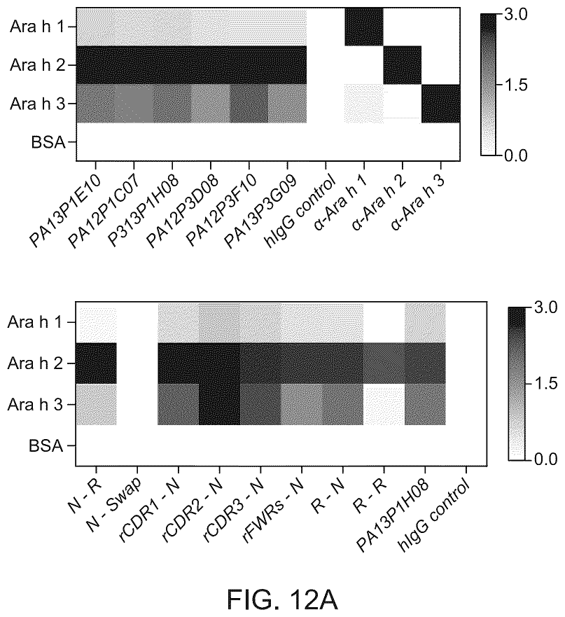

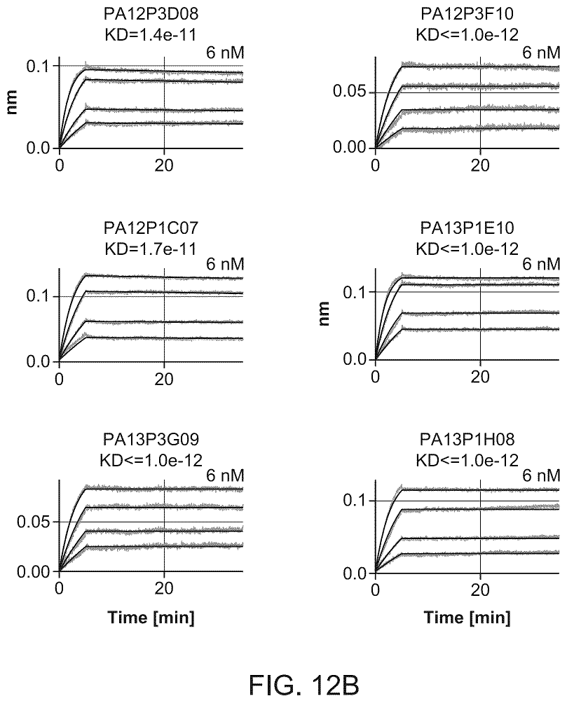

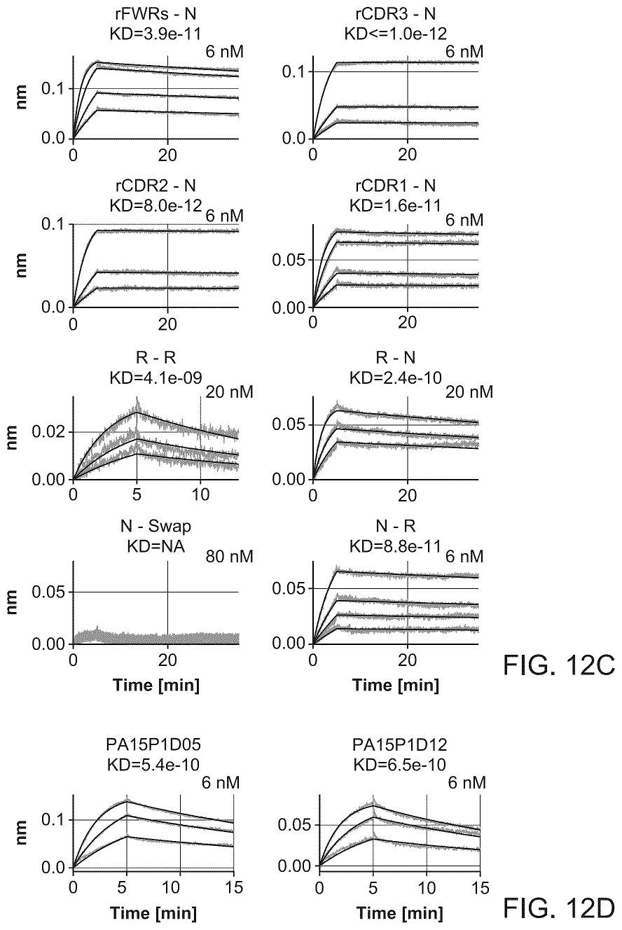

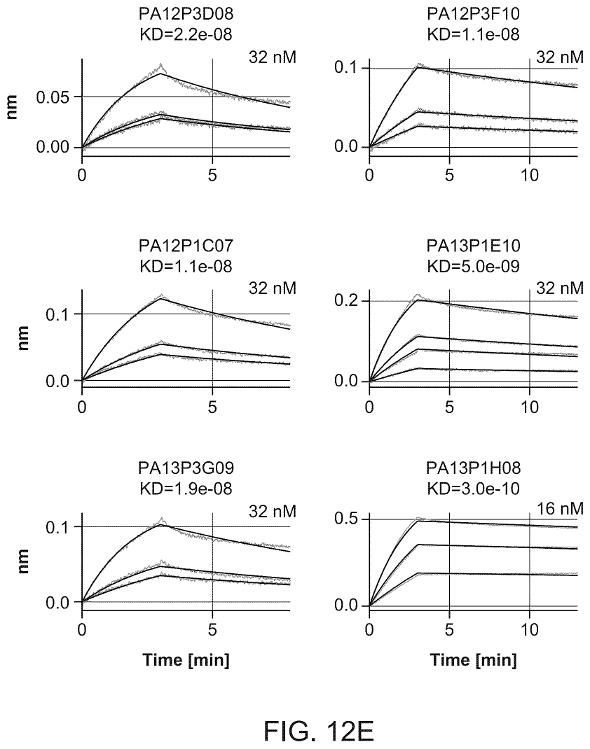

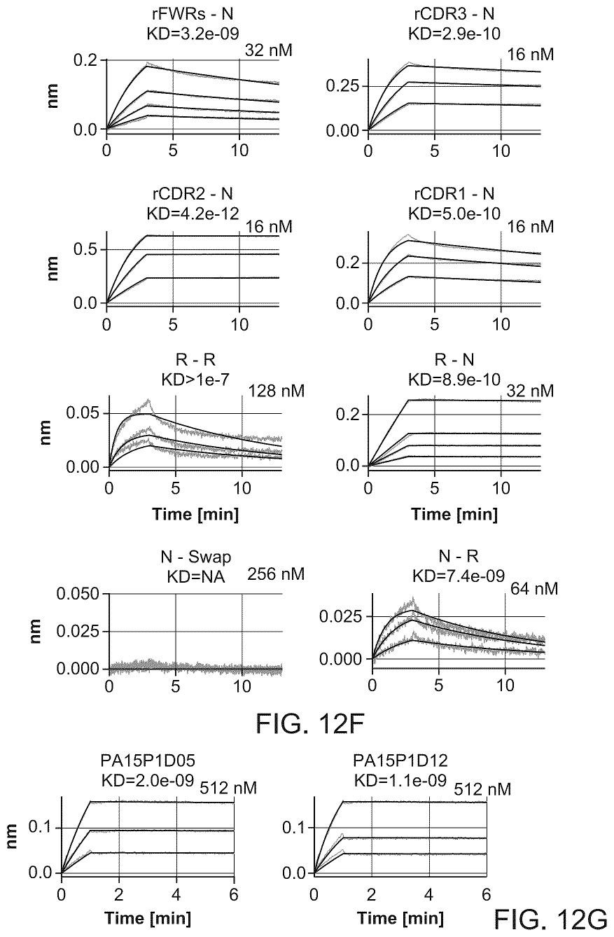

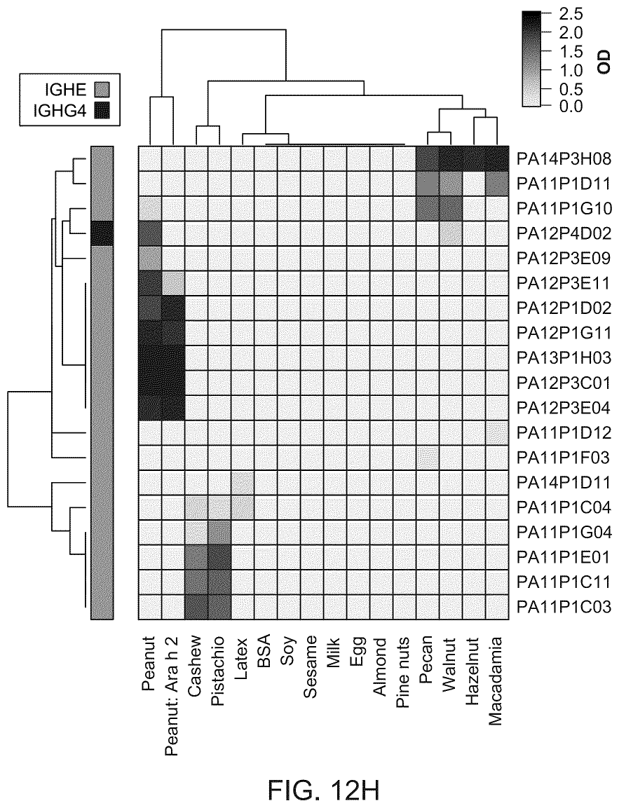

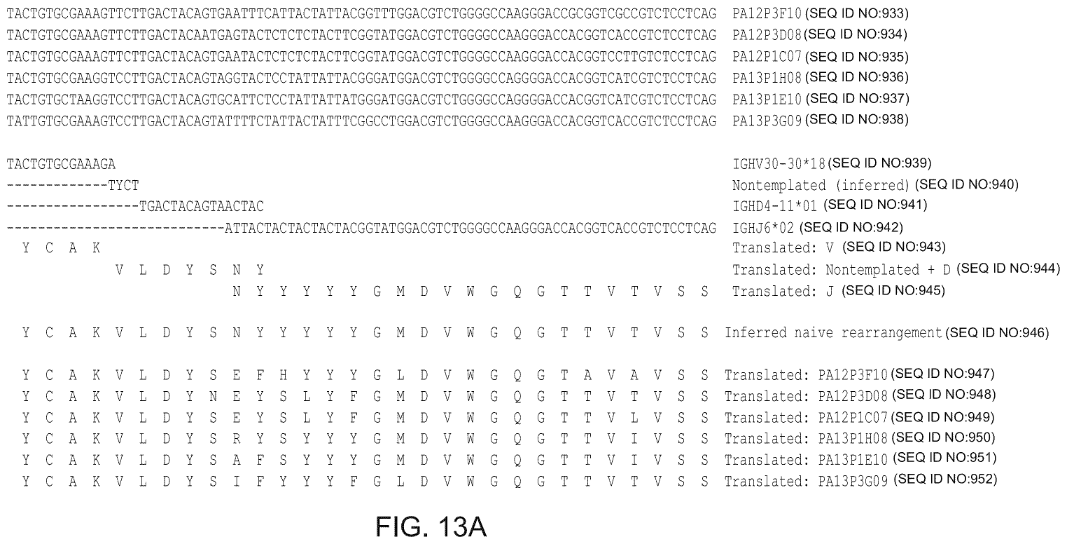

We recombinantly expressed the six IgE antibodies belonging to this convergent clonal family in order to assess whether they bind the natural forms of the major allergenic peanut (Arachis hypogaea) proteins Ara h 1, Ara h 2, or Ara h 3. Of all characterized peanut allergens, Ara h 2 is the most commonly recognized by allergic individuals and is the most clinically relevant both in terms of immunological response (36) and discriminating allergic status (37,38). Using an indirect ELISA as a semi-quantitative screen for binding, we found these six antibodies bound strongly to Ara h 2, moderately to Ara h 3, and very weakly to Ara h 1 (FIGS. 12A-12H). We then used biolayer interferometry to determine dissociation constants of each antibody for Ara h 2 and Ara h 3, with resulting affinities of 17 picomolar (pM) to sub-pM for Ara h 2 and micromolar to sub-nanomolar for Ara h 3 (FIG. 4C and FIGS. 12A-12G). These affinities are comparable to some of the highest affinity native human antibodies discovered for pathogens such as HIV, influenza, and malaria (39-43). Additionally, high affinity to multiple peanut allergens should be advantageous if such antibodies or variants thereof were to be used therapeutically as blocking antibodies intended to outcompete endogenous IgE for allergenic protein, an approach recently shown to be efficacious for treatment of cat allergy (44).

To investigate the degree to which each chain and the mutations therein affect the binding properties, we recombinantly expressed eight variants of antibody PA13P1H08, each with one or more regions in the heavy and/or light chain reverted to the inferred naïve rearrangement. Reversion of the heavy chain CDR3 was performed based on the aforementioned heavy chain V and J gene segments as well as the IGHD4-11*01 D gene and inferred nontemplated nucleotides TYCT between the V and D genes. Reversion of the light chain CDR3 was performed based on the aforementioned light chain V and J genes. Retaining the native heavy chain while swapping the light chain with another kappa light chain abrogated binding to both allergenic proteins, while reverting both chains eliminated Ara h 3 specificity and dramatically reduced Ara h 2 affinity (FIG. 4C). Reverting only the heavy or light chain reduced the affinity to Ara h 2 and Ara h 3, but disproportionately; light chain mutations contributed more to Ara h 3 affinity than did heavy chain mutations. We also found a synergistic contribution of heavy chain mutations to affinity as independent reversion of the CDR1, CDR2, CDR3 or framework region(s) each caused minor decreases in affinity. Interestingly, reversion of the heavy chain CDR2 increased Ara h 3 affinity, while only marginally decreasing Ara h 2 affinity. These results indicate that the naively recombined antibody sequence is capable of binding the most clinically relevant peanut allergen Ara h 2, but mutations, especially in the light chain, are necessary for generating high affinity antibodies which are cross-reactive towards Ara h 3. More broadly, this shows that two unrelated individuals produced an identically rearranged naive B cell that bound Ara h 2 and underwent class switching, affinity maturation, differentiation, and clonal expansion, eventually resulting in the presence of multiple circulating IgE PBs secreting high affinity antibodies with cross-reactivity towards Ara h 3.

We also expressed antibodies from two other CFs. CF2 contained three IgE PBs from individual PA16 (two of which were identical), but their recombinantly expressed antibodies did not bind Ara h 1, 2, or 3, which was unsurprising given this individual had low plasma peanut-specific IgE levels as well as IgE specific to other allergens (FIGS. 7A-7C). On the other hand, CF3 contained an IgE PB (PA15P1D05) and IgG4 PB (PA15P1D12), the recombinantly expressed antibodies from which did not bind Ara h 1, but bound Ara h 3 with nanomolar affinity and Ara h 2 with sub-nanomolar affinity (FIGS. 12A-12H). Interestingly, these two antibodies utilize the same light chain V gene and a highly similar heavy chain V gene (IGHV3-30-3*01) as the six convergent antibodies of CF1, which provides additional support for the importance of these V genes in Ara h 2 binding. Moreover, the presence of peanut-specific IgE and IgG4 in the same CF within an allergic individual provides a unique example of antagonist cell fate given the roles of IgE and IgG4 in allergic reactivity and potentially decreased sensitization, respectively.

### Polarized Germline Transcription and Class Switch Priming

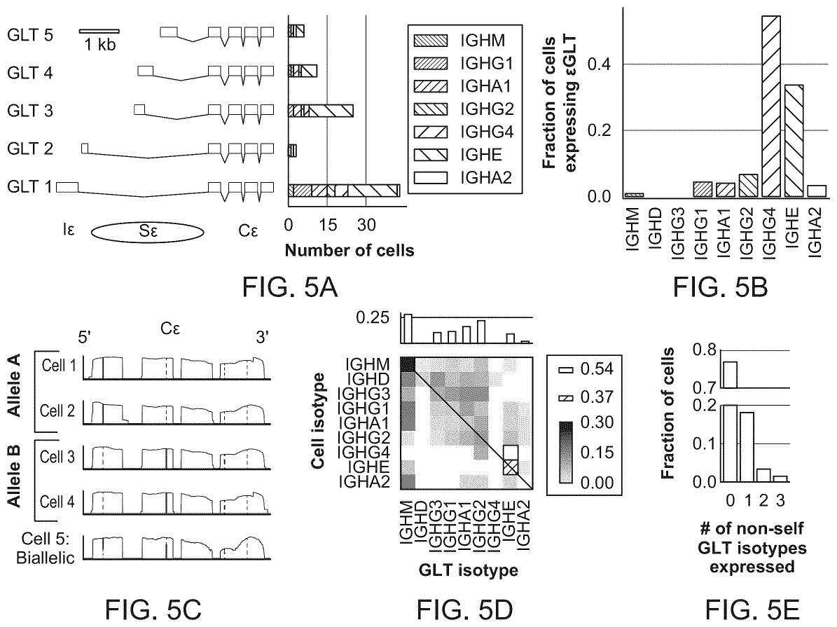

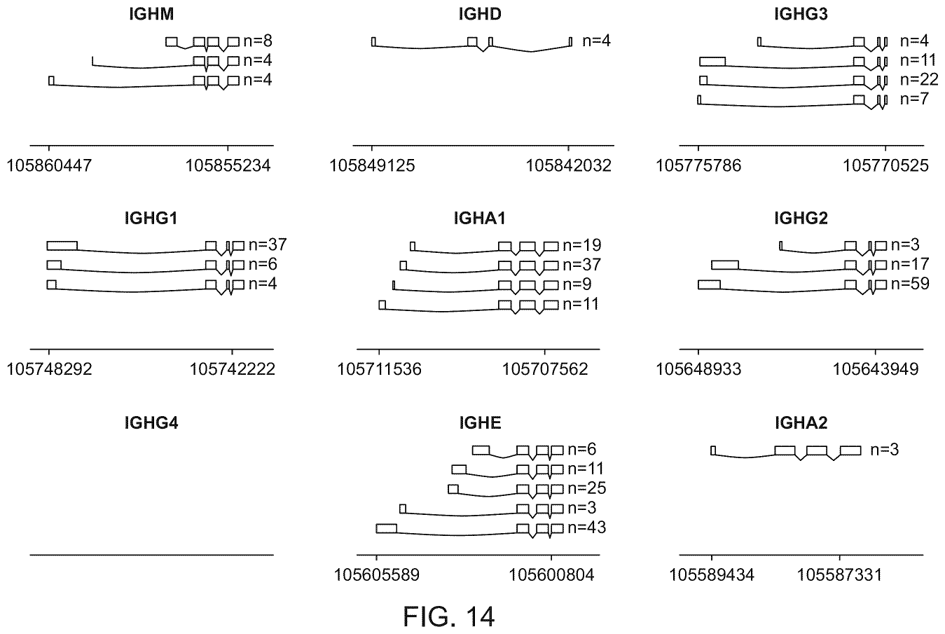

Tailored responses of the adaptive immune system are possible in part due to the ability of activation-induced cytidine deaminase (AID) to initiate class switch recombination (CSR) in B cells, leading to the production of antibodies with specific effector functions. CSR is preceded by cytokine-induced germline transcription, where nonproductive germline transcripts (GLTs) that contain an I-exon, switch (S) region, and heavy chain constant region exons guide AID to the S region (45). Importantly, GLT processing is necessary for CSR (46,47) and canonically results in two species: an intronic S region lariat and a mature polyadenylated transcript consisting of the I exon spliced to the constant region exons (48). In our scRNA-seq data, we observe multiple splice isoforms of the latter, where the proximal constant region exon serves as the exclusive splice acceptor for multiple splice donors. IgE had the largest number of distinct GLTs at five (FIG. 5A and FIG. 14), which we confirmed through Sanger sequencing; these were expressed in numerous cells of varying isotypes and across all individuals, but at nonuniform frequencies (FIG. 5A). The I-exon was the most common splice donor site (FIG. 5A, GLT #1) and it is known that I-exons can provide multiple splice donors (49-51), but EGLT splice donors within the switch region were also observed.

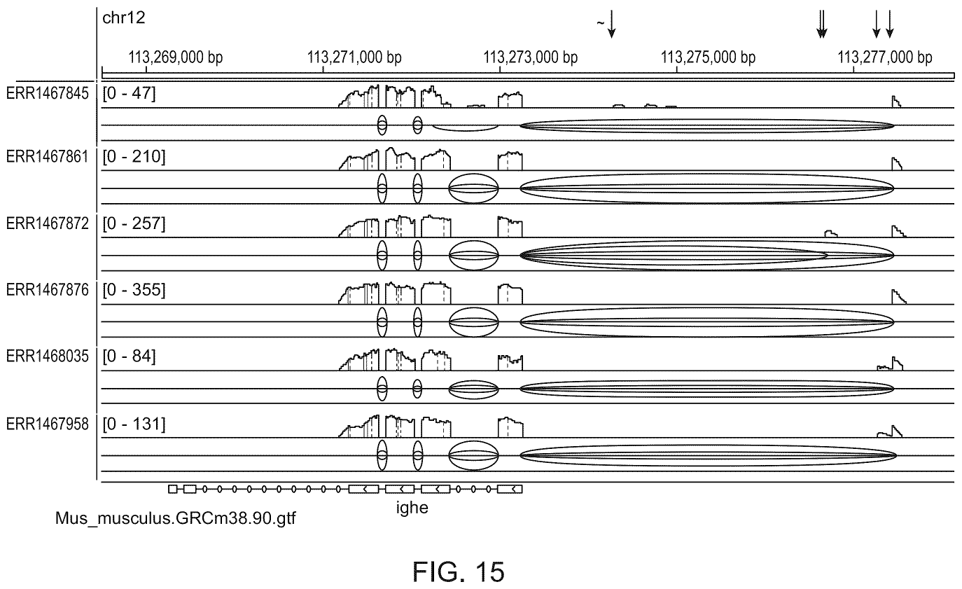

We found independent evidence for multiple IgE GLT splice donors in a previously published scRNA-seq dataset from murine B cells harvested 24 h after simulation to class switch (52) (FIG. 15). We also assessed variation in the isotypes expressing EGLTs. The IgG4 isotype had the highest proportion of cells expressing an EGLT (FIG. 5B), while IgE B cells themselves also commonly expressed EGLTs. The remainder of isotypes had fairly low expression of EGLTs.

GLT production is not limited to the IgE locus; we extended our analysis to all isotypes, enabling the construction of a global class switch priming state diagram (FIG. 5D) that illustrates the fraction of cells of each isotype that produce a GLT of their own (self) or another isotype. We observe that, in contrast to IgE, we find almost no IgG4 GLT expression in these allergic individuals. We also observe elevated IgG2 GLT production, which can be explained by splicing of the CH1 IgG2 exon to an upstream lincRNA. Interestingly, we observe that GLT expression arising from the alternate allele is common, as evidenced by common expression of IgM GLTs as well as GLTs of other isotypes upstream of a class switched isotype (signal below the diagonal in FIG. 5D). Mirroring the landscape of human class switching (53), we observe the trend for GLT production to be higher for proximal downstream isotypes rather than distant downstream isotypes. Unlike previous reports (54), we found that cells with GLT expression tend to be polarized towards the expression of a single GLT isotype, although we did not detect any non-self GLT production in most cells (FIG. 5E).

The study of B lymphocyte transcriptomes at single cell resolution offers other advantages; for example, we discovered multiple instances of biallelic GLT expression though heavy chain constant region haplotype phasing in single B cells from in individuals who had heterozygous single nucleotide variants within these loci. An example of this process that demonstrates biallelic EGLT expression is shown in FIG. 5C.

### Characterization of Tree Nut-Specific IgE and IgG4 Antibodies

Given that some subjects had plasma IgE against other allergens in addition to peanut (FIG. 7A), we assessed whether recombinant monoclonal antibodies from subjects PA11, PA12, PA13, PA14, PA15, and PA16 bound to allergen extracts derived not only from peanut, but other allergents as well, including cashew, pistachio, latex, BSA, soy, sesame, milk, egg, almond, pine nuts, pecan, walnut, hazelnut, and macadamia. The results of an indirect ELISA are shown in FIG. 12H. A total of 89 antibodies were tested, although only those with any OD value above 0.25 are shown in FIG. 12H. Antibodies not depicted include: PA12P1D04, PA12P1G02, PA16P1B09, PA16P1E11, PA16P1E12, PA11P1C01, PA11P1C12, PA11P1C06, PA11P1C08, PA11P1D07, PA11P1E08, PA11P1F10, PA11P1F02, PA11P1G06, PA11P1G07, PA13P2H10, PA15P1C03, PA15P1E01, PA15P1E02, PA13P1C01, PA13P1C09, PA13P1D02, PA13P1E06, PA14P1C10, PA14P1C12, PA14P1C02, PA14P1C04, PA14P1C06, PA14P1C07, PA14P1C08, PA14P1D10, PA14P1D02, PA14P1D07, PA14P1D09, PA14P1E10, PA14P1E11, PA14P1E12, PA14P1E04, PA14P1E06, PA14P1E08, PA14P1E09, PA14P1F10, PA14P1F11, PA14P1F05, PA14P1F07, PA14P1G01, PA14P1G11, PA14P1G12, PA14P1G03, PA14P1H01, PA14P1H11, PA14P1H12, PA14P1H02, PA14P1H05, PA14P1H09, PA12P3C05, PA12P3C09, PA12P3D11, PA12P3D09, PA12P3E06, PA12P3E07, PA12P3F02, PA12P3F07, PA13P3G04, PA14P3F10, PA14P3F02, PA14P3H10, PA14P3H12, PA12P4G03, and PA12P4G06.

As shown in FIG. 12H, several antibodies exhibited specificity for multiple tree nut allergens. The antibody PA14P3H08 bound strongly to pecan, walnut, hazelnut, and macadamia allergens; the antibody PA11P1D11 bound to pecan, walnut, and macadamia allergens; the antibodies PA11P1E01, PA11P1C11, and PA11P1C03 each strongly bound to cashew and pistachio allergens; and the antibody PA11P1C04 more weakly bound to cashew and pistachio allergens. Some antibodies exhibited specificity for both peanut and tree nut allergens. For example, the antibody PA11P1G10 strongly bound to both pecan and walnut allergens and also bound (albeit more weakly) to peanut allergen, while the antibody PA12P4D02 strongly bound to peanut allergen and more weakly bound to walnut allergen while not binding natural peanut allergen Ara h 2. Other antibodies exhibited specificity for a single tree nut allergen. For example, antibody PA11P1G04 bound to pistachio allergen, PA11P1F03 bound to pecan allergen, and PA11P1D12 bound to macadamia allergen. Furthermore, additional peanut-specific antibodies were discovered during these experiments. Antibodies PA12P3E09 and PA12P3E11 bound peanut extract with little to no binding to natural peanut allergen Ara h 2, while antibodies PA12P1D02, PA12P1G11, PA13P1H03, PA12P3C01, and PA12P3E04 bound strongly to both peanut extract and natural peanut allergen Ara h 2.

### CONCLUSION

Using scRNA-seq, we provide the first transcriptomic characterization of circulating human IgE B cells and the antibodies they produce. Our data suggests two mechanisms underlying IgE regulation in humans: membrane immunoglobulin expression deficiency and an IgE PB gene expression program suggestive of weakened activation, proliferation, and survival capacity. These results are largely consistent with extensive studies of mIgE signaling and IgE memory in murine models of allergy, and provide evidence supporting the use of animal models for this disease. (55-59). Furthermore, the ability to capture GLT splice variant, polarization, and biallelic expression information within single B cells presents an exciting application of scRNA-seq for future mechanistic studies of GLT and CSR.

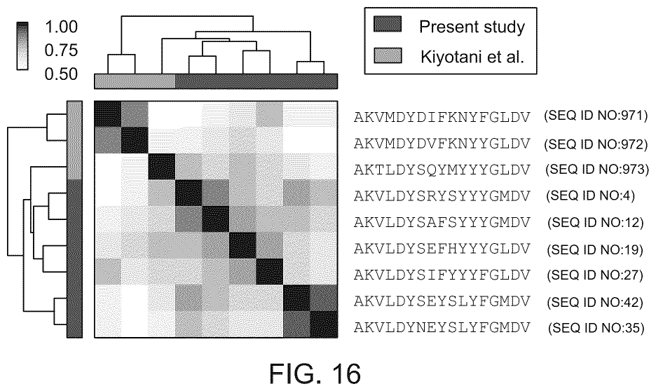

Insight into convergent evolution of high affinity antibodies in unrelated individuals can guide vaccine design and lead to strategies for population-level passive immunity; it is also a process that has been argued to occur in response to a number of pathogens such as influenza (60), HIV (43), and Streptococcus pneumoniae (61). Here we found a striking case of convergence where two unrelated individuals produced high affinity, cross-reactive, peanut-specific antibodies comprised of identical gene rearrangements within respective heavy and light chains. A third individual has Ara h 2-specific antibodies that utilize a similar heavy V gene and the same light chain V gene. Although this is a small sample size, there is evidence supporting the importance of these genes within the peanut-allergic population more broadly: one independent dataset of IgE heavy chain sequences from peanut allergic individuals (62) contains IgE heavy chains that utilize identical V and J genes and share at least 70% CDR3 identity with one or more of the six convergent antibodies in our dataset (FIG. 16); another dataset (9) contains Ara h 2 specific antibodies belonging to IgG and IgM B cells that utilize similar IGHV3-30 genes.

Cross-inhibition experiments with purified allergens and plasma IgE have shown that cross-reactivity of IgE antibodies may also be common within peanut allergic individuals (63) and the antibodies we have isolated here offer a clear example of these findings. Furthermore, the fact that these high affinity antibodies were being produced by secretory IgE PBs found in circulation contributes to an understanding of how minute amounts of allergen are capable of eliciting severe allergic reactions. We also expect that either these antibodies themselves or engineered variants of them may find application as therapeutics; recent clinical results have shown that engineered allergen-specific IgG antibodies can be administered to humans and provide effective treatment for cat-whisker allergies, perhaps by outcompeting the native IgE for antigen (44).

### Methods

### Study Subjects

All study subjects were consented and screened through the Stanford IRB approved-protocol. Participants were eligible if they had a peanut allergy confirmed by an oral food challenge and board certified allergist. Peanut allergic individuals with reported reactivity to peanut ranged in age from 8 to 17, and in some cases exhibited sensitivities to other food allergens (FIGS. 7A-7C).

### Plasma IgE Measurement and B Cell Isolation

Both plasma and cellular fractions were extracted from up to 45 mL of fresh peripheral blood collected in K2 EDTA tubes. For plasma extraction, blood was transferred to 15 mL falcon tubes and spun at 1600 g for 10 min. The upper plasma layer was extracted, transferred to 2 mL Eppendorf protein LoBind tubes and spun again at 16000 g to further purify the plasma fraction. The resulting supernatant was moved to fresh tubes before being put on dry ice and later transferred to −80° C. Allergen-specific plasma IgE measurements were performed by CLIA-licensed Johns Hopkins University Dermatology, Allergy, and Clinical Immunology (DACI) Reference Laboratory using the ImmunoCAP system. To purify B cells remaining after plasma extraction, RosetteSep human B cell enrichment cocktail (Stemcell Technologies), a negative selection antibody cocktail, was added after the plasma fraction was replaced with PBS+2% fetal bovine serum (FBS). After a 20 min incubation, the blood was then diluted two-fold with PBS+2% FBS before being transferred to Sepmate 50 mL tubes (Stemcell Technologies) containing 15 mL Ficoll-Plaque PLUS (GE Healthcare Life Sciences). An enriched B cell population was achieved after a 10 min, 1200 g spin with the brake on and transferred a fresh tube. Residual red blood cells were then removed using ACK lysis buffer (ThermoFisher) and cells were washed with stain buffer (BD Biosciences). Cells were stained on ice with the following BioLegend antibodies according to the manufacturer's instructions: PE anti-human IgE clone MHE-18, Brilliant Violent 421 anti-human CD19 clone HIB19, APC anti-human IgM clone MHM-88, and Alexa Fluor488 anti-human IgG clone M1310G05. Cells were washed twice more prior to sorting.

### Flow Cytometry and Single Cell Sorting

Single cell sorts were performed on a FACSAria II Special Order Research Product (BD Biosciences) with a 5 laser configuration (355, 405, 488, 561, and 640 nm excitation). Fluorophore compensation was performed prior to each sort using OneComp eBeads (ThermoFisher), although minimal compensation was required due to the fluorophore panel and laser configuration. Equivalent laser power settings were used for each sort. Cells were sorted using “single cell” purity mode into chilled 96 well plates (Biorad HSP9641) containing lysis buffer (11) and ERCC synthetic RNA spike-in mix (ThermoFisher). Plates were spun and put on dry ice immediately before storage at −80° C.

cDNA Generation, Library Preparation, and Sequencing

A modified version of the SmartSeq2 protocol (64) was used as previously described (11). In total, 1165 cells were sequenced across 5 runs using 2×150 bp Illumina High Output kits on an Illumina NextSeq.

### Sequencing Read Alignment, Gene Expression, and Splicing

Sequencing reads were aligned to the genome in order to determine gene expression, identify splice variants, and assess read coverage. To produce the gene expression counts table, reads were first aligned to the GRCh38 human genome using STAR v2.5.3a (12) run in 2-pass mode. Gene counts were then determined using htseq-count (65) run in intersection-nonempty mode. The GTF annotation file supplied to both STAR and htseq-count was the Ensembl 90 release manually cleaned of erroneous Ig transcripts e.g. those annotated as either a V gene or constant region but containing both V gene and constant region exons. During STAR genome generation an additional splice junction file was provided that included splicing between all combinations of heavy chain CH1 exons and IGHJ genes to improve read mapping across these junctions. Gene expression was normalized using log 2 counts per million after removing counts belonging to ERCCs. Cells with fewer than 950 expressed genes were excluded prior to analysis, as were putative basophils, identified by high FACS IgE, absent or poor quality antibody assemblies, and expression of histidine decarboxylase (HDC) and Charcot-Leyden crystal protein/Galectin-10 (CLC). Batch effects mostly affecting the naive/memory B cell subset were noted between sorts by clustering using PCA on the 500 most variable genes; this gene set was enriched in genes known to be affected by sample processing such as FOS, FOSB, JUN, JUNB, JUND, HSPA8 (66). PCA following the exclusion of genes differentially expressed between sort batches (Mann-Whitney test, p-value <0.01 after Bonferroni correction) yielded well-mixed populations within both the naive/memory and PB cell clusters not biased by sort batch, individual, or sequencing library (FIG. 9G). For differential expression analysis between IgE and non-IgE PBs, genes expressed in at least 10 PBs were analyzed by voom-limma (67) with sort batch and sequencing library were supplied as technical covariates. Constant region genes, such as IGHE and IGHA1, were excluded given these are differentially expressed by design of the comparison being made.

Analysis of splicing, including GLT expression, relied upon splice junctions called by STAR. Junctions were discarded if they contained fewer than three unique reads and GLT splice donors were only considered if observed in at least three cells. Biallelic expression of GLTs was determined based on heterozygous expression of variants discovered within heavy chain constant regions using bcftools (68).

### Antibody Heavy and Light Chain Assembly

In addition to alignment, sequencing reads were also independently assembled in order to reconstruct full length heavy and light chain transcripts. BASIC (69) was used as the primary assembler given its intended use for antibody reconstruction, while Bridger (70), a whole transcriptome assembler, was used as an alternative when BASIC did not assemble a functional heavy and/or light chain. The heavy chain isotype or light chain type (lambda or kappa) was determined using a BLAST (71) database of heavy and light chain constant regions constructed from IMGT sequences (72). Here it is important to note the necessity of isotype determination using heavy chain transcript presence rather than FACS immunoglobulin surface staining: only 30% of B cells in the IgE B cell sort gate were in fact producing IgE transcripts (FIG. 8B). This likely results from the presence of surface-bound IgE captured by CD23 on non-IgE B cells, and while acid-washing can remove IgE bound by CD23 (73), we avoided this harsh treatment in order minimize transcriptomic perturbations to the cells. Immunoglobulin variable domain gene segment assignment was performed using IgBLAST (74) v1.8.0 using a database of human germline gene segments from IMGT. IgBLAST output was parsed with Change-O and mutation frequency was called with SHazaM (75). Cells without a single productive heavy and single productive light chain, which were all members of the naive/memory cell cluster, were excluded, leaving a final total of 973 cells. The workflow engine Snakemake (76) was used to execute these analysis pipelines.

### Recombinant Antibody Expression

Select antibodies were expressed recombinantly for specificity and affinity assays. All heavy chains were expressed as human IgG1, while light chains were expressed as either lambda or kappa as appropriate. Heavy and light chain sequences were synthesized by Genscript after codon optimization and were transiently transfected in HEK293-6E cells. Antibodies were purified with RoboColumn Eshmuno® A columns (EMD Millipore) and were confirmed under reducing and non-reducing conditions by SDS-PAGE and by western blots with goat anti-human IgG-HRP and goat anti-human kappa-HRP or goat anti-human lambda-HRP as appropriate.

### Functional Antibody Characterization

ELISAs were performed one of two ways. For antibodies derived from CF1, CF2, or CF3, purified peanut allergens were used to semi-quantitatively assess peanut allergen binding. Purified natural Ara h 1 (NA-AH1-1), Ara h 2 (NA-AH2-1) and Ara h 3 (NA-AH3-1), purchased from Indoor Biotechnologies, were immobilized overnight at 4° C. using 50 μL at a concentration of 2 ng/μL. Following 3 washes, wells were blocked with 100 μL of PBST (ThermoFisher)+2% BSA for 2 hours. After two washes, 100 μL of primary antibodies were incubated for 2 hours at a concentration of 2 ng/μL in blocking buffer. Following 4 washes, 100 μL of rabbit anti-human HRP (abcam #ab6759) or rabbit anti-mouse HRP (abcam #ab6728) secondary antibodies were incubated for 2 hours at a dilution of 1/1000 in blocking buffer. After 5 washes, 150 μL of 1-Step ABTS Substrate Solution (ThermoFisher) was added to the wells. Color development was measured at 405 nm on a plate reader after 8-20 min and reported OD values are after subtraction of signal from no-antibody wells. Negative controls included immobilized BSA as an antigen, as well as a human isotype control primary antibody (abcam #ab206195). One random IgM/IgK antibody we recombinantly expressed (PA12P4H3) also did not exhibit any binding. Positive controls consisted of monoclonal mouse antibodies 2C12, 1C4, and 1E8 (Indoor Biotechnologies) specific for Ara h 1, Ara h 2, and Ara h 3, respectively.

For ELISAs testing recombinant antibodies against a broad panel of allergen extracts, the following was performed. First, the allergens were obtained. Raw nut allergens, sesame seeds, peanuts, non-fat dry milk, and soy flour were purchased at a local grocery market, while spray-dried whole egg was purchased from the National Institute of Standards and Technology (RM 8445), and liquid latex containing natural rubber centrifuged latex and water was obtained from Amazon. If necessary, a mortar and pestle was used to grind solid allergens, following which 100 mg was added to a 2 mL Eppendorf Protein LoBind tube along with a 5 mm stainless steel bead and 1.7 mL PBS. A TissueLyser system (Qiagen) was used to homogenize the sample at 30 Hz for 10 min. Subsequently, the samples were spun for 20 min at 20000 g and 4° C. The aqueous layer was then transferred to a fresh tube. The protein concentrations of these allergen extracts were then determined using the Pierce 660 nm protein assay kit (ThermoFisher) in microplate format according to the manufacturer's instructions. ELISAs were performed in 384 well format according to the following steps. First, 20 μL of 15 allergens and BSA were incubated overnight at at 4° C. at a concentration of 2 ng/μL each. The plate was then washed 3 times with 62.5 μL of 1×PBST per well per wash using an Integra VIAFLO. Wells were then blocked for 2 hrs using 50 μL of a blocking buffer consisting of 1×PBST and 2% BSA. Next, 20 μL of recombinant antibodies were incubated at a concentration of 2 ng/μL in blocking buffer. Following 4 washes, 20 μL of rabbit anti-human HRP (abcam #ab6759) diluted 1/1000 in blocking buffer was incubated for 2 hours. Following 5 washes, 40 μL of ABTS was added and 405 nm plate absorbance was measured using the BioTek Neo2.

Kinetic characterization of antibody interactions with natural purified allergenic peanut proteins was achieved using biolayer interferometry on a ForteBio Octet 96 using anti-human IgG Fc capture (AHC) biosensors with 1×PBST as the assay buffer. The assay was run with the following protocol: up to 600s baseline, 120-150s antibody load, 120-300s baseline, associations of up to 300s, and variable length dissociations that lasted up to 30 min for high affinity antibody-antigen interactions. Biosensors were regenerated by cycling between buffer and pH 1.5 glycine following each experiment. Antibodies were loaded at a concentration of 10-25 nM, while optimal peanut protein concentrations were determined experimentally (FIGS. 12A-12H). Data were processed using ForteBio software using a 1:1 binding model and global fit after reference sensor (ligand, but no analyte) subtraction.

### Example 2—Consensus Ara h 2 Binding Motif for Monoclonal Antibody PA13P1H08

### Methods

Linear epitope mapping of the recombinant IgG1 PA13P1H08 antibody was performed against Ara h 2 and Ara h 3 sequences linked and elongated with neutral GSGSGSG (SEQ ID NO:1017) linkers at the N- and C-termini to avoid truncated peptides. The linked antigen sequences were translated into linear 15 amino acid peptides with a peptide-peptide overlap of 14 amino acids. The resulting Ara h 2 and Ara h 3 peptide microarray contained 668 different peptides printed in duplicate (1,336 peptide spots) as well as 90 spots of influenza virus hemagglutinin (HA) peptide YPYDVPDYAG (SEQ ID NO: 1018) framing the microarray as internal quality controls.

The microarray was first subjected to 15 min pre-swelling in washing buffer (PBS, pH 7.4 with 0.05% Tween 20), followed by 30 min in blocking buffer (Rockland blocking buffer MB-070). The microarray was incubated with the PA13P1H08 IgG1 antibody at a concentration of 1 μg/ml in incubation buffer (washing buffer with 10% blocking buffer) for 16 h at 4° C. with shaking at 140 rpm. The microarray was then stained with secondary goat anti-human IgG (H+L) DyLight680 antibody (1:5000) and control mouse monoclonal anti-HA (12CA5) DyLight800 antibody (1:2000) for 45 min in incubation buffer at room temperature. Read-out was performed with the LI-COR Odyssey Imaging System with the following parameters: scanning offset 0.65 mm, resolution 21 μm, scanning intensities of 7/7 (red=700 nm/green=800 nm). Quantification of spot intensities and peptide annotation were done with PepSlide® Analyzer.

An identical copy of the peptide microarray was subjected to the above procedure without incubation of the PA13P1H08 antibody. This served as a control to analyze background interactions of the secondary and control antibodies with the 668 different peptides of both antigens.

### Results

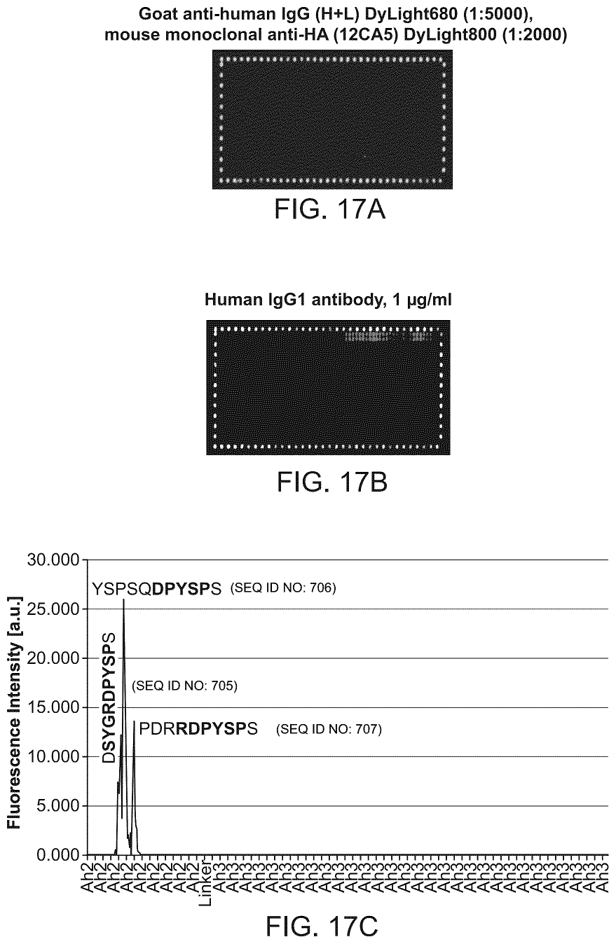

To assess whether the PA13P1H08 antibody, and by extension antibodies similar to PA13P1H08, could be binding linear peanut allergen epitope(s), we synthesized a microarray containing 15 amino acid peptides from peanut allergens Ara h 2 and Ara h 3. We found secondary and control antibody staining of the Ara h 2 and Ara h 3 peptide microarray did not highlight any background interactions that could interfere with the main assay (FIG. 17A).). In contrast, we observed a strong PA13P1H08 antibody response with high signal-to-noise ratios to the very similar consensus motifs DSYGRDPYSPS (SEQ ID NO:705), YSPSQDPYSPS (SEQ ID NO:706), and PDRRDPYSPS (SEQ ID NO:707) of Ara h 2. The similar responses were found in a region with multiple repeat units with the common DPYSPS (SEQ ID NO:704) motif (FIGS. 17B and 17C). This motif occurs three times in the Ara h 2 isoform Ara h 2.0201 (SEQ ID NO:708; WHO/JUIS allergen nomenclature; Uniprot ID: Q6PSU2-1).

### Example 3—Aspergillus-Specific Antibodies Derived from Human IgE B Cells

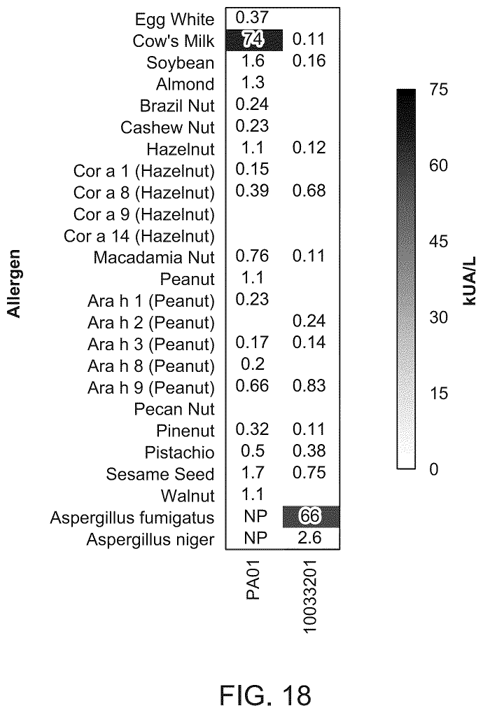

This example describes the generation and characterization of Aspergillus-specific antibodies derived from human IgE B cells. The methods of Example 1 were used to obtain Aspergillus-specific antibodies, with the following differences: the blood from which B cells were isolated originated from a subject (subject number 10033201) with allergic reactivity to Aspergillus, rather than a food allergy. Accordingly, the subject's plasma was tested for Aspergillus-specific IgE as well as for common food allergens and as shown in FIG. 18, plasma IgE levels indicate Aspergillus sensitization but no confounding food sensitizations.

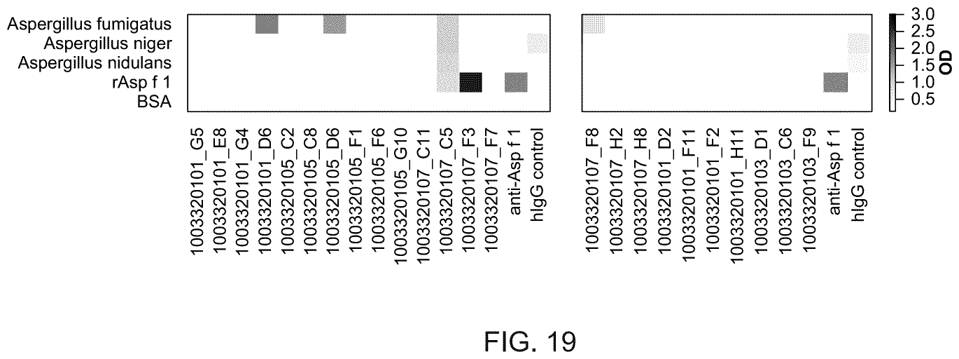

Functional assays (ELISAs) were performed as described in Example 1 to semi-quantitatively assess the obtained antibodies' specificity for statically grown, defatted, powdered, and dried Aspergillus species purchased from Stallergenes Greer as well as recombinant Aspergillus fumigatus antigen Asp f 1 purchased from Indoor Biotechnologies. As shown in FIG. 19, the monoclonal antibody clones 1003320101_D6, 1003320105_D6, and 1003320107_F8 each exhibited specific binding to Aspergillus fumigatus. Clone 1003320107_F3 exhibited specific binding to purified recombinant allergen Aspergillus fumigatus 1 (rAsp f 1). Furthermore, clone 1003320107_C5 specifically bound to each of the allergens Aspergillus fumigatus, Aspergillus niger, and Aspergillus nidulans and to rAsp F1. These results demonstrate that the method disclosed herein for generating antibodies from single IgE- or IgG4-expressing human B cells is able to robustly isolate allergen-specific antibodies independent of the type of allergic disease.

### Example 4—Milk Allergen-Specific Antibodies Derived from Human IgE B Cells

This example describes the generation and characterization of milk-specific antibodies derived from human IgE B cells. The methods of Example 1 were used to obtain milk-specific antibodies, with the following differences: the blood from which B cells were isolated originated from a subject (PA01) with allergic reactivity to cow's milk but not to other food allergens. The subject's plasma was tested for common food allergens, including milk. As shown in FIG. 18, plasma IgE levels from the subject indicate milk sensitization but no other confounding food sensitizations.

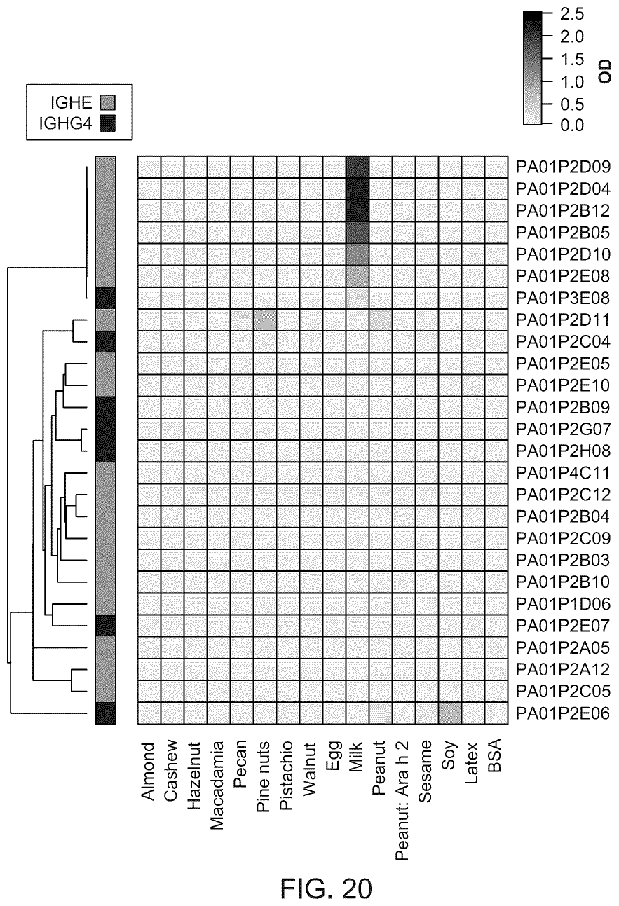

Functional assays (ELISAs) were performed as described in Example 1 to semi-quantitatively assess the specificity of the obtained IgE and IgG4 antibodies. As shown in FIG. 20, the monoclonal antibody clones PA01P2D09, PA01P2D04, PA01P2B12, PA01P2B05, and PA01P2D10 each strongly bound to milk extract, while the antibody PA01P2E08 bound moderately. These results demonstrate that the method disclosed herein for generating antibodies from single IgE- or IgG4-expressing human B cells is able to robustly isolate allergen-specific antibodies independent of the type of allergic disease.

### REFERENCES

1. Fitzsimmons C M, Falcone F H, Dunne D W. Helminth Allergens, Parasite-Specific IgE, and Its Protective Role in Human Immunity. Front Immunol. 2014 Feb. 14; 5:61.
2. Eckl-Dorna J, Niederberger V. What is the source of serum allergen-specific IgE? Curr Allergy Asthma Rep. 2013 June; 13(3):281-287.
3. Shreffler W G, Beyer K, Chu T-H T, Burks A W, Sampson H A. Microarray immunoassay: association of clinical history, in vitro IgE function, and heterogeneity of allergenic peanut epitopes. J Allergy Clin Immunol. 2004 April; 113(4):776-782.
4. Croote D, Quake S R. Food allergen detection by mass spectrometry: the role of systems biology. npj Syst Biol Appl. 2016 Sep. 29; 2:16022.
5. Heeringa J J, Rijvers L, Arends N J, Driessen G J, Pasmans S G, van Dongen J J M, et al. IgE-expressing memory B cells and plasmablasts are increased in blood of children with asthma, food allergy, and atopic dermatitis. Allergy. 2018 Jan. 30;
6. Berkowska M A, Heeringa J J, Hajdarbegovic E, van der Burg M, Thio H B, van Hagen P M, et al. Human IgE(+) B cells are derived from T cell-dependent and T cell-independent pathways. J Allergy Clin Immunol. 2014 September; 134(3):688-697.e6.
7. Looney T J, Lee J-Y, Roskin K M, Hoh R A, King J, Glanville J, et al. Human B-cell isotype switching origins of IgE. J Allergy Clin Immunol. 2016 February; 137(2):579-586.e7.
8. Patil S U, Ogunniyi A O, Calatroni A, Tadigotla V R, Ruiter B, Ma A, et al. Peanut oral immunotherapy transiently expands circulating Ara h 2-specific B cells with a homologous repertoire in unrelated subjects. J Allergy Clin Immunol. 2015 July; 136(1):125-134.e12.
9. Hoh R A, Joshi S A, Liu Y, Wang C, Roskin K M, Lee J-Y, et al. Single B-cell deconvolution of peanut-specific antibody responses in allergic patients. J Allergy Clin Immunol. 2016 January; 137(1):157-167.
10. Tordesillas L, Berin M C, Sampson H A. Immunology of food allergy. Immunity. 2017 Jul. 18; 47(1):32-50.
11. Darmanis S, Sloan S A, Croote D, Mignardi M, Chernikova S, Samghababi P, et al. Single-Cell RNA-Seq Analysis of Infiltrating Neoplastic Cells at the Migrating Front of Human Glioblastoma. Cell Rep. 2017 Oct. 31; 21(5):1399-1410.
12. Dobin A, Davis C A, Schlesinger F, Drenkow J, Zaleski C, Jha S, et al. STAR: ultrafast universal RNA-seq aligner. Bioinformatics. 2013 Jan. 1; 29(1):15-21.
13. Nutt S L, Hodgkin P D, Tarlinton D M, Corcoran L M. The generation of antibody-secreting plasma cells. Nat Rev Immunol. 2015 Mar.; 15(3):160-171.
14. Xu H, Chaudhri V K, Wu Z, Biliouris K, Dienger-Stambaugh K, Rochman Y, et al. Regulation of bifurcating B cell trajectories by mutual antagonism between transcription factors IRF4 and IRF8. Nat Immunol. 2015 Dec.; 16(12):1274-1281.
15. Erazo A, Kutchukhidze N, Leung M, Christ APG, Urban J F, Curotto de Lafaille M A, et al. Unique maturation program of the IgE response in vivo. Immunity. 2007 Feb. 8; 26(2):191-203.
16. Horst A, Hunzelmann N, Arce S, Herber M, Manz R A, Radbruch A, et al. Detection and characterization of plasma cells in peripheral blood: correlation of IgE+ plasma cell frequency with IgE serum titre. Clin Exp Immunol. 2002 December; 130(3):370-378.
17. Manz R, Löhning M, Cassese G, Thiel A, Radbruch A. Survival of long-lived plasma cells is independent of antigen. Int Immunol. 1998 Nov. 1; 10(11):1703-1711.
18. Medina F, Segundo C, Campos-Caro A, Gonzalez-Garcia I, Brieva J A. The heterogeneity shown by human plasma cells from tonsil, blood, and bone marrow reveals graded stages of increasing maturity, but local profiles of adhesion molecule expression. Blood. 2002 Mar. 15; 99(6):2154-2161.
19. Calame K L, Lin K-I, Tunyaplin C. Regulatory mechanisms that determine the development and function of plasma cells. Annu Rev Immunol. 2003; 21:205-230.
20. Gould H J, Sutton B J. IgE in allergy and asthma today. Nat Rev Immunol. 2008 Mar.; 8(3):205-217.
21. Cooper A M, Hobson P S, Jutton M R, Kao M W, Drung B, Schmidt B, et al. Soluble CD23 controls IgE synthesis and homeostasis in human B cells. J Immunol. 2012 Apr. 1; 188(7):3199-3207.
22. Weskamp G, Ford J W, Sturgill J, Martin S, Docherty A J P, Swendeman S, et al. ADAM10 is a principal “sheddase” of the low-affinity immunoglobulin E receptor CD23. Nat Immunol. 2006 Dec.; 7(12):1293-1298.
23. Ouchida R, Kurosaki T, Wang J-Y. A role for lysosomal-associated protein transmembrane 5 in the negative regulation of surface B cell receptor levels and B cell activation. J Immunol. 2010 Jul. 1; 185(1):294-301.
24. Cella M, Döhring C, Samaridis J, Dessing M, Brockhaus M, Lanzavecchia A, et al. A novel inhibitory receptor (ILT3) expressed on monocytes, macrophages, and dendritic cells involved in antigen processing. J Exp Med. 1997 May 19; 185(10):1743-1751.
25. Anginot A, Espeli M, Chasson L, Mancini S J C, Schiff C. Galectin 1 modulates plasma cell homeostasis and regulates the humoral immune response. J Immunol. 2013 Jun. 1; 190(11):5526-5533.
26. Leśniak W, Stomnicki ŁP, Filipek A. S100A6—new facts and features. Biochem Biophys Res Commun. 2009 Dec. 25; 390(4):1087-1092.
27. Boye K, Maelandsmo G M. S100A4 and metastasis: a small actor playing many roles. Am J Pathol. 2010 February; 176(2):528-535.
28. Geahlen R L. Syk and pTyr′d: Signaling through the B cell antigen receptor. Biochim Biophys Acta. 2009 July; 1793(7):1115-1127.
29. Ackermann J A, Nys J, Schweighoffer E, McCleary S, Smithers N, Tybulewicz V U. Syk tyrosine kinase is critical for B cell antibody responses and memory B cell survival. J Immunol. 2015 May 15; 194(10):4650-4656.
30. Yang Z, Sullivan B M, Allen CDC. Fluorescent in vivo detection reveals that IgE(+) B cells are restrained by an intrinsic cell fate predisposition. Immunity. 2012 May 25; 36(5):857-872.
31. He J-S, Subramaniam S, Narang V, Srinivasan K, Saunders S P, Carbajo D, et al. IgG1 memory B cells keep the memory of IgE responses. Nat Commun. 2017 Sep. 21; 8(1):641.
32. Poggianella M, Bestagno M, Burrone O R. The extracellular membrane-proximal domain of human membrane IgE controls apoptotic signaling of the B cell receptor in the mature B cell line A20. J Immunol. 2006 Sep. 15; 177(6):3597-3605.
33. Batista F D, Anand S, Presani G, Efremov D G, Burrone O R. The two membrane isoforms of human IgE assemble into functionally distinct B cell antigen receptors. J Exp Med. 1996 Dec. 1; 184(6):2197-2205.
34. Karnowski A, Achatz-Straussberger G, Klockenbusch C, Achatz G, Lamers M C. Inefficient processing of mRNA for the membrane form of IgE is a genetic mechanism to limit recruitment of IgE-secreting cells. Eur J Immunol. 2006 July; 36(7):1917-1925.
35. Wu L C, Zarrin A A. The production and regulation of IgE by the immune system. Nat Rev Immunol. 2014 April; 14(4):247-259.
36. Koppelman S J, Wensing M, Ertmann M, Knulst A C, Knol E F. Relevance of Ara h1, Ara h2 and Ara h3 in peanut-allergic patients, as determined by immunoglobulin E Western blotting, basophil-histamine release and intracutaneous testing: Ara h2 is the most important peanut allergen. Clin Exp Allergy. 2004 April; 34(4):583-590.
37. Nicolaou N, Murray C, Belgrave D, Poorafshar M, Simpson A, Custovic A. Quantification of specific IgE to whole peanut extract and peanut components in prediction of peanut allergy. Journal of Allergy and Clinical Immunology. 2011 March; 127(3):684-685.
38. Dang T D, Tang M, Choo S, Licciardi P V, Koplin J J, Martin P E, et al. Increasing the accuracy of peanut allergy diagnosis by using Ara h 2. J Allergy Clin Immunol. 2012 April; 129(4):1056-1063.
39. Wrammert J, Smith K, Miller J, Langley W A, Kokko K, Larsen C, et al. Rapid cloning of high-affinity human monoclonal antibodies against influenza virus. Nature. 2008 May 29; 453(7195):667-671.
40. Murugan R, Buchauer L, Triller G, Kreschel C, Costa G, Pidelaserra Marti G, et al. Clonal selection drives protective memory B cell responses in controlled human malaria infection. Sci Immunol. 2018 Feb. 16; 3(20).
41. Kaur K, Zheng N-Y, Smith K, Huang M, Li L, Pauli N T, et al. High Affinity Antibodies against Influenza Characterize the Plasmablast Response in SLE Patients After Vaccination. PLoS ONE. 2015 May 7; 10(5):e0125618.
42. Mouquet H, Klein F, Scheid J F, Warncke M, Pietzsch J, Oliveira TYK, et al. Memory B cell antibodies to HIV-1 gp140 cloned from individuals infected with clade A and B viruses. PLoS ONE. 2011 Sep. 8; 6(9):e24078.
43. Scheid J F, Mouquet H, Ueberheide B, Diskin R, Klein F, Oliveira TYK, et al. Sequence and structural convergence of broad and potent HIV antibodies that mimic CD4 binding. Science. 2011 Sep. 16; 333(6049):1633-1637.
44. Orengo J M, Radin A R, Kamat V, Badithe A, Ben L H, Bennett B L, et al. Treating cat allergy with monoclonal IgG antibodies that bind allergen and prevent IgE engagement. Nat Commun. 2018 Apr. 12; 9(1):1421.
45. Yewdell W T, Chaudhuri J. A transcriptional serenAID: the role of noncoding RNAs in class switch recombination. Int Immunol. 2017 Apr. 1; 29(4):183-196.
46. Hein K, Lorenz M G, Siebenkotten G, Petry K, Christine R, Radbruch A. Processing of switch transcripts is required for targeting of antibody class switch recombination. J Exp Med. 1998 Dec. 21; 188(12):2369-2374.
47. Lorenz M, Jung S, Radbruch A. Switch transcripts in immunoglobulin class switching. Science. 1995 Mar. 24; 267(5205):1825-1828.
48. Zheng S, Vuong B Q, Vaidyanathan B, Lin J-Y, Huang F-T, Chaudhuri J. Non-coding RNA Generated following Lariat Debranching Mediates Targeting of AID to DNA. Cell. 2015 May 7; 161(4):762-773.
49. Collins J T, Dunnick W A. Germline transcripts of the murine immunoglobuliny 2a gene: structure and induction by IFN-γ. Int Immunol. 1993; 5(8):885-891.
50. Gaff C, Gerondakis S. RNA splicing generates alternate forms of germline immunoglobulin alpha heavy chain transcripts. Int Immunol. 1990; 2(12):1143-1148.
51. Gauchat J F, Lebman D A, Coffman R L, Gascan H, de Vries J E. Structure and expression of germline epsilon transcripts in human B cells induced by interleukin 4 to switch to IgE production. J Exp Med. 1990 Aug. 1; 172(2):463-473.
52. Wu Y L, Stubbington M J T, Daly M, Teichmann S A, Rada C. Intrinsic transcriptional heterogeneity in B cells controls early class switching to IgE. J Exp Med. 2017 January; 214(1):183-196.
53. Horns F, Vollmers C, Croote D, Mackey S F, Swan G E, Dekker C L, et al. Lineage tracing of human B cells reveals the in vivo landscape of human antibody class switching. elife. 2016 Aug. 2; 5.
54. Fear D J, McCloskey N, O'Connor B, Felsenfeld G, Gould H J. Transcription of Ig germline genes in single human B cells and the role of cytokines in isotype determination. J Immunol. 2004 Oct. 1; 173(7):4529-4538.
55. Laffleur B, Duchez S, Tarte K, Denis-Lagache N, Peron S, Carrion C, et al. Self-Restrained B Cells Arise following Membrane IgE Expression. Cell Rep. 2015 Feb. 12;
56. Haniuda K, Fukao S, Kodama T, Hasegawa H, Kitamura D. Autonomous membrane IgE signaling prevents IgE-memory formation. Nat Immunol. 2016 Jul. 18; 17(9):1109-1117.
57. Tong P, Granato A, Zuo T, Chaudhary N, Zuiani A, Han S S, et al. IgH isotype-specific B cell receptor expression influences B cell fate. Proc Natl Acad Sci USA. 2017 Oct. 3; 114(40):E8411-E8420.
58. Yang Z, Robinson M J, Chen X, Smith G A, Taunton J, Liu W, et al. Regulation of B cell fate by chronic activity of the IgE B cell receptor. elife. 2016 Dec. 9; 5.
59. Laffleur B, Debeaupuis O, Dalloul Z, Cogné M. B cell intrinsic mechanisms constraining ige memory. Front Immunol. 2017 Nov. 13; 8:1277.
60. Jackson K J L, Liu Y, Roskin K M, Glanville J, Hoh R A, Seo K, et al. Human responses to influenza vaccination show seroconversion signatures and convergent antibody rearrangements. Cell Host Microbe. 2014 Jul. 9; 16(1):105-114.
61. Zhou J, Lottenbach K R, Barenkamp S J, Lucas A H, Reason D C. Recurrent variable region gene usage and somatic mutation in the human antibody response to the capsular polysaccharide of Streptococcus pneumoniae type 23F. Infect Immun. 2002 August; 70(8):4083-4091.
62. Kiyotani K, Mai T H, Yamaguchi R, Yew P Y, Kulis M, Orgel K, et al. Characterization of the B-cell receptor repertoires in peanut allergic subjects undergoing oral immunotherapy. J Hum Genet. 2018 February; 63(2):239-248.
63. Bublin M, Kostadinova M, Radauer C, Hafner C, Szépfalusi Z, Varga E-M, et al. IgE cross-reactivity between the major peanut allergen Ara h 2 and the nonhomologous allergens Ara h 1 and Ara h 3. J Allergy Clin Immunol. 2013 July; 132(1):118-124.
64. Picelli S, Faridani O R, Björklund A K, Winberg G, Sagasser S, Sandberg R. Full-length RNA-seq from single cells using Smart-seq2. Nat Protoc. 2014 Jan. 2; 9(1):171-181.
65. Anders S, Pyl P T, Huber W. HTSeq—a Python framework to work with high-throughput sequencing data. Bioinformatics. 2015 Jan. 15; 31(2):166-169.
66. Van den Brink S C, Sage F, Vértesy Á, Spanjaard B, Peterson-Maduro J, Baron C S, et al. Single-cell sequencing reveals dissociation-induced gene expression in tissue subpopulations. Nat Methods. 2017 Sep. 29; 14(10):935-936.
67. Ritchie M E, Phipson B, Wu D, Hu Y, Law C W, Shi W, et al. limma powers differential expression analyses for RNA-sequencing and microarray studies. Nucleic Acids Res. 2015 Apr. 20; 43(7):e47.
68. Li H. A statistical framework for SNP calling, mutation discovery, association mapping and population genetical parameter estimation from sequencing data. Bioinformatics. 2011 Nov. 1; 27(21):2987-2993.
69. Canzar S, Neu K E, Tang Q, Wilson P C, Khan A A. BASIC: BCR assembly from single cells. Bioinformatics. 2017 Feb. 1; 33(3):425-427.
70. Chang Z, Li G, Liu J, Zhang Y, Ashby C, Liu D, et al. Bridger: a new framework for de novo transcriptome assembly using RNA-seq data. Genome Biol. 2015 Feb. 11; 16:30.
71. Camacho C, Coulouris G, Avagyan V, Ma N, Papadopoulos J, Bealer K, et al. BLAST+: architecture and applications. BMC Bioinformatics. 2009 Dec. 15; 10:421.
72. Lefranc M-P, Giudicelli V, Ginestoux C, Jabado-Michaloud J, Folch G, Bellahcene F, et al. IMGT, the international ImMunoGeneTics information system. Nucleic Acids Res. 2009 January; 37(Database issue):D1006-12.
73. Katona I M, Urban J F, Scher I, Kanellopoulos-Langevin C, Finkelman F D. Induction of an IgE response in mice by Nippostrongylus brasiliensis: characterization of lymphoid cells with intracytoplasmic or surface IgE. J Immunol. 1983 January; 130(1):350-356.
74. Ye J, Ma N, Madden T L, Ostell J M. IgBLAST: an immunoglobulin variable domain sequence analysis tool. Nucleic Acids Res. 2013 July; 41 (Web Server issue):W34-40.
75. Gupta N T, Vander Heiden J A, Uduman M, Gadala-Maria D, Yaari G, Kleinstein S H. Change-O: a toolkit for analyzing large-scale B cell immunoglobulin repertoire sequencing data. Bioinformatics. 2015 Oct. 15; 31(20):3356-3358.
76. Köster J, Rahmann S. Snakemake—a scalable bioinformatics workflow engine. Bioinformatics. 2012 Oct. 1; 28(19):2520-2522.

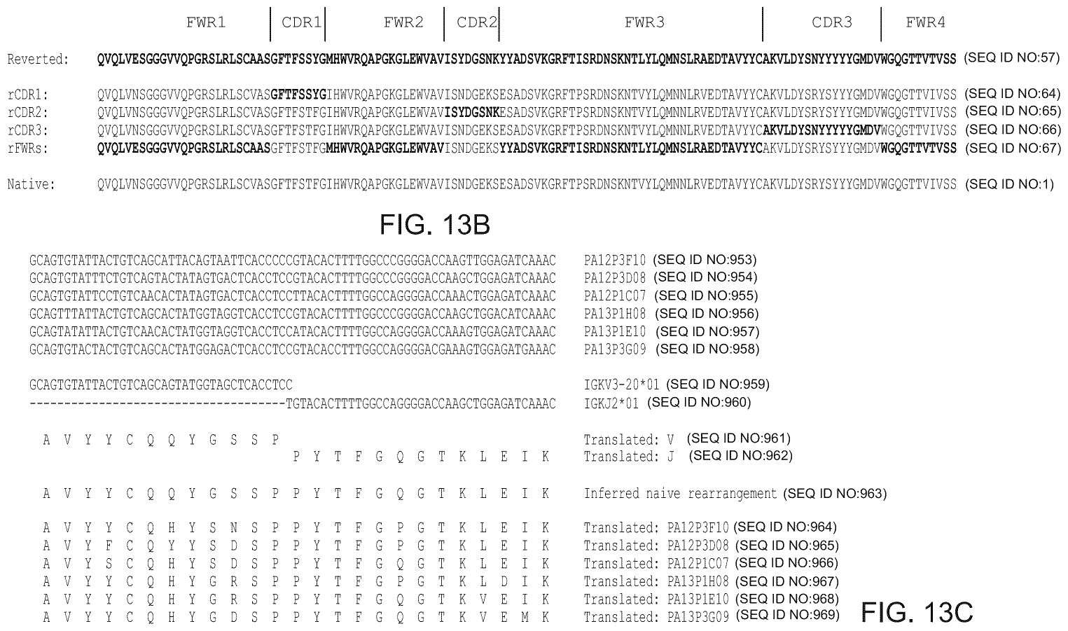

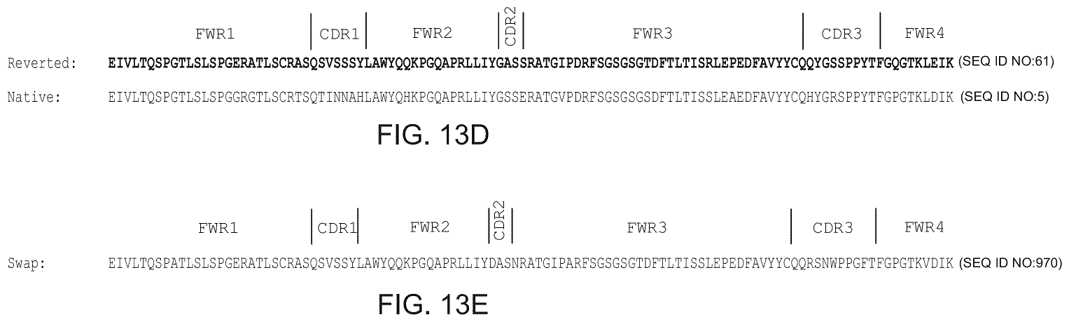

TABLE 1
Sequence Listing

| SEQ | | |
|---|---|---|
| ID | | |
| NO | Sequence | Description |
| 1 | QVQLVNSGGGVVQPGRSLRLSCVASGFTFSTFGIHWVRQAPGKGLEWVAVISNDGEKSESA | Heavy chain variable region |
| | DSVKGRFTPSRDNSKNTVYLQMNNLRVEDTAVYYCAKVLDYSRYSYYYGMDVWGQGTTVI | for clone PA13P1H08 and |
| | VSS | N-R variant of PA13P1H08 |
| 2 | GFTFSTFG | CDR-H1 for clone PA13P1H08 |
| | | and PA13P1H08 variants N-R, |
| | | rCDR2-N, rCDR3-N, rFWRs-N |
| 3 | ISNDGEKS | CDR-H2 for clone PA13P1H08 |
| | | and for PA13P1H08 variants |
| | | N-R, rCDR1-N, rCDR3-N |
| 4 | AKVLDYSRYSYYYGMDV | CDR-H3 for clone PA13P1H08 |
| | | and PA13P1H08 variants |
| | | N-R, rCDR1-N, rCDR2-N |
| 5 | EIVLTQSPGTLSLSPGGRGTLSCRTSQTINNAHLAWYQHKPGQAPRLLIYGSSERATGVPDRF | Light chain variable region |
| | SGSGSGSDFTLTISSLEAEDFAVYYCQHYGRSPPYTFGPGTKLDIK | for clone PA13P1H08 and |
| | | PA13P1H08 variants R-N, |
| | | rCDR1-N, rCDR2-N, |
| | | rCDR3-N, rFWRs-N |
| 6 | QTINNAH | CDR-L1 for clone PA13P1H08 |
| | | and PA13P1H08 variants R-N, |
| | | rCDR1-N, rCDR2-N, |
| | | rCDR3-N, rFWRs-N |
| 7 | GSS | CDR-L2 for clone PA13P1H08 |
| | | and PA13P1H08 variants |
| | | R-N, rCDR1-N, rCDR2-N, |
| | | rCDR3-N, rFWRs-N |
| 8 | QHYGRSPPYT | CDR-L3 for clone PA13P1H08 |
| | | and PA13P1H08 variants |
| | | R-N, rCDR1-N, rCDR2-N, |
| | | rCDR3-N, rFWRs-N |
| 9 | QVQLVDSGGGVVQPGKSLRLSCVGSGFTFRTFGIHWVRQAPGKGLEWVAVISNDGGNSAS | Clone PA13P1E10-Heavy chain |
| | ADSVKGRFTTSRDNSKNTVYLQINSLRPEDTAIYYCAKVLDYSAFSYYYGMDVWGQGTTVIVSS | variable region |
| 10 | GFTFRTFG | Clone PA13P1E10-CDR-H1 |
| 11 | ISNDGGNS | Clone PA13P1E10-CDR-H2 |
| 12 | AKVLDYSAFSYYYGMDV | Clone PA13P1E10-CDR-H3 |
| 13 | EIVLTQSPGTLSLSPGERGTLSCRTSQPISRAHLAWYQHKAGQAPRLLIYGSTERAAGIPERFS | Clone PA13P1E10-light chain |
| | GGGSGSDFTLTISSLEAEDFAVYYCQHYGRSPPYTFGQGTKVEIK | variable region |
| 14 | QPISRAH | Clone PA13P1E10-CDR-L1 |
| 15 | GST | Clone PA13P1E10-CDR-L2 |
| 8 | QHYGRSPPYT | Clone PA13P1E10-CDR-L3 |
| 16 | QVQLVESGGGVVQPGGSLTLSCVGSGFTFSHYAIHWVRQAPGKGLEWVAVISNVGTTRDY | Clone PA12P3F10-Heavy chain |
| | ADSLKGRLTISRENSQSTVFLQMNSLRADDTAIYYCAKVLDYSEFHYYYGLDVWGQGTAVAV | variable region |
| | SS | |
| 17 | GFTFSHYA | Clone PA12P3F10-CDR-H1 |
| 18 | ISNVGTTR | Clone PA12P3F10-CDR-H2 |
| 19 | AKVLDYSEFHYYYGLDV | Clone PA12P3F10-CDR-H3 |
| 20 | EIVLTQSPGTLSLSPGQRVTLSCRVSQAIPTMYVAWYQQRPGQAPRLLIYGTSSRATGIPDRFS | Clone PA12P3F10-light chain |
| | GGGSGTDFTLTINRLEPEDIAVYYCQHYSNSPPYTFGPGTKLEIK | variable region |
| 21 | QAIPTMY | Clone PA12P3F10-CDR-L1 |
| 22 | GTS | Clone PA12P3F10-CDR-L2 |
| 23 | QHYSNSPPYT | Clone PA12P3F10-CDR-L3 |
| 24 | QEQLVESGGGVVHPGGSLRLSCVASAFTFNRFGMHWVRQAPGKGLEWVAVISNDGRSQDY | Clone PA13P3G09-Heavy chain |
| | ADSVKGRFIISRDNSKNTLYLQLNSLRFEDTAVYYCAKVLDYSIFYYYFGLDVWGQGTTVTVSS | variable region |
| 25 | AFTFNRFG | Clone PA13P3G09-CDR-H1 |
| 26 | ISNDGRSQ | Clone PA13P3G09-CDR-H2 |
| 27 | AKVLDYSIFYYYFGLDV | Clone PA13P3G09-CDR-H3 |
| 28 | EVVLTQSPGSLSLSPGERATLSCRAGQSLSSKFLAWYQHKPGQAPRLLIYGASTRATGVPDRF | Clone PA13P3G09-light chain |
| | SGSGSGTDFSLIISRVEPEDFAVYYCQHYGDSPPYTFGQGTKVEMK | variable region |
| 29 | QSLSSKF | Clone PA13P3G09-CDR-L1 |
| 30 | GAS | CDR-L2 for clone PA13P3G09 |
| | | and PA13P1H08 variants |
| | | R-R, N-R |
| 31 | QHYGDSPPYT | Clone PA13P3G09-CDR-L3 |
| 32 | QVQLVESGGGVVQPGKSLRLSCAASAFTFRRFAMHWVRQAPGKGLEWVAVISDNGLREDY | Clone PA12P3D08-Heavy chain |
| | EDSVKGRFTISRDNSQNTLYLQMNGLRAEDTAVYYCAKVLDYNEYSLYFGMDVWGQGTTV | variable region |
| | TVSS | |
| 33 | AFTFRRFA | Clone PA12P3D08-CDR-H1 |
| 34 | ISDNGLRE | Clone PA12P3D08-CDR-H2 |
| 35 | AKVLDYNEYSLYFGMDV | Clone PA12P3D08-CDR-H3 |
| 36 | EVVLTQSPATLSLSPGERATLSCRTSQAISNNFLAWYQQRPGQPPRLLIYASSRRATDTPDRFT | Clone PA12P3D08-light chain |
| | GSGSGTDFTLTITRLEPEDFAVYFCQYYSDSPPYTFGPGTKLEIK | variable region |
| 37 | QAISNNF | Clone PA12P3D08-CDR-L1 |
| 38 | ASS | Clone PA12P3D08-CDR-L2 |
| 39 | QYYSDSPPYT | Clone PA12P3D08-CDR-L3 |
| 40 | QVQLEESGGGVVQPGKSLRLSCVASAFTFKRFAMHWVRQAPGKGLEWVAVISDNGLREDY | Clone PA12P1C07-Heavy chain |
| | EDSVKGRFTISRDNSKDTLYLQMNSLRPEDTAIYYCAKVLDYSEYSLYFGMDVWGQGTTVLV | variable region |
| | SS | |
| 41 | AFTFKRFA | Clone PA12P1C07-CDR-H1 |
| 34 | ISDNGLRE | Clone PA12P1C07-CDR-H2 |
| 42 | AKVLDYSEYSLYFGMDV | Clone PA12P1C07-CDR-H3 |
| 43 | EIVLTQSPAILSLSPGDRATLSCRTSQTVNSNFLAWYQQKPGQAPRLLIYGASRRAIDIPDRFT | Clone PA12P1C07-light chain |
| | GSGSGTEFTLTIARLEPEDFAVYSCQHYSDSPPYTFGQGTKLEIK | variable region |
| 44 | QTVNSNF | Clone PA12P1C07-CDR-L1 |
| 30 | GAS | Clone PA12P1C07-CDR-L2 |
| 45 | QHYSDSPPYT | Clone PA12P1C07-CDR-L3 |
| 46 | QVHLVESGGGVVQPGRSLGLSCAASGFTFNYYAIHWVRQAPGKGLEWVAVVSFDGNIIYYA | Clone PA15P1D12-Heavy chain |
| | DSVKGRFNISRDNSKNTVNLQMNSLRADDTAVYYCVRDGEYCSGGNCYWGDFDYWGQGT | variable region |
| | LVTVSP | |
| 47 | GFTFNYYA | Clone PA15P1D12-CDR-H1 |
| 48 | VSFDGNII | Clone PA15P1D12-CDR-H2 |
| 49 | VRDGEYCSGGNCYWGDFDY | Clone PA15P1D12-CDR-H3 |
| 50 | EIVLTQSPGTLSLSPGERATLSCRASQSISSEYLTWFQQKPGQAPRLLIYGAFNRATGIPDRFS | Clone PA15P1D12-light chain |
| | GSGSGTDFTLTISSLEPEDFAVYYCQQYANWWTFGQGTKVEIK | variable region |
| 51 | QSISSEY | Clone PA15P1D12-CDR-L1 |
| 52 | GAF | Clone PA15P1D12-CDR-L2 |
| 53 | QQYANWWT | Clone PA15P1D12-CDR-L3 |
| 54 | QVHLVESGGGVVQPGRSLGLSCVASGFTFNYYAIHWVRQAPGKGLEWVAVVSFDGNIIYYA | Clone PA15P1D05-Heavy chain |
| | DSVKGRFNISRDNSKNTVNLQMNSLRPDDTAVYYCVRDGEYCSGGNCYWGDFDHWGQGS | variable region |
| | LVTVSP | |
| 47 | GFTFNYYA | Clone PA15P1D05-CDR-H1 |
| 48 | VSFDGNII | Clone PA15P1D05-CDR-H2 |
| 55 | VRDGEYCSGGNCYWGDFDH | Clone PA15P1D05-CDR-H3 |
| 56 | EIVLTQSPATLSLSPGERATLSCRASQSISSEYLTWFQQKPGQAPRLLIYGAFNRATGIPDRFS | Clone PA15P1D05-light chain |
| | GSGSGTDFTLTISSLEPEDFAVYYCQQYANWWTFGQGTKVEIK | variable region |
| 51 | QSISSEY | Clone PA15P1D05-CDR-L1 |
| 52 | GAF | Clone PA15P1D05-CDR-L2 |
| 53 | QQYANWWT | Clone PA15P1D05-CDR-L3 |
| 57 | QVQLVESGGGVVQPGRSLRLSCAASGFTFSSYGMHWVRQAPGKGLEWVAVISYDGSNKYY | Heavy chain variable region |
| | ADSVKGRFTISRDNSKNTLYLQMNSLRAEDTAVYYCAKVLDYSNYYYYYGMDVWGQGTTVT | for R-R and R-N variants of |
| | VSS | PA13P1H08 |
| 58 | GFTFSSYG | CDR-H1 for PA13P1H08 variants |
| | | R-R, R-N, rCDR1-N |
| 59 | ISYDGSNK | CDR-H2 for PA13P1H08 variants |
| | | R-R, R-N, rCDR2-N |
| 60 | AKVLDYSNYYYYYGMDV | CDR-H3 for PA13P1H08 variants |
| | | R-R, R-N, rCDR3-N |
| 61 | EIVLTQSPGTLSLSPGERATLSCRASQSVSSSYLAWYQQKPGQAPRLLIYGASSRATGIPDRFS | Light chain variable for |
| | GSGSGTDFTLTISRLEPEDFAVYYCQQYGSSPPYTFGQGTKLEIK | region R-R and N-R variants |
| | | of PA13P1H08 |
| 62 | QSVSSSY | CDR-L1 for PA13P1H08 variants |
| | | R-R, N-R |
| 63 | QQYGSSPPYT | CDR-L3 for PA13P1H08 variants |
| | | R-R, N-R |
| 64 | QVQLVNSGGGVVQPGRSLRLSCVASGFTFSSYGIHWVRQAPGKGLEWVAVISNDGEKSESA | Heavy chain variable region |
| | DSVKGRFTPSRDNSKNTVYLQMNNLRVEDTAVYYCAKVLDYSRYSYYYGMDVWGQGTTVI | for PA13P1H08 variant rCDR1-N |
| | VSS | |
| 65 | QVQLVNSGGGVVQPGRSLRLSCVASGFTFSTFGIHWVRQAPGKGLEWVAVISYDGSNKESA | Heavy chain variable region |
| | DSVKGRFTPSRDNSKNTVYLQMNNLRVEDTAVYYCAKVLDYSRYSYYYGMDVWGQGTTVI | for PA13P1H08 variant rCDR2-N |
| | VSS | |
| 66 | QVQLVNSGGGVVQPGRSLRLSCVASGFTFSTFGIHWVRQAPGKGLEWVAVISNDGEKSESA | Heavy chain variable region |
| | DSVKGRFTPSRDNSKNTVYLQMNNLRVEDTAVYYCAKVLDYSNYYYYYGMDVWGQGTTVI | for PA13P1H08 variant rCDR3-N |
| | VSS | |
| 67 | QVQLVESGGGVVQPGRSLRLSCAASGFTFSTFGMHWVRQAPGKGLEWVAVISNDGEKSYY | Heavy chain variable region |
| | ADSVKGRFTISRDNSKNTLYLQMNSLRAEDTAVYYCAKVLDYSRYSYYYGMDVWGQGTTVT | for PA13P1H08 variant rFWRs-N |
| | VSS | |
| 68 | GCCTCCACACAGAGCCCATCCGTCTTCCCCTTGACCCGCTGCTGCAAAAACATTCCCTCCA | Nucleotide sequence for IgE |
| | ATGCCACCTCCGTGACTCTGGGCTGCCTGGCCACGGGCTACTTCCCGGAGCCGGTGATG | heavy chain constant region |
| | GTGACCTGGGACACAGGCTCCCTCAACGGGACAACTATGACCTTACCAGCCACCACCCTC | |
| | ACGCTCTCTGGTCACTATGCCACCATCAGCTTGCTGACCGTCTCGGGTGCGTGGGCCAAG | |
| | CAGATGTTCACCTGCCGTGTGGCACACACTCCATCGTCCACAGACTGGGTCGACAACAAA | |
| | ACCTTCAGCGTCTGCTCCAGGGACTTCACCCCGCCCACCGTGAAGATCTTACAGTCGTCCT | |
| | GCGACGGCGGCGGGCACTTCCCCCCGACCATCCAGCTCCTGTGCCTCGTCTCTGGGTACA | |
| | CCCCAGGGACTATCAACATCACCTGGCTGGAGGACGGGCAGGTCATGGACGTGGACTTG | |
| | TCCACCGCCTCTACCACGCAGGAGGGTGAGCTGGCCTCCACACAAAGCGAGCTCACCCTC | |
| | AGCCAGAAGCACTGGCTGTCAGACCGCACCTACACCTGCCAGGTCACCTATCAAGGTCAC | |
| | ACCTTTGAGGACAGCACCAAGAAGTGTGCAGATTCCAACCCGAGAGGGGTGAGCGCCTA | |
| | CCTAAGCCGGCCCAGCCCGTTCGACCTGTTCATCCGCAAGTCGCCCACGATCACCTGTCT | |
| | GGTGGTGGACCTGGCACCCAGCAAGGGGACCGTGAACCTGACCTGGTCCCGGGCCAGT | |
| | GGGAAGCCTGTGAACCACTCCACCAGAAAGGAGGAGAAGCAGCGCAATGGCACGTTAA | |
| | CCGTCACGTCCACCCTGCCGGTGGGCACCCGAGACTGGATCGAGGGGGAGACCTACCAG | |
| | TGCAGGGTGACCCACCCCCACCTGCCCAGGGCCCTCATGCGGTCCACGACCAAGACCAG | |
| | CGGCCCGCGTGCTGCCCCGGAAGTCTATGCGTTTGCGACGCCGGAGTGGCCGGGGAGC | |
| | CGGGACAAGCGCACCCTCGCCTGCCTGATCCAGAACTTCATGCCTGAGGACATCTCGGTG | |
| | CAGTGGCTGCACAACGAGGTGCAGCTCCCGGACGCCCGGCACAGCACGACGCAGCCCC | |
| | GCAAGACCAAGGGCTCCGGCTTCTTCGTCTTCAGCCGCCTGGAGGTGACCAGGGCCGAA | |
| | TGGGAGCAGAAAGATGAGTTCATCTGCCGTGCAGTCCATGAGGCAGCGAGCCCCTCACA | |
| | GACCGTCCAGCGAGCGGTGTCTGTAAATCCCGGTAAA | |
| 69 | ASTQSPSVFPLTRCCKNIPSNATSVTLGCLATGYFPEPVMVTWDTGSLNGTTMTLPATTLTLS | Amino acid sequence for IgE |
| | GHYATISLLTVSGAWAKQMFTCRVAHTPSSTDWVDNKTFSVCSRDFTPPTVKILQSSCDGG | heavy chain constant region |
| | GHFPPTIQLLCLVSGYTPGTINITWLEDGQVMDVDLSTASTTQEGELASTQSELTLSQKHWLS | |
| | DRTYTCQVTYQGHTFEDSTKKCADSNPRGVSAYLSRPSPFDLFIRKSPTITCLVVDLAPSKGTV | |
| | NLTWSRASGKPVNHSTRKEEKQRNGTLTVTSTLPVGTRDWIEGETYQCRVTHPHLPRALMR | |
| | STTKTSGPRAAPEVYAFATPEWPGSRDKRTLACLIQNFMPEDISVQWLHNEVQLPDARHSTT | |
| | QPRKTKGSGFFVFSRLEVTRAEWEQKDEFICRAVHEAASPSQTVQRAVSVNPGK | |
| 70 | GCTTCCACCAAGGGCCCATCCGTCTTCCCCCTGGCGCCCTGCTCCAGGAGCACCTCCGAG | Nucleotide sequence for IgG4 |
| | AGCACAGCCGCCCTGGGCTGCCTGGTCAAGGACTACTTCCCCGAACCGGTGACGGTGTC | heavy chain constant region |
| | GTGGAACTCAGGCGCCCTGACCAGCGGCGTGCACACCTTCCCGGCTGTCCTACAGTCCTC | |
| | AGGACTCTACTCCCTCAGCAGCGTGGTGACCGTGCCCTCCAGCAGCTTGGGCACGAAGA | |
| | CCTACACCTGCAACGTAGATCACAAGCCCAGCAACACCAAGGTGGACAAGAGAGTTGAG | |
| | TCCAAATATGGTCCCCCATGCCCATCATGCCCAGCACCTGAGTTCCTGGGGGGACCATCA | |
| | GTCTTCCTGTTCCCCCCAAAACCCAAGGACACTCTCATGATCTCCCGGACCCCTGAGGTCA | |
| | CGTGCGTGGTGGTGGACGTGAGCCAGGAAGACCCCGAGGTCCAGTTCAACTGGTACGT | |
| | GGATGGCGTGGAGGTGCATAATGCCAAGACAAAGCCGCGGGAGGAGCAGTTCAACAGC | |
| | ACGTACCGTGTGGTCAGCGTCCTCACCGTCCTGCACCAGGACTGGCTGAACGGCAAGGA | |
| | GTACAAGTGCAAGGTCTCCAACAAAGGCCTCCCGTCCTCCATCGAGAAAACCATCTCCAA | |
| | AGCCAAAGGGCAGCCCCGAGAGCCACAGGTGTACACCCTGCCCCCATCCCAGGAGGAG | |
| | ATGACCAAGAACCAGGTCAGCCTGACCTGCCTGGTCAAAGGCTTCTACCCCAGCGACATC | |
| | GCCGTGGAGTGGGAGAGCAATGGGCAGCCGGAGAACAACTACAAGACCACGCCTCCCG | |
| | TGCTGGACTCCGACGGCTCCTTCTTCCTCTACAGCAGGCTAACCGTGGACAAGAGCAGGT | |
| | GGCAGGAGGGGAATGTCTTCTCATGCTCCGTGATGCATGAGGCTCTGCACAACCACTAC | |
| | ACACAGAAGAGCCTCTCCCTGTCTCTGGGTAAA | |
| 71 | ASTKGPSVFPLAPCSRSTSESTAALGCLVKDYFPEPVTVSWNSGALTSGVHTFPAVLQSSGLYS | Amino acid sequence for IgG4 |
| | LSSVVTVPSSSLGTKTYTCNVDHKPSNTKVDKRVESKYGPPCPSCPAPEFLGGPSVFLFPPKPK | heavy chain constant region |
| | DTLMISRTPEVTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGNVFSCSVMHEAL | |
| | HNHYTQKSLSLSLGK | |
| 72 | QVQLVESGGGVVQPGRSLRLSCAASGFTFDTYGMHWVRQAPGKGPEWVAVIWYDGTRED | Clone PA14P1E12-Heavy chain |
| | YADSVKGRFTVSRDNSKSTLFLQMNSLRADDTAVYYCAKEHNTYFSDHIGRVGGMDVWGQ | variable region |
| | GTTVIVSS | |
| 73 | GFTFDTYG | Clone PA14P1E12-CDR-H1 |
| 74 | IWYDGTRE | Clone PA14P1E12-CDR-H2 |
| 75 | AKEHNTYFSDHIGRVGGMDV | Clone PA14P1E12-CDR-H3 |
| 76 | DIVMTQSPLSLPVTPGEPASISCRSSQSLLHSNGYNYLDWYLQKPGQSPQLLIYLGSNRASGV | Clone PA14P1E12-light chain |
| | PDRFSGSGSGTDFTLKISRVEAEDVGVYYCMQVLQTPPWTFGQGTQVEIK | variable region |
| 77 | QSLLHSNGYNY | Clone PA14P1E12-CDR-L1 |
| 78 | LGS | Clone PA14P1E12-CDR-L2 |
| 79 | MQVLQTPPWT | Clone PA14P1E12-CDR-L3 |
| 80 | QVQLVESGGGVVQPGRSLRLSCAGSGFTFNAYGLHWVRQAPGKGLEWVAGIYYDGSNKYY | Clone PA14P1E10-Heavy chain |
| | ADSVKGRFAISRDNSQNTLYLEMNSLRVEDTAVYYCAKAGPIASIGTRHTFDHWGQGTLVTV | variable region |
| | SS | |
| 81 | GFTFNAYG | Clone PA14P1E10-CDR-H1 |
| 82 | IYYDGSNK | Clone PA14P1E10-CDR-H2 |
| 83 | AKAGPIASIGTRHTFDH | Clone PA14P1E10-CDR-H3 |
| 84 | DIVMTQSPDSLAVSLGERATINCKSSQSLLLNSNNKNYLAWYQQKPGQPPKLLIYWASTRES | Clone PA14P1E10-light chain |
| | GVPGRFSGNGSVTDFTLTISGLQAEDVAVYYCHQYYTTSYTFGQGTKLEIK | variable region |
| 85 | QSLLLNSNNKNY | Clone PA14P1E10-CDR-L1 |
| 86 | WAS | Clone PA14P1E10-CDR-L2 |
| 87 | HQYYTTSYT | Clone PA14P1E10-CDR-L3 |
| 88 | QVQLVQSGAEVKKPGASVKISCKAVGYTFTSYYLHWVRQAPGQGLEWVGIIDPSRGHRNYA | Clone PA14P1E11-Heavy chain |
| | QGFQGRVTMTSDTSTSTVYMDLGSLRSEDTAVYYCARAPARDHFDNWGQGTPVTVSP | variable region |
| 89 | GYTFTSYY | Clone PA14P1E11-CDR-H1 |
| 90 | IDPSRGHR | Clone PA14P1E11-CDR-H2 |
| 91 | ARAPARDHFDN | Clone PA14P1E11-CDR-H3 |
| 92 | DIQMTQSPSSLAASVGDRVTINCQASQDIRNCLNWYQQQPGKAPKLLIYDASILETGVPSRFS | Clone PA14P1E11-light chain |
| | GSGSGTDFTFSISSLQPEDIATYYCQQCEDLPLTFGPGSKVDIK | variable region |
| 93 | QDIRNC | Clone PA14P1E11-CDR-L1 |
| 94 | DAS | Clone PA14P1E11-CDR-L2 |
| 95 | QQCEDLPLT | Clone PA14P1E11-CDR-L3 |
| 96 | QVQLVQSGAEVKQPGSSVKVSCKASGGTFRNSALSWVRQAPGQGLEWMGGIIPIFDTTNY | Clone PA13P1C01-Heavy chain |
| | AQEFQGRVTITADKSTTTAYMELSSLKSEDTAVYYCARGEGLPWLTYHYYGMDVWGQGTTV | variable region |
| | TVSS | |
| 97 | GGTFRNSA | Clone PA13P1C01-CDR-H1 |
| 98 | IIPIFDTT | Clone PA13P1C01-CDR-H2 |
| 99 | ARGEGLPWLTYHYYGMDV | Clone PA13P1C01-CDR-H3 |
| 100 | QAVLTQPSSLSASPGASASLTCTLRSGINIGTDRIYWFQQKPGSPPQYLLTYKSDSDEQRGSG | Clone PA13P1C01-light chain |
| | VPSRFSGSKDVSANAGILLISGLQSEDEADYYCMIWHSSAWVFGGGTKLTVL | variable region |
| 101 | SGINIGTDR | Clone PA13P1C01-CDR-L1 |
| 102 | YKSDSDE | Clone PA13P1C01-CDR-L2 |
| 103 | MIWHSSAWV | Clone PA13P1C01-CDR-L3 |
| 104 | QVQLQQWGAGLLKPSETLSLTCAVYGGSLSGYHWSWIRQPPGKGLQWIGEISHSGNAKYN | Clone PA14P1F11-Heavy chain |
| | PSLKSRVSISVHMSKNEFYLNLTSVTAADTAVYYCARGYCSGGSCYYKFWGQGTLVTVSS | variable region |
| 105 | GGSLSGYH | Clone PA14P1F11-CDR-H1 |
| 106 | ISHSGNA | Clone PA14P1F11-CDR-H2 |
| 107 | ARGYCSGGSCYYKF | Clone PA14P1F11-CDR-H3 |
| 108 | QSVLTQPPSVSAAPGQKVTISCSGNSSNIGNNYVSWFQQLPGTAPKLLIYDNNKRPSGIPDRF | Clone PA14P1F11-light chain |
| | SGSKSGTSATLGITGLQTGDEADYFCGTWDSSLRTGVFGGGTKLTVL | variable region |
| 109 | SSNIGNNY | Clone PA14P1F11-CDR-L1 |
| 110 | DNN | Clone PA14P1F11-CDR-L2 |
| 111 | GTWDSSLRTGV | Clone PA14P1F11-CDR-L3 |
| 112 | EVQLLESGGGLVQPGGSLRLSCAASGFTFSSYAMSWVRQAPGKGLEWVSLIYSGGSRTSYPD | Clone PA14P1F10-Heavy chain |
| | SVKGRFTISRDNSNSTLFLQMNSLRVEDTAVYYCAKGGSSWLKMDYWGQGTLVIVSS | variable region |
| 113 | GFTFSSYA | Clone PA14P1F10-CDR-H1 |
| 114 | IYSGGSRT | Clone PA14P1F10-CDR-H2 |
| 115 | AKGGSSWLKMDY | Clone PA14P1F10-CDR-H3 |
| 116 | QSVLTQPPSVSGAPGQRVTISCTGSSSNIGAGYDVQWYQHLPGTAPKLLIFANTNRPSGVPD | Clone PA14P1F10-light chain |
| | RFSGSKSGTSASLAITGLQAEDEADYYCQSYDSSLSGSVFGGGTKLTVL | variable region |
| 117 | SSNIGAGYD | Clone PA14P1F10-CDR-L1 |
| 118 | ANT | Clone PA14P1F10-CDR-L2 |
| 119 | QSYDSSLSGSV | Clone PA14P1F10-CDR-L3 |
| 120 | QVQLVQSGAEVKKPGASVRVSCSSSGYTFTGYYIHWVRQAPGQGLEYMGRINPHSGGTNY | Clone PA13P1C09-Heavy chain |
| | AQKFQGRVTMTRDTSTSTVYMELSSLRSDDTAVYYCAKEGTTAHIFNWFDPWGQGTLVTVSS | variable region |
| 121 | GYTFTGYY | Clone PA13P1C09-CDR-H1 |
| 122 | INPHSGGT | Clone PA13P1C09-CDR-H2 |
| 123 | AKEGTTAHIFNWFDP | Clone PA13P1C09-CDR-H3 |
| 124 | DIQMTQSPSSLSASVGDRVTITCRASQSINSYLNWYQQKPGKAPNLLIYTASSLQSGVPSRFS | Clone PA13P1C09-light chain |
| | GSGSGTDFTLTISSLQPEDFATYYCQQSYTSLFTFGQGTKLEIK | variable region |
| 125 | QSINSY | Clone PA13P1C09-CDR-L1 |
| 126 | TAS | Clone PA13P1C09-CDR-L2 |
| 127 | QQSYTSLFT | Clone PA13P1C09-CDR-L3 |
| 128 | QLQLQESGPGLVKPSETLSLTCTVSGVSINSTSYYWGWMRQPPGKGLEWIGNIYYTGTTYYN | Clone PA13P1D02-Heavy chain |
| | PSLNRRVSISGDTSKNQFSLSLTSVTAADTAVYYCAGPRRVTVFGILLMESFDVWSQGTMVT | variable region |
| | VSS | |
| 129 | GVSINSTSYY | Clone PA13P1D02-CDR-H1 |
| 130 | IYYTGTT | Clone PA13P1D02-CDR-H2 |
| 131 | AGPRRVTVFGILLMESFDV | Clone PA13P1D02-CDR-H3 |
| 132 | AIQMTQSPSSLSASVGDRVTITCRASQAIRDDLGWFQQKPGKAPKLLIYTASTLQSGVPSRFS | Clone PA13P1D02-light chain |
| | GGGSGTEFILTISSLQPEDIGTYYCLQDYGYPWTFGQGTKVEIK | variable region |
| 133 | QAIRDD | Clone PA13P1D02-CDR-L1 |
| 126 | TAS | Clone PA13P1D02-CDR-L2 |
| 134 | LQDYGYPWT | Clone PA13P1D02-CDR-L3 |
| 135 | EVQLLESGGGLVQPGGSLRLSCAASGFTFSNYALSWVRQAPGKGLEWVSAISGRDASTYYAD | Clone PA14P1E04-Heavy chain |
| | SVKGRFTISRDNSKNTLYLQMNSLRAEDTAVYYCTLFDYDSSGYFDFDYWGQGTLVTVSS | variable region |
| 136 | GFTFSNYA | Clone PA14P1E04-CDR-H1 |
| 137 | ISGRDAST | Clone PA14P1E04-CDR-H2 |
| 138 | TLFDYDSSGYFDFDY | Clone PA14P1E04-CDR-H3 |
| 139 | QSALTQPRSVSGSPGQSVTISCTGTGSDVGGYNYVSWYQHHPGKAPKLIIFDVTKRPSGVPD | Clone PA14P1E04-light chain |
| | RFSGSKSGYTASLTISGLQAEDEAVYYCCSYANSYTGVFGTGTKVTVL | variable region |
| 140 | GSDVGGYNY | Clone PA14P1E04-CDR-L1 |
| 141 | DVT | Clone PA14P1E04-CDR-L2 |
| 142 | CSYANSYTGV | Clone PA14P1E04-CDR-L3 |
| 143 | QVQLVESGGGVVQPGGSLRLSCAASGFTFSSHVMHWVRQAPGKGLEWVALISLDGDDKYY | Clone PA14P1E06-Heavy chain |
| | ADSVNGRVAISRDNSKNTLYLQVNSLRSDDTCVYYCARGGRWDYALDVWGQGTTVTVSS | variable region |
| 144 | GFTFSSHV | Clone PA14P1E06-CDR-H1 |
| 145 | ISLDGDDK | Clone PA14P1E06-CDR-H2 |
| 146 | ARGGRWDYALDV | Clone PA14P1E06-CDR-H3 |
| 147 | EIVMTQSPATLSVSPGERATLSCRVSQSISNSLAWYQQKPGQVPRLLIYAASTRATGIPARFSG | Clone PA14P1E06-light chain |
| | SGSGTEFTLTISSLQSEDFAVYYCQQYNNWPRALTFGGGTKVEIK | variable region |
| 148 | QSISNS | Clone PA14P1E06-CDR-L1 |
| 149 | AAS | Clone PA14P1E06-CDR-L2 |
| 150 | QQYNNWPRALT | Clone PA14P1E06-CDR-L3 |
| 151 | QVQLVESGGGVVQPGRSLRLSCAASGFTFNDYAMHWVRQAPGKGPEWVAVISYDGTNEY | Clone PA11P1G06-Heavy chain |
| | YMGSVKGRFTISRDNSKNMVNLQMNSLRPEDTAVYYCARDLAAWSRELLVFDQWGQGTL | variable region |
| | VTVSS | |
| 152 | GFTFNDYA | Clone PA11P1G06-CDR-H1 |
| 153 | ISYDGTNE | Clone PA11P1G06-CDR-H2 |
| 154 | ARDLAAWSRELLVFDQ | Clone PA11P1G06-CDR-H3 |
| 155 | ENVLTQSPGTLSLSPGEGATLSCRASQSVPNTYLAWYQQKPGQAPRLLIYGASSRAAGIPDRF | Clone PA11P1G06-light chain |
| | SGSGSGTDFTLTISRLEPEDFAVYYCQQYGRSPGTFGQGTKVEIK | variable region |
| 156 | QSVPNTY | Clone PA11P1G06-CDR-L1 |
| 30 | GAS | Clone PA11P1G06-CDR-L2 |
| 157 | QQYGRSPGT | Clone PA11P1G06-CDR-L3 |
| 158 | QAQVVESGGGVVQPGTSLRLSCEPSGFTLSDYGIHWVRQPPGKGLEWVAVIWHDGDRINY | Clone PA11P1G07-Heavy chain |
| | ADSVKGRFTISRDESDKKVHLQMESLRTEDTAVYYCARGTLPRNCRGMRCYGEFDHYYYLDV | variable region |
| | WGTGTTVTVSS | |
| 159 | GFTLSDYG | Clone PA11P1G07-CDR-H1 |
| 160 | IWHDGDRI | Clone PA11P1G07-CDR-H2 |
| 161 | ARGTLPRNCRGMRCYGEFDHYYYLDV | Clone PA11P1G07-CDR-H3 |
| 162 | QSVLTQPPSVSGAPGQRVTISCTGHSSNIGANSDVHWYQQLPLRAPKLLIFGTINRASGVPD | Clone PA11P1G07-light chain |
| | RFSGSRSGTSASLVISGLQPDDEADYYCQSYDRGLSAYVFGSGTRVDVL | variable region |
| 163 | SSNIGANSD | Clone PA11P1G07-CDR-L1 |
| 164 | GTI | Clone PA11P1G07-CDR-L2 |
| 165 | QSYDRGLSAYV | Clone PA11P1G07-CDR-L3 |
| 166 | EVQLVESGGGLVQPGGSLRLSCAASGFTFSSYWMSWVRQAPGKGLEWVANIKQDGSEKDY | Clone PA11P1G04-Heavy chain |
| | VDSVKGRFTISRDNAKNSLYLQLNSLRAEDTAVYYCARERSTQSSSWYVSSYYSYYGMDVWG | variable region |
| | QGTTVTVSS | |
| 167 | GFTFSSYW | Clone PA11P1G04-CDR-H1 |
| 168 | IKQDGSEK | Clone PA11P1G04-CDR-H2 |
| 169 | ARERSTQSSSWYVSSYYSYYGMDV | Clone PA11P1G04-CDR-H3 |
| 170 | SSELTQDPAVSVALGQTVTITCQGDSLRSFYASWYQQKPGQAPVFVIYGKYNRPSGIPDRFSG | Clone PA11P1G04-light chain |
| | SSSGNTASLTITGAQAEDEADYYCNSRDSSDNHLGVFGGGTKLTVL | variable region |
| 171 | SLRSFY | Clone PA11P1G04-CDR-L1 |
| 172 | GKY | Clone PA11P1G04-CDR-L2 |
| 173 | NSRDSSDNHLGV | Clone PA11P1G04-CDR-L3 |
| 174 | QVQLVQSGSELRKPGASVKLSCRTSGYTFIHFAMNWLRQAPGQGLEWLGWINTHSGNPTY | Clone PA11P1C11-Heavy chain |
| | AQGFTGRFVFSLDVSAGTAYLEISGLKAEDTAVYYCARERYFDFWGQGALVAVSS | variable region |
| 175 | GYTFIHFA | Clone PA11P1C11-CDR-H1 |
| 176 | INTHSGNP | Clone PA11P1C11-CDR-H2 |
| 177 | ARERYFDF | Clone PA11P1C11-CDR-H3 |
| 178 | QSVLTQPPSASGTPGQRVTISCSGTSSNIGKNFLYWYQQVPGTAPKLLIYSSNQRPSGVPDRF | Clone PA11P1C11-light chain |
| | SGSKSGTSASLAISGLRSEDEADYYCAAWDDSLSGWVFGGGTKVTVL | variable region |
| 179 | SSNIGKNF | Clone PA11P1C11-CDR-L1 |
| 180 | SSN | Clone PA11P1C11-CDR-L2 |
| 181 | AAWDDSLSGWV | Clone PA11P1C11-CDR-L3 |
| 182 | EVQLVESGGDLVQPGGSLRLSCAASGFTFSNYWMNWVRQPPGKGLVWVSRISGDGTGTSY | Clone PA11P1C12-Heavy chain |
| | ADSVRGRFTISRDNAKSTLYLQVNSLSAEDTAVYYCTRDGGRDHPTPDAFDIWGQGTMVTV | variable region |
| | SS | |
| 183 | GFTFSNYW | Clone PA11P1C12-CDR-H1 |
| 184 | ISGDGTGT | Clone PA11P1C12-CDR-H2 |
| 185 | TRDGGRDHPTPDAFDI | Clone PA11P1C12-CDR-H3 |
| 186 | DVVMAQSPLSLPVTLGQPASISCRSSQSLVHSDGNTYLNWFQQRPGQSPRRLIYKISNRDSG | Clone PA11P1C12-light chain |
| | VPDRFSGSGSGTDFTLKISRVEAEDVGVYYCMQGTHWPRTFGQGTKLEIK | variable region |
| 187 | QSLVHSDGNTY | Clone PA11P1C12-CDR-L1 |
| 188 | KIS | Clone PA11P1C12-CDR-L2 |
| 189 | MQGTHWPRT | Clone PA11P1C12-CDR-L3 |
| 190 | QVQLQQWGAGLLKPSETLSLTCVVSGGSFSTHYWNWIRQSPGKGLEWIGEINHSGNTNYN | Clone PA15P1G05-Heavy chain |
| | PSLTGRATISVATSKTQFSLRLNSVTAADTAVYFCARGPRLRYTAGRPLFDTWGQGTLVTVSS | variable region |
| 191 | GGSFSTHY | Clone PA15P1G05-CDR-H1 |
| 192 | INHSGNT | Clone PA15P1G05-CDR-H2 |
| 193 | ARGPRLRYTAGRPLFDT | Clone PA15P1G05-CDR-H3 |
| 194 | DIQMTQSPSTLSASVGDRVTITCRASQSISAFLAWYQQKPGKAPNLVIYKASSLDSGVPSTFS | Clone PA15P1G05-light chain |
| | GSGSGTEYTLTISSLQPDDFATYYCQQYFSSPPTFGQGTKVEMK | variable region |
| 195 | QSISAF | Clone PA15P1G05-CDR-L1 |
| 196 | KAS | Clone PA15P1G05-CDR-L2 |
| 197 | QQYFSSPPT | Clone PA15P1G05-CDR-L3 |
| 198 | QVQLQESGPGLVKPSQTLSLTCAVSGGSISSGGYYWSWIRQLPGKGLEWIGYIYYSGSTSYNP | Clone PA14P1F05-Heavy chain |
| | SLKSRVTISVDTSKNQLSLNLSSVTAADTAVYNCARGRRISISGVVTPLFDYWGQGTLVTVSS | variable region |
| 199 | GGSISSGGYY | Clone PA14P1F05-CDR-H1 |
| 200 | IYYSGST | Clone PA14P1F05-CDR-H2 |
| 201 | ARGRRISISGVVTPLFDY | Clone PA14P1F05-CDR-H3 |
| 202 | DIQMTQSPSSVSASVGDRVTITCRASQGISSWLAWYQLKPGKAPKLLIYAASSLQSGVPSRFS | Clone PA14P1F05-light chain |
| | GSGSGTDFTLTISSLQPEDFATYYCQQANSVPLTFGGGTKVEIK | variable region |
| 203 | QGISSW | Clone PA14P1F05-CDR-L1 |
| 149 | AAS | Clone PA14P1F05-CDR-L2 |
| 204 | QQANSVPLT | Clone PA14P1F05-CDR-L3 |
| 205 | EVQLVESGGGLVKPGGSLRLSCAASGFTFSHYYLNWVRQAPGKGLEWVACISDRSENVYYA | Clone PA14P1F07-Heavy chain |
| | DSVKGRFTISRDNAKNSLFLQMNNLRAEDTAIYYCARDMRELRPSADYWGQGTLVTVSS | variable region |
| 206 | GFTFSHYY | Clone PA14P1F07-CDR-H1 |
| 207 | ISDRSENV | Clone PA14P1F07-CDR-H2 |
| 208 | ARDMRELRPSADY | Clone PA14P1F07-CDR-H3 |
| 209 | EIVLTQSPGTLSLSPGDRATLSCRASQSVDGNSLAWYQQKPGQAPRLLISGASTRATGIPDRF | Clone PA14P1F07-light chain |
| | SGSGSGTDFTLTISRLEPEDFVLYHCQLYTVSPRYTFGQGTKLEIK | variable region |
| 210 | QSVDGNS | Clone PA14P1F07-CDR-L1 |
| 30 | GAS | Clone PA14P1F07-CDR-L2 |
| 211 | QLYTVSPRYT | Clone PA14P1F07-CDR-L3 |
| 212 | QLQLQESGPGLVKPSETLSLTCTVSGGSISSDNYYWGWIRQPPGKGPLWIGTIFYNGDTYYN | Clone PA14P3H12-Heavy chain |
| | PSLKSQLNISVDPSKNQFSLKLTSVTAADTAIYYCTRHDSYSRGWYVTHWGQGTLVTVSS | variable region |
| 213 | GGSISSDNYY | Clone PA14P3H12-CDR-H1 |
| 214 | IFYNGDT | Clone PA14P3H12-CDR-H2 |
| 215 | TRHDSYSRGWYVTH | Clone PA14P3H12-CDR-H3 |
| 216 | EIVLTQSPATLSLFPGERATLSCRASQSVTSYLAWYQQKPGQAPRLLIYDASKRATGIPARFSG | Clone PA14P3H12-light chain |
| | SGSGTDFTLTISSLEPEDFATYYCQQRSARQLFGGGTKVEIK | variable region |
| 217 | QSVTSY | Clone PA14P3H12-CDR-L1 |
| 94 | DAS | Clone PA14P3H12-CDR-L2 |
| 218 | QQRSARQL | Clone PA14P3H12-CDR-L3 |
| 219 | EVQLVESGGGLVKPGGSLRLSCVASGLTFRNAWMTWVRQAPGKGLEWVGRIKSNVNGGT | Clone PA14P3H10-Heavy chain |
| | TDYAAPVRGRFTISRDDSRDTLYLQMNSLETEDTAMYYCTKDPPYTGGGYCQHWGLGTLVT | variable region |
| | VSS | |
| 220 | GLTFRNAW | Clone PA14P3H10-CDR-H1 |
| 221 | IKSNVNGGTT | Clone PA14P3H10-CDR-H2 |
| 222 | TKDPPYTGGGYCQH | Clone PA14P3H10-CDR-H3 |
| 223 | EIVLTQSPATLSLSPGESATLSCRASQSVSSCLAWYQQKPGQAPRLLIYDASTRAPGIPGRFSG | Clone PA14P3H10-light chain |
| | SGSGTDFTLAISSLEPEDFAVYYCQQCSNWPLTFGRGTRLEIK | variable region |
| 224 | QSVSSC | Clone PA14P3H10-CDR-L1 |
| 94 | DAS | Clone PA14P3H10-CDR-L2 |
| 225 | QQCSNWPLT | Clone PÅ14P3H10-CDR-L3 |
| 226 | QVQLQESGPGLVKPSQTLSLTCTVSGGSINTGAYYWSWIRQHPGKGLEWIGYIYYSGSTYYN | Clone PA11P1G10-Heavy chain |
| | PSLKSRVTISKDTSKNQFSLRLTSVTAADTAVYYCVREKLTGAPDNWGQGTLVAVSS | variable region |
| 227 | GGSINTGAYY | Clone PA11P1G10-CDR-H1 |
| 200 | IYYSGST | Clone PA11P1G10-CDR-H2 |
| 228 | VREKLTGAPDN | Clone PA11P1G10-CDR-H3 |
| 229 | DIQMTQSPSSLSASVGDRVTITCRASQGVSNYLAWFHQKPGKAPKSLIYAASTLHDGVPSSFS | Clone PA11P1G10-light chain |
| | GSGSGTEFTLTISDLQPEHFGTYYCEQYNSYPFTFGPGTTVDFK | variable region |
| 230 | QGVSNY | Clone PA11P1G10-CDR-L1 |
| 149 | AAS | Clone PA11P1G10-CDR-L2 |
| 231 | EQYNSYPFT | Clone PA11P1G10-CDR-L3 |
| 232 | QVQLVQSGAEVKKPGSSVKVSCKASGGPLSSYNFIWVRQAPGQGLEWMGGILPVFDTTNY | Clone PA13P2H10-Heavy chain |
| | AQKFQGRVTITADKATSTSYMELSSLTSEDTAVYYCARAVGGTHYYYYGLDVWGQGTTVAV | variable region |
| | SS | |
| 233 | GGPLSSYN | Clone PA13P2H10-CDR-H1 |
| 234 | ILPVFDTT | Clone PA13P2H10-CDR-H2 |
| 235 | ARAVGGTHYYYYGLDV | Clone PA13P2H10-CDR-H3 |
| 236 | EIVMTQSPLSLPVTPGEPASISCRSSQSLLHGNGYNYVDWYLQRPGQPPQLLIYLGSRRASGV | Clone PA13P2H10-light chain |
| | PDRFSGSGSGTDFTLKISRVEADDLGVYYCMQALQTRVTFGPGTKVDIK | variable region |
| 237 | QSLLHGNGYNY | Clone PA13P2H10-CDR-L1 |
| 78 | LGS | Clone PA13P2H10-CDR-L2 |
| 238 | MQALQTRVT | Clone PA13P2H10-CDR-L3 |
| 239 | EVQLVQSGAEVKKPGESLKISCKGSGYSFMSYWIGWVRQKPGKGLEWMGIIFPGDSDTRYS | Clone PA14P1H02-Heavy chain |
| | PSFQGHVTISADKSITTAYLQWNSLEASDTAIYYCATLDGDYWGRGTLVTVSS | variable region |
| 240 | GYSFMSYW | Clone PA14P1H02-CDR-H1 |
| 241 | IFPGDSDT | Clone PA14P1H02-CDR-H2 |
| 242 | ATLDGDY | Clone PA14P1H02-CDR-H3 |
| 243 | DIVMTQSPDSLAVSLGERATINCRSSQSVLSSSSNKNYLGWYQQKPGQPPKLLIHWASTRAA | Clone PA14P1H02-light chain |
| | GVPDRFSGSGTGTDFTLNISSLQAEDVAVYYCQQYHTTLPTFGQGTKLEIK | variable region |
| 244 | QSVLSSSSNKNY | Clone PA14P1H02-CDR-L1 |
| 86 | WAS | Clone PA14P1H02-CDR-L2 |
| 245 | QQYHTTLPT | Clone PA14P1H02-CDR-L3 |
| 246 | EVQLLESGGGLVQPGGSLRLSCAASGFTFRDSAMTWVRQAPGKGLEWVSTISGNGDTTYYA | Clone PA14P1H01-Heavy chain |
| | DSVKGRFSIFRDNSRNTLYVQMNSLRAEDTAVYYCARYGDHKGWFDSWGQGTLVTVSS | variable region |
| 247 | GFTFRDSA | Clone PA14P1H01-CDR-H1 |
| 248 | ISGNGDTT | Clone PA14P1H01-CDR-H2 |
| 249 | ARYGDHKGWFDS | Clone PA14P1H01-CDR-H3 |
| 250 | ELVMTQSPASLSVSPGEGATVSCRASQSVGSNLAWYQQKPGQGPRLLIYGASTRATGVPAR | Clone PA14P1H01-light chain |
| | FSGSGSGTEFTLTISSLQSEDFAVYYCQQYNNWPRTFGQGTKVEIK | variable region |
| 251 | QSVGSN | Clone PA14P1H01-CDR-L1 |
| 30 | GAS | Clone PA14P1H01-CDR-L2 |
| 252 | QQYNNWPRT | Clone PA14P1H01-CDR-L3 |
| 253 | QVQLVQSGAEVKKPGASVKVSCKASGYTFTSYYMHWVRQAPGQGLEWMGIINPSDGSKTY | Clone PA14P1H09-Heavy chain |
| | AQKFQGRVTLTRDTSTSTVYMELSSLRSEDTAVYYCARGNGYSSSWYVNDYWGQGTLVTVSS | variable region |
| 89 | GYTFTSYY | Clone PA14P1H09-CDR-H1 |
| 254 | INPSDGSK | Clone PA14P1H09-CDR-H2 |
| 255 | ARGNGYSSSWYVNDY | Clone PA14P1H09-CDR-H3 |
| 256 | DIVLTQSPGTLSLSPGERATLSCRASQSLTNSNFAWYQQIPGQAPRLLIYGASSRATGIPDRFS | Clone PA14P1H09-light chain |
| | GSGSGTDFTLTISRLEPEDFVVYYCQQYGRSPITFGQGTRLEIK | variable region |
| 257 | QSLTNSN | Clone PA14P1H09-CDR-L1 |
| 30 | GAS | Clone PA14P1H09-CDR-L2 |
| 258 | QQYGRSPIT | Clone PA14P1H09-CDR-L3 |
| 259 | QVQLVESGGGVVQPGRSLRLSCAASGFTFTKYGMHWVRQAPGKGLEWVALISYDGNNKYY | Clone PA12P3D11-Heavy chain |
| | ADSVRGRVTISRDNSKNTLYLQMDSLRAEDTAVYYCARGQDYPFWSGSTFEYWGQGTLVTV | variable region |
| | SS | |
| 260 | GFTFTKYG | Clone PA12P3D11-CDR-H1 |
| 261 | ISYDGNNK | Clone PA12P3D11-CDR-H2 |
| 262 | ARGQDYPFWSGSTFEY | Clone PA12P3D11-CDR-H3 |
| 263 | QAVVTQEPSLTVSPGGTVTLTCGSTTGAVTGGHFPYWIQQKPGQAPRTLIYDATNRHSWTP | Clone PA12P3D11-light chain |
| | ARFSGSLLGGKAALTLSGAQPEDEADYYCLLSYSSATFLIFGGGTKLTVL | variable region |
| 264 | TGAVTGGHF | Clone PA12P3D11-CDR-L1 |
| 265 | DAT | Clone PA12P3D11-CDR-L2 |
| 266 | LLSYSSATFLI | Clone PA12P3D11-CDR-L3 |
| 267 | QVQLQESGPGLVKPSETLSLTCTVSGDSLSSGSYFWSWIRQPPGKGLEWIGYISFRGDTNYNP | Clone PA12P3F02-Heavy chain |
| | SLKSRVIISLDKSKNQFSLRLSSMTPADTAVYYCARSPWIQSWSYYFDYWGQGTLVTVSS | variable region |
| 268 | GDSLSSGSYF | Clone PA12P3F02-CDR-H1 |
| 269 | ISFRGDT | Clone PA12P3F02-CDR-H2 |
| 270 | ARSPWIQSWSYYFDY | Clone PA12P3F02-CDR-H3 |
| 271 | DIQMTQSPSTVSASVGDRVTITCRASQRISSWLAWYQQKPGKAPKLLIYKASSLEGGVPSRFS | Clone PA12P3F02-light chain |
| | GSGSGTEFTLTISSLQPDDFAIYYCQQYNGYPWTFGQGTKVEIK | variable region |
| 272 | QRISSW | Clone PA12P3F02-CDR-L1 |
| 196 | KAS | Clone PA12P3F02-CDR-L2 |
| 273 | QQYNGYPWT | Clone PA12P3F02-CDR-L3 |
| 274 | QVQLQESGPGLVKPSGTLSLTCAVSGGSISTDNWWSWVRQPPNKGLEWIGAIFQSGSTIYN | Clone PA12P3F07-Heavy chain |
| | PSLMSRVTISLDRSNNRFSLQLISVTAADTALYYCARASFHYGSGNYFEYLGQGTLVTVSS | variable region |
| 275 | GGSISTDNW | Clone PA12P3F07-CDR-H1 |
| 276 | IFQSGST | Clone PA12P3F07-CDR-H2 |
| 277 | ARASFHYGSGNYFEY | Clone PA12P3F07-CDR-H3 |
| 278 | QSVLTQPPSVSAAPGQKVTISCSGSSSNVGTHHVSWYQQLPGTAPKLLIYENDKRPSGIPNRF | Clone PA12P3F07-light chain |
| | SGSKSGTSATLAIIGLQTGDEADYYCGSWDSSLSAFWVFGGGTKLTVL | variable region |
| 279 | SSNVGTHH | Clone PA12P3F07-CDR-L1 |
| 280 | END | Clone PA12P3F07-CDR-L2 |
| 281 | GSWDSSLSAFWV | Clone PA12P3F07-CDR-L3 |
| 282 | QVQLVQSGAEVKKPGASVKVACKASGYTFTRYAMHWVRQAPGQRLEWMGWINAGNGN | Clone PA14P1G03-Heavy chain |
| | TKDSQKFQGRVTITRDTSASTVYMELSSLRSEDTAVYYCARGVPWGLGSYNFDYWGQGTLV | variable region |
| | SISS | |
| 283 | GYTFTRYA | Clone PA14P1G03-CDR-H1 |
| 284 | INAGNGNT | Clone PA14P1G03-CDR-H2 |
| 285 | ARGVPWGLGSYNFDY | Clone PA14P1G03-CDR-H3 |
| 286 | QTVVTQEPSLTVSPGGTVTLSCASNTGAVTSGYYPYWFQQKPGQAPRTLIYETSNKHPWTP | Clone PA14P1G03-light chain |
| | ARFSGSLLGGKAALTLSGVQPEDEAEYCCLLYYGGTWVFGGGTKLTVL | variable region |
| 287 | TGAVTSGYY | Clone PA14P1G03-CDR-L1 |
| 288 | ETS | Clone PA14P1G03-CDR-L2 |
| 289 | LLYYGGTWV | Clone PA14P1G03-CDR-L3 |
| 290 | EVQLLESGGGLVQPGGSLRLSCAASGFTFSSYALSWVRQAPGKGLEWVSAISGRDGNTYYAD | Clone PA14P1G01-Heavy chain |
| | SVKGRFTISRDNSKNTLYLQMNSLRAEDTAVYYCTLYDYDSSGYFDFDYWGQGTLVTVSS | variable region |
| 113 | GFTFSSYA | Clone PA14P1G01-CDR-H1 |
| 291 | ISGRDGNT | Clone PA14P1G01-CDR-H2 |
| 292 | TLYDYDSSGYFDFDY | Clone PA14P1G01-CDR-H3 |
| 293 | QSALTQPRSVSGSPGQSVTISCTGTSSDVGGFNYVSWYQQHPGKAPKLMIFDVTQRPSGVP | Clone PA14P1G01-light chain |
| | DRFSGSKSGNTASLTISGLQAEDEADYHCCSYANYYTGVFGTGTRVTVL | variable region |
| 294 | SSDVGGFNY | Clone PA14P1G01-CDR-L1 |
| 141 | DVT | Clone PA14P1G01-CDR-L2 |
| 295 | CSYANYYTGV | Clone PA14P1G01-CDR-L3 |
| 296 | EVQLVESGGGLVQPGGSLRLSCAASGFTFSNYWMHWVRQAPGKGLVWVSRINSDGSNIRF | Clone PA16P1F09-Heavy chain |
| | ADSVKGRFTFSRDNANNTLYLQMNSLRAEDTAVYYCARASRTVYGDSPLSYGIDVWGQGTT | variable region |
| | VTVSS | |
| 183 | GFTFSNYW | Clone PA16P1F09-CDR-H1 |
| 297 | INSDGSNI | Clone PA16P1F09-CDR-H2 |
| 298 | ARASRTVYGDSPLSYGIDV | Clone PA16P1F09-CDR-H3 |
| 299 | SYALTQPPSVSVSPGQTASITCSGDKLGNKFACWYQQKPGRSPVLVIYQDSQRPTGIPERFSG | Clone PA16P1F09-light chain |
| | SNSGNTATLTISGTQAMDEADYYCQAWDSNTHVLFGGGTKLTVL | variable region |
| 300 | KLGNKF | Clone PA16P1F09-CDR-L1 |
| 301 | QDS | Clone PA16P1F09-CDR-L2 |
| 302 | QAWDSNTHVL | Clone PA16P1F09-CDR-L3 |
| 303 | QVQLVESGGGVVQPGGSLRLSCAASGFTFSGYGMHWVRQAPGKGLEWVAFFSFDGSNTD | Clone PA14P1G12-Heavy chain |
| | YVDSVKGRFTISGDNSKNTLYLQMNSLRAEDTAVYYCVRDILVLPAAVSVFSGYYYGMDVW | variable region |
| | GQGTTVTVSS | |
| 304 | GFTFSGYG | Clone PA14P1G12-CDR-H1 |
| 305 | FSFDGSNT | Clone PA14P1G12-CDR-H2 |
| 306 | VRDILVLPAAVSVFSGYYYGMDV | Clone PA14P1G12-CDR-H3 |
| 307 | EIVLTQSPATLSLSPGERATLSCRASQSVRSYLAWYQQKPGQAPRLLIYDASNRATGIPARFSG | Clone PA14P1G12-light chain |
| | SGSGTDFTLTISSLEPEDFAVYYCQHRSNWPITFGQGTRLEIK | variable region |
| 308 | QSVRSY | Clone PA14P1G12-CDR-L1 |
| 94 | DAS | Clone PA14P1G12-CDR-L2 |
| 309 | QHRSNWPIT | Clone PA14P1G12-CDR-L3 |
| 310 | QVQLRVSGPGLVNPSETLSLTCIVSGDSLRDYYWSWIRQSPGKGLEWIGYVTESGGAHYNPS | Clone PA11P1D12-Heavy chain |
| | LESRVTISVDASKTQFSLNLKSVTAADTAVYYCARDAYSSTWYTVGWFDPWGPGSLVTVSS | variable region |
| 311 | GDSLRDYY | Clone PA11P1D12-CDR-H1 |
| 312 | VTESGGA | Clone PA11P1D12-CDR-H2 |
| 313 | ARDAYSSTWYTVGWFDP | Clone PA11P1D12-CDR-H3 |
| 314 | EIVLTQSPATLSLSPGERATLSCRASQDVGVYLAWYQQKPGQAPRLIIYDASDRVSGVPARFT | Clone PA11P1D12-light chain |
| | GSGSGTDFTLTITSLEPEDFAVYFCQQRTSGLTFGGGTTLEIK | variable region |
| 315 | QDVGVY | Clone PA11P1D12-CDR-L1 |
| 94 | DAS | Clone PA11P1D12-CDR-L2 |
| 316 | QQRTSGLT | Clone PA11P1D12-CDR-L3 |
| 317 | QVELVESGGGVVQPGRSLRLSCVASGFTFSDYGMHWVRQAPGKGLEWVAVIWFDGSSKYY | Clone PA11P1D11-Heavy chain |
| | ADSVKGRFTISRDDSKNTVFMQMNNVRVEDTAVYYCAREQWLGTEYFQNWGQGTLVTVSS | variable region |
| 318 | GFTFSDYG | Clone PA11P1D11-CDR-H1 |
| 319 | IWFDGSSK | Clone PA11P1D11-CDR-H2 |
| 320 | AREQWLGTEYFQN | Clone PA11P1D11-CDR-H3 |
| 321 | EIVMTQSPATLSLFPGERATLSCRASQSVAGNLAWYQQKPGQAPRLLIYEASTRATDIPARFS | Clone PA11P1D11-light chain |
| | GSGSETEFTLTISSLQSEDFAVYYCQQYKKWLITFGQGTRLEIK | variable region |
| 322 | QSVAGN | Clone PA11P1D11-CDR-L1 |
| 323 | EAS | Clone PA11P1D11-CDR-L2 |
| 324 | QQYKKWLIT | Clone PA11P1D11-CDR-L3 |
| 325 | QLQLQQWGAGLVKPSETLSLTCTVSGGSLSGHFWSWIRQSPEKGLEWIGEINHSGRKNYNP | Clone PA12P1D02-Heavy chain |
| | SLMIRVDISIDTSKNQFSMRMTSLTAADSAVYYCARVGRNIVDTDDAFDVWGRGTLVTVSS | variable region |
| 326 | GGSLSGHF | Clone PA12P1D02-CDR-H1 |
| 327 | INHSGRK | Clone PA12P1D02-CDR-H2 |
| 328 | ARVGRNIVDTDDAFDV | Clone PA12P1D02-CDR-H3 |
| 329 | EIVLTQSPGTLSLSPGDTVTLSCRASQTIDSIYLAWYQQRPGQAPRLLIYGASTRATGTPDRFS | Clone PA12P1D02-light chain |
| | GGGSGTDFTLTITRLEPEDFAVYFCQQYGTSPPITFGRGTRLEIK | variable region |
| 330 | QTIDSIY | Clone PA12P1D02-CDR-L1 |
| 30 | GAS | Clone PA12P1D02-CDR-L2 |
| 331 | QQYGTSPPIT | Clone PA12P1D02-CDR-L3 |
| 332 | QVQLLQSGAEVKKPGASVKVSCKASGYTFTSYNIHWVRQAPGQSFEWMGWIHVGNGETK | Clone PA12P1D04-Heavy chain |
| | YSQNFQDRVAITRDTSANTVYMELSPLRSEDTALYYCVRDHVTAIVVGLFDPWGQGTLVTVSS | variable region |
| 333 | GYTFTSYN | Clone PA12P1D04-CDR-H1 |
| 334 | IHVGNGET | Clone PA12P1D04-CDR-H2 |
| 335 | VRDHVTAIVVGLFDP | Clone PA12P1D04-CDR-H3 |
| 336 | QSALTQPASVSGSPGQSITISCSGTSTDVGAYKYVSWYQHHPGRSPKVILYEVDNRPSGVSIR | Clone PA12P1D04-light chain |
| | FSGSKSGNTASLTISGLRAEDEADYYCSSFTSSSTWVFGGGTKVTVL | variable region |
| 337 | STDVGAYKY | Clone PA12P1D04-CDR-L1 |
| 338 | EVD | Clone PA12P1D04-CDR-L2 |
| 339 | SSFTSSSTWV | Clone PA12P1D04-CDR-L3 |
| 340 | QVQLVQSGAEVKKPGSSVKVSCKASGGTFSSYSISWVRQAPGQGLEWMGGIIPIFGSGSYA | Clone PA12P3E09-Heavy chain |
| | QKFQGRVTITADKSTSTAYMELSSLSSDDTAVYYCARGESPSNFVYYGMDVWGQGTTVTVSS | variable region |
| 341 | GGTFSSYS | Clone PA12P3E09-CDR-H1 |
| 342 | IPIFGSG | Clone PA12P3E09-CDR-H2 |
| 343 | ARGESPSNFVYYGMDV | Clone PA12P3E09-CDR-H3 |
| 344 | DIVLTQSPLSLPVTPGEPASISCRSSHSLLHSNGYNHLDWYLQKPGQSPQLLIYLGSNRASGVP | Clone PA12P3E09-light chain |
| | DRFSGSGSGTDFTLKISRVEAEDVGVYYCMQALLVTFGPGTKVDIK | variable region |
| 345 | HSLLHSNGYNH | Clone PA12P3E09-CDR-L1 |
| 78 | LGS | Clone PA12P3E09-CDR-L2 |
| 346 | MQALLVT | Clone PA12P3E09-CDR-L3 |
| 347 | EVKVVESGGGLVQPGGSLRLSCAASEFTFTYYWMSWIRQAPGKGLEWVANVNGDATEKYY | Clone PA12P3E04-Heavy chain |
| | VDSVKGRFTISRDNPKKTVYLQMNSLRVEDTAVYYCARVGTTVVNDGFDLWGLGTMVTVSS | variable region |
| 348 | EFTFTYYW | Clone PA12P3E04-CDR-H1 |
| 349 | VNGDATEK | Clone PA12P3E04-CDR-H2 |
| 350 | ARVGTTVVNDGFDL | Clone PA12P3E04-CDR-H3 |
| 351 | SYVLTQSHSVSVAPGQTARITCGGENIGGKGVHWYQQKPGQAPLLVVSSDTGRRSVTPDRF | Clone PA12P3E04-light chain |
| | SGSNSGDTATLIISRVEAGDEADYYCQVWDPTSEYVFGSGTKVTVL | variable region |
| 352 | NIGGKG | Clone PA12P3E04-CDR-L1 |
| 353 | SDT | Clone PA12P3E04-CDR-L2 |
| 354 | QVWDPTSEYV | Clone PA12P3E04-CDR-L3 |
| 355 | QLQLQESGSGLVKPSQTLSLTCAVSGGSISSGDYSWSWIRQPPGKGLEWIGFRYYSGTTFYNP | Clone PA12P3E07-Heavy chain |
| | SLESRLTISIDRSTNQFSLQLTSVTAADTAVYFCASFRPLLRFLDPEGLFEYWGQGILVTVSS | variable region |
| 356 | GGSISSGDYS | Clone PA12P3E07-CDR-H1 |
| 357 | RYYSGTT | Clone PA12P3E07-CDR-H2 |
| 358 | ASFRPLLRFLDPEGLFEY | Clone PA12P3E07-CDR-H3 |
| 359 | ELVMTQSPATLSVSPGARATLSCRASPGANSHLAWYQQKPGQAPRLLIYGASTRATGIPARF | Clone PA12P3E07-light chain |
| | SGSGSGTEFTLTISSLQSEDFAVYYCQQYNDWPYTFGQGTKLEIK | variable region |
| 360 | PGANSH | Clone PA12P3E07-CDR-L1 |
| 30 | GAS | Clone PA12P3E07-CDR-L2 |
| 361 | QQYNDWPYT | Clone PA12P3E07-CDR-L3 |
| 362 | QVQLVQSGAAVKKPGASVRISCEASGYTFTGYNIHWVRQAPGQGLEWMGWVNPNNGGT | Clone PA12P3E06-Heavy chain |
| | KFAQKFEGWVTMTVATSINTVYMELTGLKSGDTAVYFCARDHGDSFDQWGQGTLVTVSS | variable region |
| 363 | GYTFTGYN | Clone PA12P3E06-CDR-H1 |
| 364 | VNPNNGGT | Clone PA12P3E06-CDR-H2 |
| 365 | ARDHGDSFDQ | Clone PA12P3E06-CDR-H3 |
| 366 | EIVLTQSPDTLSLSPGDRATLSCRASHSLNNDYLAWYQHRPGQAPRLLIYGTSHGATGIPDRF | Clone PA12P3E06-light chain |
| | SGSGSGTDFTLTISRLETEDFAVYYCHHYGKSLFPFGPGTKVDIK | variable region |
| 367 | HSLNNDY | Clone PA12P3E06-CDR-L1 |
| 22 | GTS | Clone PA12P3E06-CDR-L2 |
| 368 | HHYGKSLFP | Clone PA12P3E06-CDR-L3 |
| 369 | EVQLVESGGGLVQPGGSLKLSCAASGFTFSGSAMHWVRQASGKGLEWVGRIRSKANTYAT | Clone PA14P1E08-Heavy chain |
| | AYAASVKGRFTISRDDSKNTAYLQMNSLKTEDTAVYYCTRKHTSGWYDRGGDVWGQGTTV | variable region |
| | TVSS | |
| 370 | GFTFSGSA | Clone PA14P1E08-CDR-H1 |
| 371 | IRSKANTYAT | Clone PA14P1E08-CDR-H2 |
| 372 | TRKHTSGWYDRGGDV | Clone PA14P1E08-CDR-H3 |
| 373 | DIQMTQSPSSLSASVGDRVTITCQASQDISNYLNWYQQKPGKAPKLLIYDASNLETGVPSRFS | Clone PA14P1E08-light chain |
| | GSGSGTDFTFTISSLQPEDIATYYCQQYDNLPLTFGGGTKVEIK | variable region |
| 374 | QDISNY | Clone PA14P1E08-CDR-L1 |
| 94 | DAS | Clone PA14P1E08-CDR-L2 |
| 375 | QQYDNLPLT | Clone PA14P1E08-CDR-L3 |
| 376 | EVQLLESGGGLVQPGGSLRLSCAASGFTVSNYAMSWVRQAPGKGLEWVSAISGSGGSTYYA | Clone PA14P1E09-Heavy chain |
| | DSVKGRFTISRDTSKNTLYLQMNSLRAEDTAVYYCAIDCTVTDAPLSYWGQGTLVTVSS | variable region |
| 377 | GFTVSNYA | Clone PA14P1E09-CDR-H1 |
| 378 | ISGSGGST | Clone PA14P1E09-CDR-H2 |
| 379 | AIDCTVTDAPLSY | Clone PA14P1E09-CDR-H3 |
| 380 | AIQMTQSPSSLSPSVGDRVTITCRASQGIRNDLGWYQQKPGKAPKLLIYAASSLQSGVPSRFS | Clone PA14P1E09-light chain |
| | GGGSGTDFTLTISSLQPEDFATYYCLQDYNYPRTFGQGTKVEIK | variable region |
| 381 | QGIRND | Clone PA14P1E09-CDR-L1 |
| 149 | AAS | Clone PA14P1E09-CDR-L2 |
| 382 | LQDYNYPRT | Clone PA14P1809-CDR-L3 |
| 383 | QVQLEQSGAEVRKPGSSVKVSCKASGTTFSNHAMSWVRQAPGQGLEWMGGIIPLVDKSM | Clone PA14P1H05-Heavy chain |
| | YALKFQGRVTITADESRNTVYMELSSLGSEDTAVYYCARSFADITTFGFVVNFHYYYTLD | variable region |
| | VWGQGTPVTVSS | |
| 384 | GTTFSNHA | Clone PA14P1H05-CDR-H1 |
| 385 | IPLVDKS | Clone PA14P1H05-CDR-H2 |
| 386 | ARSFADITTFGFVVNFHYYYTLDV | Clone PA14P1H05-CDR-H3 |
| 387 | NFMLTQPHSVSESPGKTVTISCTRSSGSIADNYVQWFQQRPGSAPTTLIYEDNRRPSGVP | Clone PA14P1H05-light chain |
| | DRFSGSVDSSSNSASLTISGLKPEDEADYYCQSYDTTQRVFGGGTKLTVL | variable region |
| 388 | SGSIADNY | Clone PA14P1H05-CDR-L1 |
| 389 | EDN | Clone PA14P1H05-CDR-L2 |
| 390 | QSYDTTQRV | Clone PA14P1H05-CDR-L3 |
| 391 | QLQLQESGSRLVKPSQTLSLTCAVSGGSINSGGYSWSWIRQPPGKGLEWIGNIYHGETTHYN | Clone PA12P3C09-Heavy chain |
| | PSLKSRVTISIDKSKNQFSLKLTSVTAADTAVYYCARAPLGNYYDTSGYLQPFDYWGPGAL | variable region |
| | VTVSS | |
| 392 | GGSINSGGYS | Clone PA12P3C09-CDR-H1 |
| 393 | IYHGETT | Clone PA12P3C09-CDR-H2 |
| 394 | ARAPLGNYYDTSGYLQPFDY | Clone PA12P3C09-CDR-H3 |
| 395 | DIQMTQSPSSLSASVGDRVTITCRASQGIINDLGWYQQRPGRAPTRLIYAASSLQSGVPSR | Clone PA12P3C09-light chain |
| | FSGSGSGTEFTLTINSLQPADFATYFCLQYNSYPPTFGQGTKVEIK | variable region |
| 396 | QGIIND | Clone PA12P3C09-CDR-L1 |
| 149 | AAS | Clone PA12P3C09-CDR-L2 |
| 397 | LQYNSYPPT | Clone PA12P3C09-CDR-L3 |
| 398 | EVQLVESGGGVVRPGGSLRLSCAASGFIFRDHGMSWVRQAPGKGLEWVSGINWNGANTG | Clone PA12P3C05-Heavy chain |
| | YADSVKGRSTISRDNAKNSLYLQMSSLRADDTALYHCVSHDYYYGLDVWGPGTTVIVSS | variable region |
| 399 | GFIFRDHG | Clone PA12P3C05-CDR-H1 |
| 400 | INWNGANT | Clone PA12P3C05-CDR-H2 |
| 401 | VSHDYYYGLDV | Clone PA12P3C05-CDR-H3 |
| 402 | QSALTQPRSVSGSPGQSVTISCTGTSSDVGGDNYVSWYQQHPGKVPKLIIHDVSERPSGVPD | Clone PA12P3C05-light chain |
| | RFSGSKSANTASLTISGLQADDEADYYCCSYAGTYTFGGGTRLTVL | variable region |
| 403 | SSDVGGDNY | Clone PA12P3C05-CDR-L1 |
| 404 | DVS | Clone PA12P3C05-CDR-L2 |
| 405 | CSYAGTYT | Clone PA12P3C05-CDR-L3 |
| 406 | QVQLQESGPGLVKPSQTLSLTCTVSGGSISSGGYYWSWIRQLPGKGLEWIGYIYYSGSTSYNP | Clone PA14P1G11-Heavy chain |
| | SLKSRVTISVDTSKNQLSLNLSSVTAADTAVYYCARGRRISISGVVTPLFDYWGQGTLVTVSS | variable region |
| 199 | GGSISSGGYY | Clone PA14P1G11-CDR-H1 |
| 200 | IYYSGST | Clone PA14P1G11-CDR-H2 |
| 201 | ARGRRISISGVVTPLFDY | Clone PA14P1G11-CDR-H3 |
| 407 | DIQMTQSPSSVSASVGDRVTITCRASQGISSWLAWYQQKPGKAPKLLIYAASSLQSGVPSRFS | Clone PA14P1G11-light chain |
| | GSGSGTDFTLTISSLQPEDFATYYCQQANSVPLTFGGGTKVEIK | variable region |
| 203 | QGISSW | Clone PA14P1G11-CDR-L1 |
| 149 | AAS | Clone PA14P1G11-CDR-L2 |
| 204 | QQANSVPLT | Clone PA14P1G11-CDR-L3 |
| 408 | QVQLVQSGAEVKKPGASVKVSCQASGYTFTRYDINWVRQATGQGLEWMGWLNPKSGDT | Clone PA12P3C01-Heavy chain |
| | GYAQKFQGRVTMTRDTSISTAYMELTSLTSDDTAVYYCARGVDANHWGQGSLVTVSS | variable region |
| 409 | GYTFTRYD | Clone PA12P3C01-CDR-H1 |
| 410 | LNPKSGDT | Clone PA12P3C01-CDR-H2 |
| 411 | ARGVDANH | Clone PA12P3C01-CDR-H3 |
| 412 | DIVVTQSPDSLAVSLGERATINCKSSQSIFDTSSNKNYLAWFRQRPGQPPQLLIYWASTRESG | Clone PA12P3C01-light chain |
| | VPDRFSGSGSGTDFTLTISSLQAEDVAVYYCHQYYSLPHAFGQGTKLEIK | variable region |
| 413 | QSIFDTSSNKNY | Clone PA12P3C01-CDR-L1 |
| 86 | WAS | Clone PA12P3C01-CDR-L2 |
| 414 | HQYYSLPHA | Clone PA12P3C01-CDR-L3 |
| 415 | EAQLVESGGGLVQPGGSLRLSCAASGFTFSSYYIHWVRQAPGKGLVWVSRINSDGSSTRYAD | Clone PA16P1H09-Heavy chain |
| | SVKGRFTISRDNAKNTLYLQMNSLRAEDTAVYFCARASRTVYGDSPLSNGMDVWGQGTKV | variable region |
| | TVSS | |
| 416 | GFTFSSYY | Clone PA16P1H09-CDR-H1 |
| 417 | INSDGSST | Clone PA16P1H09-CDR-H2 |
| 418 | ARASRTVYGDSPLSNGMDV | Clone PA16P1H09-CDR-H3 |
| 419 | SYELTQPPSVSVSPGQTASITCSGDKLGDKFACWYQQKPGHSPVLVIYQDDKRPSGIPERFSG | Clone PA16P1H09-light chain |
| | SNSGNTATLTISGTQAMDEADYYCQAWDSSTHVVFGGGTKLTVL | variable region |
| 420 | KLGDKF | Clone PA16P1H09-CDR-L1 |
| 421 | QDD | Clone PA16P1H09-CDR-L2 |
| 422 | QAWDSSTHVV | Clone PA16P1H09-CDR-L3 |
| 423 | EVQLVESGGGLVQPGGSLRLSCAASGFSFNTYNMNWVRQAPGKGLEWISDITSSGSMRSYA | Clone PA12P3D09-Heavy chain |
| | DAVKGRFTISRDNAKNSLHLQMNSLRVEDTAVYYCTRGWHDDLWSGYSYGLDVWGQGTT | variable region |
| | VTVSS | |
| 424 | GFSFNTYN | Clone PA12P3D09-CDR-H1 |
| 425 | ITSSGSMR | Clone PA12P3D09-CDR-H2 |
| 426 | TRGWHDDLWSGYSYGLDV | Clone PA12P3D09-CDR-H3 |
| 427 | DIQMTQSPSSLSASVGDRVTITCRASQGIRNDLGWYQQRPGKAPRCLIYGASSLQSGVPSRF | Clone PA12P3D09-light chain |
| | SGSGSGTEFTLTISNLQAEDFATYYCLQHKSYPLTFGPGTKVDIK | variable region |
| 381 | QGIRND | Clone PA12P3D09-CDR-L1 |
| 30 | GAS | Clone PA12P3D09-CDR-L2 |
| 428 | LQHKSYPLT | Clone PA12P3D09-CDR-L3 |
| 429 | QLLLLGPGPGVVRPSETLSLTCNVSGHSITDSPYYWGWIRQAPGKGLEWIGHFYYSDYTYYN | Clone PA13P3G04-Heavy chain |
| | PSLKSRVNVSVDTSKNHLFLALTSVTAADTAVYYCARGFGGYDSPIWAIWGQGTLVTVSS | variable region |
| 430 | GHSITDSPYY | Clone PA13P3G04-CDR-H1 |
| 431 | FYYSDYT | Clone PA13P3G04-CDR-H2 |
| 432 | ARGFGGYDSPIWAI | Clone PA13P3G04-CDR-H3 |
| 433 | SHAVTQPPSVSVAPGQTASLTCAGDDIEENTVHWYQQKPGQAPVLVIYYTTDRPSAIPERFF | Clone PA13P3G04-light chain |
| | GSKSGNTATLSIARVEAGDEADYYCQVSDRVFGGGTKLTVL | variable region |
| 434 | DIEENT | Clone PA13P3G04-CDR-L1 |
| 435 | YTT | Clone PA13P3G04-CDR-L2 |
| 436 | QVSDRV | Clone PA13P3G04-CDR-L3 |
| 437 | EVQLVQSGGGLVKPGGSLRLSCAASGSTLTNYNINWVRQAPGKGLQWVSSISGTRDYTYYA | Clone PA11P1C03-Heavy chain |
| | DSVVGRFTISRDNAKNSVYLQMNSLRAEDTAVYYCARGREVGGDYDSYDWGQGTLVTVSS | variable region |
| 438 | GSTLTNYN | Clone PA11P1C03-CDR-H1 |
| 439 | ISGTRDYT | Clone PA11P1C03-CDR-H2 |
| 440 | ARGREVGGDYDSYD | Clone PA11P1C03-CDR-H3 |
| 441 | DIQMTQSPSSLSASVGDRVTITCQASQDISTFLHWYQQKPGKAPSVLIYGASDLKTGVPSRFS | Clone PA11P1C03-light chain |
| | GSGSGTHFTLTISSLQPEDIATYYCQQYDHLPLTFGGGTKVEIK | variable region |
| 442 | QDISTF | Clone PA11P1C03-CDR-L1 |
| 30 | GAS | Clone PA11P1C03-CDR-L2 |
| 443 | QQYDHLPLT | Clone PA11P1C03-CDR-L3 |
| 444 | EVQLVESGGGLIQPGGSLRLSCAASGFTVSSNYMSWVRQAPGKGLEWVSVIYSGGSTYYAD | Clone PA11P1C01-Heavy chain |
| | SVKGRFTISRDNSKNTLYLQMNSLRAEDTAVYYCARDSRLGWAYDAFDIWGQGTMVTVSS | variable region |
| 445 | GFTVSSNY | Clone PA11P1C01-CDR-H1 |
| 446 | IYSGGST | Clone PA11P1C01-CDR-H2 |
| 447 | ARDSRLGWAYDAFDI | Clone PA11P1C01-CDR-H3 |
| 448 | DIVMTQSPLSLPVTPGEPASISCRSSQSLLHSNGYNYLDWYLQKPGQSPQLLIYLGSNRASGV | Clone PA11P1C01-light chain |
| | PDRFSGSGSGTDFTLKISRVEAEDVGVYYCMQDGTFGQGTKVEIK | variable region |
| 77 | QSLLHSNGYNY | Clone PA11P1C01-CDR-L1 |
| 78 | LGS | Clone PA11P1C01-CDR-L2 |
| 449 | MQDGT | Clone PA11P1C01-CDR-L3 |
| 450 | QVQLVQSGSELKKPGASVKASCKASGYTFSNYAVNWVRQAPGQGLEWMGWINTKTGNPT | Clone PA11P1C06-Heavy chain |
| | YGQGFTGRFVFSLDTSVSTAYLQISSLKAEDTAVYYCARAADYGEPYYYGMDVWGQGTTVT | variable region |
| | VSS | |
| 451 | GYTFSNYA | Clone PA11P1C06-CDR-H1 |
| 452 | INTKTGNP | Clone PA11P1C06-CDR-H2 |
| 453 | ARAADYGEPYYYGMDV | Clone PA11P1C06-CDR-H3 |
| 454 | QSALTQPASVSGSPGQSITISCTGTNSDVGSYNLVSWYQQHPGKAPKFMIYEGTKRPSGVSN | Clone PA11P1C06-light chain |
| | RFSGSKSGHTASLTISGLQAEDEADYYCCSYAGTSTLVFGGGTKLTVL | variable region |
| 455 | NSDVGSYNL | Clone PA11P1C06-CDR-L1 |
| 456 | EGT | Clone PA11P1C06-CDR-L2 |
| 457 | CSYAGTSTLV | Clone PA11P1C06-CDR-L3 |
| 458 | EVQLVESGGGLVQPGGSLRLSCAASGFTFRNYWMNWVRQAPGKGLVWVSRINSEGSSTSY | Clone PA13P1H03-Heavy chain |
| | ADPVKGRFTISRDNAKDTLYLQMDSLRAEDSAVYYCARIFNGYIHVGRDYWGQGTRVTVSS | variable region |
| 459 | GFTFRNYW | Clone PA13P1H03-CDR-H1 |
| 460 | INSEGSST | Clone PA13P1H03-CDR-H2 |
| 461 | ARIFNGYIHVGRDY | Clone PA13P1H03-CDR-H3 |
| 462 | DIQMTQSPSTLSASIGDRVTITCRASESISNWLAWFQQKPGKAPKLLIYKASNLESGVPSRFSG | Clone PA13P1H03-light chain |
| | SGSGTEFTLTISSLQPDDFATYYCQQYNSNSQTFGQGTKLDLK | variable region |
| 463 | ESISNW | Clone PA13P1H03-CDR-L1 |
| 196 | KAS | Clone PA13P1H03-CDR-L2 |
| 464 | QQYNSNSQT | Clone PA13P1H03-CDR-L3 |
| 465 | QVQLQESGPGLVKPSETLSLTCTVSGGSISSYYWNWIRQPPGKGLEWIGYIYYSGSTNYNPSL | Clone PA11P1C04-Heavy chain |
| | KSRATISVDTSKNQFSLKLSSVTAADTAVYYCARANLFGVALRRVLGPFDYWGQGTLVTVSS | variable region |
| 466 | GGSISSYY | Clone PA11P1C04-CDR-H1 |
| 200 | IYYSGST | Clone PA11P1C04-CDR-H2 |
| 467 | ARANLFGVALRRVLGPFDY | Clone PA11P1C04-CDR-H3 |
| 468 | DIQMTQSPSSLSASVGDRVTIACRASQSIANYLNWYQQKPGKAPKLLIYAASNLQSGVPSRFS | Clone PA11P1C04-light chain |
| | GSGSGTDFTLTISSLQPEDFATYYCQQSYSTPYTFGQGTKLEIK | variable region |
| 469 | QSIANY | Clone PA11P1C04-CDR-L1 |
| 149 | AAS | Clone PA11P1C04-CDR-L2 |
| 470 | QQSYSTPYT | Clone PA11P1C04-CDR-L3 |
| 471 | QVQLVESGGGVVQPGRSLRLSCAASGFSFRSYGMHWVRQAPGKGLEWVAVISYDGSNKYY | Clone PA14P1D10-Heavy chain |
| | VDSVKGRFTISRDNSKNTLYVQMNSLTDEDTAVYYCARDRGVTTRQFSYYYYGMDVWGQG | variable region |
| | TTVTVSS | |
| 472 | GFSFRSYG | Clone PA14P1D10-CDR-H1 |
| 59 | ISYDGSNK | Clone PA14P1D10-CDR-H2 |
| 473 | ARDRGVTTRQFSYYYYGMDV | Clone PA14P1D10-CDR-H3 |
| 474 | AIRMTQSPSSFSASTGDRVTITCRASQSITSYLAWYQQKPGKAPKLLIYAASTLQSGLPSRFSG | Clone PA14P1D10-light chain |
| | SGSGTDFTLTISGLQSEDFATYYCQQYYNYPQTFGQGTRVEIK | variable region |
| 475 | QSITSY | Clone PA14P1D10-CDR-L1 |
| 149 | AAS | Clone PA14P1D10-CDR-L2 |
| 476 | QQYYNYPQT | Clone PA14P1D10-CDR-L3 |
| 477 | EVQLLESGGQLVQPGGSLRLSCGAFGFTFGDAAMTWVRQAPGKGLEWVSTISGRGDETFS | Clone PA14P1C10-Heavy chain |
| | ADSVKGRFTISRDNFKNMLYVQMNSLRAEDTATYYCARLGHLRGWFDSWGQGTLVTVSS | variable region |
| 478 | GFTFGDAA | Clone PA14P1C10-CDR-H1 |
| 479 | ISGRGDET | Clone PA14P1C10-CDR-H2 |
| 480 | ARLGHLRGWFDS | Clone PA14P1C10-CDR-H3 |
| 481 | EIVMTQSPATLSVSPGERVTLSCRASQSVSSNLAWYQQKPGQAPRLLIYGASTRATGIPAGFS | Clone PA14P1C10-light chain |
| | GSGSGTEFTLTISSLQSEDFAVYYCQQYNNWPRTFGQGTKVEIK | variable region |
| 482 | QSVSSN | Clone PA14P1C10-CDR-L1 |
| 30 | GAS | Clone PA14P1C10-CDR-L2 |
| 252 | QQYNNWPRT | Clone PA14P1C10-CDR-L3 |
| 483 | EVQLVESGGGLVKPGGSLRLSCAASGFTFSDAWMTWVRQAPGKGLEWVGRIKSKTDGGTT | Clone PA11P1C08-Heavy chain |
| | DYGAPVKGRFSISRDDSKNTLYLHMNSLKTEDTAVYYCTTKSPNSNWFPFYYYYYMDVWGK | variable region |
| | GTTVTVSS | |
| 484 | GFTFSDAW | Clone PA11P1C08-CDR-H1 |
| 485 | IKSKTDGGTT | Clone PA11P1C08-CDR-H2 |
| 486 | TTKSPNSNWFPFYYYYYMDV | Clone PA11P1C08-CDR-H3 |
| 487 | QSALTQPRSVSGSPGQSVTISCTGTSSDVGGYNFVSWYQQHPGKAPQLMIYDVTKRPSGVP | Clone PA11P1C08-light chain |
| | DRFSGSKSGNTASLTISGLQAEDEGDYYCYSYAASSLYVFGTGTKVTVL | variable region |
| 488 | SSDVGGYNF | Clone PA11P1C08-CDR-L1 |
| 141 | DVT | Clone PA11P1C08-CDR-L2 |
| 489 | YSYAASSLYV | Clone PA11P1C08-CDR-L3 |
| 490 | QLQLQESGPGLVKPSETLSLICTVSGGAITSSTFYWAWIRQPPGRGLEWIGSMYYSGSTYYNL | Clone PA14P3F10-Heavy chain |
| | SLKSRVIISVNTSKNQFSLTLTSATATDMAVYYCVRHTLHDYGSGSFPDYSYGMDVWGQGTT | variable region |
| | VTVSS | |
| 491 | GGAITSSTFY | Clone PA14P3F10-CDR-H1 |
| 492 | MYYSGST | Clone PA14P3F10-CDR-H2 |
| 493 | VRHTLHDYGSGSFPDYSYGMDV | Clone PA14P3F10-CDR-H3 |
| 494 | EIVLTQSPATLSLFPGERGTLSCRASQSVSSHLIWYQQKPGQAPRVLIFDATNRATGIPARFSG | Clone PA14P3F10-light chain |
| | SGSGTDFTLTISNLEPEDYGVYYCQQRSNWPLTFGGGTKVEIK | variable region |
| 495 | QSVSSH | Clone PA14P3F10-CDR-L1 |
| 265 | DAT | Clone PA14P3F10-CDR-L2 |
| 496 | QQRSNWPLT | Clone PA14P3F10-CDR-L3 |
| 497 | EVQLLESGGGLVQPGGSLKLSCVASGFTFSSYAMMWVRQAPGKGLEWISSISSSGGSTYYAD | Clone PA14P1D11-Heavy chain |
| | SVKGRFTISRDNSKNTLYLQMNSLRVEDTAVYYCAKSHCSTTSCPRAFYYYGMDVWGQGTT | variable region |
| | VTVSS | |
| 113 | GFTFSSYA | Clone PA14P1D11-CDR-H1 |
| 498 | ISSSGGST | Clone PA14P1D11-CDR-H2 |
| 499 | AKSHCSTTSCPRAFYYYGMDV | Clone PA14P1D11-CDR-H3 |
| 500 | DIQMTQSPSSLSASVGDRVTITCRASQTITTYLNWYQQKPGKAPKLLIYAASSLQSGVPSRFSG | Clone PA14P1D11-light chain |
| | SGSGTDFTLSLSSLQPEDSATYYCQQSYSTLGAFGGGTKVEIK | variable region |
| 501 | QTITTY | Clone PA14P1D11-CDR-L1 |
| 149 | AAS | Clone PA14P1D11-CDR-L2 |
| 502 | QQSYSTLGA | Clone PA14P1D11-CDR-L3 |
| 503 | QVHLQESGPGLVKPSGTLSLTCTVSGGSISTYYWSWIRQPPGKGLEWIGYIYYGGTTNYNPSL | Clone PA14P3F02-Heavy chain |
| | KSRVTISVDTSKNQFSLRLRSVTAADTAVYYCAREIDSRMDRWGQGTLVTVSS | variable region |
| 504 | GGSISTYY | Clone PA14P3F02-CDR-H1 |
| 505 | IYYGGTT | Clone PA14P3F02-CDR-H2 |
| 506 | AREIDSRMDR | Clone PA14P3F02-CDR-H3 |
| 507 | SYALTQPPSVSVAPGKTARITCGGDNIGSKTVHWYHQKPGQAPVLVIYYDSNRPSGISERFSG | Clone PA14P3F02-light chain |
| | SNSGNTATLTISRVEAGDEADYYCQVWDSNSDHRIFGGGTKLTVL | variable region |
| 508 | NIGSKT | Clone PA14P3F02-CDR-L1 |
| 509 | YDS | Clone PA14P3F02-CDR-L2 |
| 510 | QVWDSNSDHRI | Clone PA14P3F02-CDR-L3 |
| 511 | QVQLVQSGAEVRKPGSSVKVSCKASGGTFSNNPITWVRQAPGQGLEWMGWIIPIFNTTNY | Clone PA12P1G11-Heavy chain |
| | AQKFQGRVTITADESTSTAYMELSSLKSEDTALFYCARDRAHAYCNNGVCYTTDAFDVWGQ | variable region |
| | GTLVTVSS | |
| 512 | GGTFSNNP | Clone PA12P1G11-CDR-H1 |
| 513 | IIPIFNTT | Clone PA12P1G11-CDR-H2 |
| 514 | ARDRAHAYCNNGVCYTTDAFDV | Clone PA12P1G11-CDR-H3 |
| 515 | ETVLTQSPATLSLSPGERATLSCRASQSVGRYLAWYQHKPGQAPRLLIYDASNRATGIPARFS | Clone PA12P1G11-light chain |
| | GSGSGTDFTLTISSLEPEDSAVYYCQQGTDWLTFGGGTKVEIK | variable region |
| 516 | QSVGRY | Clone PA12P1G11-CDR-L1 |
| 94 | DAS | Clone PA12P1G11-CDR-L2 |
| 517 | QQGTDWLT | Clone PA12P1G11-CDR-L3 |
| 518 | QVQLVESGGGLVKPGGSLRLSCAASGITFSDNYMTWIRQAPGKGLEWVSYISSSGTNIFYAD | Clone PA13P1E06-Heavy chain |
| | SLKGRFTISRDNAKNSLYLQMNSLRAEDTAVYYCARTLMTGSSLYFDYWGQGTQVTVSS | variable region |
| 519 | GITFSDNY | Clone PA13P1E06-CDR-H1 |
| 520 | ISSSGTNI | Clone PA13P1E06-CDR-H2 |
| 521 | ARTLMTGSSLYFDY | Clone PA13P1E06-CDR-H3 |
| 522 | SYELTQPPSVSVSPGQTARITCSGDALPKQYAYWYQQKPGQAPVLVIYKDSERPSGIPERFSG | Clone PA13P1E06-light chain |
| | SSSGTTVTLAISGVQAEDEADYYCQSADIRVTESVLFGGGTKLTVL | variable region |
| 523 | ALPKQY | Clone PA13P1E06-CDR-L1 |
| 524 | KDS | Clone PA13P1E06-CDR-L2 |
| 525 | QSADIRVTESVL | Clone PA13P1E06-CDR-L3 |
| 526 | EVHLLESGGHLVQPGGSLRLACAVSGFTFSDSAMTWVRQAPGKGLEWVSTISGRGDETFFA | Clone PA14P1C12-Heavy chain |
| | DSVKGRFSIFRDNSNSVLYVQMNSLRAEDTATYYCARYGHHKGWFDSWGQGTLVTVSS | variable region |
| 527 | GFTFSDSA | Clone PA14P1C12-CDR-H1 |
| 479 | ISGRGDET | Clone PA14P1C12-CDR-H2 |
| 528 | ARYGHHKGWFDS | Clone PA14P1C12-CDR-H3 |
| 529 | EIVMTQSPATLSVSPGERATLSCRASQSVSSNLAWYQQKPGQAPRLLIYGASTRAIGIPAGFS | Clone PA14P1C12-light chain |
| | GSGSGTEFTLTISSLQSEDSAVYYCQQYNNWPRTFGQGTKVEIK | variable region |
| 482 | QSVSSN | Clone PA14P1C12-CDR-L1 |
| 30 | GAS | Clone PA14P1C12-CDR-L2 |
| 252 | QQYNNWPRT | Clone PA14P1C12-CDR-L3 |
| 530 | QVQLVQSGTEVKKPGASVKVSCKASGYTFSSFGITWVRQAPGQGLEWMGWISAYNGNTKY | Clone PA11P1D07-Heavy chain |
| | AQAVQGRVTLTTDTSTTTAYMELRSLRSNDTAVYFCAREGIEHLVVEGRGPGGDCWGQGTL | variable region |
| | VIVSS | |
| 531 | GYTFSSFG | Clone PA11P1D07-CDR-H1 |
| 532 | ISAYNGNT | Clone PA11P1D07-CDR-H2 |
| 533 | AREGIEHLVVEGRGPGGDC | Clone PA11P1D07-CDR-H3 |
| 534 | SYELTQPPSVSVSPGQTARITCSGDALPKEYTSWYQQKSGQAPVLVIYEDIKRPSGIPERFSGS | Clone PA11P1D07-light chain |
| | SSGTMASLTISGAQVDDEADYYCYSTDTSGDHKVFGGGTKLTVL | variable region |
| 535 | ALPKEY | Clone PA11P1D07-CDR-L1 |
| 536 | EDI | Clone PA11P1D07-CDR-L2 |
| 537 | YSTDTSGDHKV | Clone PA11P1D07-CDR-L3 |
| 538 | QVQLVESGGGVVQPGRSLRLSCAASGFTFSSYAMHWVRQAPGKGREWVAALSYDGSSTYY | Clone PA12P4D02-Heavy chain |
| | ADSVKGRLTISRDNSKNTLYLQMNSLRAEDTAVYFCTRVPYGEGRAANDYWGQGTLVTVSS | variable region |
| 113 | GFTFSSYA | Clone PA12P4D02-CDR-H1 |
| 539 | LSYDGSST | Clone PA12P4D02-CDR-H2 |
| 540 | TRVPYGEGRAANDY | Clone PA12P4D02-CDR-H3 |
| 541 | DIQMTQSPSTLSASVGDRVTITCRASQSIGSWLAWYQQKPGKAPKLLIYKASNIESGVPSRFS | Clone PA12P4D02-light chain |
| | GSGSGTEFTLTISSLQPDDFATYYCQHYNTYSRSFGGGTEVAIK | variable region |
| 542 | QSIGSW | Clone PA12P4D02-CDR-L1 |
| 196 | KAS | Clone PA12P4D02-CDR-L2 |
| 543 | QHYNTYSRS | Clone PA12P4D02-CDR-L3 |
| 544 | QVQLVQSGAEVKTPGSSVKVSCTASGDSFSRYAINWVRQAPGQGLEWVGKIVPVFGAASYA | Clone PA15P1C03-heavy chain |
| | QKFQGRVTITADESTSTVYMELSSLRSEDTAVYYCARGIVKLSTMPPVYWGQGTLVTVSS | variable region |
| 545 | GDSFSRYA | Clone PA15P1C03-CDR-H1 |
| 546 | IVPVFGAA | Clone PA15P1C03-CDR-H2 |
| 547 | ARGIVKLSTMPPVY | Clone PA15P1C03-CDR-H3 |
| 548 | DIVMTQTPLSLSVTPGQPASISCKSSQSLLHSDGKTYLYWYLQKPGQSPQLLISEVSSRFSGVP | Clone PA15P1C03-light chain |
| | DRFSGSGSGTDFTLKISRVEAEDVGVYYCMQGIHHLTFGPGTKVDIK | variable region |
| 549 | QSLLHSDGKTY | Clone PA15P1C03-CDR-L1 |
| 550 | EVS | Clone PA15P1C03-CDR-L2 |
| 551 | MQGIHHLT | Clone PA15P1C03-CDR-L3 |
| 552 | QVQLQESGPGLVKPSETLSLTCSVSGGSVSDSAYYWSWIRQPPGGGLEFIGYVYNSGSTNYN | Clone PA12P4G06-heavy chain |
| | PSLKSRVTISVDTSKNQFSLSLSSLTAADTAVYYCARYCSSTSCYVRSSDVNWFDPWGQGTLVI | variable region |
| | VSS | |
| 553 | GGSVSDSAYY | Clone PA12P4G06-CDR-H1 |
| 554 | VYNSGST | Clone PA12P4G06-CDR-H2 |
| 555 | ARYCSSTSCYVRSSDVNWFDP | Clone PA12P4G06-CDR-H3 |
| 556 | EIVLTQSPGTLSSSPGESATLSCRASQSLGTYLAWYQQKPGQAPRLLIYDASKRATGIPARFSG | Clone PA12P4G06-light chain |
| | SGSGTDFTLTISSLEPEDFAVYYCHQRSHWLTFGGGTKVEIK | variable region |
| 557 | QSLGTY | Clone PA12P4G06-CDR-L1 |
| 94 | DAS | Clone PA12P4G06-CDR-L2 |
| 558 | HQRSHWLT | Clone PA12P4G06-CDR-L3 |
| 559 | EAQLLESGGGLVQPGGSLRLSCAASGFNFSNYAMTWVRQAPGKGLEWVSAISSGGGTTYYA | Clone PA16P1809-heavy chain |
| | DSVKGRFTISRDNSKNTVYLQMNSLKDADSALYYCAKPGRAVVVRLSYFDSWGQGTLVTVSS | variable region |
| 560 | GFNFSNYA | Clone PA16P1B09-CDR-H1 |
| 561 | ISSGGGTT | Clone PA16P1B09-CDR-H2 |
| 562 | AKPGRAVVVRLSYFDS | Clone PA16P1809-CDR-H3 |
| 563 | QSVLTQPPSVSAAPGQKVSISCSGSGSNIANHYVSWYQHLPGTAPKLLIYDNNKRPSGIPDRF | Clone PA16P1B09-light chain |
| | SGSKSGTSATLGITGLQTGDEADYYCGTWDSSLTVVVFGGGTKLTVL | variable region |
| 564 | GSNIANHY | Clone PA16P1B09-CDR-L1 |
| 110 | DNN | Clone PA16P1B09-CDR-L2 |
| 565 | GTWDSSLTVVV | Clone PA16P1B09-CDR-L3 |
| 566 | QITLKESGPTLVKPTETLTLTCTFSGFSLTTSGVAVGWVRQPPGKALEWLALIYWDDDERYTP | Clone PA12P4G03-heavy chain |
| | SLKSRLTITKDTSKSQVVLTMTNMDPVDTATYFCVHCEGPDILLVPAAYFFDFWGQGTLVTV | variable region |
| | SS | |
| 567 | GFSLTTSGVA | Clone PA12P4G03-CDR-H1 |
| 568 | IYWDDDE | Clone PA12P4G03-CDR-H2 |
| 569 | VHCEGPDILLVPAAYFFDF | Clone PA12P4G03-CDR-H3 |
| 570 | EIVLTQSPGTLSLSPGDRATLSCRASQSVSRRYLAWYQQSPGQAPRLLISGASSRATGIPDRFS | Clone PA12P4G03-light chain |
| | GSGSGTDFTLTISRLEPEDFAMYYCQQYGSSTGTFGQGTKVEMK | variable region |
| 571 | QSVSRRY | Clone PA12P4G03-CDR-L1 |
| 30 | GAS | Clone PA12P4G03-CDR-L2 |
| 572 | QQYGSSTGT | Clone PA12P4G03-CDR-L3 |
| 573 | QVQLQESGPGLVKPSQTLSLTCTVSGGSISSSGYYWSWIRQHPGKGLEWIGYINYIGGTYYNP | Clone PA11P1F10-heavy chain |
| | SLRSRVTMSVDTSKNQFSLRLSSVSAADTAVYYCASTHSYGDYSRDYYYGVDVWGQGTTVTI | variable region |
| | SS | |
| 574 | GGSISSSGYY | Clone PA11P1F10-CDR-H1 |
| 575 | INYIGGT | Clone PA11P1F10-CDR-H2 |
| 576 | ASTHSYGDYSRDYYYGVDV | Clone PA11P1F10-CDR-H3 |
| 577 | EIVLTQSPATLSLSPGDRATLSCRTSQSVSSSYLAWYQQKPGQAPRLLIYAASSRATGIPDRFS | Clone PA11P1F10-light chain |
| | GSGSGTDFTLTISRLEPEDFAVYYCQQCAGSPFTFGPGTKVDLK | variable region |
| 62 | QSVSSSY | Clone PA11P1F10-CDR-L1 |
| 149 | AAS | Clone PA11P1F10-CDR-L2 |
| 578 | QQCAGSPFT | Clone PA11P1F10-CDR-L3 |
| 579 | QVQLVESGGGVVQPGRSLRLSCAASGFTFSDYAMHWVRQAPGKGLEWVAVISYDGNHRY | Clone PA12P1G02-heavy chain |
| | YADSVKGRFTISRDNSKNTLYLQMNSLRAEDTAVYYCARHPGLSIAVAGPFDYWGQGTLVTV | variable region |
| | SS | |
| 580 | GFTFSDYA | Clone PA12P1G02-CDR-H1 |
| 581 | ISYDGNHR | Clone PA12P1G02-CDR-H2 |
| 582 | ARHPGLSIAVAGPFDY | Clone PA12P1G02-CDR-H3 |
| 583 | EIVMTQSPATLSVSPGERATLSCGASQSVSSNLAWYQQKPGQAPRLLFYGASTRATGIPARFS | Clone PA12P1G02-light chain |
| | GSGSGTEFTLTISSLQSEDFALYYCQQYNNWPWTFGQGTKVDIK | variable region |
| 482 | QSVSSN | Clone PA12P1G02-CDR-L1 |
| 30 | GAS | Clone PA12P1G02-CDR-L2 |
| 584 | QQYNNWPWT | Clone PA12P1G02-CDR-L3 |
| 585 | QVQLVQSGAEVKKPGASVKVSCKASGYTFNRDGITWVRQAPGQGLEWMGWISANNDFT | Clone PA16P1E12-heavy chain |
| | DYAQKFQGRLTMTTDTSTNTAYMELRSLRSDDTAVYYCARQVITVLQYSYGMDVWGQGTT | variable region |
| | VTVSS | |
| 586 | GYTFNRDG | Clone PA16P1E12-CDR-H1 |
| 587 | ISANNDFT | Clone PA16P1E12-CDR-H2 |
| 588 | ARQVITVLQYSYGMDV | Clone PA16P1E12-CDR-H3 |
| 589 | DIQMTQFPSSLSASVGDRVTITCRASQSISRYLNWYQQTPGKAPKLLIYGASSLQSGVPSRFSG | Clone PA16P1E12-light chain |
| | SGSGTDFTLTISSLQPEDFATYYCQQSDTAPLTFGGGTRVEIK | variable region |
| 590 | QSISRY | Clone PA16P1E12-CDR-L1 |
| 30 | GAS | Clone PA16P1E12-CDR-L2 |
| 591 | QQSDTAPLT | Clone PA16P1E12-CDR-L3 |
| 415 | EAQLVESGGGLVQPGGSLRLSCAASGFTFSSYYIHWVRQAPGKGLVWVSRINSDGSSTRYAD | Clone PA16P1E11-heavy chain |
| | SVKGRFTISRDNAKNTLYLQMNSLRAEDTAVYFCARASRTVYGDSPLSNGMDVWGQGTKV | variable region |
| | TVSS | |
| 416 | GFTFSSYY | Clone PA16P1E11-CDR-H1 |
| 417 | INSDGSST | Clone PA16P1E11-CDR-H2 |
| 418 | ARASRTVYGDSPLSNGMDV | Clone PA16P1E11-CDR-H3 |
| 419 | SYELTQPPSVSVSPGQTASITCSGDKLGDKFACWYQQKPGHSPVLVIYQDDKRPSGIPERFSG | Clone PA16P1E11-light chain |
| | SNSGNTATLTISGTQAMDEADYYCQAWDSSTHVVFGGGTKLTVL | variable region |
| 420 | KLGDKF | Clone PA16P1E11-CDR-L1 |
| 421 | QDD | Clone PA16P1E11-CDR-L2 |
| 422 | QAWDSSTHVV | Clone PA16P1E11-CDR-L3 |
| 592 | QVQLVESGGGLVTPGGSLRLSCTVSGFTLSDYYMSWIRQAPGKGLDWLSYISGSGDNKNYA | Clone PA12P3E11-heavy chain |
| | DSVRGRFTISRDNSKNSLYLQMNSLRAEDTAVYYCAREFPSGGYSPGVVLWGQGTLVTVSS | variable region |
| 593 | GFTLSDYY | Clone PA12P3E11-CDR-H1 |
| 594 | ISGSGDNK | Clone PA12P3E11-CDR-H2 |
| 595 | AREFPSGGYSPGVVL | Clone PA12P3E11-CDR-H3 |
| 596 | NFVLTQPHSVSESPGKTVTISCARSSGSIAGSFVQWYQQRPGSSPTTVIYEDTRRPSGVPDRF | Clone PA12P3E11-light chain |
| | SGSIDSSSNSASLTISGLKTEDEADYYCQSYDSTNPWVFGGGTKLTVL | variable region |
| 597 | SGSIAGSF | Clone PA12P3E11-CDR-L1 |
| 598 | EDT | Clone PA12P3E11-CDR-L2 |
| 599 | QSYDSTNPWV | Clone PA12P3E11-CDR-L3 |
| 600 | EVQLLESGGGLVQPGGSLRLSCAASGFTFSSYAMSWVRQAPGKGLEWVSVIYSGGSRTYYA | Clone PA14P1C06-heavy chain |
| | DSAKGRFTISRDNSKNMLYLQMNSLRAEDTAVYYCTKNDYDSSGYFDFDNWGQGTLVTVSS | variable region |
| 113 | GFTFSSYA | Clone PA14P1C06-CDR-H1 |
| 114 | IYSGGSRT | Clone PA14P1C06-CDR-H2 |
| 601 | TKNDYDSSGYFDFDN | Clone PA14P1C06-CDR-H3 |
| 602 | QSALTQPASVSGSPGQSITISCTGTSSDVGGYNYVSWYQQHPGKAPKLMIYDVSNRPSGVSN | Clone PA14P1C06-light chain |
| | RFSGSKSGNTASLTISGLQAEDEADYHCSSYTSSSTWVFGGGTKLTVL | variable region |
| 603 | SSDVGGYNY | Clone PA14P1C06-CDR-L1 |
| 404 | DVS | Clone PA14P1C06-CDR-L2 |
| 604 | SSYTSSSTWV | Clone PA14P1C06-CDR-L3 |
| 605 | EVQLLESGGGLVQPGGSLRLSCAASGFTFSSYAMSWVRQAPGKGLEWVSVVYSGGSGTYYA | Clone PA14P1C07-heavy chain |
| | DSVKGRFTISRDDSTNTLYLQMNSLRAEDTAVYYCAKDRDSFGELDLDSWGQGTLVSVSS | variable region |
| 113 | GFTFSSYA | Clone PA14P1C07-CDR-H1 |
| 606 | VYSGGSGT | Clone PA14P1C07-CDR-H2 |
| 607 | AKDRDSFGELDLDS | Clone PA14P1C07-CDR-H3 |
| 608 | DIVMTQSPDSLAVSLGERATINCKSSQSVLYSSKNKNYLAWYQQRPGQPPKLLIYWASTRES | Clone PA14P1C07-light chain |
| | GVPDRFSGSGSGTDFTLTISSLQAEDVAVYYCLQYYNIPRTFGQGTKLEIK | variable region |
| 609 | QSVLYSSKNKNY | Clone PA14P1C07-CDR-L1 |
| 86 | WAS | Clone PA14P1C07-CDR-L2 |
| 610 | LQYYNIPRT | Clone PA14P1C07-CDR-L3 |
| 611 | EVQLLESGGGLVQPGGSLRLSCAASGFTFSSYALSWVRQPPGKGLEWVSVIYSGGSRTYYAD | Clone PA14P1C04-heavy chain |
| | AAKGRFTISRDNSKNMLYLQMNSLRAEDTAVYYCTKHDYDSSGYFDFDNWGQGTLVTVSS | variable region |
| 113 | GFTFSSYA | Clone PA14P1C04-CDR-H1 |
| 114 | IYSGGSRT | Clone PA14P1C04-CDR-H2 |
| 612 | TKHDYDSSGYFDFDN | Clone PA14P1C04-CDR-H3 |
| 602 | QSALTQPASVSGSPGQSITISCTGTSSDVGGYNYVSWYQQHPGKAPKLMIYDVSNRPSGVSN | Clone PA14P1C04-light chain |
| | RFSGSKSGNTASLTISGLQAEDEADYHCSSYTSSSTWVFGGGTKLTVL | variable region |
| 603 | SSDVGGYNY | Clone PA14P1C04-CDR-L1 |
| 404 | DVS | Clone PA14P1C04-CDR-L2 |
| 604 | SSYTSSSTWV | Clone PA14P1C04-CDR-L3 |
| 613 | EVQLVESGGGVARPGGSLRLSCAASGFTFDDYGMSWVRQAPGKGLEWVSGINWNGGSTN | Clone PA14P1C02-heavy chain |
| | YADSVKGRFIISRDNGKNSLYLQMNSLRAEDTAFYHCARRGNFYYYGMDVWGQGTTVTVSS | variable region |
| 614 | GFTFDDYG | Clone PA14P1C02-CDR-H1 |
| 615 | INWNGGST | Clone PA14P1C02-CDR-H2 |
| 616 | ARRGNFYYYGMDV | Clone PA14P1C02-CDR-H3 |
| 617 | DIQMTQSPSSVSASVGDRVTITCRASQGNSTWLAWYQQKPGKAPELLIFDASNLQSGVPSR | Clone PA14P1C02-light chain |
| | FSGSGSGTDFTLTISSLQPEDFATYYCQQAQRFPLTFGGGTKVEIK | variable region |
| 618 | QGNSTW | Clone PA14P1C02-CDR-L1 |
| 94 | DAS | Clone PA14P1C02-CDR-L2 |
| 619 | QQAQRFPLT | Clone PA14P1C02-CDR-L3 |
| 620 | QVQLVQSGSELRRPGASVKVSCKTSGYAFTHFAMNWLRQAPGQGLEWLGWINTHSGNPT | Clone PA11P1E01-heavy chain |
| | YAQGFTGRIVFSLDTSAGTAYLEISSLKAEDTAVYYCARERYFDFWGQGTLVAVSS | variable region |
| 621 | GYAFTHFA | Clone PA11P1E01-CDR-H1 |
| 176 | INTHSGNP | Clone PA11P1E01-CDR-H2 |
| 177 | ARERYFDF | Clone PA11P1E01-CDR-H3 |
| 622 | QSVLTQPPSASGTPGQRVTISCSGTNSNIGKNFLYWYQQLPGTAPKLLIFSSNQRPSGVPDRF | Clone PA11P1E01-light chain |
| | SGSKSGTSASLAISGLRSEDEADYYCAAWDDNLSGWVFGGGTKVTVL | variable region |
| 623 | NSNIGKNF | Clone PA11P1E01-CDR-L1 |
| 180 | SSN | Clone PA11P1E01-CDR-L2 |
| 624 | AAWDDNLSGWV | Clone PA11P1E01-CDR-L3 |
| 625 | QVQLQESGPGLVKPSEALSLTCSVSDGSVSSGSYYWTWIRQPPGKGLEWIGCIHYSGRTNYN | Clone PA11P1E08-heavy chain |
| | PSLKSRVTISIDTSKNQFSLQLSSVTAVDTAVYYCARDRGEYDFWRVRYYGMDVWGQGTTV | variable region |
| | TVSS | |
| 626 | DGSVSSGSYY | Clone PA11P1E08-CDR-H1 |
| 627 | IHYSGRT | Clone PA11P1E08-CDR-H2 |
| 628 | ARDRGEYDFWRVRYYGMDV | Clone PA11P1E08-CDR-H3 |
| 629 | QSALTQPASVSGSPGQSITISCTGTSSDVGDYNYVSWYQQHPGKAPKLLIYDFSNRPSGVSDR | Clone PA11P1E08-light chain |
| | FSGSKSGNTASLTISGLRAEDESDYYCTSYTNTNTRLFGGGTKLTVL | variable region |
| 630 | SSDVGDYNY | Clone PA11P1E08-CDR-L1 |
| 631 | DFS | Clone PA11P1E08-CDR-L2 |
| 632 | TSYTNTNTRL | Clone PA11P1E08-CDR-L3 |
| 633 | QVQLVQSGAEVKKPGASVKVSCKASGYTFISYGLHWVRQAPGQRPEWMGWINAGNGNR | Clone PA14P1C08-heavy chain |
| | KYSERFQARVTFTRDTSATTAYMELSSLRSEDTAVYYCARDRLTAAAHFDYWGQGTQVTVSS | variable region |
| 634 | GYTFISYG | Clone PA14P1C08-CDR-H1 |
| 635 | INAGNGNR | Clone PA14P1C08-CDR-H2 |
| 636 | ARDRLTAAAHFDY | Clone PA14P1C08-CDR-H3 |
| 637 | EIVMTQSPATLSVSLGERATLSCRASQSVSSDLAWYQQKPGQAPRLLMYGASTRATGFPARF | Clone PA14P1C08-light chain |
| | TGSGSGPEFTLTISSLQSEDFAVYYCQQYNNWPFTFGGGTKVEIK | variable region |
| 638 | QSVSSD | Clone PA14P1C08-CDR-L1 |
| 30 | GAS | Clone PA14P1C08-CDR-L2 |
| 639 | QQYNNWPFT | Clone PA14P1C08-CDR-L3 |
| 640 | QITLKESGPTLVKPTQTLTLTCTFSGFSLTSSAVGVGWIRQPPGKALEWLALIYGDDDKRYSPS | Clone PA14P1H12-heavy chain |
| | LKRRLTITKDTSKNQVVLTMTDVDPVDTATYYCAHRRLTIPLLMVAADAFDIWGPGTMVIVSS | variable region |
| 641 | GFSLTSSAVG | Clone PA14P1H12-CDR-H1 |
| 642 | IYGDDDK | Clone PA14P1H12-CDR-H2 |
| 643 | AHRRLTIPLLMVAADAFDI | Clone PA14P1H12-CDR-H3 |
| 644 | DIQMTQSPSTLSASVGDRVTITCRASQSVSRWLAWYQQKPGKAPKLLIYRASSLQSGVPSRFS | Clone PA14P1H12-light chain |
| | GSGSGTEFTLTISSLQPDDFATYYCQQYSSFHTFGQGTKLEIK | variable region |
| 645 | QSVSRW | Clone PA14P1H12-CDR-L1 |
| 646 | RAS | Clone PA14P1H12-CDR-L2 |
| 647 | QQYSSFHT | Clone PA14P1H12-CDR-L3 |
| 648 | EVQLVESGGGLVQPGGSLRLSCAASGFIFSTYSMNWVRQAPGKGLEWVSYISSSSNTIYYAD | Clone PA14P1H11-heavy chain |
| | SVKGRFTISRDNAKNSLYLQMNSLRDADTAVYYCARDGGRSGYFDDYWGQGTLVTVSS | variable region |
| 649 | GFIFSTYS | Clone PA14P1H11-CDR-H1 |
| 650 | ISSSSNTI | Clone PA14P1H11-CDR-H2 |
| 651 | ARDGGRSGYFDDY | Clone PA14P1H11-CDR-H3 |
| 652 | QLVLTQSPSASASLGASVKLTCTLSNGHINYAIAWHQQQPDKGPRYLLNLKSDGSHSKGDGI | Clone PA14P1H11-light chain |
| | PDRFSGSSSGAERYLTISGLQSEDEADYYCQTWGTGIQVFGGGTKLTVL | variable region |
| 653 | NGHINYA | Clone PA14P1H11-CDR-L1 |
| 654 | LKSDGSH | Clone PA14P1H11-CDR-L2 |
| 655 | QTWGTGIQV | Clone PA14P1H11-CDR-L3 |
| 656 | QVQLQQWGAGLLKPSETLSLTCAVYGGSFSGYYWSWIRQPPGKGLEWIGEINHSGSTNYNP | Clone PA14P1D02-heavy chain |
| | SLKSRVTISVDTSKNQFSLKLSSVTAADTAVYYCARGRGVWFGELFPFDYWGQGTLGTVSS | variable region |
| 657 | GGSFSGYY | Clone PA14P1D02-CDR-H1 |
| 658 | INHSGST | Clone PA14P1D02-CDR-H2 |
| 659 | ARGRGVWFGELFPFDY | Clone PA14P1D02-CDR-H3 |
| 660 | QGGLTQPPSVSKGLRQTATLTCTGNSNNVGNQGAAWLQQHQGHPPKLLSYRNNNRPSGIS | Clone PA14P1D02-light chain |
| | ERFSASRSGNTASLTITGLQPEDEADYYCSAWDSSLSAVVFGGGTKLTVL | variable region |
| 661 | SNNVGNQG | Clone PA14P1D02-CDR-L1 |
| 662 | RNN | Clone PA14P1D02-CDR-L2 |
| 663 | SAWDSSLSAVV | Clone PA14P1D02-CDR-L3 |
| 664 | QVQLQQWGAGLLKPSETLSLNCTVYHGSLSTSYWSWIRQPPGRGLEWIGEINDSGATNYNP | Clone PA11P1F03-heavy chain |
| | SLKSRVIISVDTSKDQFSLKLTSVTAADTAMYYCARAPLLWVGESFFYYFDSWGQGILVTVSS | variable region |
| 665 | HGSLSTSY | Clone PA11P1F03-CDR-H1 |
| 666 | INDSGAT | Clone PA11P1F03-CDR-H2 |
| 667 | ARAPLLWVGESFFYYFDS | Clone PA11P1F03-CDR-H3 |
| 668 | DIQMTQSPSSLSASVGDRVSITCRAGQSIDTYLNWYQHKPGKAPDLLIYTTSTLHSGVPSRFS | Clone PA11P1F03-light chain |
| | GSGSGTDFTLTITSLQPEDFAIYYCQQSYKSPYTFGQGTKVEIK | variable region |
| 669 | QSIDTY | Clone PA11P1F03-CDR-L1 |
| 670 | TTS | Clone PA11P1F03-CDR-L2 |
| 671 | QQSYKSPYT | Clone PA11P1F03-CDR-L3 |
| 672 | QVQLVQSGAEVKKPGASVKVSCKASGYTFTSYYMHWVRQAPGQGLEWMGIINPSGGTTSS | Clone PA11P1F02-heavy chain |
| | AQKFQGRVTMTRDTSTSTVYMELSSLRSEDTALYYCARDREQKVGGAPLHWGQGTLVTVSS | variable region |
| 89 | GYTFTSYY | Clone PA11P1F02-CDR-H1 |
| 673 | INPSGGTT | Clone PA11P1F02-CDR-H2 |
| 674 | ARDREQKVGGAPLH | Clone PA11P1F02-CDR-H3 |
| 675 | DIQMTQSPSSLSASVGDRVTITCQASQDISNYLNWYQQKPGKAPKLLIYDASNLETGVPSRFS | Clone PA11P1F02-light chain |
| | GSGSGTDFSFTISSLQPEDIATYYCQQYDNFALTFGGGTKVEIK | variable region |
| 374 | QDISNY | Clone PA11P1F02-CDR-L1 |
| 94 | DAS | Clone PA11P1F02-CDR-L2 |
| 676 | QQYDNFALT | Clone PA11P1F02-CDR-L3 |
| 677 | EVQLLESGGGLVQPGVSLRLSCAASGFTFSSYAMSWVRQAPGKGLEWVSVIYSGGNIIYYAD | Clone PA14P1D07-heavy chain |
| | SVKGRFTISRDNSKNTLYLQIDNLRAEDTALYYCAKHDYDSSGYFDFDYWGHGTLVTVSS | variable region |
| 113 | GFTFSSYA | Clone PA14P1D07-CDR-H1 |
| 678 | IYSGGNII | Clone PA14P1D07-CDR-H2 |
| 679 | AKHDYDSSGYFDFDY | Clone PA14P1D07-CDR-H3 |
| 680 | QSALTQPASVSGSPGQSITISCTGTSRDVGGYNYVSWYQQHPGKAPKLMIYDVNNRPSGVS | Clone PA14P1D07-light chain |
| | NRFSGSKSGNTASLTISGLQAEDEADYFCCSYTSSSTWVFGGGTKLTVL | variable region |
| 681 | SRDVGGYNY | Clone PA14P1D07-CDR-L1 |
| 682 | DVN | Clone PA14P1D07-CDR-L2 |
| 683 | CSYTSSSTWV | Clone PA14P1D07-CDR-L3 |
| 684 | QVQLQESGPGLVKPSGTLSLTCAVSGASISNSAWWNWVRQPPRGGLEWVGEIYPSGSTNY | Clone PA14P1D09-heavy chain |
| | TPSLKSRATILLDESRNEFSLKLNSVTAADTAVYYCARGRLEDCNGGVCYFFDNWGQGILVSV | variable region |
| | SS | |
| 685 | GASISNSAW | Clone PA14P1D09-CDR-H1 |
| 686 | IYPSGST | Clone PA14P1D09-CDR-H2 |
| 687 | ARGRLEDCNGGVCYFFDN | Clone PA14P1D09-CDR-H3 |
| 688 | DIEMTQSPSTLSASVGDRVTITCRANYGIGAWLAWYQQKPGKAPKLLIYKASTLESGVPLRFS | Clone PA14P1D09-light chain |
| | GSGSGTEFTLSISGLQPDDFATYYCHQYSTYPITFGQGTRLEIK | variable region |
| 689 | YGIGAW | Clone PA14P1D09-CDR-L1 |
| 196 | KAS | Clone PA14P1D09-CDR-L2 |
| 690 | HQYSTYPIT | Clone PA14P1D09-CDR-L3 |
| 691 | QVQLVESGGGVVQPGRSLTLSCAASGFNFKTYGMHWVRQAPGKGLEWVAVIYHDGNDKF | Clone PA14P3H08-heavy chain |
| | YADSVKGRFTISRDNSKNTLYVQMSSLRADDTAIYYCAKGIFSSGYHYGMDVWGQGTAVIVSS | variable region |
| 692 | GFNFKTYG | Clone PA14P3H08-CDR-H1 |
| 693 | IYHDGNDK | Clone PA14P3H08-CDR-H2 |
| 694 | AKGIFSSGYHYGMDV | Clone PA14P3H08-CDR-H3 |
| 695 | DIQMTQSPSSLSASLGDSVTITCLASQGIKEFLSWFQQKPGQAPKLLIYDASSSHSGVPSRFSG | Clone PA14P3H08-light chain |
| | SGSATHFTLTISSLQPDDIATYYCQQYHQVPLTFGQGTRLEIK | variable region |
| 696 | QGIKEF | Clone PA14P3H08-CDR-L1 |
| 94 | DAS | Clone PA14P3H08-CDR-L2 |
| 697 | QQYHQVPLT | Clone PA14P3H08-CDR-L3 |
| 190 | QVQLQQWGAGLLKPSETLSLTCVVSGGSFSTHYWNWIRQSPGKGLEWIGEINHSGNTNYN | Clone PA15P1E01-heavy chain |
| | PSLTGRATISVATSKTQFSLRLNSVTAADTAVYFCARGPRLRYTAGRPLFDTWGQGTLVTVSS | variable region |
| 191 | GGSFSTHY | Clone PA15P1E01-CDR-H1 |
| 192 | INHSGNT | Clone PA15P1E01-CDR-H2 |
| 193 | ARGPRLRYTAGRPLFDT | Clone PA15P1E01-CDR-H3 |
| 194 | DIQMTQSPSTLSASVGDRVTITCRASQSISAFLAWYQQKPGKAPNLVIYKASSLDSGVPSTFS | Clone PA15P1E01-light chain |
| | GSGSGTEYTLTISSLQPDDFATYYCQQYFSSPPTFGQGTKVEMK | variable region |
| 195 | QSISAF | Clone PA15P1E01-CDR-L1 |
| 196 | KAS | Clone PA15P1E01-CDR-L2 |
| 197 | QQYFSSPPT | Clone PA15P1E01-CDR-L3 |
| 698 | EVRLVESGGGLIQPGGSLRLSCAASGFNVSSDYMNWVRQAPGKGLEWVSVLYSSGFTYYAD | Clone PA15P1E02-heavy chain |
| | SVRGRFTISRDNSKNTLYLQMNSLRAEDTAVYYCARVALFGEPLVDSWGQGTLVTVSS | variable region |
| 699 | GFNVSSDY | Clone PA15P1E02-CDR-H1 |
| 700 | LYSSGFT | Clone PA15P1E02-CDR-H2 |
| 701 | ARVALFGEPLVDS | Clone PA15P1E02-CDR-H3 |
| 702 | EIVMTQSPATLSVSPGERATLSCRASQSVSSNLAWYQQKPGQAPRLLIYGAISRATGIPARFS | Clone PA15P1E02-light chain |
| | GSGSGTEFTLTISSLQSEDFAIYYCQQYNNWPWTFGQGTKVEIK | variable region |
| 482 | QSVSSN | Clone PA15P1E02-CDR-L1 |
| 703 | GAI | Clone PA15P1E02-CDR-L2 |
| 584 | QQYNNWPWT | Clone PA15P1E02-CDR-L3 |
| 704 | DPYSPS | Binding motif sequence for |
| | | PA13P1H08 |
| 705 | DSYGRDPYSPS | Binding motif sequence for |
| | | PA13P1H08 |
| 706 | YSPSQDPYSPS | Binding motif sequence for |
| | | PA13P1H08 |
| 707 | PDRRDPYSPS | Binding motif sequence for |
| | | PA13P1H08 |
| 708 | RQQWELQGDRRCQSQLERANLRPCEQHLMQKIQRDEDSYGRDPYSPSQDPYSPSQDPDRR | Ara h 2 isoform Ara h 2.0201 |
| | DPYSPSPYDRRGAGSSQHQERCCNELNEFENNQRCMCEALQQIMENQSDRLQGR | (DPYSPS motif underlined) |
| | QQEQQFKRELRNLPQQCGLRAPQRCDLEVESGGRDRY | |
| 709 | EVQLVASGGGLIHPGGSLRLSCEASGFSFSRFWMYWVRQSPGEGLVWVARLSGDGT | Clone 1003320101_D6 heavy |
| | VTNYADSMEGRVTISRDNVKNTLFLEMNSLREGDTGIYYCARKDCPSLSCQLDY | chain variable region sequence |
| | WGQGVQVTVSS | |
| 710 | GFSFSRFW | Clone 1003320101_D6 CDR-H1 |
| 711 | LSGDGTVT | Clone 1003320101_D6 CDR-H2 |
| 712 | ARKDCPSLSCQLDY | Clone 1003320101_D6 CDR-H3 |
| 713 | QSVLTQPPSVSAAPGQKVTISCSGSTSNIGKNYVSWYQHFPGAAPKLLIFDNDKRPSGI | Clone 1003320101_D6 light chain |
| | PDRFSGSRSGTSATLDITGLQTGDEADYFCATWDSRLSADVFGSGTTVSVL | variable region sequence |
| 714 | TSNIGKNY | Clone 1003320101_D6 CDR-L1 |
| 715 | DND | Clone 1003320101_D6 CDR-L2 |
| 716 | ATWDSRLSADV | Clone 1003320101_D6 CDR-L3 |
| 717 | EVQLLESGGGLVQPGGSLRLSCAASGFNFSNFAVSWVRQTPGKGLEWVSAILGSRSVT | Clone 1003320105_D6 heavy chain |
| | YYADSVKGRFTISRDKSKNALYLQMDSLRAEDTAIYYCAKLFFMPYSHDDSGDYWGQG | variable region sequence |
| | TLVAVSS | |
| 718 | GFNFSNFA | Clone 1003320105_D6 CDR-H1 |
| 719 | ILGSRSVT | Clone 1003320105_D6 CDR-H2 |
| 720 | AKLFFMPYSHDDSGDY | Clone 1003320105_D6 CDR-H3 |
| 721 | QLVLTQSPSASASLGASVKLTCTLSSDHRSYAIAWHQQQPGKGPRYLMKVNRDGSHIKG | Clone 1003320105_D6 light chain |
| | DGIPHRFSGSSSVTERYLIISSLQSEDEADYYCQSWDTGIQVFGGGTRLTVV | variable region sequence |
| 722 | SDHRSYA | Clone 1003320105_D6 CDR-L1 |
| 723 | VNRDGSH | Clone 1003320105_D6 CDR-L2 |
| 724 | QSWDTGIQV | Clone 1003320105_D6 CDR-L3 |
| 725 | QVQLQESGLGLVKPSGTLSLTCAVSGGPMNSSYWWSWVRQSPGGGLEWIGQISHYTNT | Clone 1003320107_C5 heavy chain |
| | KYNPSFKNRVSISIDKSKNEFSLRLTYVTGADTGVYYCVGERDWKDPNWFDPWGQGRL | variable region sequence |
| | VTVSS | |
| 726 | GGPMNSSYW | Clone 1003320107_C5 CDR-H1 |
| 727 | ISHYTNT | Clone 1003320107_C5 CDR-H2 |
| 728 | VGERDWKDPNWFDP | Clone 1003320107_C5 CDR-H3 |
| 729 | QSVLTQPPSVSGAPGQRVTISCTGSNSNIGAGQDVHWYQHFPGTAPKLVIYGNSNRPS | Clone 1003320107_C5 light chain |
| | GVPDRFSGSKSGTSASLAISGLQADDEADYYCQSYDKSLSSSLFGGGTKLTVL | variable region |
| 730 | NSNIGAGQD | Clone 1003320107_C5 CDR-L1 |
| 731 | GNS | Clone 1003320107_C5 CDR-L2 |
| 732 | QSYDKSLSSSL | Clone 1003320107_C5 CDR-L3 |
| 733 | QVQLQESGPGLVKPSETLSLSCNVSGGSIRGHYWSWIRQSPGKRLEWLGYIYQSGYTK | Clone 1003320107_F3 heavy chain |
| | YNPSLKSRVSISLDTSKNKFSLNLKSVTTADTAVYYCAGRVAERGGDQFDFWGQGTLV | variable region sequence |
| | TVSS | |
| 734 | GGSIRGHY | Clone 1003320107_F3 CDR-H1 |
| 735 | IYQSGYT | Clone 1003320107_F3 CDR-H2 |
| 736 | AGRVAERGGDQFDF | Clone 1003320107_F3 CDR-H3 |
| 737 | SYELTQSPSLSVSPGQTASITCSGENLGEKHASWYQQKSGQSPVLVIYQDTKRPAGIPER | Clone 1003320107_F3 light chain |
| | FSGSNSGSTATLTISGTQPMDEADYFCQAWDANTANVIFGGGTMLTVL | variable region sequence |
| 738 | NLGEKH | Clone 1003320107_F3 CDR-L1 |
| 739 | QDT | Clone 1003320107_F3 CDR-L2 |
| 740 | QAWDANTANVI | Clone 1003320107_F3 CDR-L3 |
| 741 | EVQLVESGGGLVQPGGSLKLSCAASGFTFSGSTIHWVRQTSGKGLEWVGRIGSKATSY | Clone 1003320107_F8 heavy chain |
| | ATAYAASVKGRFTISRDDSKNTAYLQMNSLKTEDTAVYFCTRRYYDTTKSVLVVSDS | variable region sequence |
| | WGQGTLVTVSS | |
| 742 | GFTFSGST | Clone 1003320107_F8 CDR-H1 |
| 743 | IGSKATSYAT | Clone 1003320107_F8 CDR-H2 |
| 744 | TRRYYDTTKSVLVVSDS | Clone 1003320107_F8 CDR-H3 |
| 745 | SYELTQPPSMSVSPGQTARITCSGDVLAKQFAYWYQQKPGQAPVLVIYKDSERPSGIPER | Clone 1003320107_F8 light chain |
| | FSGSSSGTIITLTISGVQAEDEADYYCQSADSSGTSWVFGGGTKLTVL | variable region sequence |
| 746 | VLAKQF | Clone 1003320107_F8 CDR-L1 |
| 747 | KDS | Clone 1003320107_F8 CDR-L2 |
| 748 | QSADSSGTSWV | Clone 1003320107_F8 CDR-L3 |
| 749 | QLLLQESGPGLVKPSETLSLSCTVSAGSITSINYSWGWIRQPPGKGLEWIASVYFSGSI | Clone PA01P2C05 heavy chain |
| | YYNPSLKSRVAISVDTSKNTFSLNLTSVTAADTAVYYCARLRLDTGRDSSGLSYREHFD | variable region sequence |
| | YWAQGTLVTVSS | |
| 750 | AGSITSINYS | Clone PA01P2C05 CDR-H1 |
| 751 | VYFSGSI | Clone PA01P2C05 CDR-H2 |
| 752 | ARLRLDTGRDSSGLSYREHFDY | Clone PA01P2C05 CDR-H3 |
| 753 | DIQMTQSPSTLSASVGDRVTITCRASQSIGMWLAWFQQKPGKAPKLLIYKASTLESGV | Clone PA01P2C05 light chain |
| | PSRFSGSGSGTEFTLTINSLQPDDFATYYCQQYNSYLFTFGPGTKVDIK | variable region sequence |
| 754 | QSIGMW | Clone PA01P2C05 CDR-L1 |
| 196 | KAS | Clone PA01P2C05 CDR-L2 |
| 755 | QQYNSYLFT | Clone PA01P2C05 CDR-L3 |
| 756 | EVQLVQSGAEVKKPGESLKISCKGSGYNFTSSWIGWVRQMPGKGLEWMGIIHPGDSD | Clone PA01P2B03 heavy chain |
| | TRYSPSFQGQVTISADKSLTTAFLQWSSLKTSDTAIYYCARHGSTMLWGDAFDIWGQ | variable region sequence |
| | GTMVTVSS | |
| 757 | GYNFTSSW | Clone PA01P2B03 CDR-H1 |
| 758 | IHPGDSDT | Clone PA01P2803 CDR-H2 |
| 759 | ARHGSTMLWGDAFDI | Clone PA01P2B03 CDR-H3 |
| 760 | SYELTQPPSVSLSPGQTARITCSGDALPKHYAYWYQQKPGQAPVLVIYKDTERPSGIPE | Clone PA01P2B03 light chain |
| | RFSGSSSGTTVTLTISGVQAEDEADYYCQSSDSTGEVFGGGTKLTVL | variable region sequence |
| 761 | ALPKHY | Clone PA01P2B03 CDR-L1 |
| 762 | KDT | Clone PA01P2B03 CDR-L2 |
| 763 | QSSDSTGEV | Clone PA01P2B03 CDR-L3 |
| 764 | QVQLVQSGAEVKKPGASVMLSCKASGYIFTNSDINWVRQAPGQGPEWMGWMNPKSGNT | Clone PA01P2A12 heavy chain |
| | GYEQKFQGRVTMTTNTSISTAYMELSRLRSEDTAVYYCARSTGAVAGNFDYWGQGTP | variable region sequence |
| | VTVSS | |
| 765 | GYIFTNSD | Clone PA01P2A12 CDR-H1 |
| 766 | MNPKSGNT | Clone PA01P2A12 CDR-H2 |
| 767 | ARSTGAVAGNFDY | Clone PA01P2A12 CDR-H3 |
| 768 | EIVMTQSPATLSVSLGDRATLSCRASQSISRNLAWYQQKPGQAPRLLIYGASIRITDI | Clone PA01P2A12 light chain |
| | PARFSGSGSGTEFTLTISSLQSEDFAIYFCQQYNNWRTFGQGTRVELK | variable region sequence |
| 769 | QSISRN | Clone PA01P2A12 CDR-L1 |
| 30 | GAS | Clone PA01P2A12 CDR-L2 |
| 770 | QQYNNWRT | Clone PA01P2A12 CDR-L3 |
| 771 | HVQLQESGPGLVKSSETLSLTCNVSSDSFSDHYWSWVRQPAGKGLQWLGRIYNTGTTTYNP | Clone PA01P2C12 heavy chain |
| | SLNRRITMSVDTSKNQFSLRLTSVTAADTAVYYCAARHYHYDKTIWGQGTLVTVSS | variable region sequence |
| 772 | SDSFSDHY | Clone PA01P2C12 CDR-H1 |
| 773 | IYNTGTT | Clone PA01P2C12 CDR-H2 |
| 774 | AARHYHYDKTI | Clone PA01P2C12 CDR-H3 |
| 775 | NFMLTQPHSVSESPGKTVTISCTRSSGSIASNYVQWYQQRPGSPPTTVIYEDNQRPSGV | Clone PA01P2C12 light chain |
| | PDRFSGSIDSSSNSASLTISGLKPEDEADFYCQSYDSDDREVFGGGTRLTVL | variable region sequence |
| 776 | SGSIASNY | Clone PA01P2C12 CDR-L1 |
| 389 | EDN | Clone PA01P2C12 CDR-L2 |
| 777 | QSYDSDDREV | Clone PA01P2C12 CDR-L3 |
| 778 | QVQLVQSGAEVKKPGASVKVSCKASGYTFTTYGISWVRQAPGQGLEWMGWISAYNGNTT | Clone PA01P2E10 heavy chain |
| | YAQNFHARVTMTTDTSTSTAYMELRSLRSDDTAVYFCARTSARTITIFGVLIPA | variable region sequence |
| | GLNLDYWGQGTLVTVSS | |
| 779 | GYTFTTYG | Clone PA01P2E10 CDR-H1 |
| 532 | ISAYNGNT | Clone PA01P2E10 CDR-H2 |
| 780 | ARTSARTITIFGVLIPAGLNLDY | Clone PA01P2E10 CDR-H3 |
| 781 | QSALTQPASVSGSPGQSITISCTGTGSDVGRYNYVSWYQQHPGKAPKFMIYDVSNRPS | Clone PA01P2E10 light chain |
| | GVSNRFSASKSGNTASLTISGLQAEDEADYYCSSYTSTSTLVFGGGTKLTVL | variable region sequence |
| 782 | GSDVGRYNY | Clone PA01P2E10 CDR-L1 |
| 404 | DVS | Clone PA01P2E10 CDR-L2 |
| 783 | SSYTSTSTLV | Clone PA01P2E10 CDR-L3 |
| 784 | QVDLVESGGGYVKSGGSLRLSCAASGFRFSDYYMSWVRQAPGKGLEWLSHISSDSSDT | Clone PA01P2C09 heavy chain |
| | NYADSVKGRFSISRDNAKNSVFLQMNTLRAEDTAVYYCARNALTNAYDMSGFRNWGQG | variable region sequence |
| | ILVTVSS | |
| 785 | GFRFSDYY | Clone PA01P2C09 CDR-H1 |
| 786 | ISSDSSDT | Clone PA01P2C09 CDR-H2 |
| 787 | ARNALTNAYDMSGFRN | Clone PA01P2C09 CDR-H3 |
| 788 | NFMLTQPHSVSESPGKTVILSCTRSSGSIATNYVRWYQQRPGSAPTTVIYEDSRRPSSVP | Clone PA01P2C09 light chain |
| | DRFSGSIDSSSNSASLTISGLRTEDEADYYCQSFDTSSRKVVFGGGTKLTVL | variable region sequence |
| 789 | SGSIATNY | Clone PA01P2C09 CDR-L1 |
| 790 | EDS | Clone PA01P2C09 CDR-L2 |
| 791 | QSFDTSSRKVV | Clone PA01P2C09 CDR-L3 |
| 792 | QVTLRESGPALVEVTQTVTLTCNFSGFSLHTRGMYVNWIRQPPGKALEWLAVINWDDD | Clone PA01P1D06 heavy chain |
| | KYYTPSLKNRLTISKDTSKNQVVLTMTNMDPVDTATYYCARTDYGGYGPEGFDYWGQGT | variable region sequence |
| | LVTVSS | |
| 793 | GFSLHTRGMY | Clone PA01P1D06 CDR-H1 |
| 794 | INWDDDK | Clone PA01P1D06 CDR-H2 |
| 795 | ARTDYGGYGPEGFDY | Clone PA01P1D06 CDR-H3 |
| 796 | EIVMTQSPATLSVSPGESATLSCRASQSVRSNLAWYQQKPGQAPRLLIYGASTRAPGVP | Clone PA01P1D06 light chain |
| | ARFTGSESGREFTLTISSLQSEDFAVYYCQQYNNWPPYTFGQGTKLEIK | variable region sequence |
| 797 | QSVRSN | Clone PA01P1D06 CDR-L1 |
| 30 | GAS | Clone PA01P1D06 CDR-L2 |
| 798 | QQYNNWPPYT | Clone PA01P1D06 CDR-L3 |
| 799 | QVQLVESGGGVVQPGRSLRLSCVASGFDLNAYGMHWVRQAPGKGLDWVAATSRGGTKKY | Clone PA01P2E08 heavy chain |
| | YADSVKGRFTISRDVSKNTLYLQMGSLRTGDTAIYYCGVGMEDVFDIWGQGTMVTVSP | variable region sequence |
| 800 | GFDLNAYG | Clone PA01P2E08 CDR-H1 |
| 801 | TSRGGTKK | Clone PA01P2E08 CDR-H2 |
| 802 | GVGMEDVFDI | Clone PA01P2E08 CDR-H3 |
| 803 | QSVLTQPPSVSAAPGQKVTISCSENNSNIGNRNVSWYQQLPGKAPKLFIYDNNERPS | Clone PA01P2E08 light chain |
| | GIPARFSGSKSGTSATLVITGLQTGDEADYYCGTWDRSLSVWVFGGGT | variable region sequence |
| | KLTVL | |
| 804 | NSNIGNRN | Clone PA01P2E08 CDR-L1 |
| 110 | DNN | Clone PA01P2E08 CDR-L2 |
| 805 | GTWDRSLSVWV | Clone PA01P2E08 CDR-L3 |
| 806 | QVQLQESGPGLVKPSQTLSLTCTVSGGSMRSGDYYWSWIRQPPGKGLEWIGYIYFTG | Clone PA01P2A05 heavy chain |
| | SSYYNPSLKSRATISVDTSKNQFSLKLNSVTAADTAVYFCARGVDVDLTFFDCW | variable region sequence |
| | GHGTLVTVSS | |
| 807 | GGSMRSGDYY | Clone PA01P2A05 CDR-H1 |
| 808 | IYFTGSS | Clone PA01P2A05 CDR-H2 |
| 809 | ARGVDVDLTFFDC | Clone PA01P2A05 CDR-H3 |
| 810 | SYVLTQPPSVSLAPGKTARITCGGNNIGNKSVHWYQQKPGQAPVLVIYYDSDRPSGIP | Clone PA01P2A05 light chain |
| | ERFSGSNSGNTATLTINRVEAGDEADYHCQVWDSSTDHRVFGEGTKLTVL | variable region sequence |
| 811 | NIGNKS | Clone PA01P2A05 CDR-L1 |
| 509 | YDS | Clone PA01P2A05 CDR-L2 |
| 812 | QVWDSSTDHRV | Clone PA01P2A05 CDR-L3 |
| 813 | QVQLQQWGAGLLKPSETLSLTCTVIGTSFSNYYWSWIRQPPGKGLQWIGEITHSDSANY | Clone PA01P2B04 heavy chain |
| | NPSLKSRVIISIDSSKNQLSLNLSSVTAADTAVYYCARGSKDYYDRSTFSWFDPWGQ | variable region sequence |
| | GTLVTVSS | |
| 814 | GTSFSNYY | Clone PA01P2B04 CDR-H1 |
| 815 | ITHSDSA | Clone PA01P2B04 CDR-H2 |
| 816 | ARGSKDYYDRSTFSWFDP | Clone PA01P2B04 CDR-H3 |
| 817 | EIVMTQSPATLSVSPGERATLSCRASQNISNKLAWYQQKPGQAPRLLIYDASTRATGVP | Clone PA01P2B04 light chain |
| | ARFSCSVSGTAFTLTINRLQSEDFAVYYCQQYYYWPPPYTFGHGTKLEIK | variable region sequence |
| 818 | QNISNK | Clone PA01P2B04 CDR-L1 |
| 94 | DAS | Clone PA01P2B04 CDR-L2 |
| 819 | QQYYYWPPPYT | Clone PA01P2B04 CDR-L3 |
| 820 | QVQLVESGGGFVKPGGSLRLSCAVSGFTFSDYYMSWVRQAPGKGLEWLSHISSDGSDT | Clone PA01P2E05 heavy chain |
| | NYADSVKGRFSISRDNAKNSVFLQMNTLRVEDTAVYYCARNALTNAYDMSGFRNWGQG | variable region sequence |
| | TLVTVSS | |
| 821 | GFTFSDYY | Clone PA01P2E05 CDR-H1 |
| 822 | ISSDGSDT | Clone PA01P2E05 CDR-H2 |
| 787 | ARNALTNAYDMSGFRN | Clone PA01P2E05 CDR-H3 |
| 823 | NFMLTQPHSVSESPGKTVILSCTRSSGSIASNYVRWYQQRPGSAPTTVIYEDSRRPSSVP | Clone PA01P2E05 light chain |
| | DRFSGSIDSSSNSASLTISGLKTEDEADYYCQSFDSSSRKVVFGGGTKLTVL | variable region sequence |
| 776 | SGSIASNY | Clone PA01P2E05 CDR-L1 |
| 790 | EDS | Clone PA01P2E05 CDR-L2 |
| 824 | QSFDSSSRKVV | Clone PA01P2E05 CDR-L3 |
| 825 | QVQLLQSGPEVKQPGASVQVSCQTSGYTFTGYYIHWVRQAPGQGLEWVGWINPNRGHTN | Clone PA01P2D04 heavy chain |
| | YGPAFQGRLTLTADTSSSTAYLELTRLRSDDTAVYYCARDRLTGGRDAFEIWGQGTML | variable region sequence |
| | IVSS | |
| 121 | GYTFTGYY | Clone PA01P2D04 CDR-H1 |
| 826 | INPNRGHT | Clone PA01P2D04 CDR-H2 |
| 827 | ARDRLTGGRDAFEI | Clone PA01P2D04 CDR-H3 |
| 828 | DIQLTQSPSSLSASVGDRVTITCRASQSISSYLNWYQQKPGKAPKLLISVASSLQDGVP | Clone PA01P2D04 light chain |
| | PRFSGSRSGTEFTLTISSLQPEDFAIYYCQQSYSLSWTFGQGTKVEIK | variable region sequence |
| 829 | QSISSY | Clone PA01P2D04 CDR-L1 |
| 830 | VAS | Clone PA01P2D04 CDR-L2 |
| 831 | QQSYSLSWT | Clone PA01P2D04 CDR-L3 |
| 832 | QVQLVQSGAEVKQPGASVQVACQTSGYIFTAYYIHWLRQAPGQGLEWVGWINPNRGHTN | Clone PA01P2B12 heavy chain |
| | YAPGFQGRLTLTADTSSSTAYLALTRLASDDTAVYYCARDRLTGGRDAFEIWGQGTML | variable region sequence |
| | IVSS | |
| 833 | GYIFTAYY | Clone PA01P2B12 CDR-H1 |
| 826 | INPNRGHT | Clone PA01P2B12 CDR-H2 |
| 827 | ARDRLTGGRDAFEI | Clone PA01P2B12 CDR-H3 |
| 834 | DIQLTQSPSSLSASVGDRVTITCRASQSVSSYLNWYQQKPGEAPKLLISAASSLQDGVPP | Clone PA01P2B12 light chain |
| | RFSGSRSGTEFTLTISSLQPEDFAIYYCQQSYSLWWTFGQGTKVEIK | variable region sequence |
| 835 | QSVSSY | Clone PA01P2B12 CDR-L1 |
| 149 | AAS | Clone PA01P2B12 CDR-L2 |
| 836 | QQSYSLWWT | Clone PA01P2B12 CDR-L3 |
| 837 | QLQLQESGSGLVKPSQTLSLTCDVSGDSMNDDVYTWSWIRQPPGRGLEWIGYISHTGN | Clone PA01P2D11 heavy chain |
| | TFYNSSLQSRVTMSVDTSKNQFSLKLSSVTIADTAVYYCARLTFLFSAPFSSFNWFDPW | variable region sequence |
| | GQGILVTVSS | |
| 838 | GDSMNDDVYT | Clone PA01P2D11 CDR-H1 |
| 839 | ISHTGNT | Clone PA01P2D11 CDR-H2 |
| 840 | ARLTFLFSAPFSSFNWFDP | Clone PA01P2D11 CDR-H3 |
| 841 | QSVLTQPPSVSGAPGQTITISCTGTPSNFGADYDVHWYQQRPGTAPKLLIFADKHRPS | Clone PA01P2D11 light chain |
| | GVPDRFSGSRSGTSASLAISGLQAEDEADYYCQSYDSGVVGLWVFGGGTKVTVL | variable region sequence |
| 842 | PSNFGADYD | Clone PA01P2D11 CDR-L1 |
| 843 | ADK | Clone PA01P2D11 CDR-L2 |
| 844 | QSYDSGVVGLWV | Clone PA01P2D11 CDR-L3 |
| 845 | QVQLQQWGAGLLKPSETLSLTCGVHGGSLNNYYWSWIRQPPGKGLEWIGEVYHSGSI | Clone PA01P2B10 heavy chain |
| | NYNPSLKSRVTMSVDTSKNQFSFNLSSVTAADTAVYYCARGAYDSRGFWTLDAFNTW | variable region sequence |
| | GQGTMVIVSS | |
| 846 | GGSLNNYY | Clone PA01P2B10 CDR-H1 |
| 847 | VYHSGSI | Clone PA01P2B10 CDR-H2 |
| 848 | ARGAYDSRGFWTLDAFNT | Clone PA01P2B10 CDR-H3 |
| 849 | DIQMTQSPSALSASLGDRVTITCRASESINSWLAWYQQKPGKAPKLLIYKASTLESGVP | Clone PA01P2B10 light chain |
| | SRFSGSGSGTEFTLTISSLQPDDFATYYCHQYNRYSYTFGQGTKLDIK | variable region sequence |
| 850 | ESINSW | Clone PA01P2B10 CDR-L1 |
| 196 | KAS | Clone PA01P2B10 CDR-L2 |
| 851 | HQYNRYSYT | Clone PA01P2B10 CDR-L3 |
| 852 | EVLLLESGGGLVHPGGTLRLSCAASGFTFRNSAMTWVRQAPGKGLEWVSSIGGSGAKS | Clone PA01P2D10 heavy chain |
| | YYADSVKGRFTISRDNSKNTLYLEMNTLRVDDTAIYYCAKDQLNCYDLWSGDYCWFDT | variable region sequence |
| | WGQGTLVTVSS | |
| 853 | GFTFRNSA | Clone PA01P2D10 CDR-H1 |
| 854 | IGGSGAKS | Clone PA01P2D10 CDR-H2 |
| 855 | AKDQLNCYDLWSGDYCWFDT | Clone PA01P2D10 CDR-H3 |
| 856 | QSVLIQPPSASGTPGQRVTISCSGSNSNIGSNYVCWYQHLPGGAPKLLIYRNNQRPSGV | Clone PA01P2D10 light chain |
| | PDRFSGSKSGTSASLAISGLRSEDEADYYCAAWDDSLSGSWVFGGGTKLTVL | variable region sequence |
| 857 | NSNIGSNY | Clone PA01P2D10 CDR-L1 |
| 662 | RNN | Clone PA01P2D10 CDR-L2 |
| 858 | AAWDDSLSGSWV | Clone PA01P2D10 CDR-L3 |
| 859 | EVQLLESGGGLVQPGGSLRLSCAVSGLKFSSYAMSWVRQAPGKGLEWVSVVSGSSGST | Clone PA01P2D09 heavy chain |
| | FYAVSVEGRFSISRDNSNNMLYMDMHSLRVEDTAKYYCAKVVGWYYDRNGNRRPKGFR | variable region sequence |
| | AFDVWGQGTMVIVSS | |
| 860 | GLKFSSYA | Clone PA01P2D09 CDR-H1 |
| 861 | VSGSSGST | Clone PA01P2D09 CDR-H2 |
| 862 | AKVVGWYYDRNGNRRPKGFRAFDV | Clone PA01P2D09 CDR-H3 |
| 863 | QSVLTQPPSASGTPGQRITIACSGTTSNIGGNSVNWYQQFPGAAPRLLIFDYDQRPSGV | Clone PA01P2D09 light chain |
| | PARFSGSSSGSSGYLAISGLQSEDEADYYCSSWDDNLNGWVFGGGTKLTVL | variable region sequence |
| 864 | TSNIGGNS | Clone PA01P2D09 CDR-L1 |
| 865 | DYD | Clone PA01P2D09 CDR-L2 |
| 866 | SSWDDNLNGWV | Clone PA01P2D09 CDR-L3 |
| 867 | EVQLLESGGGLVQPGGSLRLSCVASGFTFSGHAMSWVRQAPGKGLEWVSGISGSGGST | Clone PA01P2B05 heavy chain |
| | YYADSVKGRFTISRDNSKKTVDLQMNNLRAEDTAIYYCAKDLHFDTSGYYYSMIFDYW | variable region sequence |
| | GQGTLVPVSS | |
| 868 | GFTFSGHA | Clone PA01P2805 CDR-H1 |
| 378 | ISGSGGST | Clone PA01P2B05 CDR-H2 |
| 869 | AKDLHFDTSGYYYSMIFDY | Clone PA01P2B05 CDR-H3 |
| 870 | QSALAQPASVSGSPGQSITISCTGTSSDISDYNYVSWYQQHPGKAPKLILYDVNNRPSGV | Clone PA01P2B05 light chain |
| | SSRFSGSKSGDTASLTISGLQPEDEADYYCSSYTSTKIFGGGTKVTVL | variable region sequence |
| 871 | SSDISDYNY | Clone PA01P2B05 CDR-L1 |
| 682 | DVN | Clone PA01P2B05 CDR-L2 |
| 872 | SSYTSTKI | Clone PA01P2B05 CDR-L3 |
| 873 | EVQLLESGGGLLQLGGSLRLSCAASGFTFSSYVMSWVRQAPGKGLEWVSLITGSGGNTY | Clone PA01P4C11 heavy chain |
| | YADSVKGRFTISRDNSKNTLFLQMNSLRVEDTAIYYCVKTDFYDSSGYYFHDAFHIWGQG | variable region sequence |
| | TMVTVSS | |
| 874 | GFTFSSYV | Clone PA01P4C11 CDR-H1 |
| 875 | ITGSGGNT | Clone PA01P4C11 CDR-H2 |
| 876 | VKTDFYDSSGYYFHDAFHI | Clone PA01P4C11 CDR-H3 |
| 877 | QTVVTQEPSLTVSPGGTVTLTCASSTGSVTSGYYPNWFQQKPGQAPRTLIYGTSNKHSW | Clone PA01P4C11 light chain |
| | TPARFSGSLLGGKAALTLSDVQPEDEAEYYCLLYYGGAYVFGTGTKVTVL | variable region sequence |
| 878 | TGSVTSGYY | Clone PA01P4C11 CDR-L1 |
| 22 | GTS | Clone PA01P4C11 CDR-L2 |
| 879 | LLYYGGAYV | Clone PA01P4C11 CDR-L3 |
| 880 | QVQLVQSGAEVKKPGASVKVSCKASGYTFIRYDIHWVRQATGQGLEWMGWMNPNNGKSGF | Clone PA01P3E08 heavy chain |
| | AQKFEGRVTLTRNTSVTSTYMQLSSLGLEDTAVYYCVRAGYSYGWGFDYWGQGSLVTVSS | variable region sequence |
| 881 | GYTFIRYD | Clone PA01P3E08 CDR-H1 |
| 882 | MNPNNGKS | Clone PA01P3E08 CDR-H2 |
| 883 | VRAGYSYGWGFDY | Clone PA01P3E08 CDR-H3 |
| 884 | NFTLTQPHSVSGSPGKTVTISCTRSSGGIASSHVQWYQQRPASAPTTLIFEDDQRSSGVP | Clone PA01P3E08 light chain |
| | DRFSGSIDTSSNSAYLTISGLEAEDEADYYCQSYDNSMWVFGGGSKVTVL | variable region sequence |
| 885 | SGGIASSH | Clone PA01P3E08 CDR-L1 |
| 886 | EDD | Clone PA01P3E08 CDR-L2 |
| 887 | QSYDNSMWV | Clone PA01P3E08 CDR-L3 |
| 888 | HVQLVQSGADVKKPGSSVKVSCKFSGGTFNNDSINWVRQAPGQGLEWMGVIMPFFGATR | Clone PA01P2E06 heavy chain |
| | FAPKFQGRVTLTADKFTSTGYMELGSLKSDDTAVYYCARDKPPDDKWADYGMDVWGQGT | variable region sequence |
| | TVTVSS | |
| 889 | GGTFNNDS | Clone PA01P2E06 CDR-H1 |
| 890 | IMPFFGAT | Clone PA01P2E06 CDR-H2 |
| 891 | ARDKPPDDKWADYGMDV | Clone PA01P2E06 CDR-H3 |
| 892 | SYELTQPPSVSVSPGQTARITCSGDALPKQYVYWYQQKTGQAPVLVIYKDTERPSGIPER | Clone PA01P2E06 light chain |
| | FSGSTSGTTVTLTISGVQADDEADYFCQSADRNANYRVFGGGTKLTVL | variable region sequence |
| 523 | ALPKQY | Clone PA01P2E06 CDR-L1 |
| 762 | KDT | Clone PA01P2E06 CDR-L2 |
| 893 | QSADRNANYRV | Clone PA01P2E06 CDR-L3 |
| 894 | QLQLQESGSGLVKPSQTLSLTCAVSGGSITSGTYSWTWIRQSPEKGLEWIGYIYYTGSTYY | Clone PA01P2E07 heavy chain |
| | NPSLGRRVTISGDTSNNEFSLNLKSVTAADTAVYYCARGIHRGGVLDFWGQGILVTVSS | variable region sequence |
| 895 | GGSITSGTYS | Clone PA01P2E07 CDR-H1 |
| 896 | IYYTGST | Clone PA01P2E07 CDR-H2 |
| 897 | ARGIHRGGVLDF | Clone PA01P2E07 CDR-H3 |
| 898 | EIVLTQSPATLPLSPGERATLSCRASQSLDKYLAWYQQKPGQAPRLLIYDTSKRATGIPAR | Clone PA01P2E07 light chain |
| | FSGSGSGTDFTLTISSLEPEDFAVYFCQQRNNWPPYTFGQGTKVEMK | variable region sequence |
| 899 | QSLDKY | Clone PA01P2E07 CDR-L1 |
| 900 | DTS | Clone PA01P2E07 CDR-L2 |
| 901 | QQRNNWPPYT | Clone PA01P2E07 CDR-L3 |
| 902 | QVLLVQSGSEVKNPGASIRVSCKTSGYMFTNNGIAWVREVPTQGLEWMGWISTYSGATHY | Clone PA01P2G07 heavy chain |
| | APNLHGRITMTADTSASTAYMELRSLQSGDTGVYYCARLWFGKLGLDFWGQGTQVTVSS | variable region sequence |
| 903 | GYMFTNNG | Clone PA01P2G07 CDR-H1 |
| 904 | ISTYSGAT | Clone PA01P2G07 CDR-H2 |
| 905 | ARLWFGKLGLDF | Clone PA01P2G07 CDR-H3 |
| 906 | QSVLTQPPSASGTPGQRVIISCSGSTSNIGTKTVNWYQHLPGTAPKLLIYNNNQRPSGVP | Clone PA01P2G07 light chain |
| | DRFSGSKSGTSASLTISGLQSEDEADYYCAAWDDSLNGRGLFGPGTKVTVL | variable region sequence |
| 907 | TSNIGTKT | Clone PA01P2G07 CDR-L1 |
| 908 | NNN | Clone PA01P2G07 CDR-L2 |
| 909 | AAWDDSLNGRGL | Clone PA01P2G07 CDR-L3 |
| 910 | QVEVVESGGGVVQPGKSLRLSCAASGFKFNVYGIHWVRQAPGKGLEWVAVVWYDGSNKY | Clone PA01P2B09 heavy chain |
| | YADSVKGRFTISRDNSKNTTYLQMDSLRVDDTAVYYCARELQYSNYDYFYAMDVWGQGTT | variable region sequence |
| | VTVSS | |
| 911 | GFKFNVYG | Clone PA01P2B09 CDR-H1 |
| 912 | VWYDGSNK | Clone PA01P2B09 CDR-H2 |
| 913 | ARELQYSNYDYFYAMDV | Clone PA01P2B09 CDR-H3 |
| 914 | DIQMTQSPPSLSASVGDRVTITCRASQDIDNYLVWFQQKPGRAPKSLIYAASSLQSGVPS | Clone PA01P2B09 light chain |
| | KFSGSGSGTEFTLTISSLQPEDFATYYCQQYNSFPYTFGQGTKLEIK | variable region sequence |
| 915 | QDIDNY | Clone PA01P2B09 CDR-L1 |
| 149 | AAS | Clone PA01P2B09 CDR-L2 |
| 916 | QQYNSFPYT | Clone PA01P2B09 CDR-L3 |
| 917 | QVQLQESGPGLVKPSETLSLTCSVSGGSISSHYWSWIRQPPGRGLEWIAYISYSGRTKYNP | Clone PA01P2C04 heavy chain |
| | SLKSRVTISEDTSKNQFSLKLSSVTPADTAVYYCARIYGDYGPFIDYWGQGTLVTVSS | variable region sequence |
| 918 | GGSISSHY | Clone PA01P2C04 CDR-H1 |
| 919 | ISYSGRT | Clone PA01P2C04 CDR-H2 |
| 920 | ARIYGDYGPFIDY | Clone PA01P2C04 CDR-H3 |
| 921 | DIQMTQSPSSLSASVGDRVTITCRASQTISTYLNWYQQKPGTAPMLLIYGAYSLHSGVPS | Clone PA01P2C04 light chain |
| | RFSGSGSGTDFTLTISSLQPEDFATYYCQQSSSLPLTFGGGTKVEIK | variable region sequence |
| 922 | QTISTY | Clone PA01P2C04 CDR-L1 |
| 923 | GAY | Clone PA01P2C04 CDR-L2 |
| 924 | QQSSSLPLT | Clone PA01P2C04 CDR-L3 |
| 925 | QEQLQESGPGLVKPSQTLSLTCTVSGGSISSGDHYWSWLRQTPGKGLEWIGYIYYRGNT | Clone PA01P2H08 heavy chain |
| | NYNPSLESRITMSVDTSKNQFSLKLSSVTAADTGVYYCARDRRLLFWFGQGPETFDIWG | variable region sequence |
| | PGTMVTVSS | |
| 926 | GGSISSGDHY | Clone PA01P2H08 CDR-H1 |
| 927 | IYYRGNT | Clone PA01P2H08 CDR-H2 |
| 928 | ARDRRLLFWFGQGPETFDI | Clone PA01P2H08 CDR-H3 |
| 929 | DIQMTQSPSILSASVGDRVTITCRASQNINHWLAWYQQKPGKAPKLLIYMASSLENGVPS | Clone PA01P2H08 light chain |
| | RFSGSGSGTEFTLTISSLQPDDFATYYCQQYNSYSGTFGHGTKVDIK | variable region sequence |
| 930 | QNINHW | Clone PA01P2H08 CDR-L1 |
| 931 | MAS | Clone PA01P2H08 CDR-L2 |
| 932 | QQYNSYSGT | Clone PA01P2H08 CDR-L3 |
| 933 | TACTGTGCGAAAGTTCTTGACTACAGTGAATTTCATTACTATTACGGTTTGGACGTCTGG | FIG. 13A PA12P3F10 |
| | GGCCAAGGGACCGCGGTCGCCGTCTCCTCAG | |
| 934 | TACTGTGCGAAAGTTCTTGACTACAATGAGTACTCTCTCTACTTCGGTATGGACGTCTGG | FIG. 13A PA12P3D08 |
| | GGCCAAGGGACCACGGTCACCGTCTCCTCAG | |
| 935 | TACTGTGCGAAAGTTCTTGACTACAGTGAATACTCTCTCTACTTCGGTATGGACGTCTGG | FIG. 13A PA12P1C07 |
| | GGCCAAGGGACCACGGTCCTTGTCTCCTCAG | |
| 936 | TACTGTGCGAAGGTCCTTGACTACAGTAGGTACTCCTATTATTACGGGATGGACGTCTGG | FIG. 13A PA13P1H08 |
| | GGCCAGGGGACCACGGTCATCGTCTCCTCAG | |
| 937 | TACTGTGCTAAGGTCCTTGACTACAGTGCATTCTCCTATTATTATGGGATGGACGTCTGG | FIG. 13A PA13P1E10 |
| | GGCCAGGGGACCACGGTCATCGTCTCCTCAG | |
| 938 | TATTGTGCGAAAGTCCTTGACTACAGTATTTTCTATTACTATTTCGGCCTGGACGTCTGGG | FIG. 13A PA13P3G09 |
| | GCCAAGGGACCACGGTCACCGTCTCCTCAG | |
| 939 | TACTGTGCGAAAGA | FIG. 13A IGHV30-30*18 |
| 940 | TYCT | FIG. 13A Nontemplated |
| | | (inferred) |
| 941 | TGACTACAGTAACTAC | FIG. 13A IGHD4-11*01 |
| 942 | ATTACTACTACTACTACGGTATGGACGTCTGGGGCCAAGGGACCACGGTCACCGTCTCCT | FIG. 13A IGHJ6*02 |
| | CAG | |
| 943 | YCAK | FIG. 13A Translated: V |
| 944 | VLDYSNY | FIG. 13A Translated: |
| | | Nontemplated + D |
| 945 | NYYYYYGMDVWGQGTTVTVSS | FIG. 13A Translated: J |
| 946 | YCAKVLDYSNYYYYYGMDVWGQGTTVTVSS | FIG. 13A Inferred naive |
| | | rearrangement |
| 947 | YCAKVLDYSEFHYYYGLDVWGQGTAVAVSS | FIG. 13A Translated: PA12P3F10 |
| 948 | YCAKVLDYNEYSLYFGMDVWGQGTTVTVSS | FIG. 13A Translated: PA12P3D08 |
| 949 | YCAKVLDYSEYSLYFGMDVWGQGTTVLVSS | FIG. 13A Translated: PA12P1C07 |
| 950 | YCAKVLDYSRYSYYYGMDVWGQGTTVIVSS | FIG. 13A Translated: PA13P1H08 |
| 951 | YCAKVLDYSAFSYYYGMDVWGQGTTVIVSS | FIG. 13A Translated: PA13P1E10 |
| 952 | YCAKVLDYSIFYYYFGLDVWGQGTTVTVSS | FIG. 13A Translated: PA13P3G09 |
| 953 | GCAGTGTATTACTGTCAGCATTACAGTAATTCACCCCCGTACACTTTTGGCCCGGGGACC | FIG. 13C PA12P3F10 |
| | AAGTTGGAGATCAAAC | |
| 954 | GCAGTGTATTTCTGTCAGTACTATAGTGACTCACCTCCGTACACTTTTGGCCCGGGGACC | FIG. 13C PA12P3D08 |
| | AAGCTGGAGATCAAAC | |
| 955 | GCAGTGTATTCCTGTCAACACTATAGTGACTCACCTCCTTACACTTTTGGCCAGGGGACCA | FIG. 13C PA12P1C07 |
| | AACTGGAGATCAAAC | |
| 956 | GCAGTTTATTACTGTCAGCACTATGGTAGGTCACCTCCGTACACTTTTGGCCCGGGGACC | FIG. 13C PA13P1H08 |
| | AAGCTGGACATCAAAC | |
| 957 | GCAGTATATTACTGTCAACACTATGGTAGGTCACCTCCATACACTTTTGGCCAGGGGACC | FIG. 13C PA13P1E10 |
| | AAAGTGGAGATCAAAC | |
| 958 | GCAGTGTACTACTGTCAGCACTATGGAGACTCACCTCCGTACACCTTTGGCCAGGGGACG | FIG. 13C PA13P3G09 |
| | AAAGTGGAGATGAAAC | |
| 959 | GCAGTGTATTACTGTCAGCAGTATGGTAGCTCACCTCC | FIG. 13C IGKV3-20*01 |
| 960 | TGTACACTTTTGGCCAGGGGACCAAGCTGGAGATCAAAC | FIG. 13C IGKJ2*01 |
| 961 | AVYYCQQYGSSP | FIG. 13C Translated: V |
| 962 | PYTFGQGTKLEIK | FIG. 13C Translated: J |
| 963 | AVYYCQQYGSSPPYTFGQGTKLEIK | FIG. 13C Inferred naive |
| | | rearrangement |
| 964 | AVYYCQHYSNSPPYTFGPGTKLEIK | FIG. 13C Translated: PA12P3F10 |
| 965 | AVYFCQYYSDSPPYTFGPGTKLEIK | FIG. 13C Translated: PA12P3D08 |
| 966 | AVYSCQHYSDSPPYTFGQGTKLEIK | FIG. 13C Translated: PA12P1C07 |
| 967 | AVYYCQHYGRSPPYTFGPGTKLDIK | FIG. 13C Translated: PA13P1H08 |
| 968 | AVYYCQHYGRSPPYTFGQGTKVEIK | FIG. 13C Translated: PA13P1E10 |
| 969 | AVYYCQHYGDSPPYTFGQGTKVEMK | FIG. 13C Translated: PA13P3G09 |
| 970 | EIVLTQSPATLSLSPGERATLSCRASQSVSSYLAWYQQKPGQAPRLLIYDASNRATGIPAR | FIG. 13E swap |
| | FSGSGSGTDFTLTISSLEPEDFAVYYCQQRSNWPPGFTFGPGTKVDIK | |
| 971 | AKVMDYDIFKNYFGLDV | FIG. 16 |
| 972 | AKVMDYDVFKNYYGLDV | FIG. 16 |
| 973 | AKTLDYSQYMYYYGLDV | FIG. 16 |
| 974 | QHYGRSPPYT | FIG. 12G |
| 975 | QHYGSSPPFT | FIG. 12G |
| 976 | QHYGSLPPFT | FIG. 12G |
| 977 | AAWDDTLVGV | FIG. 12G |
| 978 | AAWDDLVVGV | FIG. 12G |
| 979 | QSTDSSGDYVV | FIG. 12G |
| 980 | QSTDSSLRDVV | FIG. 12G |
| 981 | TSYAGRNIQV | FIG. 12G |
| 982 | SSYAGSNIAV | FIG. 12G |
| 983 | QSYDGSSPVI | FIG. 12G |
| 984 | QSYDTNIVV | FIG. 12G |
| 985 | QSYDSANVV | FIG. 12G |
| 986 | QSYDADNAV | FIG. 12G |
| 987 | SSYTRETALGGV | FIG. 12G |
| 988 | QQYYTTPRT | FIG. 12G |
| 989 | QQYYTTPYT | FIG. 12G |
| 990 | QQYLTTPYT | FIG. 12G |
| 991 | QQYDEWPPFT | FIG. 12G |
| 992 | QQYNHWPPYT | FIG. 12G |
| 993 | GSYKSGSTWV | FIG. 12G |
| 994 | SSYRSGSTWV | FIG. 12G |
| 995 | SSYTSGRTWV | FIG. 12G |
| 996 | SSYTTGRTWV | FIG. 12G |
| 997 | ASRYCTDSGCYLGSFDY | FIG. 12G |
| 998 | ASRYCTDDGCYLGSFDF | FIG. 12G |
| 999 | TRDHGYY | FIG. 12G |
| 1000 | ARDHGYY | FIG. 12G |
| 1001 | ARDPAAGTWWFDP | FIG. 12G |
| 1002 | ARPSAHYYDRGGYNDAFDM | FIG. 12G |
| 1003 | TTGYRTTTTYHGDDAFDI | FIG. 12G |
| 1004 | TTGYRTSTSYHGDDAFDI | FIG. 12G |
| 1005 | ARGPPAVQGYFYYMYV | FIG. 12G |
| 1006 | ARGPPGVHGYFYYTDV | FIG. 12G |
| 1007 | ARDVVRPGSGPRLGFDP | FIG. 12G |
| 1008 | ARDVVRPGRGPRLGFDP | FIG. 12G |
| 1009 | AKEGGSSTSWYSLYHEYEMDV | FIG. 12G |
| 1010 | AHKAAEPGSRDRWFDS | FIG. 12G |
| 1011 | AGGYNNSSFYFDS | FIG. 12G |
| 1012 | AVGYNNSWFYFDY | FIG. 12G |
| 1013 | ARLGHLRGWFDS | FIG. 12G |
| 1014 | VLSQYEFGSSWFYYYRMDV | FIG. 12G |
| 1015 | VLSKYEFGSSWFYYYRMDV | FIG. 12G |
| 1016 | VLSKYEFHSSWFYYYRMDV | FIG. 12G |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of identifying a human monoclonal antibody that specifically binds to an allergen, the method comprising:
isolating B cells from a sample from a subject known to have an allergy to the allergen by negative selection from blood cells;
selecting the B cells that express an immunoglobulin isotype;
sequencing nucleic acids from individual B cells of the B cells to obtain sequence data of heavy chain transcripts from the individual B cells;
analyzing the sequence data to select a candidate B cell that expresses an immunoglobulin heavy chain that comprises an IgE constant region; and,
identifying, from the candidate B cell, (i) a heavy chain variable region sequence, and (ii) a light chain variable region sequence that are co-expressed with the immunoglobulin heavy chain in the candidate B cell;
thereby identifying the human monoclonal antibody comprising an IgE constant region that specifically binds to the allergen.

2. The method of claim 1 wherein the sequencing comprises performing whole transcriptome sequencing.

3. The method of claim 1, wherein the sequencing comprises performing single cell RNA sequencing.

4. The method of claim 1, wherein the sequencing is performed to a depth of at least 500 thousand reads/cell.

5. The method of claim 1, wherein the selecting the B cells that express the immunoglobulin isotype comprises selecting for IgE positive cells.

6. The method of claim 1, wherein the nucleic acids comprise mRNA.

7. The method of claim 6, further comprising:
generating cDNA from the B cells; and,
sequencing the cDNA.

8. The method of claim 1, wherein the nucleic acids comprise DNA.

9. The method of claim 1, further comprising expressing an antibody comprising the immunoglobulin heavy chain expressed by the candidate B cell.

10. The method of claim 9, further comprising determining that the antibody expressed binds to the allergen.

11. The method of claim 1, wherein the sample is a body fluid sample.

12. The method of claim 1, wherein the sample is a tissue sample.

13. The method of claim 1, wherein the isolating comprises sorting cells by fluorescent activated cell sorting (FACS).

14. The method of claim 1, wherein the isolating comprises:
contacting cells from the sample with an anti-human CD19 antibody, and an anti-human IgE antibody; and,
selecting for CD19+ IgE-expressing B cells.

15. The method of claim 1, wherein the isolating comprises:
contacting cells from the sample with an anti-human CD19 antibody, an anti-human IgE antibody, an anti-human IgM antibody, and an anti-human IgG antibody; and,
selecting for CD19+ IgM-IgG-IgE-expressing B cells.

16. The method of claim 1, wherein the isolated B cells express a B cell marker and are negative for non-IgE isotypes.

17. The method of claim 1, wherein the isolating comprises:
contacting cells from the sample with an anti-human CD19 antibody, an anti-human IgM antibody, an anti-human IgG antibody, an anti-human IgA antibody, and an anti-human IgD antibody; and,
selecting for CD19+IgM−IgG−IgA−IgD− B cells.

18. The method of claim 1, further comprising substituting the IgE constant region with a wild-type IgG4 constant region or a modified IgG4 constant region.

19. The method of claim 1, wherein the allergen is a food allergen, a plant allergen, a fungal allergen, an animal allergen, a drug allergen, a cosmetic allergen, or a latex allergen.

20. The method of claim 19, wherein the allergen is the food allergen selected from the group consisting of a milk allergen, an egg allergen, a nut allergen, a fish allergen, a shellfish allergen, a soy allergen, a legume allergen, a seed allergen, and a wheat allergen.


SEQUENCE LISTING (1019 sequences)

SEQ ID NO: 1 - protein, 124 aa, synthetic construct
  REGION 1..124: note = Description of Artificial Sequence: Synthetic polypeptide
QVQLVNSGGGVVQPGRSLRLSCVASGFTFSTFGIHWVRQAPGKGLEWVAVISNDGEKSESADSVKGRFTPSRDNSKNTVYLQMNNLRVEDTAVYYCAKVLDYSRYSYYYGMDVWGQGTTVIVSS

SEQ ID NO: 2 - protein, 8 aa, synthetic construct
  REGION 1..8: note = Description of Artificial Sequence: Synthetic peptide
GFTFSTFG

SEQ ID NO: 3 - protein, 8 aa, synthetic construct
  REGION 1..8: note = Description of Artificial Sequence: Synthetic peptide
ISNDGEKS

SEQ ID NO: 4 - protein, 17 aa, synthetic construct
  REGION 1..17: note = Description of Artificial Sequence: Synthetic peptide
AKVLDYSRYSYYYGMDV

SEQ ID NO: 5 - protein, 109 aa, synthetic construct
  REGION 1..109: note = Description of Artificial Sequence: Synthetic polypeptide
EIVLTQSPGTLSLSPGGRGTLSCRTSQTINNAHLAWYQHKPGQAPRLLIYGSSERATGVPDRFSGSGSGSDFTLTISSLEAEDFAVYYCQHYGRSPPYTFGPGTKLDIK

SEQ ID NO: 6 - protein, 7 aa, synthetic construct
  REGION 1..7: note = Description of Artificial Sequence: Synthetic peptide
QTINNAH

SEQ ID NO: 7 - skipped

SEQ ID NO: 8 - protein, 10 aa, synthetic construct
  REGION 1..10: note = Description of Artificial Sequence: Synthetic peptide
QHYGRSPPYT

SEQ ID NO: 9 - protein, 124 aa, synthetic construct
  REGION 1..124: note = Description of Artificial Sequence: Synthetic polypeptide
QVQLVDSGGGVVQPGKSLRLSCVGSGFTFRTFGIHWVRQAPGKGLEWVAVISNDGGNSASADSVKGRFTTSRDNSKNTVYLQINSLRPEDTAIYYCAKVLDYSAFSYYYGMDVWGQGTTVIVSS

SEQ ID NO: 10 - protein, 8 aa, synthetic construct
  REGION 1..8: note = Description of Artificial Sequence: Synthetic peptide
GFTFRTFG

SEQ ID NO: 11 - protein, 8 aa, synthetic construct
  REGION 1..8: note = Description of Artificial Sequence: Synthetic peptide
ISNDGGNS

SEQ ID NO: 12 - protein, 17 aa, synthetic construct
  REGION 1..17: note = Description of Artificial Sequence: Synthetic peptide
AKVLDYSAFSYYYGMDV

SEQ ID NO: 13 - protein, 109 aa, synthetic construct
  REGION 1..109: note = Description of Artificial Sequence: Synthetic polypeptide
EIVLTQSPGTLSLSPGERGTLSCRTSQPISRAHLAWYQHKAGQAPRLLIYGSTERAAGIPERFSGGGSGSDFTLTISSLEAEDFAVYYCQHYGRSPPYTFGQGTKVEIK

SEQ ID NO: 14 - protein, 7 aa, synthetic construct
  REGION 1..7: note = Description of Artificial Sequence: Synthetic peptide
QPISRAH

SEQ ID NO: 15 - skipped

SEQ ID NO: 16 - protein, 124 aa, synthetic construct
  REGION 1..124: note = Description of Artificial Sequence: Synthetic polypeptide
QVQLVESGGGVVQPGGSLTLSCVGSGFTFSHYAIHWVRQAPGKGLEWVAVISNVGTTRDYADSLKGRLTISRENSQSTVFLQMNSLRADDTAIYYCAKVLDYSEFHYYYGLDVWGQGTAVAVSS

SEQ ID NO: 17 - protein, 8 aa, synthetic construct
  REGION 1..8: note = Description of Artificial Sequence: Synthetic peptide
GFTFSHYA

SEQ ID NO: 18 - protein, 8 aa, synthetic construct
  REGION 1..8: note = Description of Artificial Sequence: Synthetic peptide
ISNVGTTR

SEQ ID NO: 19 - protein, 17 aa, synthetic construct
  REGION 1..17: note = Description of Artificial Sequence: Synthetic peptide
AKVLDYSEFHYYYGLDV

SEQ ID NO: 20 - protein, 109 aa, synthetic construct
  REGION 1..109: note = Description of Artificial Sequence: Synthetic polypeptide
EIVLTQSPGTLSLSPGQRVTLSCRVSQAIPTMYVAWYQQRPGQAPRLLIYGTSSRATGIPDRFSGGGSGTDFTLTINRLEPEDIAVYYCQHYSNSPPYTFGPGTKLEIK

SEQ ID NO: 21 - protein, 7 aa, synthetic construct
  REGION 1..7: note = Description of Artificial Sequence: Synthetic peptide
QAIPTMY

SEQ ID NO: 22 - skipped

SEQ ID NO: 23 - protein, 10 aa, synthetic construct
  REGION 1..10: note = Description of Artificial Sequence: Synthetic peptide
QHYSNSPPYT

SEQ ID NO: 24 - protein, 124 aa, synthetic construct
  REGION 1..124: note = Description of Artificial Sequence: Synthetic polypeptide
QEQLVESGGGVVHPGGSLRLSCVASAFTFNRFGMHWVRQAPGKGLEWVAVISNDGRSQDYADSVKGRFIISRDNSKNTLYLQLNSLRFEDTAVYYCAKVLDYSIFYYYFGLDVWGQGTTVTVSS

SEQ ID NO: 25 - protein, 8 aa, synthetic construct
  REGION 1..8: note = Description of Artificial Sequence: Synthetic peptide
AFTFNRFG

SEQ ID NO: 26 - protein, 8 aa, synthetic construct
  REGION 1..8: note = Description of Artificial Sequence: Synthetic peptide
ISNDGRSQ

SEQ ID NO: 27 - protein, 17 aa, synthetic construct
  REGION 1..17: note = Description of Artificial Sequence: Synthetic peptide
AKVLDYSIFYYYFGLDV

SEQ ID NO: 28 - protein, 109 aa, synthetic construct
  REGION 1..109: note = Description of Artificial Sequence: Synthetic polypeptide
EVVLTQSPGSLSLSPGERATLSCRAGQSLSSKFLAWYQHKPGQAPRLLIYGASTRATGVPDRFSGSGSGTDFSLIISRVEPEDFAVYYCQHYGDSPPYTFGQGTKVEMK

SEQ ID NO: 29 - protein, 7 aa, synthetic construct
  REGION 1..7: note = Description of Artificial Sequence: Synthetic peptide
QSLSSKF

SEQ ID NO: 30 - skipped

SEQ ID NO: 31 - protein, 10 aa, synthetic construct
  REGION 1..10: note = Description of Artificial Sequence: Synthetic peptide
QHYGDSPPYT

SEQ ID NO: 32 - protein, 124 aa, synthetic construct
  REGION 1..124: note = Description of Artificial Sequence: Synthetic polypeptide
QVQLVESGGGVVQPGKSLRLSCAASAFTFRRFAMHWVRQAPGKGLEWVAVISDNGLREDYEDSVKGRFTISRDNSQNTLYLQMNGLRAEDTAVYYCAKVLDYNEYSLYFGMDVWGQGTTVTVSS

SEQ ID NO: 33 - protein, 8 aa, synthetic construct
  REGION 1..8: note = Description of Artificial Sequence: Synthetic peptide
AFTFRRFA

SEQ ID NO: 34 - protein, 8 aa, synthetic construct
  REGION 1..8: note = Description of Artificial Sequence: Synthetic peptide
ISDNGLRE

SEQ ID NO: 35 - protein, 17 aa, synthetic construct
  REGION 1..17: note = Description of Artificial Sequence: Synthetic peptide
AKVLDYNEYSLYFGMDV

SEQ ID NO: 36 - protein, 109 aa, synthetic construct
  REGION 1..109: note = Description of Artificial Sequence: Synthetic polypeptide
EVVLTQSPATLSLSPGERATLSCRTSQAISNNFLAWYQQRPGQPPRLLIYASSRRATDTPDRFTGSGSGTDFTLTITRLEPEDFAVYFCQYYSDSPPYTFGPGTKLEIK

SEQ ID NO: 37 - protein, 7 aa, synthetic construct
  REGION 1..7: note = Description of Artificial Sequence: Synthetic peptide
QAISNNF

SEQ ID NO: 38 - skipped

SEQ ID NO: 39 - protein, 10 aa, synthetic construct
  REGION 1..10: note = Description of Artificial Sequence: Synthetic peptide
QYYSDSPPYT

SEQ ID NO: 40 - protein, 124 aa, synthetic construct
  REGION 1..124: note = Description of Artificial Sequence: Synthetic polypeptide
QVQLEESGGGVVQPGKSLRLSCVASAFTFKRFAMHWVRQAPGKGLEWVAVISDNGLREDYEDSVKGRFTISRDNSKDTLYLQMNSLRPEDTAIYYCAKVLDYSEYSLYFGMDVWGQGTTVLVSS

SEQ ID NO: 41 - protein, 8 aa, synthetic construct
  REGION 1..8: note = Description of Artificial Sequence: Synthetic peptide
AFTFKRFA

SEQ ID NO: 42 - protein, 17 aa, synthetic construct
  REGION 1..17: note = Description of Artificial Sequence: Synthetic peptide
AKVLDYSEYSLYFGMDV

SEQ ID NO: 43 - protein, 109 aa, synthetic construct
  REGION 1..109: note = Description of Artificial Sequence: Synthetic polypeptide
EIVLTQSPAILSLSPGDRATLSCRTSQTVNSNFLAWYQQKPGQAPRLLIYGASRRAIDIPDRFTGSGSGTEFTLTIARLEPEDFAVYSCQHYSDSPPYTFGQGTKLEIK

SEQ ID NO: 44 - protein, 7 aa, synthetic construct
  REGION 1..7: note = Description of Artificial Sequence: Synthetic peptide
QTVNSNF

SEQ ID NO: 45 - protein, 10 aa, synthetic construct
  REGION 1..10: note = Description of Artificial Sequence: Synthetic peptide
QHYSDSPPYT

SEQ ID NO: 46 - protein, 126 aa, synthetic construct
  REGION 1..126: note = Description of Artificial Sequence: Synthetic polypeptide
QVHLVESGGGVVQPGRSLGLSCAASGFTFNYYAIHWVRQAPGKGLEWVAVVSFDGNIIYYADSVKGRFNISRDNSKNTVNLQMNSLRADDTAVYYCVRDGEYCSGGNCYWGDFDYWGQGTLVTVSP

SEQ ID NO: 47 - protein, 8 aa, synthetic construct
  REGION 1..8: note = Description of Artificial Sequence: Synthetic peptide
GFTFNYYA

SEQ ID NO: 48 - protein, 8 aa, synthetic construct
  REGION 1..8: note = Description of Artificial Sequence: Synthetic peptide
VSFDGNII

SEQ ID NO: 49 - protein, 19 aa, synthetic construct
  REGION 1..19: note = Description of Artificial Sequence: Synthetic peptide
VRDGEYCSGGNCYWGDFDY

SEQ ID NO: 50 - protein, 107 aa, synthetic construct
  REGION 1..107: note = Description of Artificial Sequence: Synthetic polypeptide
EIVLTQSPGTLSLSPGERATLSCRASQSISSEYLTWFQQKPGQAPRLLIYGAFNRATGIPDRFSGSGSGTDFTLTISSLEPEDFAVYYCQQYANWWTFGQGTKVEIK

SEQ ID NO: 51 - protein, 7 aa, synthetic construct
  REGION 1..7: note = Description of Artificial Sequence: Synthetic peptide
QSISSEY

SEQ ID NO: 52 - skipped

SEQ ID NO: 53 - protein, 8 aa, synthetic construct
  REGION 1..8: note = Description of Artificial Sequence: Synthetic peptide
QQYANWWT

SEQ ID NO: 54 - protein, 126 aa, synthetic construct
  REGION 1..126: note = Description of Artificial Sequence: Synthetic polypeptide
QVHLVESGGGVVQPGRSLGLSCVASGFTFNYYAIHWVRQAPGKGLEWVAVVSFDGNIIYYADSVKGRFNISRDNSKNTVNLQMNSLRPDDTAVYYCVRDGEYCSGGNCYWGDFDHWGQGSLVTVSP

SEQ ID NO: 55 - protein, 19 aa, synthetic construct
  REGION 1..19: note = Description of Artificial Sequence: Synthetic peptide
VRDGEYCSGGNCYWGDFDH

SEQ ID NO: 56 - protein, 107 aa, synthetic construct
  REGION 1..107: note = Description of Artificial Sequence: Synthetic polypeptide
EIVLTQSPATLSLSPGERATLSCRASQSISSEYLTWFQQKPGQAPRLLIYGAFNRATGIPDRFSGSGSGTDFTLTISSLEPEDFAVYYCQQYANWWTFGQGTKVEIK

SEQ ID NO: 57 - protein, 124 aa, synthetic construct
  REGION 1..124: note = Description of Artificial Sequence: Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKVLDYSNYYYYYGMDVWGQGTTVTVSS

SEQ ID NO: 58 - protein, 8 aa, synthetic construct
  REGION 1..8: note = Description of Artificial Sequence: Synthetic peptide
GFTFSSYG

SEQ ID NO: 59 - protein, 8 aa, synthetic construct
  REGION 1..8: note = Description of Artificial Sequence: Synthetic peptide
ISYDGSNK

SEQ ID NO: 60 - protein, 17 aa, synthetic construct
  REGION 1..17: note = Description of Artificial Sequence: Synthetic peptide
AKVLDYSNYYYYYGMDV

SEQ ID NO: 61 - protein, 109 aa, synthetic construct
  REGION 1..109: note = Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKLEIK

SEQ ID NO: 62 - protein, 7 aa, synthetic construct
  REGION 1..7: note = Description of Artificial Sequence: Synthetic peptide
QSVSSSY

SEQ ID NO: 63 - protein, 10 aa, synthetic construct
  REGION 1..10: note = Description of Artificial Sequence: Synthetic peptide
QQYGSSPPYT

SEQ ID NO: 64 - protein, 124 aa, synthetic construct
  REGION 1..124: note = Description of Artificial Sequence: Synthetic polypeptide
QVQLVNSGGGVVQPGRSLRLSCVASGFTFSSYGIHWVRQAPGKGLEWVAVISNDGEKSESADSVKGRFTPSRDNSKNTVYLQMNNLRVEDTAVYYCAKVLDYSRYSYYYGMDVWGQGTTVIVSS

SEQ ID NO: 65 - protein, 124 aa, synthetic construct
  REGION 1..124: note = Description of Artificial Sequence: Synthetic polypeptide
QVQLVNSGGGVVQPGRSLRLSCVASGFTFSTFGIHWVRQAPGKGLEWVAVISYDGSNKESADSVKGRFTPSRDNSKNTVYLQMNNLRVEDTAVYYCAKVLDYSRYSYYYGMDVWGQGTTVIVSS

SEQ ID NO: 66 - protein, 124 aa, synthetic construct
  REGION 1..124: note = Description of Artificial Sequence: Synthetic polypeptide
QVQLVNSGGGVVQPGRSLRLSCVASGFTFSTFGIHWVRQAPGKGLEWVAVISNDGEKSESADSVKGRFTPSRDNSKNTVYLQMNNLRVEDTAVYYCAKVLDYSNYYYYYGMDVWGQGTTVIVSS

SEQ ID NO: 67 - protein, 124 aa, synthetic construct
  REGION 1..124: note = Description of Artificial Sequence: Synthetic polypeptide
QVQLVESGGGVVQPGRSLRLSCAASGFTFSTFGMHWVRQAPGKGLEWVAVISNDGEKSYYADSVKGRFTISRDNSKNTLYLQMNSLRAEDTAVYYCAKVLDYSRYSYYYGMDVWGQGTTVTVSS

SEQ ID NO: 68 - DNA, 1284 bp, unidentified
  misc_feature 1..1284: note = Description of Unknown: Nucleotide sequence for IgE heavy chain constant region
  source 1..1284: mol_type = other DNA
gcctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaa

SEQ ID NO: 69 - protein, 428 aa, unidentified
  REGION 1..428: note = Description of Unknown: Amino acid sequence for IgE heavy chain constant region
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 70 - DNA, 981 bp, unidentified
  misc_feature 1..981: note = Description of Unknown: Nucleotide sequence for IgG4 heavy chain constant region
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 71 - protein, 327 aa, unidentified
  REGION 1..327: note = Description of Unknown: Amino acid sequence for IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 127 aa, synthetic construct
  REGION 1..127: note = Description of Artificial Sequence: Synthetic polypeptide
QVQLVESGGGVVQPGRSLRLSCAASGFTFDTYGMHWVRQAPGKGPEWVAVIWYDGTREDYADSVKGRFTVSRDNSKSTLFLQMNSLRADDTAVYYCAKEHNTYFSDHIGRVGGMDVWGQGTTVIVSS

SEQ ID NO: 73 - protein, 8 aa, synthetic construct
  REGION 1..8: note = Description of Artificial Sequence: Synthetic peptide
GFTFDTYG

SEQ ID NO: 74 - protein, 8 aa, synthetic construct
  REGION 1..8: note = Description of Artificial Sequence: Synthetic peptide
IWYDGTRE

SEQ ID NO: 75 - protein, 20 aa, synthetic construct
  REGION 1..20: note = Description of Artificial Sequence: Synthetic peptide
AKEHNTYFSDHIGRVGGMDV

SEQ ID NO: 76 - protein, 113 aa, synthetic construct
  REGION 1..113: note = Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQVLQTPPWTFGQGTQVEIK

SEQ ID NO: 77 - protein, 11 aa, synthetic construct
  REGION 1..11: note = Description of Artificial Sequence: Synthetic peptide
QSLLHSNGYNY

SEQ ID NO: 78 - skipped

SEQ ID NO: 79 - protein, 10 aa, synthetic construct
  REGION 1..10: note = Description of Artificial Sequence: Synthetic peptide
MQVLQTPPWT

SEQ ID NO: 80 - protein, 124 aa, synthetic construct
  REGION 1..124: note = Description of Artificial Sequence: Synthetic polypeptide
QVQLVESGGGVVQPGRSLRLSCAGSGFTFNAYGLHWVRQAPGKGLEWVAGIYYDGSNKYYADSVKGRFAISRDNSQNTLYLEMNSLRVEDTAVYYCAKAGPIASIGTRHTFDHWGQGTLVTVSS

SEQ ID NO: 81 - protein, 8 aa, synthetic construct
  REGION 1..8: note = Description of Artificial Sequence: Synthetic peptide
GFTFNAYG

SEQ ID NO: 82 - protein, 8 aa, synthetic construct
  REGION 1..8: note = Description of Artificial Sequence: Synthetic peptide
IYYDGSNK

SEQ ID NO: 83 - protein, 17 aa, synthetic construct
  REGION 1..17: note = Description of Artificial Sequence: Synthetic peptide
AKAGPIASIGTRHTFDH

SEQ ID NO: 84 - protein, 113 aa, synthetic construct
  REGION 1..113: note = Description of Artificial Sequence: Synthetic polypeptide
DIVMTQSPDSLAVSLGERATINCKSSQSLLLNSNNKNYLAWYQQKPGQPPKLLIYWASTRESGVPGRFSGNGSVTDFTLTISGLQAEDVAVYYCHQYYTTSYTFGQGTKLEIK

SEQ ID NO: 85 - protein, 12 aa, synthetic construct
  REGION 1..12: note = Description of Artificial Sequence: Synthetic peptide
QSLLLNSNNKNY

SEQ ID NO: 86 - skipped

SEQ ID NO: 87 - protein, 9 aa, synthetic construct
  REGION 1..9: note = Description of Artificial Sequence: Synthetic peptide
HQYYTTSYT

SEQ ID NO: 88 - protein, 118 aa, synthetic construct
  REGION 1..118: note = Description of Artificial Sequence: Synthetic polypeptide
QVQLVQSGAEVKKPGASVKISCKAVGYTFTSYYLHWVRQAPGQGLEWVGIIDPSRGHRNYAQGFQGRVTMTSDTSTSTVYMDLGSLRSEDTAVYYCARAPARDHFDNWGQGTPVTVSP

SEQ ID NO: 89 - protein, 8 aa, synthetic construct
  REGION 1..8: note = Description of Artificial Sequence: Synthetic peptide
GYTFTSYY

SEQ ID NO: 90 - protein, 8 aa, synthetic construct
  REGION 1..8: note = Description of Artificial Sequence: Synthetic peptide
IDPSRGHR

SEQ ID NO: 91 - protein, 11 aa, synthetic construct
  REGION 1..11: note = Description of Artificial Sequence: Synthetic peptide
ARAPARDHFDN

SEQ ID NO: 92 - protein, 107 aa, synthetic construct
  REGION 1..107: note = Description of Artificial Sequence: Synthetic polypeptide
DIQMTQSPSSLAASVGDRVTINCQASQDIRNCLNWYQQQPGKAPKLLIYDASILETGVPSRFSGSGSGTDFTFSISSLQPEDIATYYCQQCEDLPLTFGPGSKVDIK

SEQ ID NO: 93 - protein, 6 aa, synthetic construct
  REGION 1..6: note = Description of Artificial Sequence: Synthetic peptide
QDIRNC

SEQ ID NO: 94 - skipped

SEQ ID NO: 95 - protein, 9 aa, synthetic construct
  REGION 1..9: note = Description of Artificial Sequence: Synthetic peptide
QQCEDLPLT

SEQ ID NO: 96 - protein, 125 aa, synthetic construct
  REGION 1..125: note = Description of Artificial Sequence: Synthetic polypeptide
QVQLVQSGAEVKQPGSSVKVSCKASGGTFRNSALSWVRQAPGQGLEWMGGIIPIFDTTNYAQEFQGRVTITADKSTTTAYMELSSLKSEDTAVYYCARGEGLPWLTYHYYGMDVWGQGTTVTVSS

SEQ ID NO: 97 - protein, 8 aa, synthetic construct
  REGION 1..8: note = Description of Artificial Sequence: Synthetic peptide
GGTFRNSA

SEQ ID NO: 98 - protein, 8 aa, synthetic construct
  REGION 1..8: note = Description of Artificial Sequence: Synthetic peptide
IIPIFDTT

SEQ ID NO: 99 - protein, 18 aa, synthetic construct
  REGION 1..18: note = Description of Artificial Sequence: Synthetic peptide
ARGEGLPWLTYHYYGMDV

SEQ ID NO: 100 - protein, 115 aa, synthetic construct
  REGION 1..115: note = Description of Artificial Sequence: Synthetic polypeptide
QAVLTQPSSLSASPGASASLTCTLRSGINIGTDRIYWFQQKPGSPPQYLLTYKSDSDEQRGSGVPSRFSGSKDVSANAGILLISGLQSEDEADYYCMIWHSSAWVFGGGTKLTVL

SEQ ID NO: 101 - protein, 9 aa, synthetic construct
  REGION 1..9: note = Description of Artificial Sequence: Synthetic peptide
SGINIGTDR

SEQ ID NO: 102 - protein, 7 aa, synthetic construct
  REGION 1..7: note = Description of Artificial Sequence: Synthetic peptide
YKSDSDE

SEQ ID NO: 103 - protein, 9 aa, synthetic construct
  REGION 1..9: note = Description of Artificial Sequence: Synthetic peptide
MIWHSSAWV

SEQ ID NO: 104 - protein, 120 aa, synthetic construct
  REGION 1..120: note = Description of Artificial Sequence: Synthetic polypeptide
QVQLQQWGAGLLKPSETLSLTCAVYGGSLSGYHWSWIRQPPGKGLQWIGEISHSGNAKYNPSLKSRVSISVHMSKNEFYLNLTSVTAADTAVYYCARGYCSGGSCYYKFWGQGTLVTVSS

SEQ ID NO: 105 - protein, 8 aa, synthetic construct
  REGION 1..8: note = Description of Artificial Sequence: Synthetic peptide
GGSLSGYH

SEQ ID NO: 106 - protein, 7 aa, synthetic construct
  REGION 1..7: note = Description of Artificial Sequence: Synthetic peptide
ISHSGNA

SEQ ID NO: 107 - protein, 14 aa, synthetic construct
  REGION 1..14: note = Description of Artificial Sequence: Synthetic peptide
ARGYCSGGSCYYKF

SEQ ID NO: 108 - protein, 110 aa, synthetic construct
  REGION 1..110: note = Description of Artificial Sequence: Synthetic polypeptide
QSVLTQPPSVSAAPGQKVTISCSGNSSNIGNNYVSWFQQLPGTAPKLLIYDNNKRPSGIPDRFSGSKSGTSATLGITGLQTGDEADYFCGTWDSSLRTGVFGGGTKLTVL

SEQ ID NO: 109 - protein, 8 aa, synthetic construct
  REGION 1..8: note = Description of Artificial Sequence: Synthetic peptide
SSNIGNNY

SEQ ID NO: 110 - skipped

SEQ ID NO: 111 - protein, 11 aa, synthetic construct
  REGION 1..11: note = Description of Artificial Sequence: Synthetic peptide
GTWDSSLRTGV

SEQ ID NO: 112 - protein, 119 aa, synthetic construct
  REGION 1..119: note = Description of Artificial Sequence: Synthetic polypeptide
EVQLLESGGGLVQPGGSLRLSCAASGFTFSSYAMSWVRQAPGKGLEWVSLIYSGGSRTSYPDSVKGRFTISRDNSNSTLFLQMNSLRVEDTAVYYCAKGGSSWLKMDYWGQGTLVIVSS

SEQ ID NO: 113 - protein, 8 aa, synthetic construct
  REGION 1..8: note = Description of Artificial Sequence: Synthetic peptide
GFTFSSYA

SEQ ID NO: 114 - protein, 8 aa, synthetic construct
  REGION 1..8: note = Description of Artificial Sequence: Synthetic peptide
IYSGGSRT

SEQ ID NO: 115 - protein, 12 aa, synthetic construct
  REGION 1..12: note = Description of Artificial Sequence: Synthetic peptide
AKGGSSWLKMDY

SEQ ID NO: 116 - protein, 111 aa, synthetic construct
  REGION 1..111: note = Description of Artificial Sequence: Synthetic polypeptide
QSVLTQPPSVSGAPGQRVTISCTGSSSNIGAGYDVQWYQHLPGTAPKLLIFANTNRPSGVPDRFSGSKSGTSASLAITGLQAEDEADYYCQSYDSSLSGSVFGGGTKLTVL

SEQ ID NO: 117 - protein, 9 aa, synthetic construct
  REGION 1..9: note = Description of Artificial Sequence: Synthetic peptide
SSNIGAGYD

SEQ ID NO: 118 - skipped

SEQ ID NO: 119 - protein, 11 aa, synthetic construct
  REGION 1..11: note = Description of Artificial Sequence: Synthetic peptide
QSYDSSLSGSV

SEQ ID NO: 120 - protein, 122 aa, synthetic construct
  REGION 1..122: note = Description of Artificial Sequence: Synthetic polypeptide
QVQLVQSGAEVKKPGASVRVSCSSSGYTFTGYYIHWVRQAPGQGLEYMGRINPHSGGTNYAQKFQGRVTMTRDTSTSTVYMELSSLRSDDTAVYYCAKEGTTAHIFNWFDPWGQGTLVTVSS

SEQ ID NO: 121 - protein, 8 aa, synthetic construct
  REGION 1..8: note = Description of Artificial Sequence: Synthetic peptide
GYTFTGYY

SEQ ID NO: 122 - protein, 8 aa, synthetic construct
  REGION 1..8: note = Description of Artificial Sequence: Synthetic peptide
INPHSGGT

SEQ ID NO: 123 - protein, 15 aa, synthetic construct
  REGION 1..15: note = Description of Artificial Sequence: Synthetic peptide
AKEGTTAHIFNWFDP

SEQ ID NO: 124 - protein, 107 aa, synthetic construct
  REGION 1..107: note = Description of Artificial Sequence: Synthetic polypeptide
DIQMTQSPSSLSASVGDRVTITCRASQSINSYLNWYQQKPGKAPNLLIYTASSLQSGVPSRFSGSGSGTDFTLTISSLQPEDFATYYCQQSYTSLFTFGQGTKLEIK

SEQ ID NO: 125 - protein, 6 aa, synthetic construct
  REGION 1..6: note = Description of Artificial Sequence: Synthetic peptide
QSINSY

SEQ ID NO: 126 - skipped

SEQ ID NO: 127 - protein, 9 aa, synthetic construct
  REGION 1..9: note = Description of Artificial Sequence: Synthetic peptide
QQSYTSLFT

SEQ ID NO: 128 - protein, 127 aa, synthetic construct
  REGION 1..127: note = Description of Artificial Sequence: Synthetic polypeptide
QLQLQESGPGLVKPSETLSLTCTVSGVSINSTSYYWGWMRQPPGKGLEWIGNIYYTGTTYYNPSLNRRVSISGDTSKNQFSLSLTSVTAADTAVYYCAGPRRVTVFGILLMESFDVWSQGTMVTVSS

SEQ ID NO: 129 - protein, 10 aa, synthetic construct
  REGION 1..10: note = Description of Artificial Sequence: Synthetic peptide
GVSINSTSYY

SEQ ID NO: 130 - protein, 7 aa, synthetic construct
  REGION 1..7: note = Description of Artificial Sequence: Synthetic peptide
IYYTGTT

SEQ ID NO: 131 - protein, 19 aa, synthetic construct
  REGION 1..19: note = Description of Artificial Sequence: Synthetic peptide
AGPRRVTVFGILLMESFDV

SEQ ID NO: 132 - protein, 107 aa, synthetic construct
  REGION 1..107: note = Description of Artificial Sequence: Synthetic polypeptide
AIQMTQSPSSLSASVGDRVTITCRASQAIRDDLGWFQQKPGKAPKLLIYTASTLQSGVPSRFSGGGSGTEFILTISSLQPEDIGTYYCLQDYGYPWTFGQGTKVEIK

SEQ ID NO: 133 - protein, 6 aa, synthetic construct
  REGION 1..6: note = Description of Artificial Sequence: Synthetic peptide
QAIRDD

SEQ ID NO: 134 - protein, 9 aa, synthetic construct
  REGION 1..9: note = Description of Artificial Sequence: Synthetic peptide
LQDYGYPWT

SEQ ID NO: 135 - protein, 122 aa, synthetic construct
  REGION 1..122: note = Description of Artificial Sequence: Synthetic polypeptide
EVQLLESGGGLVQPGGSLRLSCAASGFTFSNYALSWVRQAPGKGLEWVSAISGRDASTYYADSVKGRFTISRDNSKNTLYLQMNSLRAEDTAVYYCTLFDYDSSGYFDFDYWGQGTLVTVSS

SEQ ID NO: 136 - protein, 8 aa, synthetic construct
  REGION 1..8: note = Description of Artificial Sequence: Synthetic peptide
GFTFSNYA

SEQ ID NO: 137 - protein, 8 aa, synthetic construct
  REGION 1..8: note = Description of Artificial Sequence: Synthetic peptide
ISGRDAST

SEQ ID NO: 138 - protein, 15 aa, synthetic construct
  REGION 1..15: note = Description of Artificial Sequence: Synthetic peptide
TLFDYDSSGYFDFDY

SEQ ID NO: 139 - protein, 110 aa, synthetic construct
  REGION 1..110: note = Description of Artificial Sequence: Synthetic polypeptide
QSALTQPRSVSGSPGQSVTISCTGTGSDVGGYNYVSWYQHHPGKAPKLIIFDVTKRPSGVPDRFSGSKSGYTASLTISGLQAEDEAVYYCCSYANSYTGVFGTGTKVTVL

SEQ ID NO: 140 - protein, 9 aa, synthetic construct
  REGION 1..9: note = Description of Artificial Sequence: Synthetic peptide
GSDVGGYNY

SEQ ID NO: 141 - skipped

SEQ ID NO: 142 - protein, 10 aa, synthetic construct
  REGION 1..10: note = Description of Artificial Sequence: Synthetic peptide
CSYANSYTGV

SEQ ID NO: 143 - protein, 119 aa, synthetic construct
  REGION 1..119: note = Description of Artificial Sequence: Synthetic polypeptide
QVQLVESGGGVVQPGGSLRLSCAASGFTFSSHVMHWVRQAPGKGLEWVALISLDGDDKYYADSVNGRVAISRDNSKNTLYLQVNSLRSDDTCVYYCARGGRWDYALDVWGQGTTVTVSS

SEQ ID NO: 144 - protein, 8 aa, synthetic construct
  REGION 1..8: note = Description of Artificial Sequence: Synthetic peptide
GFTFSSHV

SEQ ID NO: 145 - protein, 8 aa, synthetic construct
  REGION 1..8: note = Description of Artificial Sequence: Synthetic peptide
ISLDGDDK

SEQ ID NO: 146 - protein, 12 aa, synthetic construct
  REGION 1..12: note = Description of Artificial Sequence: Synthetic peptide
ARGGRWDYALDV

SEQ ID NO: 147 - protein, 109 aa, synthetic construct
  REGION 1..109: note = Description of Artificial Sequence: Synthetic polypeptide
EIVMTQSPATLSVSPGERATLSCRVSQSISNSLAWYQQKPGQVPRLLIYAASTRATGIPARFSGSGSGTEFTLTISSLQSEDFAVYYCQQYNNWPRALTFGGGTKVEIK

SEQ ID NO: 148 - protein, 6 aa, synthetic construct
  REGION 1..6: note = Description of Artificial Sequence: Synthetic peptide
QSISNS

SEQ ID NO: 149 - skipped

SEQ ID NO: 150 - protein, 11 aa, synthetic construct
  REGION 1..11: note = Description of Artificial Sequence: Synthetic peptide
QQYNNWPRALT

SEQ ID NO: 151 - protein, 123 aa, synthetic construct
  REGION 1..123: note = Description of Artificial Sequence: Synthetic polypeptide
QVQLVESGGGVVQPGRSLRLSCAASGFTFNDYAMHWVRQAPGKGPEWVAVISYDGTNEYYMGSVKGRFTISRDNSKNMVNLQMNSLRPEDTAVYYCARDLAAWSRELLVFDQWGQGTLVTVSS

SEQ ID NO: 152 - protein, 8 aa, synthetic construct
  REGION 1..8: note = Description of Artificial Sequence: Synthetic peptide
GFTFNDYA

SEQ ID NO: 153 - protein, 8 aa, synthetic construct
  REGION 1..8: note = Description of Artificial Sequence: Synthetic peptide
ISYDGTNE

SEQ ID NO: 154 - protein, 16 aa, synthetic construct
  REGION 1..16: note = Description of Artificial Sequence: Synthetic peptide
ARDLAAWSRELLVFDQ

SEQ ID NO: 155 - protein, 108 aa, synthetic construct
  REGION 1..108: note = Description of Artificial Sequence: Synthetic polypeptide
ENVLTQSPGTLSLSPGEGATLSCRASQSVPNTYLAWYQQKPGQAPRLLIYGASSRAAGIPDRFSGSGSGTDFTLTISRLEPEDFAVYYCQQYGRSPGTFGQGTKVEIK

SEQ ID NO: 156 - protein, 7 aa, synthetic construct
  REGION 1..7: note = Description of Artificial Sequence: Synthetic peptide
QSVPNTY

SEQ ID NO: 157 - protein, 9 aa, synthetic construct
  REGION 1..9: note = Description of Artificial Sequence: Synthetic peptide
QQYGRSPGT

SEQ ID NO: 158 - protein, 133 aa, synthetic construct
  REGION 1..133: note = Description of Artificial Sequence: Synthetic polypeptide
QAQVVESGGGVVQPGTSLRLSCEPSGFTLSDYGIHWVRQPPGKGLEWVAVIWHDGDRINYADSVKGRFTISRDESDKKVHLQMESLRTEDTAVYYCARGTLPRNCRGMRCYGEFDHYYYLDVWGTGTTVTVSS

SEQ ID NO: 159 - protein, 8 aa, synthetic construct
  REGION 1..8: note = Description of Artificial Sequence: Synthetic peptide
GFTLSDYG

SEQ ID NO: 160 - protein, 8 aa, synthetic construct
  REGION 1..8: note = Description of Artificial Sequence: Synthetic peptide
IWHDGDRI

SEQ ID NO: 161 - protein, 26 aa, synthetic construct
  REGION 1..26: note = Description of Artificial Sequence: Synthetic peptide
ARGTLPRNCRGMRCYGEFDHYYYLDV

SEQ ID NO: 162 - protein, 111 aa, synthetic construct
  REGION 1..111: note = Description of Artificial Sequence: Synthetic polypeptide
QSVLTQPPSVSGAPGQRVTISCTGHSSNIGANSDVHWYQQLPLRAPKLLIFGTINRASGVPDRFSGSRSGTSASLVISGLQPDDEADYYCQSYDRGLSAYVFGSGTRVDVL

SEQ ID NO: 163 - protein, 9 aa, synthetic construct
  REGION 1..9: note = Description of Artificial Sequence: Synthetic peptide
SSNIGANSD

SEQ ID NO: 164 - skipped

SEQ ID NO: 165 - protein, 11 aa, synthetic construct
  REGION 1..11: note = Description of Artificial Sequence: Synthetic peptide
QSYDRGLSAYV

SEQ ID NO: 166 - protein, 131 aa, synthetic construct
  REGION 1..131: note = Description of Artificial Sequence: Synthetic polypeptide
EVQLVESGGGLVQPGGSLRLSCAASGFTFSSYWMSWVRQAPGKGLEWVANIKQDGSEKDYVDSVKGRFTISRDNAKNSLYLQLNSLRAEDTAVYYCARERSTQSSSWYVSSYYSYYGMDVWGQGTTVTVSS

SEQ ID NO: 167 - protein, 8 aa, synthetic construct
  REGION 1..8: note = Description of Artificial Sequence: Synthetic peptide
GFTFSSYW

SEQ ID NO: 168 - protein, 8 aa, synthetic construct
  REGION 1..8: note = Description of Artificial Sequence: Synthetic peptide
IKQDGSEK

SEQ ID NO: 169 - protein, 24 aa, synthetic construct
  REGION 1..24: note = Description of Artificial Sequence: Synthetic peptide
ARERSTQSSSWYVSSYYSYYGMDV

SEQ ID NO: 170 - protein, 109 aa, synthetic construct
  REGION 1..109: note = Description of Artificial Sequence: Synthetic polypeptide
SSELTQDPAVSVALGQTVTITCQGDSLRSFYASWYQQKPGQAPVFVIYGKYNRPSGIPDRFSGSSSGNTASLTITGAQAEDEADYYCNSRDSSDNHLGVFGGGTKLTVL

SEQ ID NO: 171 - protein, 6 aa, synthetic construct
  REGION 1..6: note = Description of Artificial Sequence: Synthetic peptide
SLRSFY

SEQ ID NO: 172 - skipped

SEQ ID NO: 173 - protein, 12 aa, synthetic construct
  REGION 1..12: note = Description of Artificial Sequence: Synthetic peptide
NSRDSSDNHLGV

SEQ ID NO: 174 - protein, 115 aa, synthetic construct
  REGION 1..115: note = Description of Artificial Sequence: Synthetic polypeptide
QVQLVQSGSELRKPGASVKLSCRTSGYTFIHFAMNWLRQAPGQGLEWLGWINTHSGNPTYAQGFTGRFVFSLDVSAGTAYLEISGLKAEDTAVYYCARERYFDFWGQGALVAVSS

SEQ ID NO: 175 - protein, 8 aa, synthetic construct
  REGION 1..8: note = Description of Artificial Sequence: Synthetic peptide
GYTFIHFA

SEQ ID NO: 176 - protein, 8 aa, synthetic construct
  REGION 1..8: note = Description of Artificial Sequence: Synthetic peptide
INTHSGNP

SEQ ID NO: 177 - protein, 8 aa, synthetic construct
  REGION 1..8: note = Description of Artificial Sequence: Synthetic peptide
ARERYFDF

SEQ ID NO: 178 - protein, 110 aa, synthetic construct
  REGION 1..110: note = Description of Artificial Sequence: Synthetic polypeptide
QSVLTQPPSASGTPGQRVTISCSGTSSNIGKNFLYWYQQVPGTAPKLLIYSSNQRPSGVPDRFSGSKSGTSASLAISGLRSEDEADYYCAAWDDSLSGWVFGGGTKVTVL

SEQ ID NO: 179 - protein, 8 aa, synthetic construct
  REGION 1..8: note = Description of Artificial Sequence: Synthetic peptide
SSNIGKNF

SEQ ID NO: 180 - skipped

SEQ ID NO: 181 - protein, 11 aa, synthetic construct
  REGION 1..11: note = Description of Artificial Sequence: Synthetic peptide
AAWDDSLSGWV

SEQ ID NO: 182 - protein, 123 aa, synthetic construct
  REGION 1..123: note = Description of Artificial Sequence: Synthetic polypeptide
EVQLVESGGDLVQPGGSLRLSCAASGFTFSNYWMNWVRQPPGKGLVWVSRISGDGTGTSYADSVRGRFTISRDNAKSTLYLQVNSLSAEDTAVYYCTRDGGRDHPTPDAFDIWGQGTMVTVSS

SEQ ID NO: 183 - protein, 8 aa, synthetic construct
  REGION 1..8: note = Description of Artificial Sequence: Synthetic peptide
GFTFSNYW

SEQ ID NO: 184 - protein, 8 aa, synthetic construct
  REGION 1..8: note = Description of Artificial Sequence: Synthetic peptide
ISGDGTGT

SEQ ID NO: 185 - protein, 16 aa, synthetic construct
  REGION 1..16: note = Description of Artificial Sequence: Synthetic peptide
TRDGGRDHPTPDAFDI

SEQ ID NO: 186 - protein, 112 aa, synthetic construct
  REGION 1..112: note = Description of Artificial Sequence: Synthetic polypeptide
DVVMAQSPLSLPVTLGQPASISCRSSQSLVHSDGNTYLNWFQQRPGQSPRRLIYKISNRDSGVPDRFSGSGSGTDFTLKISRVEAEDVGVYYCMQGTHWPRTFGQGTKLEIK

SEQ ID NO: 187 - protein, 11 aa, synthetic construct
  REGION 1..11: note = Description of Artificial Sequence: Synthetic peptide
QSLVHSDGNTY

SEQ ID NO: 188 - skipped

SEQ ID NO: 189 - protein, 9 aa, synthetic construct
  REGION 1..9: note = Description of Artificial Sequence: Synthetic peptide
MQGTHWPRT

SEQ ID NO: 190 - protein, 123 aa, synthetic construct
  REGION 1..123: note = Description of Artificial Sequence: Synthetic polypeptide
QVQLQQWGAGLLKPSETLSLTCVVSGGSFSTHYWNWIRQSPGKGLEWIGEINHSGNTNYNPSLTGRATISVATSKTQFSLRLNSVTAADTAVYFCARGPRLRYTAGRPLFDTWGQGTLVTVSS

SEQ ID NO: 191 - protein, 8 aa, synthetic construct
  REGION 1..8: note = Description of Artificial Sequence: Synthetic peptide
GGSFSTHY

SEQ ID NO: 192 - protein, 7 aa, synthetic construct
  REGION 1..7: note = Description of Artificial Sequence: Synthetic peptide
INHSGNT

SEQ ID NO: 193 - protein, 17 aa, synthetic construct
  REGION 1..17: note = Description of Artificial Sequence: Synthetic peptide
ARGPRLRYTAGRPLFDT

SEQ ID NO: 194 - protein, 107 aa, synthetic construct
  REGION 1..107: note = Description of Artificial Sequence: Synthetic polypeptide
DIQMTQSPSTLSASVGDRVTITCRASQSISAFLAWYQQKPGKAPNLVIYKASSLDSGVPSTFSGSGSGTEYTLTISSLQPDDFATYYCQQYFSSPPTFGQGTKVEMK

SEQ ID NO: 195 - protein, 6 aa, synthetic construct
  REGION 1..6: note = Description of Artificial Sequence: Synthetic peptide
QSISAF

SEQ ID NO: 196 - skipped

SEQ ID NO: 197 - protein, 9 aa, synthetic construct
  REGION 1..9: note = Description of Artificial Sequence: Synthetic peptide
QQYFSSPPT

SEQ ID NO: 198 - protein, 126 aa, synthetic construct
  REGION 1..126: note = Description of Artificial Sequence: Synthetic polypeptide
QVQLQESGPGLVKPSQTLSLTCAVSGGSISSGGYYWSWIRQLPGKGLEWIGYIYYSGSTSYNPSLKSRVTISVDTSKNQLSLNLSSVTAADTAVYNCARGRRISISGVVTPLFDYWGQGTLVTVSS

SEQ ID NO: 199 - protein, 10 aa, synthetic construct
  REGION 1..10: note = Description of Artificial Sequence: Synthetic peptide
GGSISSGGYY

SEQ ID NO: 200 - protein, 7 aa, synthetic construct
  REGION 1..7: note = Description of Artificial Sequence: Synthetic peptide
IYYSGST

SEQ ID NO: 201 - protein, 18 aa, synthetic construct
  REGION 1..18: note = Description of Artificial Sequence: Synthetic peptide
ARGRRISISGVVTPLFDY

SEQ ID NO: 202 - protein, 107 aa, synthetic construct
  REGION 1..107: note = Description of Artificial Sequence: Synthetic polypeptide
DIQMTQSPSSVSASVGDRVTITCRASQGISSWLAWYQLKPGKAPKLLIYAASSLQSGVPSRFSGSGSGTDFTLTISSLQPEDFATYYCQQANSVPLTFGGGTKVEIK

SEQ ID NO: 203 - protein, 6 aa, synthetic construct
  REGION 1..6: note = Description of Artificial Sequence: Synthetic peptide
QGISSW

SEQ ID NO: 204 - protein, 9 aa, synthetic construct
  REGION 1..9: note = Description of Artificial Sequence: Synthetic peptide
QQANSVPLT

SEQ ID NO: 205 - protein, 120 aa, synthetic construct
  REGION 1..120: note = Description of Artificial Sequence: Synthetic polypeptide
EVQLVESGGGLVKPGGSLRLSCAASGFTFSHYYLNWVRQAPGKGLEWVACISDRSENVYYADSVKGRFTISRDNAKNSLFLQMNNLRAEDTAIYYCARDMRELRPSADYWGQGTLVTVSS

SEQ ID NO: 206 - protein, 8 aa, synthetic construct
  REGION 1..8: note = Description of Artificial Sequence: Synthetic peptide
GFTFSHYY

SEQ ID NO: 207 - protein, 8 aa, synthetic construct
  REGION 1..8: note = Description of Artificial Sequence: Synthetic peptide
ISDRSENV

SEQ ID NO: 208 - protein, 13 aa, synthetic construct
  REGION 1..13: note = Description of Artificial Sequence: Synthetic peptide
ARDMRELRPSADY

SEQ ID NO: 209 - protein, 109 aa, synthetic construct
  REGION 1..109: note = Description of Artificial Sequence: Synthetic polypeptide
EIVLTQSPGTLSLSPGDRATLSCRASQSVDGNSLAWYQQKPGQAPRLLISGASTRATGIPDRFSGSGSGTDFTLTISRLEPEDFVLYHCQLYTVSPRYTFGQGTKLEIK

SEQ ID NO: 210 - protein, 7 aa, synthetic construct
  REGION 1..7: note = Description of Artificial Sequence: Synthetic peptide
QSVDGNS

SEQ ID NO: 211 - protein, 10 aa, synthetic construct
  REGION 1..10: note = Description of Artificial Sequence: Synthetic peptide
QLYTVSPRYT

SEQ ID NO: 212 - protein, 122 aa, synthetic construct
  REGION 1..122: note = Description of Artificial Sequence: Synthetic polypeptide
QLQLQESGPGLVKPSETLSLTCTVSGGSISSDNYYWGWIRQPPGKGPLWIGTIFYNGDTYYNPSLKSQLNISVDPSKNQFSLKLTSVTAADTAIYYCTRHDSYSRGWYVTHWGQGTLVTVSS

SEQ ID NO: 213 - protein, 10 aa, synthetic construct
  REGION 1..10: note = Description of Artificial Sequence: Synthetic peptide
GGSISSDNYY

SEQ ID NO: 214 - protein, 7 aa, synthetic construct
  REGION 1..7: note = Description of Artificial Sequence: Synthetic peptide
IFYNGDT

SEQ ID NO: 215 - protein, 14 aa, synthetic construct
  REGION 1..14: note = Description of Artificial Sequence: Synthetic peptide
TRHDSYSRGWYVTH

SEQ ID NO: 216 - protein, 106 aa, synthetic construct
  REGION 1..106: note = Description of Artificial Sequence: Synthetic polypeptide
EIVLTQSPATLSLFPGERATLSCRASQSVTSYLAWYQQKPGQAPRLLIYDASKRATGIPARFSGSGSGTDFTLTISSLEPEDFATYYCQQRSARQLFGGGTKVEIK

SEQ ID NO: 217 - protein, 6 aa, synthetic construct
  REGION 1..6: note = Description of Artificial Sequence: Synthetic peptide
QSVTSY

SEQ ID NO: 218 - protein, 8 aa, synthetic construct
  REGION 1..8: note = Description of Artificial Sequence: Synthetic peptide
QQRSARQL

SEQ ID NO: 219 - protein, 123 aa, synthetic construct
  REGION 1..123: note = Description of Artificial Sequence: Synthetic polypeptide
EVQLVESGGGLVKPGGSLRLSCVASGLTFRNAWMTWVRQAPGKGLEWVGRIKSNVNGGTTDYAAPVRGRFTISRDDSRDTLYLQMNSLETEDTAMYYCTKDPPYTGGGYCQHWGLGTLVTVSS

SEQ ID NO: 220 - protein, 8 aa, synthetic construct
  REGION 1..8: note = Description of Artificial Sequence: Synthetic peptide
GLTFRNAW

SEQ ID NO: 221 - protein, 10 aa, synthetic construct
  REGION 1..10: note = Description of Artificial Sequence: Synthetic peptide
IKSNVNGGTT

SEQ ID NO: 222 - protein, 14 aa, synthetic construct
  REGION 1..14: note = Description of Artificial Sequence: Synthetic peptide
TKDPPYTGGGYCQH

SEQ ID NO: 223 - protein, 107 aa, synthetic construct
  REGION 1..107: note = Description of Artificial Sequence: Synthetic polypeptide
EIVLTQSPATLSLSPGESATLSCRASQSVSSCLAWYQQKPGQAPRLLIYDASTRAPGIPGRFSGSGSGTDFTLAISSLEPEDFAVYYCQQCSNWPLTFGRGTRLEIK

SEQ ID NO: 224 - protein, 6 aa, synthetic construct
  REGION 1..6: note = Description of Artificial Sequence: Synthetic peptide
QSVSSC

SEQ ID NO: 225 - protein, 9 aa, synthetic construct
  REGION 1..9: note = Description of Artificial Sequence: Synthetic peptide
QQCSNWPLT

SEQ ID NO: 226 - protein, 119 aa, synthetic construct
  REGION 1..119: note = Description of Artificial Sequence: Synthetic polypeptide
QVQLQESGPGLVKPSQTLSLTCTVSGGSINTGAYYWSWIRQHPGKGLEWIGYIYYSGSTYYNPSLKSRVTISKDTSKNQFSLRLTSVTAADTAVYYCVREKLTGAPDNWGQGTLVAVSS

SEQ ID NO: 227 - protein, 10 aa, synthetic construct
  REGION 1..10: note = Description of Artificial Sequence: Synthetic peptide
GGSINTGAYY

SEQ ID NO: 228 - protein, 11 aa, synthetic construct
  REGION 1..11: note = Description of Artificial Sequence: Synthetic peptide
VREKLTGAPDN

SEQ ID NO: 229 - protein, 107 aa, synthetic construct
  REGION 1..107: note = Description of Artificial Sequence: Synthetic polypeptide
DIQMTQSPSSLSASVGDRVTITCRASQGVSNYLAWFHQKPGKAPKSLIYAASTLHDGVPSSFSGSGSGTEFTLTISDLQPEHFGTYYCEQYNSYPFTFGPGTTVDFK

SEQ ID NO: 230 - protein, 6 aa, synthetic construct
  REGION 1..6: note = Description of Artificial Sequence: Synthetic peptide
QGVSNY

SEQ ID NO: 231 - protein, 9 aa, synthetic construct
  REGION 1..9: note = Description of Artificial Sequence: Synthetic peptide
EQYNSYPFT

SEQ ID NO: 232 - protein, 123 aa, synthetic construct
  REGION 1..123: note = Description of Artificial Sequence: Synthetic polypeptide
QVQLVQSGAEVKKPGSSVKVSCKASGGPLSSYNFIWVRQAPGQGLEWMGGILPVFDTTNYAQKFQGRVTITADKATSTSYMELSSLTSEDTAVYYCARAVGGTHYYYYGLDVWGQGTTVAVSS

SEQ ID NO: 233 - protein, 8 aa, synthetic construct
  REGION 1..8: note = Description of Artificial Sequence: Synthetic peptide
GGPLSSYN

SEQ ID NO: 234 - protein, 8 aa, synthetic construct
  REGION 1..8: note = Description of Artificial Sequence: Synthetic peptide
ILPVFDTT

SEQ ID NO: 235 - protein, 16 aa, synthetic construct
  REGION 1..16: note = Description of Artificial Sequence: Synthetic peptide
ARAVGGTHYYYYGLDV

SEQ ID NO: 236 - protein, 112 aa, synthetic construct
  REGION 1..112: note = Description of Artificial Sequence: Synthetic polypeptide
EIVMTQSPLSLPVTPGEPASISCRSSQSLLHGNGYNYVDWYLQRPGQPPQLLIYLGSRRASGVPDRFSGSGSGTDFTLKISRVEADDLGVYYCMQALQTRVTFGPGTKVDIK

SEQ ID NO: 237 - protein, 11 aa, synthetic construct
  REGION 1..11: note = Description of Artificial Sequence: Synthetic peptide
QSLLHGNGYNY

SEQ ID NO: 238 - protein, 9 aa, synthetic construct
  REGION 1..9: note = Description of Artificial Sequence: Synthetic peptide
MQALQTRVT

SEQ ID NO: 239 - protein, 114 aa, synthetic construct
  REGION 1..114: note = Description of Artificial Sequence: Synthetic polypeptide
EVQLVQSGAEVKKPGESLKISCKGSGYSFMSYWIGWVRQKPGKGLEWMGIIFPGDSDTRYSPSFQGHVTISADKSITTAYLQWNSLEASDTAIYYCATLDGDYWGRGTLVTVSS

SEQ ID NO: 240 - protein, 8 aa, synthetic construct
  REGION 1..8: note = Description of Artificial Sequence: Synthetic peptide
GYSFMSYW

SEQ ID NO: 241 - protein, 8 aa, synthetic construct
  REGION 1..8: note = Description of Artificial Sequence: Synthetic peptide
IFPGDSDT

SEQ ID NO: 242 - protein, 7 aa, synthetic construct
  REGION 1..7: note = Description of Artificial Sequence: Synthetic peptide
ATLDGDY

SEQ ID NO: 243 - protein, 113 aa, synthetic construct
  REGION 1..113: note = Description of Artificial Sequence: Synthetic polypeptide
DIVMTQSPDSLAVSLGERATINCRSSQSVLSSSSNKNYLGWYQQKPGQPPKLLIHWASTRAAGVPDRFSGSGTGTDFTLNISSLQAEDVAVYYCQQYHTTLPTFGQGTKLEIK

SEQ ID NO: 244 - protein, 12 aa, synthetic construct
  REGION 1..12: note = Description of Artificial Sequence: Synthetic peptide
QSVLSSSSNKNY

SEQ ID NO: 245 - protein, 9 aa, synthetic construct
  REGION 1..9: note = Description of Artificial Sequence: Synthetic peptide
QQYHTTLPT

SEQ ID NO: 246 - protein, 119 aa, synthetic construct
  REGION 1..119: note = Description of Artificial Sequence: Synthetic polypeptide
EVQLLESGGGLVQPGGSLRLSCAASGFTFRDSAMTWVRQAPGKGLEWVSTISGNGDTTYYADSVKGRFSIFRDNSRNTLYVQMNSLRAEDTAVYYCARYGDHKGWFDSWGQGTLVTVSS

SEQ ID NO: 247 - protein, 8 aa, synthetic construct
  REGION 1..8: note = Description of Artificial Sequence: Synthetic peptide
GFTFRDSA

SEQ ID NO: 248 - protein, 8 aa, synthetic construct
  REGION 1..8: note = Description of Artificial Sequence: Synthetic peptide
ISGNGDTT

SEQ ID NO: 249 - protein, 12 aa, synthetic construct
  REGION 1..12: note = Description of Artificial Sequence: Synthetic peptide
ARYGDHKGWFDS

SEQ ID NO: 250 - protein, 107 aa, synthetic construct
  REGION 1..107: note = Description of Artificial Sequence: Synthetic polypeptide
ELVMTQSPASLSVSPGEGATVSCRASQSVGSNLAWYQQKPGQGPRLLIYGASTRATGVPARFSGSGSGTEFTLTISSLQSEDFAVYYCQQYNNWPRTFGQGTKVEIK

SEQ ID NO: 251 - protein, 6 aa, synthetic construct
  REGION 1..6: note = Description of Artificial Sequence: Synthetic peptide
QSVGSN

SEQ ID NO: 252 - protein, 9 aa, synthetic construct
  REGION 1..9: note = Description of Artificial Sequence: Synthetic peptide
QQYNNWPRT

SEQ ID NO: 253 - protein, 122 aa, synthetic construct
  REGION 1..122: note = Description of Artificial Sequence: Synthetic polypeptide
QVQLVQSGAEVKKPGASVKVSCKASGYTFTSYYMHWVRQAPGQGLEWMGIINPSDGSKTYAQKFQGRVTLTRDTSTSTVYMELSSLRSEDTAVYYCARGNGYSSSWYVNDYWGQGTLVTVSS

SEQ ID NO: 254 - protein, 8 aa, synthetic construct
  REGION 1..8: note = Description of Artificial Sequence: Synthetic peptide
INPSDGSK

SEQ ID NO: 255 - protein, 15 aa, synthetic construct
  REGION 1..15: note = Description of Artificial Sequence: Synthetic peptide
ARGNGYSSSWYVNDY

SEQ ID NO: 256 - protein, 108 aa, synthetic construct
  REGION 1..108: note = Description of Artificial Sequence: Synthetic polypeptide
DIVLTQSPGTLSLSPGERATLSCRASQSLTNSNFAWYQQIPGQAPRLLIYGASSRATGIPDRFSGSGSGTDFTLTISRLEPEDFVVYYCQQYGRSPITFGQGTRLEIK

SEQ ID NO: 257 - protein, 7 aa, synthetic construct
  REGION 1..7: note = Description of Artificial Sequence: Synthetic peptide
QSLTNSN

SEQ ID NO: 258 - protein, 9 aa, synthetic construct
  REGION 1..9: note = Description of Artificial Sequence: Synthetic peptide
QQYGRSPIT

SEQ ID NO: 259 - protein, 123 aa, synthetic construct
  REGION 1..123: note = Description of Artificial Sequence: Synthetic polypeptide
QVQLVESGGGVVQPGRSLRLSCAASGFTFTKYGMHWVRQAPGKGLEWVALISYDGNNKYYADSVRGRVTISRDNSKNTLYLQMDSLRAEDTAVYYCARGQDYPFWSGSTFEYWGQGTLVTVSS

SEQ ID NO: 260 - protein, 8 aa, synthetic construct
  REGION 1..8: note = Description of Artificial Sequence: Synthetic peptide
GFTFTKYG

SEQ ID NO: 261 - protein, 8 aa, synthetic construct
  REGION 1..8: note = Description of Artificial Sequence: Synthetic peptide
ISYDGNNK

SEQ ID NO: 262 - protein, 16 aa, synthetic construct
  REGION 1..16: note = Description of Artificial Sequence: Synthetic peptide
ARGQDYPFWSGSTFEY

SEQ ID NO: 263 - protein, 111 aa, synthetic construct
  REGION 1..111: note = Description of Artificial Sequence: Synthetic polypeptide
QAVVTQEPSLTVSPGGTVTLTCGSTTGAVTGGHFPYWIQQKPGQAPRTLIYDATNRHSWTPARFSGSLLGGKAALTLSGAQPEDEADYYCLLSYSSATFLIFGGGTKLTVL

SEQ ID NO: 264 - protein, 9 aa, synthetic construct
  REGION 1..9: note = Description of Artificial Sequence: Synthetic peptide
TGAVTGGHF

SEQ ID NO: 265 - skipped

SEQ ID NO: 266 - protein, 11 aa, synthetic construct
  REGION 1..11: note = Description of Artificial Sequence: Synthetic peptide
LLSYSSATFLI

SEQ ID NO: 267 - protein, 123 aa, synthetic construct
  REGION 1..123: note = Description of Artificial Sequence: Synthetic polypeptide
QVQLQESGPGLVKPSETLSLTCTVSGDSLSSGSYFWSWIRQPPGKGLEWIGYISFRGDTNYNPSLKSRVIISLDKSKNQFSLRLSSMTPADTAVYYCARSPWIQSWSYYFDYWGQGTLVTVSS

SEQ ID NO: 268 - protein, 10 aa, synthetic construct
  REGION 1..10: note = Description of Artificial Sequence: Synthetic peptide
GDSLSSGSYF

SEQ ID NO: 269 - protein, 7 aa, synthetic construct
  REGION 1..7: note = Description of Artificial Sequence: Synthetic peptide
ISFRGDT

SEQ ID NO: 270 - protein, 15 aa, synthetic construct
  REGION 1..15: note = Description of Artificial Sequence: Synthetic peptide
ARSPWIQSWSYYFDY

SEQ ID NO: 271 - protein, 107 aa, synthetic construct
  REGION 1..107: note = Description of Artificial Sequence: Synthetic polypeptide
DIQMTQSPSTVSASVGDRVTITCRASQRISSWLAWYQQKPGKAPKLLIYKASSLEGGVPSRFSGSGSGTEFTLTISSLQPDDFAIYYCQQYNGYPWTFGQGTKVEIK

SEQ ID NO: 272 - protein, 6 aa, synthetic construct
  REGION 1..6: note = Description of Artificial Sequence: Synthetic peptide
QRISSW

SEQ ID NO: 273 - protein, 9 aa, synthetic construct
  REGION 1..9: note = Description of Artificial Sequence: Synthetic peptide
QQYNGYPWT

SEQ ID NO: 274 - protein, 122 aa, synthetic construct
  REGION 1..122: note = Description of Artificial Sequence: Synthetic polypeptide
QVQLQESGPGLVKPSGTLSLTCAVSGGSISTDNWWSWVRQPPNKGLEWIGAIFQSGSTIYNPSLMSRVTISLDRSNNRFSLQLISVTAADTALYYCARASFHYGSGNYFEYLGQGTLVTVSS

SEQ ID NO: 275 - protein, 9 aa, synthetic construct
  REGION 1..9: note = Description of Artificial Sequence: Synthetic peptide
GGSISTDNW

SEQ ID NO: 276 - protein, 7 aa, synthetic construct
  REGION 1..7: note = Description of Artificial Sequence: Synthetic peptide
IFQSGST

SEQ ID NO: 277 - protein, 15 aa, synthetic construct
  REGION 1..15: note = Description of Artificial Sequence: Synthetic peptide
ARASFHYGSGNYFEY

SEQ ID NO: 278 - protein, 111 aa, synthetic construct
  REGION 1..111: note = Description of Artificial Sequence: Synthetic polypeptide
QSVLTQPPSVSAAPGQKVTISCSGSSSNVGTHHVSWYQQLPGTAPKLLIYENDKRPSGIPNRFSGSKSGTSATLAIIGLQTGDEADYYCGSWDSSLSAFWVFGGGTKLTVL

SEQ ID NO: 279 - protein, 8 aa, synthetic construct
  REGION 1..8: note = Description of Artificial Sequence: Synthetic peptide
SSNVGTHH

SEQ ID NO: 280 - skipped

SEQ ID NO: 281 - protein, 12 aa, synthetic construct
  REGION 1..12: note = Description of Artificial Sequence: Synthetic peptide
GSWDSSLSAFWV

SEQ ID NO: 282 - protein, 122 aa, synthetic construct
  REGION 1..122: note = Description of Artificial Sequence: Synthetic polypeptide
QVQLVQSGAEVKKPGASVKVACKASGYTFTRYAMHWVRQAPGQRLEWMGWINAGNGNTKDSQKFQGRVTITRDTSASTVYMELSSLRSEDTAVYYCARGVPWGLGSYNFDYWGQGTLVSISS

SEQ ID NO: 283 - protein, 8 aa, synthetic construct
  REGION 1..8: note = Description of Artificial Sequence: Synthetic peptide
GYTFTRYA

SEQ ID NO: 284 - protein, 8 aa, synthetic construct
  REGION 1..8: note = Description of Artificial Sequence: Synthetic peptide
INAGNGNT

SEQ ID NO: 285 - protein, 15 aa, synthetic construct
  REGION 1..15: note = Description of Artificial Sequence: Synthetic peptide
ARGVPWGLGSYNFDY

SEQ ID NO: 286 - protein, 109 aa, synthetic construct
  REGION 1..109: note = Description of Artificial Sequence: Synthetic polypeptide
QTVVTQEPSLTVSPGGTVTLSCASNTGAVTSGYYPYWFQQKPGQAPRTLIYETSNKHPWTPARFSGSLLGGKAALTLSGVQPEDEAEYCCLLYYGGTWVFGGGTKLTVL

SEQ ID NO: 287 - protein, 9 aa, synthetic construct
  REGION 1..9: note = Description of Artificial Sequence: Synthetic peptide
TGAVTSGYY

SEQ ID NO: 288 - skipped

SEQ ID NO: 289 - protein, 9 aa, synthetic construct
  REGION 1..9: note = Description of Artificial Sequence: Synthetic peptide
LLYYGGTWV

SEQ ID NO: 290 - protein, 122 aa, synthetic construct
  REGION 1..122: note = Description of Artificial Sequence: Synthetic polypeptide
EVQLLESGGGLVQPGGSLRLSCAASGFTFSSYALSWVRQAPGKGLEWVSAISGRDGNTYYADSVKGRFTISRDNSKNTLYLQMNSLRAEDTAVYYCTLYDYDSSGYFDFDYWGQGTLVTVSS

SEQ ID NO: 291 - protein, 8 aa, synthetic construct
  REGION 1..8: note = Description of Artificial Sequence: Synthetic peptide
ISGRDGNT

SEQ ID NO: 292 - protein, 15 aa, synthetic construct
  REGION 1..15: note = Description of Artificial Sequence: Synthetic peptide
TLYDYDSSGYFDFDY

SEQ ID NO: 293 - protein, 110 aa, synthetic construct
  REGION 1..110: note = Description of Artificial Sequence: Synthetic polypeptide
QSALTQPRSVSGSPGQSVTISCTGTSSDVGGFNYVSWYQQHPGKAPKLMIFDVTQRPSGVPDRFSGSKSGNTASLTISGLQAEDEADYHCCSYANYYTGVFGTGTRVTVL

SEQ ID NO: 294 - protein, 9 aa, synthetic construct
  REGION 1..9: note = Description of Artificial Sequence: Synthetic peptide
SSDVGGFNY

SEQ ID NO: 295 - protein, 10 aa, synthetic construct
  REGION 1..10: note = Description of Artificial Sequence: Synthetic peptide
CSYANYYTGV

SEQ ID NO: 296 - protein, 126 aa, synthetic construct
  REGION 1..126: note = Description of Artificial Sequence: Synthetic polypeptide
EVQLVESGGGLVQPGGSLRLSCAASGFTFSNYWMHWVRQAPGKGLVWVSRINSDGSNIRFADSVKGRFTFSRDNANNTLYLQMNSLRAEDTAVYYCARASRTVYGDSPLSYGIDVWGQGTTVTVSS

SEQ ID NO: 297 - protein, 8 aa, synthetic construct
  REGION 1..8: note = Description of Artificial Sequence: Synthetic peptide
INSDGSNI

SEQ ID NO: 298 - protein, 19 aa, synthetic construct
  REGION 1..19: note = Description of Artificial Sequence: Synthetic peptide
ARASRTVYGDSPLSYGIDV

SEQ ID NO: 299 - protein, 107 aa, synthetic construct
  REGION 1..107: note = Description of Artificial Sequence: Synthetic polypeptide
SYALTQPPSVSVSPGQTASITCSGDKLGNKFACWYQQKPGRSPVLVIYQDSQRPTGIPERFSGSNSGNTATLTISGTQAMDEADYYCQAWDSNTHVLFGGGTKLTVL

SEQ ID NO: 300 - protein, 6 aa, synthetic construct
  REGION 1..6: note = Description of Artificial Sequence: Synthetic peptide
KLGNKF

SEQ ID NO: 301 - skipped

SEQ ID NO: 302 - protein, 10 aa, synthetic construct
  REGION 1..10: note = Description of Artificial Sequence: Synthetic peptide
QAWDSNTHVL

SEQ ID NO: 303 - protein, 130 aa, synthetic construct
  REGION 1..130: note = Description of Artificial Sequence: Synthetic polypeptide
QVQLVESGGGVVQPGGSLRLSCAASGFTFSGYGMHWVRQAPGKGLEWVAFFSFDGSNTDYVDSVKGRFTISGDNSKNTLYLQMNSLRAEDTAVYYCVRDILVLPAAVSVFSGYYYGMDVWGQGTTVTVSS

SEQ ID NO: 304 - protein, 8 aa, synthetic construct
  REGION 1..8: note = Description of Artificial Sequence: Synthetic peptide
GFTFSGYG

SEQ ID NO: 305 - protein, 8 aa, synthetic construct
  REGION 1..8: note = Description of Artificial Sequence: Synthetic peptide
FSFDGSNT

SEQ ID NO: 306 - protein, 23 aa, synthetic construct
  REGION 1..23: note = Description of Artificial Sequence: Synthetic peptide
VRDILVLPAAVSVFSGYYYGMDV

SEQ ID NO: 307 - protein, 107 aa, synthetic construct
  REGION 1..107: note = Description of Artificial Sequence: Synthetic polypeptide
EIVLTQSPATLSLSPGERATLSCRASQSVRSYLAWYQQKPGQAPRLLIYDASNRATGIPARFSGSGSGTDFTLTISSLEPEDFAVYYCQHRSNWPITFGQGTRLEIK

SEQ ID NO: 308 - protein, 6 aa, synthetic construct
  REGION 1..6: note = Description of Artificial Sequence: Synthetic peptide
QSVRSY

SEQ ID NO: 309 - protein, 9 aa, synthetic construct
  REGION 1..9: note = Description of Artificial Sequence: Synthetic peptide
QHRSNWPIT

SEQ ID NO: 310 - protein, 123 aa, synthetic construct
  REGION 1..123: note = Description of Artificial Sequence: Synthetic polypeptide
QVQLRVSGPGLVNPSETLSLTCIVSGDSLRDYYWSWIRQSPGKGLEWIGYVTESGGAHYNPSLESRVTISVDASKTQFSLNLKSVTAADTAVYYCARDAYSSTWYTVGWFDPWGPGSLVTVSS

SEQ ID NO: 311 - protein, 8 aa, synthetic construct
  REGION 1..8: note = Description of Artificial Sequence: Synthetic peptide
GDSLRDYY

SEQ ID NO: 312 - protein, 7 aa, synthetic construct
  REGION 1..7: note = Description of Artificial Sequence: Synthetic peptide
VTESGGA

SEQ ID NO: 313 - protein, 17 aa, synthetic construct
  REGION 1..17: note = Description of Artificial Sequence: Synthetic peptide
ARDAYSSTWYTVGWFDP

SEQ ID NO: 314 - protein, 106 aa, synthetic construct
  REGION 1..106: note = Description of Artificial Sequence: Synthetic polypeptide
EIVLTQSPATLSLSPGERATLSCRASQDVGVYLAWYQQKPGQAPRLIIYDASDRVSGVPARFTGSGSGTDFTLTITSLEPEDFAVYFCQQRTSGLTFGGGTTLEIK

SEQ ID NO: 315 - protein, 6 aa, synthetic construct
  REGION 1..6: note = Description of Artificial Sequence: Synthetic peptide
QDVGVY

SEQ ID NO: 316 - protein, 8 aa, synthetic construct
  REGION 1..8: note = Description of Artificial Sequence: Synthetic peptide
QQRTSGLT

SEQ ID NO: 317 - protein, 120 aa, synthetic construct
  REGION 1..120: note = Description of Artificial Sequence: Synthetic polypeptide
QVELVESGGGVVQPGRSLRLSCVASGFTFSDYGMHWVRQAPGKGLEWVAVIWFDGSSKYYADSVKGRFTISRDDSKNTVFMQMNNVRVEDTAVYYCAREQWLGTEYFQNWGQGTLVTVSS

SEQ ID NO: 318 - protein, 8 aa, synthetic construct
  REGION 1..8: note = Description of Artificial Sequence: Synthetic peptide
GFTFSDYG

SEQ ID NO: 319 - protein, 8 aa, synthetic construct
  REGION 1..8: note = Description of Artificial Sequence: Synthetic peptide
IWFDGSSK

SEQ ID NO: 320 - protein, 13 aa, synthetic construct
  REGION 1..13: note = Description of Artificial Sequence: Synthetic peptide
AREQWLGTEYFQN

SEQ ID NO: 321 - protein, 107 aa, synthetic construct
  REGION 1..107: note = Description of Artificial Sequence: Synthetic polypeptide
EIVMTQSPATLSLFPGERATLSCRASQSVAGNLAWYQQKPGQAPRLLIYEASTRATDIPARFSGSGSETEFTLTISSLQSEDFAVYYCQQYKKWLITFGQGTRLEIK

SEQ ID NO: 322 - protein, 6 aa, synthetic construct
  REGION 1..6: note = Description of Artificial Sequence: Synthetic peptide
QSVAGN

SEQ ID NO: 323 - skipped

SEQ ID NO: 324 - protein, 9 aa, synthetic construct
  REGION 1..9: note = Description of Artificial Sequence: Synthetic peptide
QQYKKWLIT

SEQ ID NO: 325 - protein, 122 aa, synthetic construct
  REGION 1..122: note = Description of Artificial Sequence: Synthetic polypeptide
QLQLQQWGAGLVKPSETLSLTCTVSGGSLSGHFWSWIRQSPEKGLEWIGEINHSGRKNYNPSLMIRVDISIDTSKNQFSMRMTSLTAADSAVYYCARVGRNIVDTDDAFDVWGRGTLVTVSS

SEQ ID NO: 326 - protein, 8 aa, synthetic construct
  REGION 1..8: note = Description of Artificial Sequence: Synthetic peptide
GGSLSGHF

SEQ ID NO: 327 - protein, 7 aa, synthetic construct
  REGION 1..7: note = Description of Artificial Sequence: Synthetic peptide
INHSGRK

SEQ ID NO: 328 - protein, 16 aa, synthetic construct
  REGION 1..16: note = Description of Artificial Sequence: Synthetic peptide
ARVGRNIVDTDDAFDV

SEQ ID NO: 329 - protein, 109 aa, synthetic construct
  REGION 1..109: note = Description of Artificial Sequence: Synthetic polypeptide
EIVLTQSPGTLSLSPGDTVTLSCRASQTIDSIYLAWYQQRPGQAPRLLIYGASTRATGTPDRFSGGGSGTDFTLTITRLEPEDFAVYFCQQYGTSPPITFGRGTRLEIK

SEQ ID NO: 330 - protein, 7 aa, synthetic construct
  REGION 1..7: note = Description of Artificial Sequence: Synthetic peptide
QTIDSIY

SEQ ID NO: 331 - protein, 10 aa, synthetic construct
  REGION 1..10: note = Description of Artificial Sequence: Synthetic peptide
QQYGTSPPIT

SEQ ID NO: 332 - protein, 122 aa, synthetic construct
  REGION 1..122: note = Description of Artificial Sequence: Synthetic polypeptide
QVQLLQSGAEVKKPGASVKVSCKASGYTFTSYNIHWVRQAPGQSFEWMGWIHVGNGETKYSQNFQDRVAITRDTSANTVYMELSPLRSEDTALYYCVRDHVTAIVVGLFDPWGQGTLVTVSS

SEQ ID NO: 333 - protein, 8 aa, synthetic construct
  REGION 1..8: note = Description of Artificial Sequence: Synthetic peptide
GYTFTSYN

SEQ ID NO: 334 - protein, 8 aa, synthetic construct
  REGION 1..8: note = Description of Artificial Sequence: Synthetic peptide
IHVGNGET

SEQ ID NO: 335 - protein, 15 aa, synthetic construct
  REGION 1..15: note = Description of Artificial Sequence: Synthetic peptide
VRDHVTAIVVGLFDP

SEQ ID NO: 336 - protein, 110 aa, synthetic construct
  REGION 1..110: note = Description of Artificial Sequence: Synthetic polypeptide
QSALTQPASVSGSPGQSITISCSGTSTDVGAYKYVSWYQHHPGRSPKVILYEVDNRPSGVSIRFSGSKSGNTASLTISGLRAEDEADYYCSSFTSSSTWVFGGGTKVTVL

SEQ ID NO: 337 - protein, 9 aa, synthetic construct
  REGION 1..9: note = Description of Artificial Sequence: Synthetic peptide
STDVGAYKY

SEQ ID NO: 338 - skipped

SEQ ID NO: 339 - protein, 10 aa, synthetic construct
  REGION 1..10: note = Description of Artificial Sequence: Synthetic peptide
SSFTSSSTWV

SEQ ID NO: 340 - protein, 123 aa, synthetic construct
  REGION 1..123: note = Description of Artificial Sequence: Synthetic polypeptide
QVQLVQSGAEVKKPGSSVKVSCKASGGTFSSYSISWVRQAPGQGLEWMGGIIPIFGSGSYAQKFQGRVTITADKSTSTAYMELSSLSSDDTAVYYCARGESPSNFVYYGMDVWGQGTTVTVSS

SEQ ID NO: 341 - protein, 8 aa, synthetic construct
  REGION 1..8: note = Description of Artificial Sequence: Synthetic peptide
GGTFSSYS

SEQ ID NO: 342 - protein, 8 aa, synthetic construct
  REGION 1..8: note = Description of Artificial Sequence: Synthetic peptide
IIPIFGSG

SEQ ID NO: 343 - protein, 16 aa, synthetic construct
  REGION 1..16: note = Description of Artificial Sequence: Synthetic peptide
ARGESPSNFVYYGMDV

SEQ ID NO: 344 - protein, 110 aa, synthetic construct
  REGION 1..110: note = Description of Artificial Sequence: Synthetic polypeptide
DIVLTQSPLSLPVTPGEPASISCRSSHSLLHSNGYNHLDWYLQKPGQSPQLLIYLGSNRASGVPDRFSGSGSGTDFTLKISRVEAEDVGVYYCMQALLVTFGPGTKVDIK

SEQ ID NO: 345 - protein, 11 aa, synthetic construct
  REGION 1..11: note = Description of Artificial Sequence: Synthetic peptide
HSLLHSNGYNH

SEQ ID NO: 346 - protein, 7 aa, synthetic construct
  REGION 1..7: note = Description of Artificial Sequence: Synthetic peptide
MQALLVT

SEQ ID NO: 347 - protein, 121 aa, synthetic construct
  REGION 1..121: note = Description of Artificial Sequence: Synthetic polypeptide
EVKVVESGGGLVQPGGSLRLSCAASEFTFTYYWMSWIRQAPGKGLEWVANVNGDATEKYYVDSVKGRFTISRDNPKKTVYLQMNSLRVEDTAVYYCARVGTTVVNDGFDLWGLGTMVTVSS

SEQ ID NO: 348 - protein, 8 aa, synthetic construct
  REGION 1..8: note = Description of Artificial Sequence: Synthetic peptide
EFTFTYYW

SEQ ID NO: 349 - protein, 8 aa, synthetic construct
  REGION 1..8: note = Description of Artificial Sequence: Synthetic peptide
VNGDATEK

SEQ ID NO: 350 - protein, 14 aa, synthetic construct
  REGION 1..14: note = Description of Artificial Sequence: Synthetic peptide
ARVGTTVVNDGFDL

SEQ ID NO: 351 - protein, 107 aa, synthetic construct
  REGION 1..107: note = Description of Artificial Sequence: Synthetic polypeptide
SYVLTQSHSVSVAPGQTARITCGGENIGGKGVHWYQQKPGQAPLLVVSSDTGRRSVTPDRFSGSNSGDTATLIISRVEAGDEADYYCQVWDPTSEYVFGSGTKVTVL

SEQ ID NO: 352 - protein, 6 aa, synthetic construct
  REGION 1..6: note = Description of Artificial Sequence: Synthetic peptide
NIGGKG

SEQ ID NO: 353 - skipped

SEQ ID NO: 354 - protein, 10 aa, synthetic construct
  REGION 1..10: note = Description of Artificial Sequence: Synthetic peptide
QVWDPTSEYV

SEQ ID NO: 355 - protein, 126 aa, synthetic construct
  REGION 1..126: note = Description of Artificial Sequence: Synthetic polypeptide
QLQLQESGSGLVKPSQTLSLTCAVSGGSISSGDYSWSWIRQPPGKGLEWIGFRYYSGTTFYNPSLESRLTISIDRSTNQFSLQLTSVTAADTAVYFCASFRPLLRFLDPEGLFEYWGQGILVTVSS

SEQ ID NO: 356 - protein, 10 aa, synthetic construct
  REGION 1..10: note = Description of Artificial Sequence: Synthetic peptide
GGSISSGDYS

SEQ ID NO: 357 - protein, 7 aa, synthetic construct
  REGION 1..7: note = Description of Artificial Sequence: Synthetic peptide
RYYSGTT

SEQ ID NO: 358 - protein, 18 aa, synthetic construct
  REGION 1..18: note = Description of Artificial Sequence: Synthetic peptide
ASFRPLLRFLDPEGLFEY

SEQ ID NO: 359 - protein, 107 aa, synthetic construct
  REGION 1..107: note = Description of Artificial Sequence: Synthetic polypeptide
ELVMTQSPATLSVSPGARATLSCRASPGANSHLAWYQQKPGQAPRLLIYGASTRATGIPARFSGSGSGTEFTLTISSLQSEDFAVYYCQQYNDWPYTFGQGTKLEIK

SEQ ID NO: 360 - protein, 6 aa, synthetic construct
  REGION 1..6: note = Description of Artificial Sequence: Synthetic peptide
PGANSH

SEQ ID NO: 361 - protein, 9 aa, synthetic construct
  REGION 1..9: note = Description of Artificial Sequence: Synthetic peptide
QQYNDWPYT

SEQ ID NO: 362 - protein, 117 aa, synthetic construct
  REGION 1..117: note = Description of Artificial Sequence: Synthetic polypeptide
QVQLVQSGAAVKKPGASVRISCEASGYTFTGYNIHWVRQAPGQGLEWMGWVNPNNGGTKFAQKFEGWVTMTVATSINTVYMELTGLKSGDTAVYFCARDHGDSFDQWGQGTLVTVSS

SEQ ID NO: 363 - protein, 8 aa, synthetic construct
  REGION 1..8: note = Description of Artificial Sequence: Synthetic peptide
GYTFTGYN

SEQ ID NO: 364 - protein, 8 aa, synthetic construct
  REGION 1..8: note = Description of Artificial Sequence: Synthetic peptide
VNPNNGGT

SEQ ID NO: 365 - protein, 10 aa, synthetic construct
  REGION 1..10: note = Description of Artificial Sequence: Synthetic peptide
ARDHGDSFDQ

SEQ ID NO: 366 - protein, 108 aa, synthetic construct
  REGION 1..108: note = Description of Artificial Sequence: Synthetic polypeptide
EIVLTQSPDTLSLSPGDRATLSCRASHSLNNDYLAWYQHRPGQAPRLLIYGTSHGATGIPDRFSGSGSGTDFTLTISRLETEDFAVYYCHHYGKSLFPFGPGTKVDIK

SEQ ID NO: 367 - protein, 7 aa, synthetic construct
  REGION 1..7: note = Description of Artificial Sequence: Synthetic peptide
HSLNNDY

SEQ ID NO: 368 - protein, 9 aa, synthetic construct
  REGION 1..9: note = Description of Artificial Sequence: Synthetic peptide
HHYGKSLFP

SEQ ID NO: 369 - protein, 124 aa, synthetic construct
  REGION 1..124: note = Description of Artificial Sequence: Synthetic polypeptide
EVQLVESGGGLVQPGGSLKLSCAASGFTFSGSAMHWVRQASGKGLEWVGRIRSKANTYATAYAASVKGRFTISRDDSKNTAYLQMNSLKTEDTAVYYCTRKHTSGWYDRGGDVWGQGTTVTVSS

SEQ ID NO: 370 - protein, 8 aa, synthetic construct
  REGION 1..8: note = Description of Artificial Sequence: Synthetic peptide
GFTFSGSA

SEQ ID NO: 371 - protein, 10 aa, synthetic construct
  REGION 1..10: note = Description of Artificial Sequence: Synthetic peptide
IRSKANTYAT

SEQ ID NO: 372 - protein, 15 aa, synthetic construct
  REGION 1..15: note = Description of Artificial Sequence: Synthetic peptide
TRKHTSGWYDRGGDV

SEQ ID NO: 373 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 374 - protein, 6 aa, synthetic construct
  REGION 1..6: note = Description of Artificial Sequence: Synthetic peptide
QDISNY

SEQ ID NO: 375 - protein, 9 aa, synthetic construct
  REGION 1..9: note = Description of Artificial Sequence: Synthetic peptide
QQYDNLPLT

SEQ ID NO: 376 - protein, 120 aa, synthetic construct
  REGION 1..120: note = Description of Artificial Sequence: Synthetic polypeptide
EVQLLESGGGLVQPGGSLRLSCAASGFTVSNYAMSWVRQAPGKGLEWVSAISGSGGSTYYADSVKGRFTISRDTSKNTLYLQMNSLRAEDTAVYYCAIDCTVTDAPLSYWGQGTLVTVSS

SEQ ID NO: 377 - protein, 8 aa, synthetic construct
  REGION 1..8: note = Description of Artificial Sequence: Synthetic peptide
GFTVSNYA

SEQ ID NO: 378 - protein, 8 aa, synthetic construct
  REGION 1..8: note = Description of Artificial Sequence: Synthetic peptide
ISGSGGST

SEQ ID NO: 379 - protein, 13 aa, synthetic construct
  REGION 1..13: note = Description of Artificial Sequence: Synthetic peptide
AIDCTVTDAPLSY

SEQ ID NO: 380 - protein, 107 aa, synthetic construct
  REGION 1..107: note = Description of Artificial Sequence: Synthetic polypeptide
AIQMTQSPSSLSPSVGDRVTITCRASQGIRNDLGWYQQKPGKAPKLLIYAASSLQSGVPSRFSGGGSGTDFTLTISSLQPEDFATYYCLQDYNYPRTFGQGTKVEIK

SEQ ID NO: 381 - protein, 6 aa, synthetic construct
  REGION 1..6: note = Description of Artificial Sequence: Synthetic peptide
QGIRND

SEQ ID NO: 382 - protein, 9 aa, synthetic construct
  REGION 1..9: note = Description of Artificial Sequence: Synthetic peptide
LQDYNYPRT

SEQ ID NO: 383 - protein, 131 aa, synthetic construct
  REGION 1..131: note = Description of Artificial Sequence: Synthetic polypeptide
QVQLEQSGAEVRKPGSSVKVSCKASGTTFSNHAMSWVRQAPGQGLEWMGGIIPLVDKSMYALKFQGRVTITADESRNTVYMELSSLGSEDTAVYYCARSFADITTFGFVVNFHYYYTLDVWGQGTPVTVSS

SEQ ID NO: 384 - protein, 8 aa, synthetic construct
  REGION 1..8: note = Description of Artificial Sequence: Synthetic peptide
GTTFSNHA

SEQ ID NO: 385 - protein, 8 aa, synthetic construct
  REGION 1..8: note = Description of Artificial Sequence: Synthetic peptide
IIPLVDKS

SEQ ID NO: 386 - protein, 24 aa, synthetic construct
  REGION 1..24: note = Description of Artificial Sequence: Synthetic peptide
ARSFADITTFGFVVNFHYYYTLDV

SEQ ID NO: 387 - protein, 110 aa, synthetic construct
  REGION 1..110: note = Description of Artificial Sequence: Synthetic polypeptide
NFMLTQPHSVSESPGKTVTISCTRSSGSIADNYVQWFQQRPGSAPTTLIYEDNRRPSGVPDRFSGSVDSSSNSASLTISGLKPEDEADYYCQSYDTTQRVFGGGTKLTVL

SEQ ID NO: 388 - protein, 8 aa, synthetic construct
  REGION 1..8: note = Description of Artificial Sequence: Synthetic peptide
SGSIADNY

SEQ ID NO: 389 - skipped

SEQ ID NO: 390 - protein, 9 aa, synthetic construct
  REGION 1..9: note = Description of Artificial Sequence: Synthetic peptide
QSYDTTQRV

SEQ ID NO: 391 - protein, 128 aa, synthetic construct
  REGION 1..128: note = Description of Artificial Sequence: Synthetic polypeptide
QLQLQESGSRLVKPSQTLSLTCAVSGGSINSGGYSWSWIRQPPGKGLEWIGNIYHGETTHYNPSLKSRVTISIDKSKNQFSLKLTSVTAADTAVYYCARAPLGNYYDTSGYLQPFDYWGPGALVTVSS

SEQ ID NO: 392 - protein, 10 aa, synthetic construct
  REGION 1..10: note = Description of Artificial Sequence: Synthetic peptide
GGSINSGGYS

SEQ ID NO: 393 - protein, 7 aa, synthetic construct
  REGION 1..7: note = Description of Artificial Sequence: Synthetic peptide
IYHGETT

SEQ ID NO: 394 - protein, 20 aa, synthetic construct
  REGION 1..20: note = Description of Artificial Sequence: Synthetic peptide
ARAPLGNYYDTSGYLQPFDY

SEQ ID NO: 395 - protein, 107 aa, synthetic construct
  REGION 1..107: note = Description of Artificial Sequence: Synthetic polypeptide
DIQMTQSPSSLSASVGDRVTITCRASQGIINDLGWYQQRPGRAPTRLIYAASSLQSGVPSRFSGSGSGTEFTLTINSLQPADFATYFCLQYNSYPPTFGQGTKVEIK

SEQ ID NO: 396 - protein, 6 aa, synthetic construct
  REGION 1..6: note = Description of Artificial Sequence: Synthetic peptide
QGIIND

SEQ ID NO: 397 - protein, 9 aa, synthetic construct
  REGION 1..9: note = Description of Artificial Sequence: Synthetic peptide
LQYNSYPPT

SEQ ID NO: 398 - protein, 118 aa, synthetic construct
  REGION 1..118: note = Description of Artificial Sequence: Synthetic polypeptide
EVQLVESGGGVVRPGGSLRLSCAASGFIFRDHGMSWVRQAPGKGLEWVSGINWNGANTGYADSVKGRSTISRDNAKNSLYLQMSSLRADDTALYHCVSHDYYYGLDVWGPGTTVIVSS

SEQ ID NO: 399 - protein, 8 aa, synthetic construct
  REGION 1..8: note = Description of Artificial Sequence: Synthetic peptide
GFIFRDHG

SEQ ID NO: 400 - protein, 8 aa, synthetic construct
  REGION 1..8: note = Description of Artificial Sequence: Synthetic peptide
INWNGANT

SEQ ID NO: 401 - protein, 11 aa, synthetic construct
  REGION 1..11: note = Description of Artificial Sequence: Synthetic peptide
VSHDYYYGLDV

SEQ ID NO: 402 - protein, 108 aa, synthetic construct
  REGION 1..108: note = Description of Artificial Sequence: Synthetic polypeptide
QSALTQPRSVSGSPGQSVTISCTGTSSDVGGDNYVSWYQQHPGKVPKLIIHDVSERPSGVPDRFSGSKSANTASLTISGLQADDEADYYCCSYAGTYTFGGGTRLTVL

SEQ ID NO: 403 - protein, 9 aa, synthetic construct
  REGION 1..9: note = Description of Artificial Sequence: Synthetic peptide
SSDVGGDNY

SEQ ID NO: 404 - skipped

SEQ ID NO: 405 - protein, 8 aa, synthetic construct
  REGION 1..8: note = Description of Artificial Sequence: Synthetic peptide
CSYAGTYT

SEQ ID NO: 406 - protein, 126 aa, synthetic construct
  REGION 1..126: note = Description of Artificial Sequence: Synthetic polypeptide
QVQLQESGPGLVKPSQTLSLTCTVSGGSISSGGYYWSWIRQLPGKGLEWIGYIYYSGSTSYNPSLKSRVTISVDTSKNQLSLNLSSVTAADTAVYYCARGRRISISGVVTPLFDYWGQGTLVTVSS

SEQ ID NO: 407 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQANSVPLTFGGGTKVEIK

SEQ ID NO: 408 - protein, 115 aa, synthetic construct
  REGION 1..115: note = Description of Artificial Sequence: Synthetic polypeptide
QVQLVQSGAEVKKPGASVKVSCQASGYTFTRYDINWVRQATGQGLEWMGWLNPKSGDTGYAQKFQGRVTMTRDTSISTAYMELTSLTSDDTAVYYCARGVDANHWGQGSLVTVSS

SEQ ID NO: 409 - protein, 8 aa, synthetic construct
  REGION 1..8: note = Description of Artificial Sequence: Synthetic peptide
GYTFTRYD

SEQ ID NO: 410 - protein, 8 aa, synthetic construct
  REGION 1..8: note = Description of Artificial Sequence: Synthetic peptide
LNPKSGDT

SEQ ID NO: 411 - protein, 8 aa, synthetic construct
  REGION 1..8: note = Description of Artificial Sequence: Synthetic peptide
ARGVDANH

SEQ ID NO: 412 - protein, 113 aa, synthetic construct
  REGION 1..113: note = Description of Artificial Sequence: Synthetic polypeptide
DIVVTQSPDSLAVSLGERATINCKSSQSIFDTSSNKNYLAWFRQRPGQPPQLLIYWASTRESGVPDRFSGSGSGTDFTLTISSLQAEDVAVYYCHQYYSLPHAFGQGTKLEIK

SEQ ID NO: 413 - protein, 12 aa, synthetic construct
  REGION 1..12: note = Description of Artificial Sequence: Synthetic peptide
QSIFDTSSNKNY

SEQ ID NO: 414 - protein, 9 aa, synthetic construct
  REGION 1..9: note = Description of Artificial Sequence: Synthetic peptide
HQYYSLPHA

SEQ ID NO: 415 - protein, 126 aa, synthetic construct
  REGION 1..126: note = Description of Artificial Sequence: Synthetic polypeptide
EAQLVESGGGLVQPGGSLRLSCAASGFTFSSYYIHWVRQAPGKGLVWVSRINSDGSSTRYADSVKGRFTISRDNAKNTLYLQMNSLRAEDTAVYFCARASRTVYGDSPLSNGMDVWGQGTKVTVSS

SEQ ID NO: 416 - protein, 8 aa, synthetic construct
  REGION 1..8: note = Description of Artificial Sequence: Synthetic peptide
GFTFSSYY

SEQ ID NO: 417 - protein, 8 aa, synthetic construct
  REGION 1..8: note = Description of Artificial Sequence: Synthetic peptide
INSDGSST

SEQ ID NO: 418 - protein, 19 aa, synthetic construct
  REGION 1..19: note = Description of Artificial Sequence: Synthetic peptide
ARASRTVYGDSPLSNGMDV

SEQ ID NO: 419 - protein, 107 aa, synthetic construct
  REGION 1..107: note = Description of Artificial Sequence: Synthetic polypeptide
SYELTQPPSVSVSPGQTASITCSGDKLGDKFACWYQQKPGHSPVLVIYQDDKRPSGIPERFSGSNSGNTATLTISGTQAMDEADYYCQAWDSSTHVVFGGGTKLTVL

SEQ ID NO: 420 - protein, 6 aa, synthetic construct
  REGION 1..6: note = Description of Artificial Sequence: Synthetic peptide
KLGDKF

SEQ ID NO: 421 - skipped

SEQ ID NO: 422 - protein, 10 aa, synthetic construct
  REGION 1..10: note = Description of Artificial Sequence: Synthetic peptide
QAWDSSTHVV

SEQ ID NO: 423 - protein, 125 aa, synthetic construct
  REGION 1..125: note = Description of Artificial Sequence: Synthetic polypeptide
EVQLVESGGGLVQPGGSLRLSCAASGFSFNTYNMNWVRQAPGKGLEWISDITSSGSMRSYADAVKGRFTISRDNAKNSLHLQMNSLRVEDTAVYYCTRGWHDDLWSGYSYGLDVWGQGTTVTVSS

SEQ ID NO: 424 - protein, 8 aa, synthetic construct
  REGION 1..8: note = Description of Artificial Sequence: Synthetic peptide
GFSFNTYN

SEQ ID NO: 425 - protein, 8 aa, synthetic construct
  REGION 1..8: note = Description of Artificial Sequence: Synthetic peptide
ITSSGSMR

SEQ ID NO: 426 - protein, 18 aa, synthetic construct
  REGION 1..18: note = Description of Artificial Sequence: Synthetic peptide
TRGWHDDLWSGYSYGLDV

SEQ ID NO: 427 - protein, 107 aa, synthetic construct
  REGION 1..107: note = Description of Artificial Sequence: Synthetic polypeptide
DIQMTQSPSSLSASVGDRVTITCRASQGIRNDLGWYQQRPGKAPRCLIYGASSLQSGVPSRFSGSGSGTEFTLTISNLQAEDFATYYCLQHKSYPLTFGPGTKVDIK

SEQ ID NO: 428 - protein, 9 aa, synthetic construct
  REGION 1..9: note = Description of Artificial Sequence: Synthetic peptide
LQHKSYPLT

SEQ ID NO: 429 - protein, 122 aa, synthetic construct
  REGION 1..122: note = Description of Artificial Sequence: Synthetic polypeptide
QLLLLGPGPGVVRPSETLSLTCNVSGHSITDSPYYWGWIRQAPGKGLEWIGHFYYSDYTYYNPSLKSRVNVSVDTSKNHLFLALTSVTAADTAVYYCARGFGGYDSPIWAIWGQGTLVTVSS

SEQ ID NO: 430 - protein, 10 aa, synthetic construct
  REGION 1..10: note = Description of Artificial Sequence: Synthetic peptide
GHSITDSPYY

SEQ ID NO: 431 - protein, 7 aa, synthetic construct
  REGION 1..7: note = Description of Artificial Sequence: Synthetic peptide
FYYSDYT

SEQ ID NO: 432 - protein, 14 aa, synthetic construct
  REGION 1..14: note = Description of Artificial Sequence: Synthetic peptide
ARGFGGYDSPIWAI

SEQ ID NO: 433 - protein, 103 aa, synthetic construct
  REGION 1..103: note = Description of Artificial Sequence: Synthetic polypeptide
SHAVTQPPSVSVAPGQTASLTCAGDDIEENTVHWYQQKPGQAPVLVIYYTTDRPSAIPERFFGSKSGNTATLSIARVEAGDEADYYCQVSDRVFGGGTKLTVL

SEQ ID NO: 434 - protein, 6 aa, synthetic construct
  REGION 1..6: note = Description of Artificial Sequence: Synthetic peptide
DIEENT

SEQ ID NO: 435 - skipped

SEQ ID NO: 436 - protein, 6 aa, synthetic construct
  REGION 1..6: note = Description of Artificial Sequence: Synthetic peptide
QVSDRV

SEQ ID NO: 437 - protein, 121 aa, synthetic construct
  REGION 1..121: note = Description of Artificial Sequence: Synthetic polypeptide
EVQLVQSGGGLVKPGGSLRLSCAASGSTLTNYNINWVRQAPGKGLQWVSSISGTRDYTYYADSVVGRFTISRDNAKNSVYLQMNSLRAEDTAVYYCARGREVGGDYDSYDWGQGTLVTVSS

SEQ ID NO: 438 - protein, 8 aa, synthetic construct
  REGION 1..8: note = Description of Artificial Sequence: Synthetic peptide
GSTLTNYN

SEQ ID NO: 439 - protein, 8 aa, synthetic construct
  REGION 1..8: note = Description of Artificial Sequence: Synthetic peptide
ISGTRDYT

SEQ ID NO: 440 - protein, 14 aa, synthetic construct
  REGION 1..14: note = Description of Artificial Sequence: Synthetic peptide
ARGREVGGDYDSYD

SEQ ID NO: 441 - protein, 107 aa, synthetic construct
  REGION 1..107: note = Description of Artificial Sequence: Synthetic polypeptide
DIQMTQSPSSLSASVGDRVTITCQASQDISTFLHWYQQKPGKAPSVLIYGASDLKTGVPSRFSGSGSGTHFTLTISSLQPEDIATYYCQQYDHLPLTFGGGTKVEIK

SEQ ID NO: 442 - protein, 6 aa, synthetic construct
  REGION 1..6: note = Description of Artificial Sequence: Synthetic peptide
QDISTF

SEQ ID NO: 443 - protein, 9 aa, synthetic construct
  REGION 1..9: note = Description of Artificial Sequence: Synthetic peptide
QQYDHLPLT

SEQ ID NO: 444 - protein, 121 aa, synthetic construct
  REGION 1..121: note = Description of Artificial Sequence: Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDSRLGWAYDAFDIWGQGTMVTVSS

SEQ ID NO: 445 - protein, 8 aa, synthetic construct
  REGION 1..8: note = Description of Artificial Sequence: Synthetic peptide
GFTVSSNY

SEQ ID NO: 446 - protein, 7 aa, synthetic construct
  REGION 1..7: note = Description of Artificial Sequence: Synthetic peptide
IYSGGST

SEQ ID NO: 447 - protein, 15 aa, synthetic construct
  REGION 1..15: note = Description of Artificial Sequence: Synthetic peptide
ARDSRLGWAYDAFDI

SEQ ID NO: 448 - protein, 108 aa, synthetic construct
  REGION 1..108: note = Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQDGTFGQGTKVEIK

SEQ ID NO: 449 - protein, 5 aa, synthetic construct
  REGION 1..5: note = Description of Artificial Sequence: Synthetic peptide
MQDGT

SEQ ID NO: 450 - protein, 123 aa, synthetic construct
  REGION 1..123: note = Description of Artificial Sequence: Synthetic polypeptide
QVQLVQSGSELKKPGASVKASCKASGYTFSNYAVNWVRQAPGQGLEWMGWINTKTGNPTYGQGFTGRFVFSLDTSVSTAYLQISSLKAEDTAVYYCARAADYGEPYYYGMDVWGQGTTVTVSS

SEQ ID NO: 451 - protein, 8 aa, synthetic construct
  REGION 1..8: note = Description of Artificial Sequence: Synthetic peptide
GYTFSNYA

SEQ ID NO: 452 - protein, 8 aa, synthetic construct
  REGION 1..8: note = Description of Artificial Sequence: Synthetic peptide
INTKTGNP

SEQ ID NO: 453 - protein, 16 aa, synthetic construct
  REGION 1..16: note = Description of Artificial Sequence: Synthetic peptide
ARAADYGEPYYYGMDV

SEQ ID NO: 454 - protein, 110 aa, synthetic construct
  REGION 1..110: note = Description of Artificial Sequence: Synthetic polypeptide
QSALTQPASVSGSPGQSITISCTGTNSDVGSYNLVSWYQQHPGKAPKFMIYEGTKRPSGVSNRFSGSKSGHTASLTISGLQAEDEADYYCCSYAGTSTLVFGGGTKLTVL

SEQ ID NO: 455 - protein, 9 aa, synthetic construct
  REGION 1..9: note = Description of Artificial Sequence: Synthetic peptide
NSDVGSYNL

SEQ ID NO: 456 - skipped

SEQ ID NO: 457 - protein, 10 aa, synthetic construct
  REGION 1..10: note = Description of Artificial Sequence: Synthetic peptide
CSYAGTSTLV

SEQ ID NO: 458 - protein, 121 aa, synthetic construct
  REGION 1..121: note = Description of Artificial Sequence: Synthetic polypeptide
EVQLVESGGGLVQPGGSLRLSCAASGFTFRNYWMNWVRQAPGKGLVWVSRINSEGSSTSYADPVKGRFTISRDNAKDTLYLQMDSLRAEDSAVYYCARIFNGYIHVGRDYWGQGTRVTVSS

SEQ ID NO: 459 - protein, 8 aa, synthetic construct
  REGION 1..8: note = Description of Artificial Sequence: Synthetic peptide
GFTFRNYW

SEQ ID NO: 460 - protein, 8 aa, synthetic construct
  REGION 1..8: note = Description of Artificial Sequence: Synthetic peptide
INSEGSST

SEQ ID NO: 461 - protein, 14 aa, synthetic construct
  REGION 1..14: note = Description of Artificial Sequence: Synthetic peptide
ARIFNGYIHVGRDY

SEQ ID NO: 462 - protein, 107 aa, synthetic construct
  REGION 1..107: note = Description of Artificial Sequence: Synthetic polypeptide
DIQMTQSPSTLSASIGDRVTITCRASESISNWLAWFQQKPGKAPKLLIYKASNLESGVPSRFSGSGSGTEFTLTISSLQPDDFATYYCQQYNSNSQTFGQGTKLDLK

SEQ ID NO: 463 - protein, 6 aa, synthetic construct
  REGION 1..6: note = Description of Artificial Sequence: Synthetic peptide
ESISNW

SEQ ID NO: 464 - protein, 9 aa, synthetic construct
  REGION 1..9: note = Description of Artificial Sequence: Synthetic peptide
QQYNSNSQT

SEQ ID NO: 465 - protein, 125 aa, synthetic construct
  REGION 1..125: note = Description of Artificial Sequence: Synthetic polypeptide
QVQLQESGPGLVKPSETLSLTCTVSGGSISSYYWNWIRQPPGKGLEWIGYIYYSGSTNYNPSLKSRATISVDTSKNQFSLKLSSVTAADTAVYYCARANLFGVALRRVLGPFDYWGQGTLVTVSS

SEQ ID NO: 466 - protein, 8 aa, synthetic construct
  REGION 1..8: note = Description of Artificial Sequence: Synthetic peptide
GGSISSYY

SEQ ID NO: 467 - protein, 19 aa, synthetic construct
  REGION 1..19: note = Description of Artificial Sequence: Synthetic peptide
ARANLFGVALRRVLGPFDY

SEQ ID NO: 468 - protein, 107 aa, synthetic construct
  REGION 1..107: note = Description of Artificial Sequence: Synthetic polypeptide
DIQMTQSPSSLSASVGDRVTIACRASQSIANYLNWYQQKPGKAPKLLIYAASNLQSGVPSRFSGSGSGTDFTLTISSLQPEDFATYYCQQSYSTPYTFGQGTKLEIK

SEQ ID NO: 469 - protein, 6 aa, synthetic construct
  REGION 1..6: note = Description of Artificial Sequence: Synthetic peptide
QSIANY

SEQ ID NO: 470 - protein, 9 aa, synthetic construct
  REGION 1..9: note = Description of Artificial Sequence: Synthetic peptide
QQSYSTPYT

SEQ ID NO: 471 - protein, 127 aa, synthetic construct
  REGION 1..127: note = Description of Artificial Sequence: Synthetic polypeptide
QVQLVESGGGVVQPGRSLRLSCAASGFSFRSYGMHWVRQAPGKGLEWVAVISYDGSNKYYVDSVKGRFTISRDNSKNTLYVQMNSLTDEDTAVYYCARDRGVTTRQFSYYYYGMDVWGQGTTVTVSS

SEQ ID NO: 472 - protein, 8 aa, synthetic construct
  REGION 1..8: note = Description of Artificial Sequence: Synthetic peptide
GFSFRSYG

SEQ ID NO: 473 - protein, 20 aa, synthetic construct
  REGION 1..20: note = Description of Artificial Sequence: Synthetic peptide
ARDRGVTTRQFSYYYYGMDV

SEQ ID NO: 474 - protein, 107 aa, synthetic construct
  REGION 1..107: note = Description of Artificial Sequence: Synthetic polypeptide
AIRMTQSPSSFSASTGDRVTITCRASQSITSYLAWYQQKPGKAPKLLIYAASTLQSGLPSRFSGSGSGTDFTLTISGLQSEDFATYYCQQYYNYPQTFGQGTRVEIK

SEQ ID NO: 475 - protein, 6 aa, synthetic construct
  REGION 1..6: note = Description of Artificial Sequence: Synthetic peptide
QSITSY

SEQ ID NO: 476 - protein, 9 aa, synthetic construct
  REGION 1..9: note = Description of Artificial Sequence: Synthetic peptide
QQYYNYPQT

SEQ ID NO: 477 - protein, 119 aa, synthetic construct
  REGION 1..119: note = Description of Artificial Sequence: Synthetic polypeptide
EVQLLESGGQLVQPGGSLRLSCGAFGFTFGDAAMTWVRQAPGKGLEWVSTISGRGDETFSADSVKGRFTISRDNFKNMLYVQMNSLRAEDTATYYCARLGHLRGWFDSWGQGTLVTVSS

SEQ ID NO: 478 - protein, 8 aa, synthetic construct
  REGION 1..8: note = Description of Artificial Sequence: Synthetic peptide
GFTFGDAA

SEQ ID NO: 479 - protein, 8 aa, synthetic construct
  REGION 1..8: note = Description of Artificial Sequence: Synthetic peptide
ISGRGDET

SEQ ID NO: 480 - protein, 12 aa, synthetic construct
  REGION 1..12: note = Description of Artificial Sequence: Synthetic peptide
ARLGHLRGWFDS

SEQ ID NO: 481 - protein, 107 aa, synthetic construct
  REGION 1..107: note = Description of Artificial Sequence: Synthetic polypeptide
EIVMTQSPATLSVSPGERVTLSCRASQSVSSNLAWYQQKPGQAPRLLIYGASTRATGIPAGFSGSGSGTEFTLTISSLQSEDFAVYYCQQYNNWPRTFGQGTKVEIK

SEQ ID NO: 482 - protein, 6 aa, synthetic construct
  REGION 1..6: note = Description of Artificial Sequence: Synthetic peptide
QSVSSN

SEQ ID NO: 483 - protein, 129 aa, synthetic construct
  REGION 1..129: note = Description of Artificial Sequence: Synthetic polypeptide
EVQLVESGGGLVKPGGSLRLSCAASGFTFSDAWMTWVRQAPGKGLEWVGRIKSKTDGGTTDYGAPVKGRFSISRDDSKNTLYLHMNSLKTEDTAVYYCTTKSPNSNWFPFYYYYYMDVWGKGTTVTVSS

SEQ ID NO: 484 - protein, 8 aa, synthetic construct
  REGION 1..8: note = Description of Artificial Sequence: Synthetic peptide
GFTFSDAW

SEQ ID NO: 485 - protein, 10 aa, synthetic construct
  REGION 1..10: note = Description of Artificial Sequence: Synthetic peptide
IKSKTDGGTT

SEQ ID NO: 486 - protein, 20 aa, synthetic construct
  REGION 1..20: note = Description of Artificial Sequence: Synthetic peptide
TTKSPNSNWFPFYYYYYMDV

SEQ ID NO: 487 - protein, 110 aa, synthetic construct
  REGION 1..110: note = Description of Artificial Sequence: Synthetic polypeptide
QSALTQPRSVSGSPGQSVTISCTGTSSDVGGYNFVSWYQQHPGKAPQLMIYDVTKRPSGVPDRFSGSKSGNTASLTISGLQAEDEGDYYCYSYAASSLYVFGTGTKVTVL

SEQ ID NO: 488 - protein, 9 aa, synthetic construct
  REGION 1..9: note = Description of Artificial Sequence: Synthetic peptide
SSDVGGYNF

SEQ ID NO: 489 - protein, 10 aa, synthetic construct
  REGION 1..10: note = Description of Artificial Sequence: Synthetic peptide
YSYAASSLYV

SEQ ID NO: 490 - protein, 130 aa, synthetic construct
  REGION 1..130: note = Description of Artificial Sequence: Synthetic polypeptide
QLQLQESGPGLVKPSETLSLICTVSGGAITSSTFYWAWIRQPPGRGLEWIGSMYYSGSTYYNLSLKSRVIISVNTSKNQFSLTLTSATATDMAVYYCVRHTLHDYGSGSFPDYSYGMDVWGQGTTVTVSS

SEQ ID NO: 491 - protein, 10 aa, synthetic construct
  REGION 1..10: note = Description of Artificial Sequence: Synthetic peptide
GGAITSSTFY

SEQ ID NO: 492 - protein, 7 aa, synthetic construct
  REGION 1..7: note = Description of Artificial Sequence: Synthetic peptide
MYYSGST

SEQ ID NO: 493 - protein, 22 aa, synthetic construct
  REGION 1..22: note = Description of Artificial Sequence: Synthetic peptide
VRHTLHDYGSGSFPDYSYGMDV

SEQ ID NO: 494 - protein, 107 aa, synthetic construct
  REGION 1..107: note = Description of Artificial Sequence: Synthetic polypeptide
EIVLTQSPATLSLFPGERGTLSCRASQSVSSHLIWYQQKPGQAPRVLIFDATNRATGIPARFSGSGSGTDFTLTISNLEPEDYGVYYCQQRSNWPLTFGGGTKVEIK

SEQ ID NO: 495 - protein, 6 aa, synthetic construct
  REGION 1..6: note = Description of Artificial Sequence: Synthetic peptide
QSVSSH

SEQ ID NO: 496 - protein, 9 aa, synthetic construct
  REGION 1..9: note = Description of Artificial Sequence: Synthetic peptide
QQRSNWPLT

SEQ ID NO: 497 - protein, 128 aa, synthetic construct
  REGION 1..128: note = Description of Artificial Sequence: Synthetic polypeptide
EVQLLESGGGLVQPGGSLKLSCVASGFTFSSYAMMWVRQAPGKGLEWISSISSSGGSTYYADSVKGRFTISRDNSKNTLYLQMNSLRVEDTAVYYCAKSHCSTTSCPRAFYYYGMDVWGQGTTVTVSS

SEQ ID NO: 498 - protein, 8 aa, synthetic construct
  REGION 1..8: note = Description of Artificial Sequence: Synthetic peptide
ISSSGGST

SEQ ID NO: 499 - protein, 21 aa, synthetic construct
  REGION 1..21: note = Description of Artificial Sequence: Synthetic peptide
AKSHCSTTSCPRAFYYYGMDV

SEQ ID NO: 500 - protein, 107 aa, synthetic construct
  REGION 1..107: note = Description of Artificial Sequence: Synthetic polypeptide
DIQMTQSPSSLSASVGDRVTITCRASQTITTYLNWYQQKPGKAPKLLIYAASSLQSGVPSRFSGSGSGTDFTLSLSSLQPEDSATYYCQQSYSTLGAFGGGTKVEIK

SEQ ID NO: 501 - protein, 6 aa, synthetic construct
  REGION 1..6: note = Description of Artificial Sequence: Synthetic peptide
QTITTY

SEQ ID NO: 502 - protein, 9 aa, synthetic construct
  REGION 1..9: note = Description of Artificial Sequence: Synthetic peptide
QQSYSTLGA

SEQ ID NO: 503 - protein, 116 aa, synthetic construct
  REGION 1..116: note = Description of Artificial Sequence: Synthetic polypeptide
QVHLQESGPGLVKPSGTLSLTCTVSGGSISTYYWSWIRQPPGKGLEWIGYIYYGGTTNYNPSLKSRVTISVDTSKNQFSLRLRSVTAADTAVYYCAREIDSRMDRWGQGTLVTVSS

SEQ ID NO: 504 - protein, 8 aa, synthetic construct
  REGION 1..8: note = Description of Artificial Sequence: Synthetic peptide
GGSISTYY

SEQ ID NO: 505 - protein, 7 aa, synthetic construct
  REGION 1..7: note = Description of Artificial Sequence: Synthetic peptide
IYYGGTT

SEQ ID NO: 506 - protein, 10 aa, synthetic construct
  REGION 1..10: note = Description of Artificial Sequence: Synthetic peptide
AREIDSRMDR

SEQ ID NO: 507 - protein, 108 aa, synthetic construct
  REGION 1..108: note = Description of Artificial Sequence: Synthetic polypeptide
SYALTQPPSVSVAPGKTARITCGGDNIGSKTVHWYHQKPGQAPVLVIYYDSNRPSGISERFSGSNSGNTATLTISRVEAGDEADYYCQVWDSNSDHRIFGGGTKLTVL

SEQ ID NO: 508 - protein, 6 aa, synthetic construct
  REGION 1..6: note = Description of Artificial Sequence: Synthetic peptide
NIGSKT

SEQ ID NO: 509 - skipped

SEQ ID NO: 510 - protein, 11 aa, synthetic construct
  REGION 1..11: note = Description of Artificial Sequence: Synthetic peptide
QVWDSNSDHRI

SEQ ID NO: 511 - protein, 129 aa, synthetic construct
  REGION 1..129: note = Description of Artificial Sequence: Synthetic polypeptide
QVQLVQSGAEVRKPGSSVKVSCKASGGTFSNNPITWVRQAPGQGLEWMGWIIPIFNTTNYAQKFQGRVTITADESTSTAYMELSSLKSEDTALFYCARDRAHAYCNNGVCYTTDAFDVWGQGTLVTVSS

SEQ ID NO: 512 - protein, 8 aa, synthetic construct
  REGION 1..8: note = Description of Artificial Sequence: Synthetic peptide
GGTFSNNP

SEQ ID NO: 513 - protein, 8 aa, synthetic construct
  REGION 1..8: note = Description of Artificial Sequence: Synthetic peptide
IIPIFNTT

SEQ ID NO: 514 - protein, 22 aa, synthetic construct
  REGION 1..22: note = Description of Artificial Sequence: Synthetic peptide
ARDRAHAYCNNGVCYTTDAFDV

SEQ ID NO: 515 - protein, 106 aa, synthetic construct
  REGION 1..106: note = Description of Artificial Sequence: Synthetic polypeptide
ETVLTQSPATLSLSPGERATLSCRASQSVGRYLAWYQHKPGQAPRLLIYDASNRATGIPARFSGSGSGTDFTLTISSLEPEDSAVYYCQQGTDWLTFGGGTKVEIK

SEQ ID NO: 516 - protein, 6 aa, synthetic construct
  REGION 1..6: note = Description of Artificial Sequence: Synthetic peptide
QSVGRY

SEQ ID NO: 517 - protein, 8 aa, synthetic construct
  REGION 1..8: note = Description of Artificial Sequence: Synthetic peptide
QQGTDWLT

SEQ ID NO: 518 - protein, 121 aa, synthetic construct
  REGION 1..121: note = Description of Artificial Sequence: Synthetic polypeptide
QVQLVESGGGLVKPGGSLRLSCAASGITFSDNYMTWIRQAPGKGLEWVSYISSSGTNIFYADSLKGRFTISRDNAKNSLYLQMNSLRAEDTAVYYCARTLMTGSSLYFDYWGQGTQVTVSS

SEQ ID NO: 519 - protein, 8 aa, synthetic construct
  REGION 1..8: note = Description of Artificial Sequence: Synthetic peptide
GITFSDNY

SEQ ID NO: 520 - protein, 8 aa, synthetic construct
  REGION 1..8: note = Description of Artificial Sequence: Synthetic peptide
ISSSGTNI

SEQ ID NO: 521 - protein, 14 aa, synthetic construct
  REGION 1..14: note = Description of Artificial Sequence: Synthetic peptide
ARTLMTGSSLYFDY

SEQ ID NO: 522 - protein, 109 aa, synthetic construct
  REGION 1..109: note = Description of Artificial Sequence: Synthetic polypeptide
SYELTQPPSVSVSPGQTARITCSGDALPKQYAYWYQQKPGQAPVLVIYKDSERPSGIPERFSGSSSGTTVTLAISGVQAEDEADYYCQSADIRVTESVLFGGGTKLTVL

SEQ ID NO: 523 - protein, 6 aa, synthetic construct
  REGION 1..6: note = Description of Artificial Sequence: Synthetic peptide
ALPKQY

SEQ ID NO: 524 - skipped

SEQ ID NO: 525 - protein, 12 aa, synthetic construct
  REGION 1..12: note = Description of Artificial Sequence: Synthetic peptide
QSADIRVTESVL

SEQ ID NO: 526 - protein, 119 aa, synthetic construct
  REGION 1..119: note = Description of Artificial Sequence: Synthetic polypeptide
EVHLLESGGHLVQPGGSLRLACAVSGFTFSDSAMTWVRQAPGKGLEWVSTISGRGDETFFADSVKGRFSIFRDNSNSVLYVQMNSLRAEDTATYYCARYGHHKGWFDSWGQGTLVTVSS

SEQ ID NO: 527 - protein, 8 aa, synthetic construct
  REGION 1..8: note = Description of Artificial Sequence: Synthetic peptide
GFTFSDSA

SEQ ID NO: 528 - protein, 12 aa, synthetic construct
  REGION 1..12: note = Description of Artificial Sequence: Synthetic peptide
ARYGHHKGWFDS

SEQ ID NO: 529 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGASTRAIGIPAGFSGSGSGTEFTLTISSLQSEDSAVYYCQQYNNWPRTFGQGTKVEIK

SEQ ID NO: 530 - protein, 126 aa, synthetic construct
  REGION 1..126: note = Description of Artificial Sequence: Synthetic polypeptide
QVQLVQSGTEVKKPGASVKVSCKASGYTFSSFGITWVRQAPGQGLEWMGWISAYNGNTKYAQAVQGRVTLTTDTSTTTAYMELRSLRSNDTAVYFCAREGIEHLVVEGRGPGGDCWGQGTLVIVSS

SEQ ID NO: 531 - protein, 8 aa, synthetic construct
  REGION 1..8: note = Description of Artificial Sequence: Synthetic peptide
GYTFSSFG

SEQ ID NO: 532 - protein, 8 aa, synthetic construct
  REGION 1..8: note = Description of Artificial Sequence: Synthetic peptide
ISAYNGNT

SEQ ID NO: 533 - protein, 19 aa, synthetic construct
  REGION 1..19: note = Description of Artificial Sequence: Synthetic peptide
AREGIEHLVVEGRGPGGDC

SEQ ID NO: 534 - protein, 108 aa, synthetic construct
  REGION 1..108: note = Description of Artificial Sequence: Synthetic polypeptide
SYELTQPPSVSVSPGQTARITCSGDALPKEYTSWYQQKSGQAPVLVIYEDIKRPSGIPERFSGSSSGTMASLTISGAQVDDEADYYCYSTDTSGDHKVFGGGTKLTVL

SEQ ID NO: 535 - protein, 6 aa, synthetic construct
  REGION 1..6: note = Description of Artificial Sequence: Synthetic peptide
ALPKEY

SEQ ID NO: 536 - skipped

SEQ ID NO: 537 - protein, 11 aa, synthetic construct
  REGION 1..11: note = Description of Artificial Sequence: Synthetic peptide
YSTDTSGDHKV

SEQ ID NO: 538 - protein, 121 aa, synthetic construct
  REGION 1..121: note = Description of Artificial Sequence: Synthetic polypeptide
QVQLVESGGGVVQPGRSLRLSCAASGFTFSSYAMHWVRQAPGKGREWVAALSYDGSSTYYADSVKGRLTISRDNSKNTLYLQMNSLRAEDTAVYFCTRVPYGEGRAANDYWGQGTLVTVSS

SEQ ID NO: 539 - protein, 8 aa, synthetic construct
  REGION 1..8: note = Description of Artificial Sequence: Synthetic peptide
LSYDGSST

SEQ ID NO: 540 - protein, 14 aa, synthetic construct
  REGION 1..14: note = Description of Artificial Sequence: Synthetic peptide
TRVPYGEGRAANDY

SEQ ID NO: 541 - protein, 107 aa, synthetic construct
  REGION 1..107: note = Description of Artificial Sequence: Synthetic polypeptide
DIQMTQSPSTLSASVGDRVTITCRASQSIGSWLAWYQQKPGKAPKLLIYKASNIESGVPSRFSGSGSGTEFTLTISSLQPDDFATYYCQHYNTYSRSFGGGTEVAIK

SEQ ID NO: 542 - protein, 6 aa, synthetic construct
  REGION 1..6: note = Description of Artificial Sequence: Synthetic peptide
QSIGSW

SEQ ID NO: 543 - protein, 9 aa, synthetic construct
  REGION 1..9: note = Description of Artificial Sequence: Synthetic peptide
QHYNTYSRS

SEQ ID NO: 544 - protein, 121 aa, synthetic construct
  REGION 1..121: note = Description of Artificial Sequence: Synthetic polypeptide
QVQLVQSGAEVKTPGSSVKVSCTASGDSFSRYAINWVRQAPGQGLEWVGKIVPVFGAASYAQKFQGRVTITADESTSTVYMELSSLRSEDTAVYYCARGIVKLSTMPPVYWGQGTLVTVSS

SEQ ID NO: 545 - protein, 8 aa, synthetic construct
  REGION 1..8: note = Description of Artificial Sequence: Synthetic peptide
GDSFSRYA

SEQ ID NO: 546 - protein, 8 aa, synthetic construct
  REGION 1..8: note = Description of Artificial Sequence: Synthetic peptide
IVPVFGAA

SEQ ID NO: 547 - protein, 14 aa, synthetic construct
  REGION 1..14: note = Description of Artificial Sequence: Synthetic peptide
ARGIVKLSTMPPVY

SEQ ID NO: 548 - protein, 111 aa, synthetic construct
  REGION 1..111: note = Description of Artificial Sequence: Synthetic polypeptide
DIVMTQTPLSLSVTPGQPASISCKSSQSLLHSDGKTYLYWYLQKPGQSPQLLISEVSSRFSGVPDRFSGSGSGTDFTLKISRVEAEDVGVYYCMQGIHHLTFGPGTKVDIK

SEQ ID NO: 549 - protein, 11 aa, synthetic construct
  REGION 1..11: note = Description of Artificial Sequence: Synthetic peptide
QSLLHSDGKTY

SEQ ID NO: 550 - skipped

SEQ ID NO: 551 - protein, 8 aa, synthetic construct
  REGION 1..8: note = Description of Artificial Sequence: Synthetic peptide
MQGIHHLT

SEQ ID NO: 552 - protein, 129 aa, synthetic construct
  REGION 1..129: note = Description of Artificial Sequence: Synthetic polypeptide
QVQLQESGPGLVKPSETLSLTCSVSGGSVSDSAYYWSWIRQPPGGGLEFIGYVYNSGSTNYNPSLKSRVTISVDTSKNQFSLSLSSLTAADTAVYYCARYCSSTSCYVRSSDVNWFDPWGQGTLVIVSS

SEQ ID NO: 553 - protein, 10 aa, synthetic construct
  REGION 1..10: note = Description of Artificial Sequence: Synthetic peptide
GGSVSDSAYY

SEQ ID NO: 554 - protein, 7 aa, synthetic construct
  REGION 1..7: note = Description of Artificial Sequence: Synthetic peptide
VYNSGST

SEQ ID NO: 555 - protein, 21 aa, synthetic construct
  REGION 1..21: note = Description of Artificial Sequence: Synthetic peptide
ARYCSSTSCYVRSSDVNWFDP

SEQ ID NO: 556 - protein, 106 aa, synthetic construct
  REGION 1..106: note = Description of Artificial Sequence: Synthetic polypeptide
EIVLTQSPGTLSSSPGESATLSCRASQSLGTYLAWYQQKPGQAPRLLIYDASKRATGIPARFSGSGSGTDFTLTISSLEPEDFAVYYCHQRSHWLTFGGGTKVEIK

SEQ ID NO: 557 - protein, 6 aa, synthetic construct
  REGION 1..6: note = Description of Artificial Sequence: Synthetic peptide
QSLGTY

SEQ ID NO: 558 - protein, 8 aa, synthetic construct
  REGION 1..8: note = Description of Artificial Sequence: Synthetic peptide
HQRSHWLT

SEQ ID NO: 559 - protein, 123 aa, synthetic construct
  REGION 1..123: note = Description of Artificial Sequence: Synthetic polypeptide
EAQLLESGGGLVQPGGSLRLSCAASGFNFSNYAMTWVRQAPGKGLEWVSAISSGGGTTYYADSVKGRFTISRDNSKNTVYLQMNSLKDADSALYYCAKPGRAVVVRLSYFDSWGQGTLVTVSS

SEQ ID NO: 560 - protein, 8 aa, synthetic construct
  REGION 1..8: note = Description of Artificial Sequence: Synthetic peptide
GFNFSNYA

SEQ ID NO: 561 - protein, 8 aa, synthetic construct
  REGION 1..8: note = Description of Artificial Sequence: Synthetic peptide
ISSGGGTT

SEQ ID NO: 562 - protein, 16 aa, synthetic construct
  REGION 1..16: note = Description of Artificial Sequence: Synthetic peptide
AKPGRAVVVRLSYFDS

SEQ ID NO: 563 - protein, 110 aa, synthetic construct
  REGION 1..110: note = Description of Artificial Sequence: Synthetic polypeptide
QSVLTQPPSVSAAPGQKVSISCSGSGSNIANHYVSWYQHLPGTAPKLLIYDNNKRPSGIPDRFSGSKSGTSATLGITGLQTGDEADYYCGTWDSSLTVVVFGGGTKLTVL

SEQ ID NO: 564 - protein, 8 aa, synthetic construct
  REGION 1..8: note = Description of Artificial Sequence: Synthetic peptide
GSNIANHY

SEQ ID NO: 565 - protein, 11 aa, synthetic construct
  REGION 1..11: note = Description of Artificial Sequence: Synthetic peptide
GTWDSSLTVVV

SEQ ID NO: 566 - protein, 127 aa, synthetic construct
  REGION 1..127: note = Description of Artificial Sequence: Synthetic polypeptide
QITLKESGPTLVKPTETLTLTCTFSGFSLTTSGVAVGWVRQPPGKALEWLALIYWDDDERYTPSLKSRLTITKDTSKSQVVLTMTNMDPVDTATYFCVHCEGPDILLVPAAYFFDFWGQGTLVTVSS

SEQ ID NO: 567 - protein, 10 aa, synthetic construct
  REGION 1..10: note = Description of Artificial Sequence: Synthetic peptide
GFSLTTSGVA

SEQ ID NO: 568 - protein, 7 aa, synthetic construct
  REGION 1..7: note = Description of Artificial Sequence: Synthetic peptide
IYWDDDE

SEQ ID NO: 569 - protein, 19 aa, synthetic construct
  REGION 1..19: note = Description of Artificial Sequence: Synthetic peptide
VHCEGPDILLVPAAYFFDF

SEQ ID NO: 570 - protein, 108 aa, synthetic construct
  REGION 1..108: note = Description of Artificial Sequence: Synthetic polypeptide
EIVLTQSPGTLSLSPGDRATLSCRASQSVSRRYLAWYQQSPGQAPRLLISGASSRATGIPDRFSGSGSGTDFTLTISRLEPEDFAMYYCQQYGSSTGTFGQGTKVEMK

SEQ ID NO: 571 - protein, 7 aa, synthetic construct
  REGION 1..7: note = Description of Artificial Sequence: Synthetic peptide
QSVSRRY

SEQ ID NO: 572 - protein, 9 aa, synthetic construct
  REGION 1..9: note = Description of Artificial Sequence: Synthetic peptide
QQYGSSTGT

SEQ ID NO: 573 - protein, 127 aa, synthetic construct
  REGION 1..127: note = Description of Artificial Sequence: Synthetic polypeptide
QVQLQESGPGLVKPSQTLSLTCTVSGGSISSSGYYWSWIRQHPGKGLEWIGYINYIGGTYYNPSLRSRVTMSVDTSKNQFSLRLSSVSAADTAVYYCASTHSYGDYSRDYYYGVDVWGQGTTVTISS

SEQ ID NO: 574 - protein, 10 aa, synthetic construct
  REGION 1..10: note = Description of Artificial Sequence: Synthetic peptide
GGSISSSGYY

SEQ ID NO: 575 - protein, 7 aa, synthetic construct
  REGION 1..7: note = Description of Artificial Sequence: Synthetic peptide
INYIGGT

SEQ ID NO: 576 - protein, 19 aa, synthetic construct
  REGION 1..19: note = Description of Artificial Sequence: Synthetic peptide
ASTHSYGDYSRDYYYGVDV

SEQ ID NO: 577 - protein, 108 aa, synthetic construct
  REGION 1..108: note = Description of Artificial Sequence: Synthetic polypeptide
EIVLTQSPATLSLSPGDRATLSCRTSQSVSSSYLAWYQQKPGQAPRLLIYAASSRATGIPDRFSGSGSGTDFTLTISRLEPEDFAVYYCQQCAGSPFTFGPGTKVDLK

SEQ ID NO: 578 - protein, 9 aa, synthetic construct
  REGION 1..9: note = Description of Artificial Sequence: Synthetic peptide
QQCAGSPFT

SEQ ID NO: 579 - protein, 123 aa, synthetic construct
  REGION 1..123: note = Description of Artificial Sequence: Synthetic polypeptide
QVQLVESGGGVVQPGRSLRLSCAASGFTFSDYAMHWVRQAPGKGLEWVAVISYDGNHRYYADSVKGRFTISRDNSKNTLYLQMNSLRAEDTAVYYCARHPGLSIAVAGPFDYWGQGTLVTVSS

SEQ ID NO: 580 - protein, 8 aa, synthetic construct
  REGION 1..8: note = Description of Artificial Sequence: Synthetic peptide
GFTFSDYA

SEQ ID NO: 581 - protein, 8 aa, synthetic construct
  REGION 1..8: note = Description of Artificial Sequence: Synthetic peptide
ISYDGNHR

SEQ ID NO: 582 - protein, 16 aa, synthetic construct
  REGION 1..16: note = Description of Artificial Sequence: Synthetic peptide
ARHPGLSIAVAGPFDY

SEQ ID NO: 583 - protein, 107 aa, synthetic construct
  REGION 1..107: note = Description of Artificial Sequence: Synthetic polypeptide
EIVMTQSPATLSVSPGERATLSCGASQSVSSNLAWYQQKPGQAPRLLFYGASTRATGIPARFSGSGSGTEFTLTISSLQSEDFALYYCQQYNNWPWTFGQGTKVDIK

SEQ ID NO: 584 - protein, 9 aa, synthetic construct
  REGION 1..9: note = Description of Artificial Sequence: Synthetic peptide
QQYNNWPWT

SEQ ID NO: 585 - protein, 123 aa, synthetic construct
  REGION 1..123: note = Description of Artificial Sequence: Synthetic polypeptide
QVQLVQSGAEVKKPGASVKVSCKASGYTFNRDGITWVRQAPGQGLEWMGWISANNDFTDYAQKFQGRLTMTTDTSTNTAYMELRSLRSDDTAVYYCARQVITVLQYSYGMDVWGQGTTVTVSS

SEQ ID NO: 586 - protein, 8 aa, synthetic construct
  REGION 1..8: note = Description of Artificial Sequence: Synthetic peptide
GYTFNRDG

SEQ ID NO: 587 - protein, 8 aa, synthetic construct
  REGION 1..8: note = Description of Artificial Sequence: Synthetic peptide
ISANNDFT

SEQ ID NO: 588 - protein, 16 aa, synthetic construct
  REGION 1..16: note = Description of Artificial Sequence: Synthetic peptide
ARQVITVLQYSYGMDV

SEQ ID NO: 589 - protein, 107 aa, synthetic construct
  REGION 1..107: note = Description of Artificial Sequence: Synthetic polypeptide
DIQMTQFPSSLSASVGDRVTITCRASQSISRYLNWYQQTPGKAPKLLIYGASSLQSGVPSRFSGSGSGTDFTLTISSLQPEDFATYYCQQSDTAPLTFGGGTRVEIK

SEQ ID NO: 590 - protein, 6 aa, synthetic construct
  REGION 1..6: note = Description of Artificial Sequence: Synthetic peptide
QSISRY

SEQ ID NO: 591 - protein, 9 aa, synthetic construct
  REGION 1..9: note = Description of Artificial Sequence: Synthetic peptide
QQSDTAPLT

SEQ ID NO: 592 - protein, 122 aa, synthetic construct
  REGION 1..122: note = Description of Artificial Sequence: Synthetic polypeptide
QVQLVESGGGLVTPGGSLRLSCTVSGFTLSDYYMSWIRQAPGKGLDWLSYISGSGDNKNYADSVRGRFTISRDNSKNSLYLQMNSLRAEDTAVYYCAREFPSGGYSPGVVLWGQGTLVTVSS

SEQ ID NO: 593 - protein, 8 aa, synthetic construct
  REGION 1..8: note = Description of Artificial Sequence: Synthetic peptide
GFTLSDYY

SEQ ID NO: 594 - protein, 8 aa, synthetic construct
  REGION 1..8: note = Description of Artificial Sequence: Synthetic peptide
ISGSGDNK

SEQ ID NO: 595 - protein, 15 aa, synthetic construct
  REGION 1..15: note = Description of Artificial Sequence: Synthetic peptide
AREFPSGGYSPGVVL

SEQ ID NO: 596 - protein, 111 aa, synthetic construct
  REGION 1..111: note = Description of Artificial Sequence: Synthetic polypeptide
NFVLTQPHSVSESPGKTVTISCARSSGSIAGSFVQWYQQRPGSSPTTVIYEDTRRPSGVPDRFSGSIDSSSNSASLTISGLKTEDEADYYCQSYDSTNPWVFGGGTKLTVL

SEQ ID NO: 597 - protein, 8 aa, synthetic construct
  REGION 1..8: note = Description of Artificial Sequence: Synthetic peptide
SGSIAGSF

SEQ ID NO: 598 - skipped

SEQ ID NO: 599 - protein, 10 aa, synthetic construct
  REGION 1..10: note = Description of Artificial Sequence: Synthetic peptide
QSYDSTNPWV

SEQ ID NO: 600 - protein, 122 aa, synthetic construct
  REGION 1..122: note = Description of Artificial Sequence: Synthetic polypeptide
EVQLLESGGGLVQPGGSLRLSCAASGFTFSSYAMSWVRQAPGKGLEWVSVIYSGGSRTYYADSAKGRFTISRDNSKNMLYLQMNSLRAEDTAVYYCTKNDYDSSGYFDFDNWGQGTLVTVSS

SEQ ID NO: 601 - protein, 15 aa, synthetic construct
  REGION 1..15: note = Description of Artificial Sequence: Synthetic peptide
TKNDYDSSGYFDFDN

SEQ ID NO: 602 - protein, 110 aa, synthetic construct
  REGION 1..110: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYHCSSYTSSSTWVFGGGTKLTVL

SEQ ID NO: 603 - protein, 9 aa, synthetic construct
  REGION 1..9: note = Description of Artificial Sequence: Synthetic peptide
SSDVGGYNY

SEQ ID NO: 604 - protein, 10 aa, synthetic construct
  REGION 1..10: note = Description of Artificial Sequence: Synthetic peptide
SSYTSSSTWV

SEQ ID NO: 605 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKGLEWVSVVYSGGSGTYYADSVKGRFTISRDDSTNTLYLQMNSLRAEDTAVYYCAKDRDSFGELDLDSWGQGTLVSVSS

SEQ ID NO: 606 - protein, 8 aa, synthetic construct
  REGION 1..8: note = Description of Artificial Sequence: Synthetic peptide
VYSGGSGT

SEQ ID NO: 607 - protein, 14 aa, synthetic construct
  REGION 1..14: note = Description of Artificial Sequence: Synthetic peptide
AKDRDSFGELDLDS

SEQ ID NO: 608 - protein, 113 aa, synthetic construct
  REGION 1..113: note = Description of Artificial Sequence: Synthetic polypeptide
DIVMTQSPDSLAVSLGERATINCKSSQSVLYSSKNKNYLAWYQQRPGQPPKLLIYWASTRESGVPDRFSGSGSGTDFTLTISSLQAEDVAVYYCLQYYNIPRTFGQGTKLEIK

SEQ ID NO: 609 - protein, 12 aa, synthetic construct
  REGION 1..12: note = Description of Artificial Sequence: Synthetic peptide
QSVLYSSKNKNY

SEQ ID NO: 610 - protein, 9 aa, synthetic construct
  REGION 1..9: note = Description of Artificial Sequence: Synthetic peptide
LQYYNIPRT

SEQ ID NO: 611 - protein, 122 aa, synthetic construct
  REGION 1..122: note = Description of Artificial Sequence: Synthetic polypeptide
EVQLLESGGGLVQPGGSLRLSCAASGFTFSSYALSWVRQPPGKGLEWVSVIYSGGSRTYYADAAKGRFTISRDNSKNMLYLQMNSLRAEDTAVYYCTKHDYDSSGYFDFDNWGQGTLVTVSS

SEQ ID NO: 612 - protein, 15 aa, synthetic construct
  REGION 1..15: note = Description of Artificial Sequence: Synthetic peptide
TKHDYDSSGYFDFDN

SEQ ID NO: 613 - protein, 120 aa, synthetic construct
  REGION 1..120: note = Description of Artificial Sequence: Synthetic polypeptide
EVQLVESGGGVARPGGSLRLSCAASGFTFDDYGMSWVRQAPGKGLEWVSGINWNGGSTNYADSVKGRFIISRDNGKNSLYLQMNSLRAEDTAFYHCARRGNFYYYGMDVWGQGTTVTVSS

SEQ ID NO: 614 - protein, 8 aa, synthetic construct
  REGION 1..8: note = Description of Artificial Sequence: Synthetic peptide
GFTFDDYG

SEQ ID NO: 615 - protein, 8 aa, synthetic construct
  REGION 1..8: note = Description of Artificial Sequence: Synthetic peptide
INWNGGST

SEQ ID NO: 616 - protein, 13 aa, synthetic construct
  REGION 1..13: note = Description of Artificial Sequence: Synthetic peptide
ARRGNFYYYGMDV

SEQ ID NO: 617 - protein, 107 aa, synthetic construct
  REGION 1..107: note = Description of Artificial Sequence: Synthetic polypeptide
DIQMTQSPSSVSASVGDRVTITCRASQGNSTWLAWYQQKPGKAPELLIFDASNLQSGVPSRFSGSGSGTDFTLTISSLQPEDFATYYCQQAQRFPLTFGGGTKVEIK

SEQ ID NO: 618 - protein, 6 aa, synthetic construct
  REGION 1..6: note = Description of Artificial Sequence: Synthetic peptide
QGNSTW

SEQ ID NO: 619 - protein, 9 aa, synthetic construct
  REGION 1..9: note = Description of Artificial Sequence: Synthetic peptide
QQAQRFPLT

SEQ ID NO: 620 - protein, 115 aa, synthetic construct
  REGION 1..115: note = Description of Artificial Sequence: Synthetic polypeptide
QVQLVQSGSELRRPGASVKVSCKTSGYAFTHFAMNWLRQAPGQGLEWLGWINTHSGNPTYAQGFTGRIVFSLDTSAGTAYLEISSLKAEDTAVYYCARERYFDFWGQGTLVAVSS

SEQ ID NO: 621 - protein, 8 aa, synthetic construct
  REGION 1..8: note = Description of Artificial Sequence: Synthetic peptide
GYAFTHFA

SEQ ID NO: 622 - protein, 110 aa, synthetic construct
  REGION 1..110: note = Description of Artificial Sequence: Synthetic polypeptide
QSVLTQPPSASGTPGQRVTISCSGTNSNIGKNFLYWYQQLPGTAPKLLIFSSNQRPSGVPDRFSGSKSGTSASLAISGLRSEDEADYYCAAWDDNLSGWVFGGGTKVTVL

SEQ ID NO: 623 - protein, 8 aa, synthetic construct
  REGION 1..8: note = Description of Artificial Sequence: Synthetic peptide
NSNIGKNF

SEQ ID NO: 624 - protein, 11 aa, synthetic construct
  REGION 1..11: note = Description of Artificial Sequence: Synthetic peptide
AAWDDNLSGWV

SEQ ID NO: 625 - protein, 127 aa, synthetic construct
  REGION 1..127: note = Description of Artificial Sequence: Synthetic polypeptide
QVQLQESGPGLVKPSEALSLTCSVSDGSVSSGSYYWTWIRQPPGKGLEWIGCIHYSGRTNYNPSLKSRVTISIDTSKNQFSLQLSSVTAVDTAVYYCARDRGEYDFWRVRYYGMDVWGQGTTVTVSS

SEQ ID NO: 626 - protein, 10 aa, synthetic construct
  REGION 1..10: note = Description of Artificial Sequence: Synthetic peptide
DGSVSSGSYY

SEQ ID NO: 627 - protein, 7 aa, synthetic construct
  REGION 1..7: note = Description of Artificial Sequence: Synthetic peptide
IHYSGRT

SEQ ID NO: 628 - protein, 19 aa, synthetic construct
  REGION 1..19: note = Description of Artificial Sequence: Synthetic peptide
ARDRGEYDFWRVRYYGMDV

SEQ ID NO: 629 - protein, 110 aa, synthetic construct
  REGION 1..110: note = Description of Artificial Sequence: Synthetic polypeptide
QSALTQPASVSGSPGQSITISCTGTSSDVGDYNYVSWYQQHPGKAPKLLIYDFSNRPSGVSDRFSGSKSGNTASLTISGLRAEDESDYYCTSYTNTNTRLFGGGTKLTVL

SEQ ID NO: 630 - protein, 9 aa, synthetic construct
  REGION 1..9: note = Description of Artificial Sequence: Synthetic peptide
SSDVGDYNY

SEQ ID NO: 631 - skipped

SEQ ID NO: 632 - protein, 10 aa, synthetic construct
  REGION 1..10: note = Description of Artificial Sequence: Synthetic peptide
TSYTNTNTRL

SEQ ID NO: 633 - protein, 120 aa, synthetic construct
  REGION 1..120: note = Description of Artificial Sequence: Synthetic polypeptide
QVQLVQSGAEVKKPGASVKVSCKASGYTFISYGLHWVRQAPGQRPEWMGWINAGNGNRKYSERFQARVTFTRDTSATTAYMELSSLRSEDTAVYYCARDRLTAAAHFDYWGQGTQVTVSS

SEQ ID NO: 634 - protein, 8 aa, synthetic construct
  REGION 1..8: note = Description of Artificial Sequence: Synthetic peptide
GYTFISYG

SEQ ID NO: 635 - protein, 8 aa, synthetic construct
  REGION 1..8: note = Description of Artificial Sequence: Synthetic peptide
INAGNGNR

SEQ ID NO: 636 - protein, 13 aa, synthetic construct
  REGION 1..13: note = Description of Artificial Sequence: Synthetic peptide
ARDRLTAAAHFDY

SEQ ID NO: 637 - protein, 107 aa, synthetic construct
  REGION 1..107: note = Description of Artificial Sequence: Synthetic polypeptide
EIVMTQSPATLSVSLGERATLSCRASQSVSSDLAWYQQKPGQAPRLLMYGASTRATGFPARFTGSGSGPEFTLTISSLQSEDFAVYYCQQYNNWPFTFGGGTKVEIK

SEQ ID NO: 638 - protein, 6 aa, synthetic construct
  REGION 1..6: note = Description of Artificial Sequence: Synthetic peptide
QSVSSD

SEQ ID NO: 639 - protein, 9 aa, synthetic construct
  REGION 1..9: note = Description of Artificial Sequence: Synthetic peptide
QQYNNWPFT

SEQ ID NO: 640 - protein, 127 aa, synthetic construct
  REGION 1..127: note = Description of Artificial Sequence: Synthetic polypeptide
QITLKESGPTLVKPTQTLTLTCTFSGFSLTSSAVGVGWIRQPPGKALEWLALIYGDDDKRYSPSLKRRLTITKDTSKNQVVLTMTDVDPVDTATYYCAHRRLTIPLLMVAADAFDIWGPGTMVIVSS

SEQ ID NO: 641 - protein, 10 aa, synthetic construct
  REGION 1..10: note = Description of Artificial Sequence: Synthetic peptide
GFSLTSSAVG

SEQ ID NO: 642 - protein, 7 aa, synthetic construct
  REGION 1..7: note = Description of Artificial Sequence: Synthetic peptide
IYGDDDK

SEQ ID NO: 643 - protein, 19 aa, synthetic construct
  REGION 1..19: note = Description of Artificial Sequence: Synthetic peptide
AHRRLTIPLLMVAADAFDI

SEQ ID NO: 644 - protein, 106 aa, synthetic construct
  REGION 1..106: note = Description of Artificial Sequence: Synthetic polypeptide
DIQMTQSPSTLSASVGDRVTITCRASQSVSRWLAWYQQKPGKAPKLLIYRASSLQSGVPSRFSGSGSGTEFTLTISSLQPDDFATYYCQQYSSFHTFGQGTKLEIK

SEQ ID NO: 645 - protein, 6 aa, synthetic construct
  REGION 1..6: note = Description of Artificial Sequence: Synthetic peptide
QSVSRW

SEQ ID NO: 646 - skipped

SEQ ID NO: 647 - protein, 8 aa, synthetic construct
  REGION 1..8: note = Description of Artificial Sequence: Synthetic peptide
QQYSSFHT

SEQ ID NO: 648 - protein, 120 aa, synthetic construct
  REGION 1..120: note = Description of Artificial Sequence: Synthetic polypeptide
EVQLVESGGGLVQPGGSLRLSCAASGFIFSTYSMNWVRQAPGKGLEWVSYISSSSNTIYYADSVKGRFTISRDNAKNSLYLQMNSLRDADTAVYYCARDGGRSGYFDDYWGQGTLVTVSS

SEQ ID NO: 649 - protein, 8 aa, synthetic construct
  REGION 1..8: note = Description of Artificial Sequence: Synthetic peptide
GFIFSTYS

SEQ ID NO: 650 - protein, 8 aa, synthetic construct
  REGION 1..8: note = Description of Artificial Sequence: Synthetic peptide
ISSSSNTI

SEQ ID NO: 651 - protein, 13 aa, synthetic construct
  REGION 1..13: note = Description of Artificial Sequence: Synthetic peptide
ARDGGRSGYFDDY

SEQ ID NO: 652 - protein, 111 aa, synthetic construct
  REGION 1..111: note = Description of Artificial Sequence: Synthetic polypeptide
QLVLTQSPSASASLGASVKLTCTLSNGHINYAIAWHQQQPDKGPRYLLNLKSDGSHSKGDGIPDRFSGSSSGAERYLTISGLQSEDEADYYCQTWGTGIQVFGGGTKLTVL

SEQ ID NO: 653 - protein, 7 aa, synthetic construct
  REGION 1..7: note = Description of Artificial Sequence: Synthetic peptide
NGHINYA

SEQ ID NO: 654 - protein, 7 aa, synthetic construct
  REGION 1..7: note = Description of Artificial Sequence: Synthetic peptide
LKSDGSH

SEQ ID NO: 655 - protein, 9 aa, synthetic construct
  REGION 1..9: note = Description of Artificial Sequence: Synthetic peptide
QTWGTGIQV

SEQ ID NO: 656 - protein, 122 aa, synthetic construct
  REGION 1..122: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRGVWFGELFPFDYWGQGTLGTVSS

SEQ ID NO: 657 - protein, 8 aa, synthetic construct
  REGION 1..8: note = Description of Artificial Sequence: Synthetic peptide
GGSFSGYY

SEQ ID NO: 658 - protein, 7 aa, synthetic construct
  REGION 1..7: note = Description of Artificial Sequence: Synthetic peptide
INHSGST

SEQ ID NO: 659 - protein, 16 aa, synthetic construct
  REGION 1..16: note = Description of Artificial Sequence: Synthetic peptide
ARGRGVWFGELFPFDY

SEQ ID NO: 660 - protein, 110 aa, synthetic construct
  REGION 1..110: note = Description of Artificial Sequence: Synthetic polypeptide
QGGLTQPPSVSKGLRQTATLTCTGNSNNVGNQGAAWLQQHQGHPPKLLSYRNNNRPSGISERFSASRSGNTASLTITGLQPEDEADYYCSAWDSSLSAVVFGGGTKLTVL

SEQ ID NO: 661 - protein, 8 aa, synthetic construct
  REGION 1..8: note = Description of Artificial Sequence: Synthetic peptide
SNNVGNQG

SEQ ID NO: 662 - skipped

SEQ ID NO: 663 - protein, 11 aa, synthetic construct
  REGION 1..11: note = Description of Artificial Sequence: Synthetic peptide
SAWDSSLSAVV

SEQ ID NO: 664 - protein, 124 aa, synthetic construct
  REGION 1..124: note = Description of Artificial Sequence: Synthetic polypeptide
QVQLQQWGAGLLKPSETLSLNCTVYHGSLSTSYWSWIRQPPGRGLEWIGEINDSGATNYNPSLKSRVIISVDTSKDQFSLKLTSVTAADTAMYYCARAPLLWVGESFFYYFDSWGQGILVTVSS

SEQ ID NO: 665 - protein, 8 aa, synthetic construct
  REGION 1..8: note = Description of Artificial Sequence: Synthetic peptide
HGSLSTSY

SEQ ID NO: 666 - protein, 7 aa, synthetic construct
  REGION 1..7: note = Description of Artificial Sequence: Synthetic peptide
INDSGAT

SEQ ID NO: 667 - protein, 18 aa, synthetic construct
  REGION 1..18: note = Description of Artificial Sequence: Synthetic peptide
ARAPLLWVGESFFYYFDS

SEQ ID NO: 668 - protein, 107 aa, synthetic construct
  REGION 1..107: note = Description of Artificial Sequence: Synthetic polypeptide
DIQMTQSPSSLSASVGDRVSITCRAGQSIDTYLNWYQHKPGKAPDLLIYTTSTLHSGVPSRFSGSGSGTDFTLTITSLQPEDFAIYYCQQSYKSPYTFGQGTKVEIK

SEQ ID NO: 669 - protein, 6 aa, synthetic construct
  REGION 1..6: note = Description of Artificial Sequence: Synthetic peptide
QSIDTY

SEQ ID NO: 670 - skipped

SEQ ID NO: 671 - protein, 9 aa, synthetic construct
  REGION 1..9: note = Description of Artificial Sequence: Synthetic peptide
QQSYKSPYT

SEQ ID NO: 672 - protein, 121 aa, synthetic construct
  REGION 1..121: note = Description of Artificial Sequence: Synthetic polypeptide
QVQLVQSGAEVKKPGASVKVSCKASGYTFTSYYMHWVRQAPGQGLEWMGIINPSGGTTSSAQKFQGRVTMTRDTSTSTVYMELSSLRSEDTALYYCARDREQKVGGAPLHWGQGTLVTVSS

SEQ ID NO: 673 - protein, 8 aa, synthetic construct
  REGION 1..8: note = Description of Artificial Sequence: Synthetic peptide
INPSGGTT

SEQ ID NO: 674 - protein, 14 aa, synthetic construct
  REGION 1..14: note = Description of Artificial Sequence: Synthetic peptide
ARDREQKVGGAPLH

SEQ ID NO: 675 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYDASNLETGVPSRFSGSGSGTDFSFTISSLQPEDIATYYCQQYDNFALTFGGGTKVEIK

SEQ ID NO: 676 - protein, 9 aa, synthetic construct
  REGION 1..9: note = Description of Artificial Sequence: Synthetic peptide
QQYDNFALT

SEQ ID NO: 677 - protein, 122 aa, synthetic construct
  REGION 1..122: note = Description of Artificial Sequence: Synthetic polypeptide
EVQLLESGGGLVQPGVSLRLSCAASGFTFSSYAMSWVRQAPGKGLEWVSVIYSGGNIIYYADSVKGRFTISRDNSKNTLYLQIDNLRAEDTALYYCAKHDYDSSGYFDFDYWGHGTLVTVSS

SEQ ID NO: 678 - protein, 8 aa, synthetic construct
  REGION 1..8: note = Description of Artificial Sequence: Synthetic peptide
IYSGGNII

SEQ ID NO: 679 - protein, 15 aa, synthetic construct
  REGION 1..15: note = Description of Artificial Sequence: Synthetic peptide
AKHDYDSSGYFDFDY

SEQ ID NO: 680 - protein, 110 aa, synthetic construct
  REGION 1..110: note = Description of Artificial Sequence: Synthetic polypeptide
QSALTQPASVSGSPGQSITISCTGTSRDVGGYNYVSWYQQHPGKAPKLMIYDVNNRPSGVSNRFSGSKSGNTASLTISGLQAEDEADYFCCSYTSSSTWVFGGGTKLTVL

SEQ ID NO: 681 - protein, 9 aa, synthetic construct
  REGION 1..9: note = Description of Artificial Sequence: Synthetic peptide
SRDVGGYNY

SEQ ID NO: 682 - skipped

SEQ ID NO: 683 - protein, 10 aa, synthetic construct
  REGION 1..10: note = Description of Artificial Sequence: Synthetic peptide
CSYTSSSTWV

SEQ ID NO: 684 - protein, 125 aa, synthetic construct
  REGION 1..125: note = Description of Artificial Sequence: Synthetic polypeptide
QVQLQESGPGLVKPSGTLSLTCAVSGASISNSAWWNWVRQPPRGGLEWVGEIYPSGSTNYTPSLKSRATILLDESRNEFSLKLNSVTAADTAVYYCARGRLEDCNGGVCYFFDNWGQGILVSVSS

SEQ ID NO: 685 - protein, 9 aa, synthetic construct
  REGION 1..9: note = Description of Artificial Sequence: Synthetic peptide
GASISNSAW

SEQ ID NO: 686 - protein, 7 aa, synthetic construct
  REGION 1..7: note = Description of Artificial Sequence: Synthetic peptide
IYPSGST

SEQ ID NO: 687 - protein, 18 aa, synthetic construct
  REGION 1..18: note = Description of Artificial Sequence: Synthetic peptide
ARGRLEDCNGGVCYFFDN

SEQ ID NO: 688 - protein, 107 aa, synthetic construct
  REGION 1..107: note = Description of Artificial Sequence: Synthetic polypeptide
DIEMTQSPSTLSASVGDRVTITCRANYGIGAWLAWYQQKPGKAPKLLIYKASTLESGVPLRFSGSGSGTEFTLSISGLQPDDFATYYCHQYSTYPITFGQGTRLEIK

SEQ ID NO: 689 - protein, 6 aa, synthetic construct
  REGION 1..6: note = Description of Artificial Sequence: Synthetic peptide
YGIGAW

SEQ ID NO: 690 - protein, 9 aa, synthetic construct
  REGION 1..9: note = Description of Artificial Sequence: Synthetic peptide
HQYSTYPIT

SEQ ID NO: 691 - protein, 122 aa, synthetic construct
  REGION 1..122: note = Description of Artificial Sequence: Synthetic polypeptide
QVQLVESGGGVVQPGRSLTLSCAASGFNFKTYGMHWVRQAPGKGLEWVAVIYHDGNDKFYADSVKGRFTISRDNSKNTLYVQMSSLRADDTAIYYCAKGIFSSGYHYGMDVWGQGTAVIVSS

SEQ ID NO: 692 - protein, 8 aa, synthetic construct
  REGION 1..8: note = Description of Artificial Sequence: Synthetic peptide
GFNFKTYG

SEQ ID NO: 693 - protein, 8 aa, synthetic construct
  REGION 1..8: note = Description of Artificial Sequence: Synthetic peptide
IYHDGNDK

SEQ ID NO: 694 - protein, 15 aa, synthetic construct
  REGION 1..15: note = Description of Artificial Sequence: Synthetic peptide
AKGIFSSGYHYGMDV

SEQ ID NO: 695 - protein, 107 aa, synthetic construct
  REGION 1..107: note = Description of Artificial Sequence: Synthetic polypeptide
DIQMTQSPSSLSASLGDSVTITCLASQGIKEFLSWFQQKPGQAPKLLIYDASSSHSGVPSRFSGSGSATHFTLTISSLQPDDIATYYCQQYHQVPLTFGQGTRLEIK

SEQ ID NO: 696 - protein, 6 aa, synthetic construct
  REGION 1..6: note = Description of Artificial Sequence: Synthetic peptide
QGIKEF

SEQ ID NO: 697 - protein, 9 aa, synthetic construct
  REGION 1..9: note = Description of Artificial Sequence: Synthetic peptide
QQYHQVPLT

SEQ ID NO: 698 - protein, 119 aa, synthetic construct
  REGION 1..119: note = Description of Artificial Sequence: Synthetic polypeptide
EVRLVESGGGLIQPGGSLRLSCAASGFNVSSDYMNWVRQAPGKGLEWVSVLYSSGFTYYADSVRGRFTISRDNSKNTLYLQMNSLRAEDTAVYYCARVALFGEPLVDSWGQGTLVTVSS

SEQ ID NO: 699 - protein, 8 aa, synthetic construct
  REGION 1..8: note = Description of Artificial Sequence: Synthetic peptide
GFNVSSDY

SEQ ID NO: 700 - protein, 7 aa, synthetic construct
  REGION 1..7: note = Description of Artificial Sequence: Synthetic peptide
LYSSGFT

SEQ ID NO: 701 - protein, 13 aa, synthetic construct
  REGION 1..13: note = Description of Artificial Sequence: Synthetic peptide
ARVALFGEPLVDS

SEQ ID NO: 702 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGAISRATGIPARFSGSGSGTEFTLTISSLQSEDFAIYYCQQYNNWPWTFGQGTKVEIK

SEQ ID NO: 703 - skipped

SEQ ID NO: 704 - protein, 6 aa, Arachis hypogaea
DPYSPS

SEQ ID NO: 705 - protein, 11 aa, Arachis hypogaea
DSYGRDPYSPS

SEQ ID NO: 706 - protein, 11 aa, Arachis hypogaea
YSPSQDPYSPS

SEQ ID NO: 707 - protein, 10 aa, Arachis hypogaea
PDRRDPYSPS

SEQ ID NO: 708 - protein, 151 aa, Arachis hypogaea
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 709 - protein, 121 aa, synthetic construct
  REGION 1..121: note = Description of Artificial Sequence: Synthetic polypeptide
EVQLVASGGGLIHPGGSLRLSCEASGFSFSRFWMYWVRQSPGEGLVWVARLSGDGTVTNYADSMEGRVTISRDNVKNTLFLEMNSLREGDTGIYYCARKDCPSLSCQLDYWGQGVQVTVSS

SEQ ID NO: 710 - protein, 8 aa, synthetic construct
  REGION 1..8: note = Description of Artificial Sequence: Synthetic peptide
GFSFSRFW

SEQ ID NO: 711 - protein, 8 aa, synthetic construct
  REGION 1..8: note = Description of Artificial Sequence: Synthetic peptide
LSGDGTVT

SEQ ID NO: 712 - protein, 14 aa, synthetic construct
  REGION 1..14: note = Description of Artificial Sequence: Synthetic peptide
ARKDCPSLSCQLDY

SEQ ID NO: 713 - protein, 110 aa, synthetic construct
  REGION 1..110: note = Description of Artificial Sequence: Synthetic polypeptide
QSVLTQPPSVSAAPGQKVTISCSGSTSNIGKNYVSWYQHFPGAAPKLLIFDNDKRPSGIPDRFSGSRSGTSATLDITGLQTGDEADYFCATWDSRLSADVFGSGTTVSVL

SEQ ID NO: 714 - protein, 8 aa, synthetic construct
  REGION 1..8: note = Description of Artificial Sequence: Synthetic peptide
TSNIGKNY

SEQ ID NO: 715 - skipped

SEQ ID NO: 716 - protein, 11 aa, synthetic construct
  REGION 1..11: note = Description of Artificial Sequence: Synthetic peptide
ATWDSRLSADV

SEQ ID NO: 717 - protein, 123 aa, synthetic construct
  REGION 1..123: note = Description of Artificial Sequence: Synthetic polypeptide
EVQLLESGGGLVQPGGSLRLSCAASGFNFSNFAVSWVRQTPGKGLEWVSAILGSRSVTYYADSVKGRFTISRDKSKNALYLQMDSLRAEDTAIYYCAKLFFMPYSHDDSGDYWGQGTLVAVSS

SEQ ID NO: 718 - protein, 8 aa, synthetic construct
  REGION 1..8: note = Description of Artificial Sequence: Synthetic peptide
GFNFSNFA

SEQ ID NO: 719 - protein, 8 aa, synthetic construct
  REGION 1..8: note = Description of Artificial Sequence: Synthetic peptide
ILGSRSVT

SEQ ID NO: 720 - protein, 16 aa, synthetic construct
  REGION 1..16: note = Description of Artificial Sequence: Synthetic peptide
AKLFFMPYSHDDSGDY

SEQ ID NO: 721 - protein, 111 aa, synthetic construct
  REGION 1..111: note = Description of Artificial Sequence: Synthetic polypeptide
QLVLTQSPSASASLGASVKLTCTLSSDHRSYAIAWHQQQPGKGPRYLMKVNRDGSHIKGDGIPHRFSGSSSVTERYLIISSLQSEDEADYYCQSWDTGIQVFGGGTRLTVV

SEQ ID NO: 722 - protein, 7 aa, synthetic construct
  REGION 1..7: note = Description of Artificial Sequence: Synthetic peptide
SDHRSYA

SEQ ID NO: 723 - protein, 7 aa, synthetic construct
  REGION 1..7: note = Description of Artificial Sequence: Synthetic peptide
VNRDGSH

SEQ ID NO: 724 - protein, 9 aa, synthetic construct
  REGION 1..9: note = Description of Artificial Sequence: Synthetic peptide
QSWDTGIQV

SEQ ID NO: 725 - protein, 121 aa, synthetic construct
  REGION 1..121: note = Description of Artificial Sequence: Synthetic polypeptide
QVQLQESGLGLVKPSGTLSLTCAVSGGPMNSSYWWSWVRQSPGGGLEWIGQISHYTNTKYNPSFKNRVSISIDKSKNEFSLRLTYVTGADTGVYYCVGERDWKDPNWFDPWGQGRLVTVSS

SEQ ID NO: 726 - protein, 9 aa, synthetic construct
  REGION 1..9: note = Description of Artificial Sequence: Synthetic peptide
GGPMNSSYW

SEQ ID NO: 727 - protein, 7 aa, synthetic construct
  REGION 1..7: note = Description of Artificial Sequence: Synthetic peptide
ISHYTNT

SEQ ID NO: 728 - protein, 14 aa, synthetic construct
  REGION 1..14: note = Description of Artificial Sequence: Synthetic peptide
VGERDWKDPNWFDP

SEQ ID NO: 729 - protein, 111 aa, synthetic construct
  REGION 1..111: note = Description of Artificial Sequence: Synthetic polypeptide
QSVLTQPPSVSGAPGQRVTISCTGSNSNIGAGQDVHWYQHFPGTAPKLVIYGNSNRPSGVPDRFSGSKSGTSASLAISGLQADDEADYYCQSYDKSLSSSLFGGGTKLTVL

SEQ ID NO: 730 - protein, 9 aa, synthetic construct
  REGION 1..9: note = Description of Artificial Sequence: Synthetic peptide
NSNIGAGQD

SEQ ID NO: 731 - skipped

SEQ ID NO: 732 - protein, 11 aa, synthetic construct
  REGION 1..11: note = Description of Artificial Sequence: Synthetic peptide
QSYDKSLSSSL

SEQ ID NO: 733 - protein, 120 aa, synthetic construct
  REGION 1..120: note = Description of Artificial Sequence: Synthetic polypeptide
QVQLQESGPGLVKPSETLSLSCNVSGGSIRGHYWSWIRQSPGKRLEWLGYIYQSGYTKYNPSLKSRVSISLDTSKNKFSLNLKSVTTADTAVYYCAGRVAERGGDQFDFWGQGTLVTVSS

SEQ ID NO: 734 - protein, 8 aa, synthetic construct
  REGION 1..8: note = Description of Artificial Sequence: Synthetic peptide
GGSIRGHY

SEQ ID NO: 735 - protein, 7 aa, synthetic construct
  REGION 1..7: note = Description of Artificial Sequence: Synthetic peptide
IYQSGYT

SEQ ID NO: 736 - protein, 14 aa, synthetic construct
  REGION 1..14: note = Description of Artificial Sequence: Synthetic peptide
AGRVAERGGDQFDF

SEQ ID NO: 737 - protein, 108 aa, synthetic construct
  REGION 1..108: note = Description of Artificial Sequence: Synthetic polypeptide
SYELTQSPSLSVSPGQTASITCSGENLGEKHASWYQQKSGQSPVLVIYQDTKRPAGIPERFSGSNSGSTATLTISGTQPMDEADYFCQAWDANTANVIFGGGTMLTVL

SEQ ID NO: 738 - protein, 6 aa, synthetic construct
  REGION 1..6: note = Description of Artificial Sequence: Synthetic peptide
NLGEKH

SEQ ID NO: 739 - skipped

SEQ ID NO: 740 - protein, 11 aa, synthetic construct
  REGION 1..11: note = Description of Artificial Sequence: Synthetic peptide
QAWDANTANVI

SEQ ID NO: 741 - protein, 126 aa, synthetic construct
  REGION 1..126: note = Description of Artificial Sequence: Synthetic polypeptide
EVQLVESGGGLVQPGGSLKLSCAASGFTFSGSTIHWVRQTSGKGLEWVGRIGSKATSYATAYAASVKGRFTISRDDSKNTAYLQMNSLKTEDTAVYFCTRRYYDTTKSVLVVSDSWGQGTLVTVSS

SEQ ID NO: 742 - protein, 8 aa, synthetic construct
  REGION 1..8: note = Description of Artificial Sequence: Synthetic peptide
GFTFSGST

SEQ ID NO: 743 - protein, 10 aa, synthetic construct
  REGION 1..10: note = Description of Artificial Sequence: Synthetic peptide
IGSKATSYAT

SEQ ID NO: 744 - protein, 17 aa, synthetic construct
  REGION 1..17: note = Description of Artificial Sequence: Synthetic peptide
TRRYYDTTKSVLVVSDS

SEQ ID NO: 745 - protein, 108 aa, synthetic construct
  REGION 1..108: note = Description of Artificial Sequence: Synthetic polypeptide
SYELTQPPSMSVSPGQTARITCSGDVLAKQFAYWYQQKPGQAPVLVIYKDSERPSGIPERFSGSSSGTIITLTISGVQAEDEADYYCQSADSSGTSWVFGGGTKLTVL

SEQ ID NO: 746 - protein, 6 aa, synthetic construct
  REGION 1..6: note = Description of Artificial Sequence: Synthetic peptide
VLAKQF

SEQ ID NO: 747 - skipped

SEQ ID NO: 748 - protein, 11 aa, synthetic construct
  REGION 1..11: note = Description of Artificial Sequence: Synthetic peptide
QSADSSGTSWV

SEQ ID NO: 749 - protein, 130 aa, synthetic construct
  REGION 1..130: note = Description of Artificial Sequence: Synthetic polypeptide
QLLLQESGPGLVKPSETLSLSCTVSAGSITSINYSWGWIRQPPGKGLEWIASVYFSGSIYYNPSLKSRVAISVDTSKNTFSLNLTSVTAADTAVYYCARLRLDTGRDSSGLSYREHFDYWAQGTLVTVSS

SEQ ID NO: 750 - protein, 10 aa, synthetic construct
  REGION 1..10: note = Description of Artificial Sequence: Synthetic peptide
AGSITSINYS

SEQ ID NO: 751 - protein, 7 aa, synthetic construct
  REGION 1..7: note = Description of Artificial Sequence: Synthetic peptide
VYFSGSI

SEQ ID NO: 752 - protein, 22 aa, synthetic construct
  REGION 1..22: note = Description of Artificial Sequence: Synthetic peptide
ARLRLDTGRDSSGLSYREHFDY

SEQ ID NO: 753 - protein, 107 aa, synthetic construct
  REGION 1..107: note = Description of Artificial Sequence: Synthetic polypeptide
DIQMTQSPSTLSASVGDRVTITCRASQSIGMWLAWFQQKPGKAPKLLIYKASTLESGVPSRFSGSGSGTEFTLTINSLQPDDFATYYCQQYNSYLFTFGPGTKVDIK

SEQ ID NO: 754 - protein, 6 aa, synthetic construct
  REGION 1..6: note = Description of Artificial Sequence: Synthetic peptide
QSIGMW

SEQ ID NO: 755 - protein, 9 aa, synthetic construct
  REGION 1..9: note = Description of Artificial Sequence: Synthetic peptide
QQYNSYLFT

SEQ ID NO: 756 - protein, 122 aa, synthetic construct
  REGION 1..122: note = Description of Artificial Sequence: Synthetic polypeptide
EVQLVQSGAEVKKPGESLKISCKGSGYNFTSSWIGWVRQMPGKGLEWMGIIHPGDSDTRYSPSFQGQVTISADKSLTTAFLQWSSLKTSDTAIYYCARHGSTMLWGDAFDIWGQGTMVTVSS

SEQ ID NO: 757 - protein, 8 aa, synthetic construct
  REGION 1..8: note = Description of Artificial Sequence: Synthetic peptide
GYNFTSSW

SEQ ID NO: 758 - protein, 8 aa, synthetic construct
  REGION 1..8: note = Description of Artificial Sequence: Synthetic peptide
IHPGDSDT

SEQ ID NO: 759 - protein, 15 aa, synthetic construct
  REGION 1..15: note = Description of Artificial Sequence: Synthetic peptide
ARHGSTMLWGDAFDI

SEQ ID NO: 760 - protein, 106 aa, synthetic construct
  REGION 1..106: note = Description of Artificial Sequence: Synthetic polypeptide
SYELTQPPSVSLSPGQTARITCSGDALPKHYAYWYQQKPGQAPVLVIYKDTERPSGIPERFSGSSSGTTVTLTISGVQAEDEADYYCQSSDSTGEVFGGGTKLTVL

SEQ ID NO: 761 - protein, 6 aa, synthetic construct
  REGION 1..6: note = Description of Artificial Sequence: Synthetic peptide
ALPKHY

SEQ ID NO: 762 - skipped

SEQ ID NO: 763 - protein, 9 aa, synthetic construct
  REGION 1..9: note = Description of Artificial Sequence: Synthetic peptide
QSSDSTGEV

SEQ ID NO: 764 - protein, 120 aa, synthetic construct
  REGION 1..120: note = Description of Artificial Sequence: Synthetic polypeptide
QVQLVQSGAEVKKPGASVMLSCKASGYIFTNSDINWVRQAPGQGPEWMGWMNPKSGNTGYEQKFQGRVTMTTNTSISTAYMELSRLRSEDTAVYYCARSTGAVAGNFDYWGQGTPVTVSS

SEQ ID NO: 765 - protein, 8 aa, synthetic construct
  REGION 1..8: note = Description of Artificial Sequence: Synthetic peptide
GYIFTNSD

SEQ ID NO: 766 - protein, 8 aa, synthetic construct
  REGION 1..8: note = Description of Artificial Sequence: Synthetic peptide
MNPKSGNT

SEQ ID NO: 767 - protein, 13 aa, synthetic construct
  REGION 1..13: note = Description of Artificial Sequence: Synthetic peptide
ARSTGAVAGNFDY

SEQ ID NO: 768 - protein, 106 aa, synthetic construct
  REGION 1..106: note = Description of Artificial Sequence: Synthetic polypeptide
EIVMTQSPATLSVSLGDRATLSCRASQSISRNLAWYQQKPGQAPRLLIYGASIRITDIPARFSGSGSGTEFTLTISSLQSEDFAIYFCQQYNNWRTFGQGTRVELK

SEQ ID NO: 769 - protein, 6 aa, synthetic construct
  REGION 1..6: note = Description of Artificial Sequence: Synthetic peptide
QSISRN

SEQ ID NO: 770 - protein, 8 aa, synthetic construct
  REGION 1..8: note = Description of Artificial Sequence: Synthetic peptide
QQYNNWRT

SEQ ID NO: 771 - protein, 117 aa, synthetic construct
  REGION 1..117: note = Description of Artificial Sequence: Synthetic polypeptide
HVQLQESGPGLVKSSETLSLTCNVSSDSFSDHYWSWVRQPAGKGLQWLGRIYNTGTTTYNPSLNRRITMSVDTSKNQFSLRLTSVTAADTAVYYCAARHYHYDKTIWGQGTLVTVSS

SEQ ID NO: 772 - protein, 8 aa, synthetic construct
  REGION 1..8: note = Description of Artificial Sequence: Synthetic peptide
SDSFSDHY

SEQ ID NO: 773 - protein, 7 aa, synthetic construct
  REGION 1..7: note = Description of Artificial Sequence: Synthetic peptide
IYNTGTT

SEQ ID NO: 774 - protein, 11 aa, synthetic construct
  REGION 1..11: note = Description of Artificial Sequence: Synthetic peptide
AARHYHYDKTI

SEQ ID NO: 775 - protein, 111 aa, synthetic construct
  REGION 1..111: note = Description of Artificial Sequence: Synthetic polypeptide
NFMLTQPHSVSESPGKTVTISCTRSSGSIASNYVQWYQQRPGSPPTTVIYEDNQRPSGVPDRFSGSIDSSSNSASLTISGLKPEDEADFYCQSYDSDDREVFGGGTRLTVL

SEQ ID NO: 776 - protein, 8 aa, synthetic construct
  REGION 1..8: note = Description of Artificial Sequence: Synthetic peptide
SGSIASNY

SEQ ID NO: 777 - protein, 10 aa, synthetic construct
  REGION 1..10: note = Description of Artificial Sequence: Synthetic peptide
QSYDSDDREV

SEQ ID NO: 778 - protein, 130 aa, synthetic construct
  REGION 1..130: note = Description of Artificial Sequence: Synthetic polypeptide
QVQLVQSGAEVKKPGASVKVSCKASGYTFTTYGISWVRQAPGQGLEWMGWISAYNGNTTYAQNFHARVTMTTDTSTSTAYMELRSLRSDDTAVYFCARTSARTITIFGVLIPAGLNLDYWGQGTLVTVSS

SEQ ID NO: 779 - protein, 8 aa, synthetic construct
  REGION 1..8: note = Description of Artificial Sequence: Synthetic peptide
GYTFTTYG

SEQ ID NO: 780 - protein, 23 aa, synthetic construct
  REGION 1..23: note = Description of Artificial Sequence: Synthetic peptide
ARTSARTITIFGVLIPAGLNLDY

SEQ ID NO: 781 - protein, 110 aa, synthetic construct
  REGION 1..110: note = Description of Artificial Sequence: Synthetic polypeptide
QSALTQPASVSGSPGQSITISCTGTGSDVGRYNYVSWYQQHPGKAPKFMIYDVSNRPSGVSNRFSASKSGNTASLTISGLQAEDEADYYCSSYTSTSTLVFGGGTKLTVL

SEQ ID NO: 782 - protein, 9 aa, synthetic construct
  REGION 1..9: note = Description of Artificial Sequence: Synthetic peptide
GSDVGRYNY

SEQ ID NO: 783 - protein, 10 aa, synthetic construct
  REGION 1..10: note = Description of Artificial Sequence: Synthetic peptide
SSYTSTSTLV

SEQ ID NO: 784 - protein, 123 aa, synthetic construct
  REGION 1..123: note = Description of Artificial Sequence: Synthetic polypeptide
QVDLVESGGGYVKSGGSLRLSCAASGFRFSDYYMSWVRQAPGKGLEWLSHISSDSSDTNYADSVKGRFSISRDNAKNSVFLQMNTLRAEDTAVYYCARNALTNAYDMSGFRNWGQGILVTVSS

SEQ ID NO: 785 - protein, 8 aa, synthetic construct
  REGION 1..8: note = Description of Artificial Sequence: Synthetic peptide
GFRFSDYY

SEQ ID NO: 786 - protein, 8 aa, synthetic construct
  REGION 1..8: note = Description of Artificial Sequence: Synthetic peptide
ISSDSSDT

SEQ ID NO: 787 - protein, 16 aa, synthetic construct
  REGION 1..16: note = Description of Artificial Sequence: Synthetic peptide
ARNALTNAYDMSGFRN

SEQ ID NO: 788 - protein, 112 aa, synthetic construct
  REGION 1..112: note = Description of Artificial Sequence: Synthetic polypeptide
NFMLTQPHSVSESPGKTVILSCTRSSGSIATNYVRWYQQRPGSAPTTVIYEDSRRPSSVPDRFSGSIDSSSNSASLTISGLRTEDEADYYCQSFDTSSRKVVFGGGTKLTVL

SEQ ID NO: 789 - protein, 8 aa, synthetic construct
  REGION 1..8: note = Description of Artificial Sequence: Synthetic peptide
SGSIATNY

SEQ ID NO: 790 - skipped

SEQ ID NO: 791 - protein, 11 aa, synthetic construct
  REGION 1..11: note = Description of Artificial Sequence: Synthetic peptide
QSFDTSSRKVV

SEQ ID NO: 792 - protein, 123 aa, synthetic construct
  REGION 1..123: note = Description of Artificial Sequence: Synthetic polypeptide
QVTLRESGPALVEVTQTVTLTCNFSGFSLHTRGMYVNWIRQPPGKALEWLAVINWDDDKYYTPSLKNRLTISKDTSKNQVVLTMTNMDPVDTATYYCARTDYGGYGPEGFDYWGQGTLVTVSS

SEQ ID NO: 793 - protein, 10 aa, synthetic construct
  REGION 1..10: note = Description of Artificial Sequence: Synthetic peptide
GFSLHTRGMY

SEQ ID NO: 794 - protein, 7 aa, synthetic construct
  REGION 1..7: note = Description of Artificial Sequence: Synthetic peptide
INWDDDK

SEQ ID NO: 795 - protein, 15 aa, synthetic construct
  REGION 1..15: note = Description of Artificial Sequence: Synthetic peptide
ARTDYGGYGPEGFDY

SEQ ID NO: 796 - protein, 108 aa, synthetic construct
  REGION 1..108: note = Description of Artificial Sequence: Synthetic polypeptide
EIVMTQSPATLSVSPGESATLSCRASQSVRSNLAWYQQKPGQAPRLLIYGASTRAPGVPARFTGSESGREFTLTISSLQSEDFAVYYCQQYNNWPPYTFGQGTKLEIK

SEQ ID NO: 797 - protein, 6 aa, synthetic construct
  REGION 1..6: note = Description of Artificial Sequence: Synthetic peptide
QSVRSN

SEQ ID NO: 798 - protein, 10 aa, synthetic construct
  REGION 1..10: note = Description of Artificial Sequence: Synthetic peptide
QQYNNWPPYT

SEQ ID NO: 799 - protein, 117 aa, synthetic construct
  REGION 1..117: note = Description of Artificial Sequence: Synthetic polypeptide
QVQLVESGGGVVQPGRSLRLSCVASGFDLNAYGMHWVRQAPGKGLDWVAATSRGGTKKYYADSVKGRFTISRDVSKNTLYLQMGSLRTGDTAIYYCGVGMEDVFDIWGQGTMVTVSP

SEQ ID NO: 800 - protein, 8 aa, synthetic construct
  REGION 1..8: note = Description of Artificial Sequence: Synthetic peptide
GFDLNAYG

SEQ ID NO: 801 - protein, 8 aa, synthetic construct
  REGION 1..8: note = Description of Artificial Sequence: Synthetic peptide
TSRGGTKK

SEQ ID NO: 802 - protein, 10 aa, synthetic construct
  REGION 1..10: note = Description of Artificial Sequence: Synthetic peptide
GVGMEDVFDI

SEQ ID NO: 803 - protein, 110 aa, synthetic construct
  REGION 1..110: note = Description of Artificial Sequence: Synthetic polypeptide
QSVLTQPPSVSAAPGQKVTISCSENNSNIGNRNVSWYQQLPGKAPKLFIYDNNERPSGIPARFSGSKSGTSATLVITGLQTGDEADYYCGTWDRSLSVWVFGGGTKLTVL

SEQ ID NO: 804 - protein, 8 aa, synthetic construct
  REGION 1..8: note = Description of Artificial Sequence: Synthetic peptide
NSNIGNRN

SEQ ID NO: 805 - protein, 11 aa, synthetic construct
  REGION 1..11: note = Description of Artificial Sequence: Synthetic peptide
GTWDRSLSVWV

SEQ ID NO: 806 - protein, 121 aa, synthetic construct
  REGION 1..121: note = Description of Artificial Sequence: Synthetic polypeptide
QVQLQESGPGLVKPSQTLSLTCTVSGGSMRSGDYYWSWIRQPPGKGLEWIGYIYFTGSSYYNPSLKSRATISVDTSKNQFSLKLNSVTAADTAVYFCARGVDVDLTFFDCWGHGTLVTVSS

SEQ ID NO: 807 - protein, 10 aa, synthetic construct
  REGION 1..10: note = Description of Artificial Sequence: Synthetic peptide
GGSMRSGDYY

SEQ ID NO: 808 - protein, 7 aa, synthetic construct
  REGION 1..7: note = Description of Artificial Sequence: Synthetic peptide
IYFTGSS

SEQ ID NO: 809 - protein, 13 aa, synthetic construct
  REGION 1..13: note = Description of Artificial Sequence: Synthetic peptide
ARGVDVDLTFFDC

SEQ ID NO: 810 - protein, 108 aa, synthetic construct
  REGION 1..108: note = Description of Artificial Sequence: Synthetic polypeptide
SYVLTQPPSVSLAPGKTARITCGGNNIGNKSVHWYQQKPGQAPVLVIYYDSDRPSGIPERFSGSNSGNTATLTINRVEAGDEADYHCQVWDSSTDHRVFGEGTKLTVL

SEQ ID NO: 811 - protein, 6 aa, synthetic construct
  REGION 1..6: note = Description of Artificial Sequence: Synthetic peptide
NIGNKS

SEQ ID NO: 812 - protein, 11 aa, synthetic construct
  REGION 1..11: note = Description of Artificial Sequence: Synthetic peptide
QVWDSSTDHRV

SEQ ID NO: 813 - protein, 124 aa, synthetic construct
  REGION 1..124: note = Description of Artificial Sequence: Synthetic polypeptide
QVQLQQWGAGLLKPSETLSLTCTVIGTSFSNYYWSWIRQPPGKGLQWIGEITHSDSANYNPSLKSRVIISIDSSKNQLSLNLSSVTAADTAVYYCARGSKDYYDRSTFSWFDPWGQGTLVTVSS

SEQ ID NO: 814 - protein, 8 aa, synthetic construct
  REGION 1..8: note = Description of Artificial Sequence: Synthetic peptide
GTSFSNYY

SEQ ID NO: 815 - protein, 7 aa, synthetic construct
  REGION 1..7: note = Description of Artificial Sequence: Synthetic peptide
ITHSDSA

SEQ ID NO: 816 - protein, 18 aa, synthetic construct
  REGION 1..18: note = Description of Artificial Sequence: Synthetic peptide
ARGSKDYYDRSTFSWFDP

SEQ ID NO: 817 - protein, 109 aa, synthetic construct
  REGION 1..109: note = Description of Artificial Sequence: Synthetic polypeptide
EIVMTQSPATLSVSPGERATLSCRASQNISNKLAWYQQKPGQAPRLLIYDASTRATGVPARFSCSVSGTAFTLTINRLQSEDFAVYYCQQYYYWPPPYTFGHGTKLEIK

SEQ ID NO: 818 - protein, 6 aa, synthetic construct
  REGION 1..6: note = Description of Artificial Sequence: Synthetic peptide
QNISNK

SEQ ID NO: 819 - protein, 11 aa, synthetic construct
  REGION 1..11: note = Description of Artificial Sequence: Synthetic peptide
QQYYYWPPPYT

SEQ ID NO: 820 - protein, 123 aa, synthetic construct
  REGION 1..123: note = Description of Artificial Sequence: Synthetic polypeptide
QVQLVESGGGFVKPGGSLRLSCAVSGFTFSDYYMSWVRQAPGKGLEWLSHISSDGSDTNYADSVKGRFSISRDNAKNSVFLQMNTLRVEDTAVYYCARNALTNAYDMSGFRNWGQGTLVTVSS

SEQ ID NO: 821 - protein, 8 aa, synthetic construct
  REGION 1..8: note = Description of Artificial Sequence: Synthetic peptide
GFTFSDYY

SEQ ID NO: 822 - protein, 8 aa, synthetic construct
  REGION 1..8: note = Description of Artificial Sequence: Synthetic peptide
ISSDGSDT

SEQ ID NO: 823 - protein, 112 aa, synthetic construct
  REGION 1..112: note = Description of Artificial Sequence: Synthetic polypeptide
NFMLTQPHSVSESPGKTVILSCTRSSGSIASNYVRWYQQRPGSAPTTVIYEDSRRPSSVPDRFSGSIDSSSNSASLTISGLKTEDEADYYCQSFDSSSRKVVFGGGTKLTVL

SEQ ID NO: 824 - protein, 11 aa, synthetic construct
  REGION 1..11: note = Description of Artificial Sequence: Synthetic peptide
QSFDSSSRKVV

SEQ ID NO: 825 - protein, 121 aa, synthetic construct
  REGION 1..121: note = Description of Artificial Sequence: Synthetic polypeptide
QVQLLQSGPEVKQPGASVQVSCQTSGYTFTGYYIHWVRQAPGQGLEWVGWINPNRGHTNYGPAFQGRLTLTADTSSSTAYLELTRLRSDDTAVYYCARDRLTGGRDAFEIWGQGTMLIVSS

SEQ ID NO: 826 - protein, 8 aa, synthetic construct
  REGION 1..8: note = Description of Artificial Sequence: Synthetic peptide
INPNRGHT

SEQ ID NO: 827 - protein, 14 aa, synthetic construct
  REGION 1..14: note = Description of Artificial Sequence: Synthetic peptide
ARDRLTGGRDAFEI

SEQ ID NO: 828 - protein, 107 aa, synthetic construct
  REGION 1..107: note = Description of Artificial Sequence: Synthetic polypeptide
DIQLTQSPSSLSASVGDRVTITCRASQSISSYLNWYQQKPGKAPKLLISVASSLQDGVPPRFSGSRSGTEFTLTISSLQPEDFAIYYCQQSYSLSWTFGQGTKVEIK

SEQ ID NO: 829 - protein, 6 aa, synthetic construct
  REGION 1..6: note = Description of Artificial Sequence: Synthetic peptide
QSISSY

SEQ ID NO: 830 - skipped

SEQ ID NO: 831 - protein, 9 aa, synthetic construct
  REGION 1..9: note = Description of Artificial Sequence: Synthetic peptide
QQSYSLSWT

SEQ ID NO: 832 - protein, 121 aa, synthetic construct
  REGION 1..121: note = Description of Artificial Sequence: Synthetic polypeptide
QVQLVQSGAEVKQPGASVQVACQTSGYIFTAYYIHWLRQAPGQGLEWVGWINPNRGHTNYAPGFQGRLTLTADTSSSTAYLALTRLASDDTAVYYCARDRLTGGRDAFEIWGQGTMLIVSS

SEQ ID NO: 833 - protein, 8 aa, synthetic construct
  REGION 1..8: note = Description of Artificial Sequence: Synthetic peptide
GYIFTAYY

SEQ ID NO: 834 - protein, 107 aa, synthetic construct
  REGION 1..107: note = Description of Artificial Sequence: Synthetic polypeptide
DIQLTQSPSSLSASVGDRVTITCRASQSVSSYLNWYQQKPGEAPKLLISAASSLQDGVPPRFSGSRSGTEFTLTISSLQPEDFAIYYCQQSYSLWWTFGQGTKVEIK

SEQ ID NO: 835 - protein, 6 aa, synthetic construct
  REGION 1..6: note = Description of Artificial Sequence: Synthetic peptide
QSVSSY

SEQ ID NO: 836 - protein, 9 aa, synthetic construct
  REGION 1..9: note = Description of Artificial Sequence: Synthetic peptide
QQSYSLWWT

SEQ ID NO: 837 - protein, 127 aa, synthetic construct
  REGION 1..127: note = Description of Artificial Sequence: Synthetic polypeptide
QLQLQESGSGLVKPSQTLSLTCDVSGDSMNDDVYTWSWIRQPPGRGLEWIGYISHTGNTFYNSSLQSRVTMSVDTSKNQFSLKLSSVTIADTAVYYCARLTFLFSAPFSSFNWFDPWGQGILVTVSS

SEQ ID NO: 838 - protein, 10 aa, synthetic construct
  REGION 1..10: note = Description of Artificial Sequence: Synthetic peptide
GDSMNDDVYT

SEQ ID NO: 839 - protein, 7 aa, synthetic construct
  REGION 1..7: note = Description of Artificial Sequence: Synthetic peptide
ISHTGNT

SEQ ID NO: 840 - protein, 19 aa, synthetic construct
  REGION 1..19: note = Description of Artificial Sequence: Synthetic peptide
ARLTFLFSAPFSSFNWFDP

SEQ ID NO: 841 - protein, 112 aa, synthetic construct
  REGION 1..112: note = Description of Artificial Sequence: Synthetic polypeptide
QSVLTQPPSVSGAPGQTITISCTGTPSNFGADYDVHWYQQRPGTAPKLLIFADKHRPSGVPDRFSGSRSGTSASLAISGLQAEDEADYYCQSYDSGVVGLWVFGGGTKVTVL

SEQ ID NO: 842 - protein, 9 aa, synthetic construct
  REGION 1..9: note = Description of Artificial Sequence: Synthetic peptide
PSNFGADYD

SEQ ID NO: 843 - skipped

SEQ ID NO: 844 - protein, 12 aa, synthetic construct
  REGION 1..12: note = Description of Artificial Sequence: Synthetic peptide
QSYDSGVVGLWV

SEQ ID NO: 845 - protein, 124 aa, synthetic construct
  REGION 1..124: note = Description of Artificial Sequence: Synthetic polypeptide
QVQLQQWGAGLLKPSETLSLTCGVHGGSLNNYYWSWIRQPPGKGLEWIGEVYHSGSINYNPSLKSRVTMSVDTSKNQFSFNLSSVTAADTAVYYCARGAYDSRGFWTLDAFNTWGQGTMVIVSS

SEQ ID NO: 846 - protein, 8 aa, synthetic construct
  REGION 1..8: note = Description of Artificial Sequence: Synthetic peptide
GGSLNNYY

SEQ ID NO: 847 - protein, 7 aa, synthetic construct
  REGION 1..7: note = Description of Artificial Sequence: Synthetic peptide
VYHSGSI

SEQ ID NO: 848 - protein, 18 aa, synthetic construct
  REGION 1..18: note = Description of Artificial Sequence: Synthetic peptide
ARGAYDSRGFWTLDAFNT

SEQ ID NO: 849 - protein, 107 aa, synthetic construct
  REGION 1..107: note = Description of Artificial Sequence: Synthetic polypeptide
DIQMTQSPSALSASLGDRVTITCRASESINSWLAWYQQKPGKAPKLLIYKASTLESGVPSRFSGSGSGTEFTLTISSLQPDDFATYYCHQYNRYSYTFGQGTKLDIK

SEQ ID NO: 850 - protein, 6 aa, synthetic construct
  REGION 1..6: note = Description of Artificial Sequence: Synthetic peptide
ESINSW

SEQ ID NO: 851 - protein, 9 aa, synthetic construct
  REGION 1..9: note = Description of Artificial Sequence: Synthetic peptide
HQYNRYSYT

SEQ ID NO: 852 - protein, 127 aa, synthetic construct
  REGION 1..127: note = Description of Artificial Sequence: Synthetic polypeptide
EVLLLESGGGLVHPGGTLRLSCAASGFTFRNSAMTWVRQAPGKGLEWVSSIGGSGAKSYYADSVKGRFTISRDNSKNTLYLEMNTLRVDDTAIYYCAKDQLNCYDLWSGDYCWFDTWGQGTLVTVSS

SEQ ID NO: 853 - protein, 8 aa, synthetic construct
  REGION 1..8: note = Description of Artificial Sequence: Synthetic peptide
GFTFRNSA

SEQ ID NO: 854 - protein, 8 aa, synthetic construct
  REGION 1..8: note = Description of Artificial Sequence: Synthetic peptide
IGGSGAKS

SEQ ID NO: 855 - protein, 20 aa, synthetic construct
  REGION 1..20: note = Description of Artificial Sequence: Synthetic peptide
AKDQLNCYDLWSGDYCWFDT

SEQ ID NO: 856 - protein, 111 aa, synthetic construct
  REGION 1..111: note = Description of Artificial Sequence: Synthetic polypeptide
QSVLIQPPSASGTPGQRVTISCSGSNSNIGSNYVCWYQHLPGGAPKLLIYRNNQRPSGVPDRFSGSKSGTSASLAISGLRSEDEADYYCAAWDDSLSGSWVFGGGTKLTVL

SEQ ID NO: 857 - protein, 8 aa, synthetic construct
  REGION 1..8: note = Description of Artificial Sequence: Synthetic peptide
NSNIGSNY

SEQ ID NO: 858 - protein, 12 aa, synthetic construct
  REGION 1..12: note = Description of Artificial Sequence: Synthetic peptide
AAWDDSLSGSWV

SEQ ID NO: 859 - protein, 131 aa, synthetic construct
  REGION 1..131: note = Description of Artificial Sequence: Synthetic polypeptide
EVQLLESGGGLVQPGGSLRLSCAVSGLKFSSYAMSWVRQAPGKGLEWVSVVSGSSGSTFYAVSVEGRFSISRDNSNNMLYMDMHSLRVEDTAKYYCAKVVGWYYDRNGNRRPKGFRAFDVWGQGTMVIVSS

SEQ ID NO: 860 - protein, 8 aa, synthetic construct
  REGION 1..8: note = Description of Artificial Sequence: Synthetic peptide
GLKFSSYA

SEQ ID NO: 861 - protein, 8 aa, synthetic construct
  REGION 1..8: note = Description of Artificial Sequence: Synthetic peptide
VSGSSGST

SEQ ID NO: 862 - protein, 24 aa, synthetic construct
  REGION 1..24: note = Description of Artificial Sequence: Synthetic peptide
AKVVGWYYDRNGNRRPKGFRAFDV

SEQ ID NO: 863 - protein, 110 aa, synthetic construct
  REGION 1..110: note = Description of Artificial Sequence: Synthetic polypeptide
QSVLTQPPSASGTPGQRITIACSGTTSNIGGNSVNWYQQFPGAAPRLLIFDYDQRPSGVPARFSGSSSGSSGYLAISGLQSEDEADYYCSSWDDNLNGWVFGGGTKLTVL

SEQ ID NO: 864 - protein, 8 aa, synthetic construct
  REGION 1..8: note = Description of Artificial Sequence: Synthetic peptide
TSNIGGNS

SEQ ID NO: 865 - skipped

SEQ ID NO: 866 - protein, 11 aa, synthetic construct
  REGION 1..11: note = Description of Artificial Sequence: Synthetic peptide
SSWDDNLNGWV

SEQ ID NO: 867 - protein, 126 aa, synthetic construct
  REGION 1..126: note = Description of Artificial Sequence: Synthetic polypeptide
EVQLLESGGGLVQPGGSLRLSCVASGFTFSGHAMSWVRQAPGKGLEWVSGISGSGGSTYYADSVKGRFTISRDNSKKTVDLQMNNLRAEDTAIYYCAKDLHFDTSGYYYSMIFDYWGQGTLVPVSS

SEQ ID NO: 868 - protein, 8 aa, synthetic construct
  REGION 1..8: note = Description of Artificial Sequence: Synthetic peptide
GFTFSGHA

SEQ ID NO: 869 - protein, 19 aa, synthetic construct
  REGION 1..19: note = Description of Artificial Sequence: Synthetic peptide
AKDLHFDTSGYYYSMIFDY

SEQ ID NO: 870 - protein, 108 aa, synthetic construct
  REGION 1..108: note = Description of Artificial Sequence: Synthetic polypeptide
QSALAQPASVSGSPGQSITISCTGTSSDISDYNYVSWYQQHPGKAPKLILYDVNNRPSGVSSRFSGSKSGDTASLTISGLQPEDEADYYCSSYTSTKIFGGGTKVTVL

SEQ ID NO: 871 - protein, 9 aa, synthetic construct
  REGION 1..9: note = Description of Artificial Sequence: Synthetic peptide
SSDISDYNY

SEQ ID NO: 872 - protein, 8 aa, synthetic construct
  REGION 1..8: note = Description of Artificial Sequence: Synthetic peptide
SSYTSTKI

SEQ ID NO: 873 - protein, 126 aa, synthetic construct
  REGION 1..126: note = Description of Artificial Sequence: Synthetic polypeptide
EVQLLESGGGLLQLGGSLRLSCAASGFTFSSYVMSWVRQAPGKGLEWVSLITGSGGNTYYADSVKGRFTISRDNSKNTLFLQMNSLRVEDTAIYYCVKTDFYDSSGYYFHDAFHIWGQGTMVTVSS

SEQ ID NO: 874 - protein, 8 aa, synthetic construct
  REGION 1..8: note = Description of Artificial Sequence: Synthetic peptide
GFTFSSYV

SEQ ID NO: 875 - protein, 8 aa, synthetic construct
  REGION 1..8: note = Description of Artificial Sequence: Synthetic peptide
ITGSGGNT

SEQ ID NO: 876 - protein, 19 aa, synthetic construct
  REGION 1..19: note = Description of Artificial Sequence: Synthetic peptide
VKTDFYDSSGYYFHDAFHI

SEQ ID NO: 877 - protein, 109 aa, synthetic construct
  REGION 1..109: note = Description of Artificial Sequence: Synthetic polypeptide
QTVVTQEPSLTVSPGGTVTLTCASSTGSVTSGYYPNWFQQKPGQAPRTLIYGTSNKHSWTPARFSGSLLGGKAALTLSDVQPEDEAEYYCLLYYGGAYVFGTGTKVTVL

SEQ ID NO: 878 - protein, 9 aa, synthetic construct
  REGION 1..9: note = Description of Artificial Sequence: Synthetic peptide
TGSVTSGYY

SEQ ID NO: 879 - protein, 9 aa, synthetic construct
  REGION 1..9: note = Description of Artificial Sequence: Synthetic peptide
LLYYGGAYV

SEQ ID NO: 880 - protein, 120 aa, synthetic construct
  REGION 1..120: note = Description of Artificial Sequence: Synthetic polypeptide
QVQLVQSGAEVKKPGASVKVSCKASGYTFIRYDIHWVRQATGQGLEWMGWMNPNNGKSGFAQKFEGRVTLTRNTSVTSTYMQLSSLGLEDTAVYYCVRAGYSYGWGFDYWGQGSLVTVSS

SEQ ID NO: 881 - protein, 8 aa, synthetic construct
  REGION 1..8: note = Description of Artificial Sequence: Synthetic peptide
GYTFIRYD

SEQ ID NO: 882 - protein, 8 aa, synthetic construct
  REGION 1..8: note = Description of Artificial Sequence: Synthetic peptide
MNPNNGKS

SEQ ID NO: 883 - protein, 13 aa, synthetic construct
  REGION 1..13: note = Description of Artificial Sequence: Synthetic peptide
VRAGYSYGWGFDY

SEQ ID NO: 884 - protein, 110 aa, synthetic construct
  REGION 1..110: note = Description of Artificial Sequence: Synthetic polypeptide
NFTLTQPHSVSGSPGKTVTISCTRSSGGIASSHVQWYQQRPASAPTTLIFEDDQRSSGVPDRFSGSIDTSSNSAYLTISGLEAEDEADYYCQSYDNSMWVFGGGSKVTVL

SEQ ID NO: 885 - protein, 8 aa, synthetic construct
  REGION 1..8: note = Description of Artificial Sequence: Synthetic peptide
SGGIASSH

SEQ ID NO: 886 - skipped

SEQ ID NO: 887 - protein, 9 aa, synthetic construct
  REGION 1..9: note = Description of Artificial Sequence: Synthetic peptide
QSYDNSMWV

SEQ ID NO: 888 - protein, 124 aa, synthetic construct
  REGION 1..124: note = Description of Artificial Sequence: Synthetic polypeptide
HVQLVQSGADVKKPGSSVKVSCKFSGGTFNNDSINWVRQAPGQGLEWMGVIMPFFGATRFAPKFQGRVTLTADKFTSTGYMELGSLKSDDTAVYYCARDKPPDDKWADYGMDVWGQGTTVTVSS

SEQ ID NO: 889 - protein, 8 aa, synthetic construct
  REGION 1..8: note = Description of Artificial Sequence: Synthetic peptide
GGTFNNDS

SEQ ID NO: 890 - protein, 8 aa, synthetic construct
  REGION 1..8: note = Description of Artificial Sequence: Synthetic peptide
IMPFFGAT

SEQ ID NO: 891 - protein, 17 aa, synthetic construct
  REGION 1..17: note = Description of Artificial Sequence: Synthetic peptide
ARDKPPDDKWADYGMDV

SEQ ID NO: 892 - protein, 108 aa, synthetic construct
  REGION 1..108: note = Description of Artificial Sequence: Synthetic polypeptide
SYELTQPPSVSVSPGQTARITCSGDALPKQYVYWYQQKTGQAPVLVIYKDTERPSGIPERFSGSTSGTTVTLTISGVQADDEADYFCQSADRNANYRVFGGGTKLTVL

SEQ ID NO: 893 - protein, 11 aa, synthetic construct
  REGION 1..11: note = Description of Artificial Sequence: Synthetic peptide
QSADRNANYRV

SEQ ID NO: 894 - protein, 120 aa, synthetic construct
  REGION 1..120: note = Description of Artificial Sequence: Synthetic polypeptide
QLQLQESGSGLVKPSQTLSLTCAVSGGSITSGTYSWTWIRQSPEKGLEWIGYIYYTGSTYYNPSLGRRVTISGDTSNNEFSLNLKSVTAADTAVYYCARGIHRGGVLDFWGQGILVTVSS

SEQ ID NO: 895 - protein, 10 aa, synthetic construct
  REGION 1..10: note = Description of Artificial Sequence: Synthetic peptide
GGSITSGTYS

SEQ ID NO: 896 - protein, 7 aa, synthetic construct
  REGION 1..7: note = Description of Artificial Sequence: Synthetic peptide
IYYTGST

SEQ ID NO: 897 - protein, 12 aa, synthetic construct
  REGION 1..12: note = Description of Artificial Sequence: Synthetic peptide
ARGIHRGGVLDF

SEQ ID NO: 898 - protein, 108 aa, synthetic construct
  REGION 1..108: note = Description of Artificial Sequence: Synthetic polypeptide
EIVLTQSPATLPLSPGERATLSCRASQSLDKYLAWYQQKPGQAPRLLIYDTSKRATGIPARFSGSGSGTDFTLTISSLEPEDFAVYFCQQRNNWPPYTFGQGTKVEMK

SEQ ID NO: 899 - protein, 6 aa, synthetic construct
  REGION 1..6: note = Description of Artificial Sequence: Synthetic peptide
QSLDKY

SEQ ID NO: 900 - skipped

SEQ ID NO: 901 - protein, 10 aa, synthetic construct
  REGION 1..10: note = Description of Artificial Sequence: Synthetic peptide
QQRNNWPPYT

SEQ ID NO: 902 - protein, 119 aa, synthetic construct
  REGION 1..119: note = Description of Artificial Sequence: Synthetic polypeptide
QVLLVQSGSEVKNPGASIRVSCKTSGYMFTNNGIAWVREVPTQGLEWMGWISTYSGATHYAPNLHGRITMTADTSASTAYMELRSLQSGDTGVYYCARLWFGKLGLDFWGQGTQVTVSS

SEQ ID NO: 903 - protein, 8 aa, synthetic construct
  REGION 1..8: note = Description of Artificial Sequence: Synthetic peptide
GYMFTNNG

SEQ ID NO: 904 - protein, 8 aa, synthetic construct
  REGION 1..8: note = Description of Artificial Sequence: Synthetic peptide
ISTYSGAT

SEQ ID NO: 905 - protein, 12 aa, synthetic construct
  REGION 1..12: note = Description of Artificial Sequence: Synthetic peptide
ARLWFGKLGLDF

SEQ ID NO: 906 - protein, 111 aa, synthetic construct
  REGION 1..111: note = Description of Artificial Sequence: Synthetic polypeptide
QSVLTQPPSASGTPGQRVIISCSGSTSNIGTKTVNWYQHLPGTAPKLLIYNNNQRPSGVPDRFSGSKSGTSASLTISGLQSEDEADYYCAAWDDSLNGRGLFGPGTKVTVL

SEQ ID NO: 907 - protein, 8 aa, synthetic construct
  REGION 1..8: note = Description of Artificial Sequence: Synthetic peptide
TSNIGTKT

SEQ ID NO: 908 - skipped

SEQ ID NO: 909 - protein, 12 aa, synthetic construct
  REGION 1..12: note = Description of Artificial Sequence: Synthetic peptide
AAWDDSLNGRGL

SEQ ID NO: 910 - protein, 124 aa, synthetic construct
  REGION 1..124: note = Description of Artificial Sequence: Synthetic polypeptide
QVEVVESGGGVVQPGKSLRLSCAASGFKFNVYGIHWVRQAPGKGLEWVAVVWYDGSNKYYADSVKGRFTISRDNSKNTTYLQMDSLRVDDTAVYYCARELQYSNYDYFYAMDVWGQGTTVTVSS

SEQ ID NO: 911 - protein, 8 aa, synthetic construct
  REGION 1..8: note = Description of Artificial Sequence: Synthetic peptide
GFKFNVYG

SEQ ID NO: 912 - protein, 8 aa, synthetic construct
  REGION 1..8: note = Description of Artificial Sequence: Synthetic peptide
VWYDGSNK

SEQ ID NO: 913 - protein, 17 aa, synthetic construct
  REGION 1..17: note = Description of Artificial Sequence: Synthetic peptide
ARELQYSNYDYFYAMDV

SEQ ID NO: 914 - protein, 107 aa, synthetic construct
  REGION 1..107: note = Description of Artificial Sequence: Synthetic polypeptide
DIQMTQSPPSLSASVGDRVTITCRASQDIDNYLVWFQQKPGRAPKSLIYAASSLQSGVPSKFSGSGSGTEFTLTISSLQPEDFATYYCQQYNSFPYTFGQGTKLEIK

SEQ ID NO: 915 - protein, 6 aa, synthetic construct
  REGION 1..6: note = Description of Artificial Sequence: Synthetic peptide
QDIDNY

SEQ ID NO: 916 - protein, 9 aa, synthetic construct
  REGION 1..9: note = Description of Artificial Sequence: Synthetic peptide
QQYNSFPYT

SEQ ID NO: 917 - protein, 119 aa, synthetic construct
  REGION 1..119: note = Description of Artificial Sequence: Synthetic polypeptide
QVQLQESGPGLVKPSETLSLTCSVSGGSISSHYWSWIRQPPGRGLEWIAYISYSGRTKYNPSLKSRVTISEDTSKNQFSLKLSSVTPADTAVYYCARIYGDYGPFIDYWGQGTLVTVSS

SEQ ID NO: 918 - protein, 8 aa, synthetic construct
  REGION 1..8: note = Description of Artificial Sequence: Synthetic peptide
GGSISSHY

SEQ ID NO: 919 - protein, 7 aa, synthetic construct
  REGION 1..7: note = Description of Artificial Sequence: Synthetic peptide
ISYSGRT

SEQ ID NO: 920 - protein, 13 aa, synthetic construct
  REGION 1..13: note = Description of Artificial Sequence: Synthetic peptide
ARIYGDYGPFIDY

SEQ ID NO: 921 - protein, 107 aa, synthetic construct
  REGION 1..107: note = Description of Artificial Sequence: Synthetic polypeptide
DIQMTQSPSSLSASVGDRVTITCRASQTISTYLNWYQQKPGTAPMLLIYGAYSLHSGVPSRFSGSGSGTDFTLTISSLQPEDFATYYCQQSSSLPLTFGGGTKVEIK

SEQ ID NO: 922 - protein, 6 aa, synthetic construct
  REGION 1..6: note = Description of Artificial Sequence: Synthetic peptide
QTISTY

SEQ ID NO: 923 - skipped

SEQ ID NO: 924 - protein, 9 aa, synthetic construct
  REGION 1..9: note = Description of Artificial Sequence: Synthetic peptide
QQSSSLPLT

SEQ ID NO: 925 - protein, 127 aa, synthetic construct
  REGION 1..127: note = Description of Artificial Sequence: Synthetic polypeptide
QEQLQESGPGLVKPSQTLSLTCTVSGGSISSGDHYWSWLRQTPGKGLEWIGYIYYRGNTNYNPSLESRITMSVDTSKNQFSLKLSSVTAADTGVYYCARDRRLLFWFGQGPETFDIWGPGTMVTVSS

SEQ ID NO: 926 - protein, 10 aa, synthetic construct
  REGION 1..10: note = Description of Artificial Sequence: Synthetic peptide
GGSISSGDHY

SEQ ID NO: 927 - protein, 7 aa, synthetic construct
  REGION 1..7: note = Description of Artificial Sequence: Synthetic peptide
IYYRGNT

SEQ ID NO: 928 - protein, 19 aa, synthetic construct
  REGION 1..19: note = Description of Artificial Sequence: Synthetic peptide
ARDRRLLFWFGQGPETFDI

SEQ ID NO: 929 - protein, 107 aa, synthetic construct
  REGION 1..107: note = Description of Artificial Sequence: Synthetic polypeptide
DIQMTQSPSILSASVGDRVTITCRASQNINHWLAWYQQKPGKAPKLLIYMASSLENGVPSRFSGSGSGTEFTLTISSLQPDDFATYYCQQYNSYSGTFGHGTKVDIK

SEQ ID NO: 930 - protein, 6 aa, synthetic construct
  REGION 1..6: note = Description of Artificial Sequence: Synthetic peptide
QNINHW

SEQ ID NO: 931 - skipped

SEQ ID NO: 932 - protein, 9 aa, synthetic construct
  REGION 1..9: note = Description of Artificial Sequence: Synthetic peptide
QQYNSYSGT

SEQ ID NO: 933 - DNA, 91 bp, synthetic construct
  misc_feature 1..91: note = Description of Artificial Sequence: Synthetic oligonucleotide
  source 1..91: mol_type = other DNA
tactgtgcgaaagttcttgactacagtgaatttcattactattacggtttggacgtctggggccaagggaccgcggtcgccgtctcctcag

SEQ ID NO: 934 - DNA, 91 bp, synthetic construct
  misc_feature 1..91: note = Description of Artificial Sequence: Synthetic oligonucleotide
  source 1..91: mol_type = other DNA
tactgtgcgaaagttcttgactacaatgagtactctctctacttcggtatggacgtctggggccaagggaccacggtcaccgtctcctcag

SEQ ID NO: 935 - DNA, 91 bp, synthetic construct
  misc_feature 1..91: note = Description of Artificial Sequence: Synthetic oligonucleotide
  source 1..91: mol_type = other DNA
tactgtgcgaaagttcttgactacagtgaatactctctctacttcggtatggacgtctggggccaagggaccacggtccttgtctcctcag

SEQ ID NO: 936 - DNA, 91 bp, synthetic construct
  misc_feature 1..91: note = Description of Artificial Sequence: Synthetic oligonucleotide
  source 1..91: mol_type = other DNA
tactgtgcgaaggtccttgactacagtaggtactcctattattacgggatggacgtctggggccaggggaccacggtcatcgtctcctcag

SEQ ID NO: 937 - DNA, 91 bp, synthetic construct
  misc_feature 1..91: note = Description of Artificial Sequence: Synthetic oligonucleotide
  source 1..91: mol_type = other DNA
tactgtgctaaggtccttgactacagtgcattctcctattattatgggatggacgtctggggccaggggaccacggtcatcgtctcctcag

SEQ ID NO: 938 - DNA, 91 bp, synthetic construct
  misc_feature 1..91: note = Description of Artificial Sequence: Synthetic oligonucleotide
  source 1..91: mol_type = other DNA
tattgtgcgaaagtccttgactacagtattttctattactatttcggcctggacgtctggggccaagggaccacggtcaccgtctcctcag

SEQ ID NO: 939 - DNA, 14 bp, synthetic construct
  misc_feature 1..14: note = Description of Artificial Sequence: Synthetic oligonucleotide
  source 1..14: mol_type = other DNA
tactgtgcgaaaga

SEQ ID NO: 940 - skipped

SEQ ID NO: 941 - DNA, 16 bp, synthetic construct
  misc_feature 1..16: note = Description of Artificial Sequence: Synthetic oligonucleotide
  source 1..16: mol_type = other DNA
tgactacagtaactac

SEQ ID NO: 942 - DNA, 63 bp, synthetic construct
  misc_feature 1..63: note = Description of Artificial Sequence: Synthetic oligonucleotide
  source 1..63: mol_type = other DNA
attactactactactacggtatggacgtctggggccaagggaccacggtcaccgtctcctcag

SEQ ID NO: 943 - protein, 4 aa, synthetic construct
  REGION 1..4: note = Description of Artificial Sequence: Synthetic peptide
YCAK

SEQ ID NO: 944 - protein, 7 aa, synthetic construct
  REGION 1..7: note = Description of Artificial Sequence: Synthetic peptide
VLDYSNY

SEQ ID NO: 945 - protein, 21 aa, synthetic construct
  REGION 1..21: note = Description of Artificial Sequence: Synthetic peptide
NYYYYYGMDVWGQGTTVTVSS

SEQ ID NO: 946 - protein, 30 aa, synthetic construct
  REGION 1..30: note = Description of Artificial Sequence: Synthetic polypeptide
YCAKVLDYSNYYYYYGMDVWGQGTTVTVSS

SEQ ID NO: 947 - protein, 30 aa, synthetic construct
  REGION 1..30: note = Description of Artificial Sequence: Synthetic polypeptide
YCAKVLDYSEFHYYYGLDVWGQGTAVAVSS

SEQ ID NO: 948 - protein, 30 aa, synthetic construct
  REGION 1..30: note = Description of Artificial Sequence: Synthetic polypeptide
YCAKVLDYNEYSLYFGMDVWGQGTTVTVSS

SEQ ID NO: 949 - protein, 30 aa, synthetic construct
  REGION 1..30: note = Description of Artificial Sequence: Synthetic polypeptide
YCAKVLDYSEYSLYFGMDVWGQGTTVLVSS

SEQ ID NO: 950 - protein, 30 aa, synthetic construct
  REGION 1..30: note = Description of Artificial Sequence: Synthetic polypeptide
YCAKVLDYSRYSYYYGMDVWGQGTTVIVSS

SEQ ID NO: 951 - protein, 30 aa, synthetic construct
  REGION 1..30: note = Description of Artificial Sequence: Synthetic polypeptide
YCAKVLDYSAFSYYYGMDVWGQGTTVIVSS

SEQ ID NO: 952 - protein, 30 aa, synthetic construct
  REGION 1..30: note = Description of Artificial Sequence: Synthetic polypeptide
YCAKVLDYSIFYYYFGLDVWGQGTTVTVSS

SEQ ID NO: 953 - DNA, 76 bp, synthetic construct
  misc_feature 1..76: note = Description of Artificial Sequence: Synthetic oligonucleotide
  source 1..76: mol_type = other DNA
gcagtgtattactgtcagcattacagtaattcacccccgtacacttttggcccggggaccaagttggagatcaaac

SEQ ID NO: 954 - DNA, 76 bp, synthetic construct
  misc_feature 1..76: note = Description of Artificial Sequence: Synthetic oligonucleotide
  source 1..76: mol_type = other DNA
gcagtgtatttctgtcagtactatagtgactcacctccgtacacttttggcccggggaccaagctggagatcaaac

SEQ ID NO: 955 - DNA, 76 bp, synthetic construct
  misc_feature 1..76: note = Description of Artificial Sequence: Synthetic oligonucleotide
  source 1..76: mol_type = other DNA
gcagtgtattcctgtcaacactatagtgactcacctccttacacttttggccaggggaccaaactggagatcaaac

SEQ ID NO: 956 - DNA, 76 bp, synthetic construct
  misc_feature 1..76: note = Description of Artificial Sequence: Synthetic oligonucleotide
  source 1..76: mol_type = other DNA
gcagtttattactgtcagcactatggtaggtcacctccgtacacttttggcccggggaccaagctggacatcaaac

SEQ ID NO: 957 - DNA, 76 bp, synthetic construct
  misc_feature 1..76: note = Description of Artificial Sequence: Synthetic oligonucleotide
  source 1..76: mol_type = other DNA
gcagtatattactgtcaacactatggtaggtcacctccatacacttttggccaggggaccaaagtggagatcaaac

SEQ ID NO: 958 - DNA, 76 bp, synthetic construct
  misc_feature 1..76: note = Description of Artificial Sequence: Synthetic oligonucleotide
  source 1..76: mol_type = other DNA
gcagtgtactactgtcagcactatggagactcacctccgtacacctttggccaggggacgaaagtggagatgaaac

SEQ ID NO: 959 - DNA, 38 bp, synthetic construct
  misc_feature 1..38: note = Description of Artificial Sequence: Synthetic oligonucleotide
  source 1..38: mol_type = other DNA
gcagtgtattactgtcagcagtatggtagctcacctcc

SEQ ID NO: 960 - DNA, 39 bp, synthetic construct
  misc_feature 1..39: note = Description of Artificial Sequence: Synthetic oligonucleotide
  source 1..39: mol_type = other DNA
tgtacacttttggccaggggaccaagctggagatcaaac

SEQ ID NO: 961 - protein, 12 aa, synthetic construct
  REGION 1..12: note = Description of Artificial Sequence: Synthetic peptide
AVYYCQQYGSSP

SEQ ID NO: 962 - protein, 13 aa, synthetic construct
  REGION 1..13: note = Description of Artificial Sequence: Synthetic peptide
PYTFGQGTKLEIK

SEQ ID NO: 963 - protein, 25 aa, synthetic construct
  REGION 1..25: note = Description of Artificial Sequence: Synthetic peptide
AVYYCQQYGSSPPYTFGQGTKLEIK

SEQ ID NO: 964 - protein, 25 aa, synthetic construct
  REGION 1..25: note = Description of Artificial Sequence: Synthetic peptide
AVYYCQHYSNSPPYTFGPGTKLEIK

SEQ ID NO: 965 - protein, 25 aa, synthetic construct
  REGION 1..25: note = Description of Artificial Sequence: Synthetic peptide
AVYFCQYYSDSPPYTFGPGTKLEIK

SEQ ID NO: 966 - protein, 25 aa, synthetic construct
  REGION 1..25: note = Description of Artificial Sequence: Synthetic peptide
AVYSCQHYSDSPPYTFGQGTKLEIK

SEQ ID NO: 967 - protein, 25 aa, synthetic construct
  REGION 1..25: note = Description of Artificial Sequence: Synthetic peptide
AVYYCQHYGRSPPYTFGPGTKLDIK

SEQ ID NO: 968 - protein, 25 aa, synthetic construct
  REGION 1..25: note = Description of Artificial Sequence: Synthetic peptide
AVYYCQHYGRSPPYTFGQGTKVEIK

SEQ ID NO: 969 - protein, 25 aa, synthetic construct
  REGION 1..25: note = Description of Artificial Sequence: Synthetic peptide
AVYYCQHYGDSPPYTFGQGTKVEMK

SEQ ID NO: 970 - protein, 109 aa, synthetic construct
  REGION 1..109: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPGFTFGPGTKVDIK

SEQ ID NO: 971 - protein, 17 aa, synthetic construct
  REGION 1..17: note = Description of Artificial Sequence: Synthetic peptide
AKVMDYDIFKNYFGLDV

SEQ ID NO: 972 - protein, 17 aa, synthetic construct
  REGION 1..17: note = Description of Artificial Sequence: Synthetic peptide
AKVMDYDVFKNYYGLDV

SEQ ID NO: 973 - protein, 17 aa, synthetic construct
  REGION 1..17: note = Description of Artificial Sequence: Synthetic peptide
AKTLDYSQYMYYYGLDV

SEQ ID NO: 974 - protein, 10 aa, synthetic construct
  REGION 1..10: note = Description of Artificial Sequence: Synthetic peptide
QHYGRSPPYT

SEQ ID NO: 975 - protein, 10 aa, synthetic construct
  REGION 1..10: note = Description of Artificial Sequence: Synthetic peptide
QHYGSSPPFT

SEQ ID NO: 976 - protein, 10 aa, synthetic construct
  REGION 1..10: note = Description of Artificial Sequence: Synthetic peptide
QHYGSLPPFT

SEQ ID NO: 977 - protein, 10 aa, synthetic construct
  REGION 1..10: note = Description of Artificial Sequence: Synthetic peptide
AAWDDTLVGV

SEQ ID NO: 978 - protein, 10 aa, synthetic construct
  REGION 1..10: note = Description of Artificial Sequence: Synthetic peptide
AAWDDLVVGV

SEQ ID NO: 979 - protein, 11 aa, synthetic construct
  REGION 1..11: note = Description of Artificial Sequence: Synthetic peptide
QSTDSSGDYVV

SEQ ID NO: 980 - protein, 11 aa, synthetic construct
  REGION 1..11: note = Description of Artificial Sequence: Synthetic peptide
QSTDSSLRDVV

SEQ ID NO: 981 - protein, 10 aa, synthetic construct
  REGION 1..10: note = Description of Artificial Sequence: Synthetic peptide
TSYAGRNIQV

SEQ ID NO: 982 - protein, 10 aa, synthetic construct
  REGION 1..10: note = Description of Artificial Sequence: Synthetic peptide
SSYAGSNIAV

SEQ ID NO: 983 - protein, 10 aa, synthetic construct
  REGION 1..10: note = Description of Artificial Sequence: Synthetic peptide
QSYDGSSPVI

SEQ ID NO: 984 - protein, 9 aa, synthetic construct
  REGION 1..9: note = Description of Artificial Sequence: Synthetic peptide
QSYDTNIVV

SEQ ID NO: 985 - protein, 9 aa, synthetic construct
  REGION 1..9: note = Description of Artificial Sequence: Synthetic peptide
QSYDSANVV

SEQ ID NO: 986 - protein, 9 aa, synthetic construct
  REGION 1..9: note = Description of Artificial Sequence: Synthetic peptide
QSYDADNAV

SEQ ID NO: 987 - protein, 12 aa, synthetic construct
  REGION 1..12: note = Description of Artificial Sequence: Synthetic peptide
SSYTRETALGGV

SEQ ID NO: 988 - protein, 9 aa, synthetic construct
  REGION 1..9: note = Description of Artificial Sequence: Synthetic peptide
QQYYTTPRT

SEQ ID NO: 989 - protein, 9 aa, synthetic construct
  REGION 1..9: note = Description of Artificial Sequence: Synthetic peptide
QQYYTTPYT

SEQ ID NO: 990 - protein, 9 aa, synthetic construct
  REGION 1..9: note = Description of Artificial Sequence: Synthetic peptide
QQYLTTPYT

SEQ ID NO: 991 - protein, 10 aa, synthetic construct
  REGION 1..10: note = Description of Artificial Sequence: Synthetic peptide
QQYDEWPPFT

SEQ ID NO: 992 - protein, 10 aa, synthetic construct
  REGION 1..10: note = Description of Artificial Sequence: Synthetic peptide
QQYNHWPPYT

SEQ ID NO: 993 - protein, 10 aa, synthetic construct
  REGION 1..10: note = Description of Artificial Sequence: Synthetic peptide
GSYKSGSTWV

SEQ ID NO: 994 - protein, 10 aa, synthetic construct
  REGION 1..10: note = Description of Artificial Sequence: Synthetic peptide
SSYRSGSTWV

SEQ ID NO: 995 - protein, 10 aa, synthetic construct
  REGION 1..10: note = Description of Artificial Sequence: Synthetic peptide
SSYTSGRTWV

SEQ ID NO: 996 - protein, 10 aa, synthetic construct
  REGION 1..10: note = Description of Artificial Sequence: Synthetic peptide
SSYTTGRTWV

SEQ ID NO: 997 - protein, 17 aa, synthetic construct
  REGION 1..17: note = Description of Artificial Sequence: Synthetic peptide
ASRYCTDSGCYLGSFDY

SEQ ID NO: 998 - protein, 17 aa, synthetic construct
  REGION 1..17: note = Description of Artificial Sequence: Synthetic peptide
ASRYCTDDGCYLGSFDF

SEQ ID NO: 999 - protein, 7 aa, synthetic construct
  REGION 1..7: note = Description of Artificial Sequence: Synthetic peptide
TRDHGYY

SEQ ID NO: 1000 - protein, 7 aa, synthetic construct
  REGION 1..7: note = Description of Artificial Sequence: Synthetic peptide
ARDHGYY

SEQ ID NO: 1001 - protein, 13 aa, synthetic construct
  REGION 1..13: note = Description of Artificial Sequence: Synthetic peptide
ARDPAAGTWWFDP

SEQ ID NO: 1002 - protein, 19 aa, synthetic construct
  REGION 1..19: note = Description of Artificial Sequence: Synthetic peptide
ARPSAHYYDRGGYNDAFDM

SEQ ID NO: 1003 - protein, 18 aa, synthetic construct
  REGION 1..18: note = Description of Artificial Sequence: Synthetic peptide
TTGYRTTTTYHGDDAFDI

SEQ ID NO: 1004 - protein, 18 aa, synthetic construct
  REGION 1..18: note = Description of Artificial Sequence: Synthetic peptide
TTGYRTSTSYHGDDAFDI

SEQ ID NO: 1005 - protein, 16 aa, synthetic construct
  REGION 1..16: note = Description of Artificial Sequence: Synthetic peptide
ARGPPAVQGYFYYMYV

SEQ ID NO: 1006 - protein, 16 aa, synthetic construct
  REGION 1..16: note = Description of Artificial Sequence: Synthetic peptide
ARGPPGVHGYFYYTDV

SEQ ID NO: 1007 - protein, 17 aa, synthetic construct
  REGION 1..17: note = Description of Artificial Sequence: Synthetic peptide
ARDVVRPGSGPRLGFDP

SEQ ID NO: 1008 - protein, 17 aa, synthetic construct
  REGION 1..17: note = Description of Artificial Sequence: Synthetic peptide
ARDVVRPGRGPRLGFDP

SEQ ID NO: 1009 - protein, 21 aa, synthetic construct
  REGION 1..21: note = Description of Artificial Sequence: Synthetic peptide
AKEGGSSTSWYSLYHEYEMDV

SEQ ID NO: 1010 - protein, 16 aa, synthetic construct
  REGION 1..16: note = Description of Artificial Sequence: Synthetic peptide
AHKAAEPGSRDRWFDS

SEQ ID NO: 1011 - protein, 13 aa, synthetic construct
  REGION 1..13: note = Description of Artificial Sequence: Synthetic peptide
AGGYNNSSFYFDS

SEQ ID NO: 1012 - protein, 13 aa, synthetic construct
  REGION 1..13: note = Description of Artificial Sequence: Synthetic peptide
AVGYNNSWFYFDY

SEQ ID NO: 1013 - protein, 12 aa, synthetic construct
  REGION 1..12: note = Description of Artificial Sequence: Synthetic peptide
ARLGHLRGWFDS

SEQ ID NO: 1014 - protein, 19 aa, synthetic construct
  REGION 1..19: note = Description of Artificial Sequence: Synthetic peptide
VLSQYEFGSSWFYYYRMDV

SEQ ID NO: 1015 - protein, 19 aa, synthetic construct
  REGION 1..19: note = Description of Artificial Sequence: Synthetic peptide
VLSKYEFGSSWFYYYRMDV

SEQ ID NO: 1016 - protein, 19 aa, synthetic construct
  REGION 1..19: note = Description of Artificial Sequence: Synthetic peptide
VLSKYEFHSSWFYYYRMDV

SEQ ID NO: 1017 - protein, 7 aa, synthetic construct
  REGION 1..7: note = Description of Artificial Sequence: Synthetic peptide
GSGSGSG

SEQ ID NO: 1018 - protein, 10 aa, synthetic construct
  REGION 1..10: note = Description of Artificial Sequence: Synthetic peptide
YPYDVPDYAG

SEQ ID NO: 1019 - protein, 15 aa, synthetic construct
  REGION 1..15: note = Description of Artificial Sequence: Synthetic peptide
AKHDYDSSGYFDEDY